SEQUENCE LISTING (47 sequences)

SEQ ID NO: 1 - DNA, 144 bp, synthetic construct
  source 1..144: mol_type = other DNA; note = Artificial intron
gtaagtcgacgggccgggcctgggccgggtccgggccgggtcgttggatccccactacagcccgatactcaagcttgacgaattcgagtatccaaggtagtggactagtgtgacgctgctgacccctttctttcccttctgcag

SEQ ID NO: 2 - DNA, 190 bp, synthetic construct
  source 1..190: mol_type = other DNA; note = 5'UTR1
ctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttcca

SEQ ID NO: 3 - DNA, 771 bp, synthetic construct
  source 1..771: mol_type = other DNA; note = 5'UTR1 with MAR-5 insertion
ctgccttctccctcctgtgagtttggtaagtcgacgggccgggcctgggccgggtccgggccgggtatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagtcgttggatccccactacagcccgatactcaagcttgacgaattcgagtatccaaggtagtggactagtgtgacgctgctgacccctttctttcccttctgcaggttggtgtacagtagcttcca

SEQ ID NO: 4 - DNA, 117 bp, synthetic construct
  source 1..117: mol_type = other DNA; note = 5'UTR
attgggatcttcacacagcaggtaaggttgcgggccgggcctgggccgggtccgggccgggccgcactgacccctggtgttgctttttttttttaggccgcaagctgaagcgtgtcc

SEQ ID NO: 5 - DNA, 698 bp, synthetic construct
  source 1..698: mol_type = other DNA; note = 5'UTR2 (5'UTR of SEQ ID NO:4 with MAR-5 insertion)
attgggatcttcacacagcaggtaaggttgcgggccgggcctgggccgggtccgggccgggtatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagccgcactgacccctggtgttgctttttttttttaggccgcaagctgaagcgtgtcc

SEQ ID NO: 6 - DNA, 1579 bp, synthetic construct
  source 1..1579: mol_type = other DNA; note = A2UCOE element
gcggccgcacgcgtggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgccttagtgggggggaaggttctagaaaagcggcggcagcggctctagcggcagtagcagcagcgccgggtcccgtgcggaggtgctcctcgcagagttgtttctccagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggacttcttattccctttctaaagcacgctgcttcgggggccacggcgtctcctcggacggccgggcgcgcc

SEQ ID NO: 7 - DNA, 761 bp, synthetic construct
  source 1..761: mol_type = other DNA; note = SRF-UCOE
gcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaa

SEQ ID NO: 8 - DNA, 478 bp, synthetic construct
  source 1..478: mol_type = other DNA; note = cHS4 insulator
ggggagctcacggggacagcccccccccaaagcccccagggatgtaattacgtccctcccccgctagggggcagcagcgaccgcccggggctccgctccggtccggcgctccccccgcatcccgagccggcagcgtgcggggacagcccgggcacggggaaggtggcacgggatcgctttcctctgaacgcttctcgctgctctttgagcctgcagacacctgggggatacggggaaaaggggagctcacggggacagcccccccccaaagcccccagggatgtaattacgtccctcccccgctagggggcagcagcgaccgcccggggctccgctccggtccggcgctccccccgcatcccgagccggcagcgtgcggggacagcccgggcacggggaaggtggcacgggatcgctttcctctgaacgcttctcgctgctctttgagcctgcagacacctgggggatacggggaaaa

SEQ ID NO: 9 - DNA, 581 bp, synthetic construct
  source 1..581: mol_type = other DNA; note = MAR-5
tatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatag

SEQ ID NO: 10 - DNA, 1233 bp, synthetic construct
  source 1..1233: mol_type = other DNA; note = Human CSP-B MAR (huMAR)
ggatcccattctccttgatgtactaatttttctttaaaagtgataataatagctcccatttagaatttttaaataacacaacaaatgtaaagtaactaatgtgtcctctggatcatggtaagtaatgaataaatttaactccctttaccttctccctttgctattttttccatgctaggatttatacatttttaaaaaactaaatctgctatcaaatgacagctttaaatttactttttaaaatttgttattgtatatatttatggggtataaagtgatgttatgatatatatatacacaatgtacactgattaaatcaagccaattaacattttatcatctcaaatacttaacattttttgtagtgagaacatttgaaatttacttttagcaatttcaaaacatacattattattattaactatagtcaccatgatgtaccatagatctttaaaaacttattcttcctgcctaactgaaactttgtactctttgactaacatcttttcattcccccacttcccagcctctggtaatcaccattacacactctgcttctatgagttcaattgctttagactccacgtaataaatgagatcatgcagcatttggctttctgtgcctggcttatccttgcttagcatggtgtcttacaggttcatccatgttgcaacaaataacagaatctcattctttgttaaggctgaatactattccattgggtatatataccacattttccttatccattaatccactgatggacccttaggttgttgattccatatattggctattgtaaatagtgcagcaatgaacatgagagtgcaactatctcttcaatgtactgatttcgaatccttcggatctatctcagaagtgagattgcaggatcatataattctacttttagtcttttgaggagctccatacagctttccatatggccatactaattacattctcatcaacagtgtacaatggtttccttttctccacatcctcaccaacatttataattttttgtctttttgataatagccatctgacaggtgtaaagtgatagctcattgcagttttaatttgcattttttgatgattagtaatgttgagaattttttcatatatctcttggccagttgcatgtcttctttggaaaaatgtctattcagttcctttgcccattttttaattgggatttttggtttcttgctattgagttgtttgaattc

SEQ ID NO: 11 - DNA, 597 bp, synthetic construct
  source 1..597: mol_type = other DNA; note = WPRE
tcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 12 - DNA, 111 bp, synthetic construct
  source 1..111: mol_type = other DNA; note = Enhancer-1
gggactttccggggcggggcacgtggtgcacgggactttccgtgcacgtgcacgggactttccgggactttccgggactttccgtgcaccacgtggggactttccgtgcac

SEQ ID NO: 13 - DNA, 252 bp, synthetic construct
  source 1..252: mol_type = other DNA; note = 2 copies of Xenopus leavis beta-globin polyadenylation signal (2xlBGpA)
aaccagcctcaagaacacccgaatggagtctctaagctacataataccaacttacactttacaaaatgttgtcccccaaaatgtagccattcgtatctgctcctaataaaaagaaagtttcttcacaaccagcctcaagaacacccgaatggagtctctaagctacataataccaacttacactttacaaaatgttgtcccccaaaatgtagccattcgtatctgctcctaataaaaagaaagtttcttcac

SEQ ID NO: 14 - DNA, 268 bp, synthetic construct
  source 1..268: mol_type = other DNA; note = 2 copies of human beta-globin polyadenylation signal (2huBGpA)
gctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 15 - DNA, 260 bp, synthetic construct
  source 1..260: mol_type = other DNA; note = Hybrid Xenopus leavis and human beta-globin polyadenylation signal (xlhuBGpA)
aaccagcctcaagaacacccgaatggagtctctaagctacataataccaacttacactttacaaaatgttgtcccccaaaatgtagccattcgtatctgctcctaataaaaagaaagtttcttcacgctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 16 - DNA, 372 bp, synthetic construct
  source 1..372: mol_type = other DNA; note = 2xlBGpA-A120
aaccagcctcaagaacacccgaatggagtctctaagctacataataccaacttacactttacaaaatgttgtcccccaaaatgtagccattcgtatctgctcctaataaaaagaaagtttcttcacaaccagcctcaagaacacccgaatggagtctctaagctacataataccaacttacactttacaaaatgttgtcccccaaaatgtagccattcgtatctgctcctaataaaaagaaagtttcttcacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - DNA, 388 bp, synthetic construct
  source 1..388: mol_type = other DNA; note = 2huBGpA-A120
gctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 380 bp, synthetic construct
  source 1..380: mol_type = other DNA; note = xlhuBGpA-A120
aaccagcctcaagaacacccgaatggagtctctaagctacataataccaacttacactttacaaaatgttgtcccccaaaatgtagccattcgtatctgctcctaataaaaagaaagtttcttcacgctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 7011 bp, synthetic construct
  source 1..7011: mol_type = other DNA; note = pGL2-SS*-CAG-SecNLuc-2A-eGFP-BGpA-SS*
ccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaattggtgtggaaagtccccaggctccccagcaggcagaagtatgcaaagcatgcatctcaattagtcagcaaccatggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 20 - DNA, 6991 bp, synthetic construct
  source 1..6991: mol_type = other DNA; note = pcDNA-CMV-5'UTR-SecNLuc-P2A-eGFP-bGHpA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccctgccttctccctcctgtgagtttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggttggtgtacagtagcttccactcctgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggaattctgcagatatccagcacagtggcggccgctcgagtctagaggaagcggagctactaacttcagcctgctgaagcaggctggagacgtggaggagaaccctggacctatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 21 - DNA, 4555 bp, synthetic construct
  source 1..4555: mol_type = other DNA; note = pGL2-SS*-CAG-SecNLuc-2A-eGFP-WPRE-BGpA-SS*
taaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaataaaggaaatttattttcattgcaatagtgtgttggaattttttgtgtctctcactcggaaggacatggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaattggtgtggaaagtccccaggctccccagcaggcagaagtatgcaaagcatgcatctcaattagtcagcaacca

SEQ ID NO: 22 - DNA, 3610 bp, synthetic construct
  source 1..3610: mol_type = other DNA; note = pGL2-SS*-CMV-UTR1-SecNLuc-2A-eGFP-WPRE-BGpA-SS*
taaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaataaaggaaatttattttcattgcaatagtgtgttggaattttttgtgtctctcactcggaaggacatggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaattggtgtggaaagtccccaggctccccagcaggcagaagtatgcaaagcatgcatctcaattagtcagcaacca

SEQ ID NO: 23 - DNA, 4092 bp, synthetic construct
  source 1..4092: mol_type = other DNA; note = pGL2-SS*-CMV-UTR2-SecNLuc-2A-eGFP-WPRE-BGpA-SS*
aattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctattgggatcttcacacagcaggtaaggttgcgggccgggcctgggccgggtccgggccgggtatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagccgcactgacccctggtgttgctttttttttttaggccgcaagctgaagcgtgtccgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaataaaggaaatttattttcattgcaatagtgtgttggaattttttgtgtctctcactcggaaggacatggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaattggtgtggaaagtccccaggctccccagcaggcagaagtatgcaaagcatgcatctcaattagtcagcaacca

SEQ ID NO: 24 - DNA, 6249 bp, synthetic construct
  source 1..6249: mol_type = other DNA; note = SS*-CMV-UTR1-SecNLuc-2A-eGFP-3'UTR[2huBGpA-A120]-SS *
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 25 - DNA, 6360 bp, synthetic construct
  source 1..6360: mol_type = other DNA; note = SS*-E1-CMV-UTR1-SecNLuc-2A-eGFP-3'UTR[2huBGpA-A120] -SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgggactttccggggcggggcacgtggtgcacgggactttccgtgcacgtgcacgggactttccgggactttccgggactttccgtgcaccacgtggggactttccgtgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 26 - DNA, 6949 bp, synthetic construct
  source 1..6949: mol_type = other DNA; note = SS*-E1-CMV-UTR1-SecNLuc-2A-eGFP-WPRE-3'UTR[2huBGpA- A120]-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgggactttccggggcggggcacgtggtgcacgggactttccgtgcacgtgcacgggactttccgggactttccgggactttccgtgcaccacgtggggactttccgtgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 27 - DNA, 7121 bp, synthetic construct
  source 1..7121: mol_type = other DNA; note = SS*-UCOE-E1-CMV-UTR1-SecNLuc-2A-eGFP-WPRE-3'UTR[2hu BGpA-A120]-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaagggactttccggggcggggcacgtggtgcacgggactttccgtgcacgtgcacgggactttccgggactttccgggactttccgtgcaccacgtggggactttccgtgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 28 - DNA, 7593 bp, synthetic construct
  source 1..7593: mol_type = other DNA; note = SS*-E1-CMV-UTR1-SecNLuc-2A-eGFP-huMAR-3'UTR[2huBGpA -A120]-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgggactttccggggcggggcacgtggtgcacgggactttccgtgcacgtgcacgggactttccgggactttccgggactttccgtgcaccacgtggggactttccgtgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcccattctccttgatgtactaatttttctttaaaagtgataataatagctcccatttagaatttttaaataacacaacaaatgtaaagtaactaatgtgtcctctggatcatggtaagtaatgaataaatttaactccctttaccttctccctttgctattttttccatgctaggatttatacatttttaaaaaactaaatctgctatcaaatgacagctttaaatttactttttaaaatttgttattgtatatatttatggggtataaagtgatgttatgatatatatatacacaatgtacactgattaaatcaagccaattaacattttatcatctcaaatacttaacattttttgtagtgagaacatttgaaatttacttttagcaatttcaaaacatacattattattattaactatagtcaccatgatgtaccatagatctttaaaaacttattcttcctgcctaactgaaactttgtactctttgactaacatcttttcattcccccacttcccagcctctggtaatcaccattacacactctgcttctatgagttcaattgctttagactccacgtaataaatgagatcatgcagcatttggctttctgtgcctggcttatccttgcttagcatggtgtcttacaggttcatccatgttgcaacaaataacagaatctcattctttgttaaggctgaatactattccattgggtatatataccacattttccttatccattaatccactgatggacccttaggttgttgattccatatattggctattgtaaatagtgcagcaatgaacatgagagtgcaactatctcttcaatgtactgatttcgaatccttcggatctatctcagaagtgagattgcaggatcatataattctacttttagtcttttgaggagctccatacagctttccatatggccatactaattacattctcatcaacagtgtacaatggtttccttttctccacatcctcaccaacatttataattttttgtctttttgataatagccatctgacaggtgtaaagtgatagctcattgcagttttaatttgcattttttgatgattagtaatgttgagaattttttcatatatctcttggccagttgcatgtcttctttggaaaaatgtctattcagttcctttgcccattttttaattgggatttttggtttcttgctattgagttgtttgaattcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 29 - DNA, 8354 bp, synthetic construct
  source 1..8354: mol_type = other DNA; note = SS*-UCOE-E1-CMV-UTR1-SecNLuc-2A-eGFP-huMAR-3'UTR[2h uBGpA-A120]-SS *
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaagggactttccggggcggggcacgtggtgcacgggactttccgtgcacgtgcacgggactttccgggactttccgggactttccgtgcaccacgtggggactttccgtgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcccattctccttgatgtactaatttttctttaaaagtgataataatagctcccatttagaatttttaaataacacaacaaatgtaaagtaactaatgtgtcctctggatcatggtaagtaatgaataaatttaactccctttaccttctccctttgctattttttccatgctaggatttatacatttttaaaaaactaaatctgctatcaaatgacagctttaaatttactttttaaaatttgttattgtatatatttatggggtataaagtgatgttatgatatatatatacacaatgtacactgattaaatcaagccaattaacattttatcatctcaaatacttaacattttttgtagtgagaacatttgaaatttacttttagcaatttcaaaacatacattattattattaactatagtcaccatgatgtaccatagatctttaaaaacttattcttcctgcctaactgaaactttgtactctttgactaacatcttttcattcccccacttcccagcctctggtaatcaccattacacactctgcttctatgagttcaattgctttagactccacgtaataaatgagatcatgcagcatttggctttctgtgcctggcttatccttgcttagcatggtgtcttacaggttcatccatgttgcaacaaataacagaatctcattctttgttaaggctgaatactattccattgggtatatataccacattttccttatccattaatccactgatggacccttaggttgttgattccatatattggctattgtaaatagtgcagcaatgaacatgagagtgcaactatctcttcaatgtactgatttcgaatccttcggatctatctcagaagtgagattgcaggatcatataattctacttttagtcttttgaggagctccatacagctttccatatggccatactaattacattctcatcaacagtgtacaatggtttccttttctccacatcctcaccaacatttataattttttgtctttttgataatagccatctgacaggtgtaaagtgatagctcattgcagttttaatttgcattttttgatgattagtaatgttgagaattttttcatatatctcttggccagttgcatgtcttctttggaaaaatgtctattcagttcctttgcccattttttaattgggatttttggtttcttgctattgagttgtttgaattcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 30 - DNA, 7710 bp, synthetic construct
  source 1..7710: mol_type = other DNA; note = SS*-UCOE-E1-CMV-UTR1-SecNLuc-2A-eGFP-WPRE-3'UTR[2hu BGpA-A120]-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaagggactttccggggcggggcacgtggtgcacgggactttccgtgcacgtgcacgggactttccgggactttccgggactttccgtgcaccacgtggggactttccgtgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 31 - DNA, 8182 bp, synthetic construct
  source 1..8182: mol_type = other DNA; note = SS*-E1-CMV-UTR1-SecNLuc-2A-eGFP-MAR-WPRE-3'UTR[2huB GpA-A120]-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgggactttccggggcggggcacgtggtgcacgggactttccgtgcacgtgcacgggactttccgggactttccgggactttccgtgcaccacgtggggactttccgtgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcccattctccttgatgtactaatttttctttaaaagtgataataatagctcccatttagaatttttaaataacacaacaaatgtaaagtaactaatgtgtcctctggatcatggtaagtaatgaataaatttaactccctttaccttctccctttgctattttttccatgctaggatttatacatttttaaaaaactaaatctgctatcaaatgacagctttaaatttactttttaaaatttgttattgtatatatttatggggtataaagtgatgttatgatatatatatacacaatgtacactgattaaatcaagccaattaacattttatcatctcaaatacttaacattttttgtagtgagaacatttgaaatttacttttagcaatttcaaaacatacattattattattaactatagtcaccatgatgtaccatagatctttaaaaacttattcttcctgcctaactgaaactttgtactctttgactaacatcttttcattcccccacttcccagcctctggtaatcaccattacacactctgcttctatgagttcaattgctttagactccacgtaataaatgagatcatgcagcatttggctttctgtgcctggcttatccttgcttagcatggtgtcttacaggttcatccatgttgcaacaaataacagaatctcattctttgttaaggctgaatactattccattgggtatatataccacattttccttatccattaatccactgatggacccttaggttgttgattccatatattggctattgtaaatagtgcagcaatgaacatgagagtgcaactatctcttcaatgtactgatttcgaatccttcggatctatctcagaagtgagattgcaggatcatataattctacttttagtcttttgaggagctccatacagctttccatatggccatactaattacattctcatcaacagtgtacaatggtttccttttctccacatcctcaccaacatttataattttttgtctttttgataatagccatctgacaggtgtaaagtgatagctcattgcagttttaatttgcattttttgatgattagtaatgttgagaattttttcatatatctcttggccagttgcatgtcttctttggaaaaatgtctattcagttcctttgcccattttttaattgggatttttggtttcttgctattgagttgtttgaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 32 - DNA, 8943 bp, synthetic construct
  source 1..8943: mol_type = other DNA; note = SS*-UCOE-E1-CMV-UTR1-SecNLuc-2A-eGFP-MAR-WPRE-3'UTR [2huBGpA-A120] -SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaagggactttccggggcggggcacgtggtgcacgggactttccgtgcacgtgcacgggactttccgggactttccgggactttccgtgcaccacgtggggactttccgtgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcccattctccttgatgtactaatttttctttaaaagtgataataatagctcccatttagaatttttaaataacacaacaaatgtaaagtaactaatgtgtcctctggatcatggtaagtaatgaataaatttaactccctttaccttctccctttgctattttttccatgctaggatttatacatttttaaaaaactaaatctgctatcaaatgacagctttaaatttactttttaaaatttgttattgtatatatttatggggtataaagtgatgttatgatatatatatacacaatgtacactgattaaatcaagccaattaacattttatcatctcaaatacttaacattttttgtagtgagaacatttgaaatttacttttagcaatttcaaaacatacattattattattaactatagtcaccatgatgtaccatagatctttaaaaacttattcttcctgcctaactgaaactttgtactctttgactaacatcttttcattcccccacttcccagcctctggtaatcaccattacacactctgcttctatgagttcaattgctttagactccacgtaataaatgagatcatgcagcatttggctttctgtgcctggcttatccttgcttagcatggtgtcttacaggttcatccatgttgcaacaaataacagaatctcattctttgttaaggctgaatactattccattgggtatatataccacattttccttatccattaatccactgatggacccttaggttgttgattccatatattggctattgtaaatagtgcagcaatgaacatgagagtgcaactatctcttcaatgtactgatttcgaatccttcggatctatctcagaagtgagattgcaggatcatataattctacttttagtcttttgaggagctccatacagctttccatatggccatactaattacattctcatcaacagtgtacaatggtttccttttctccacatcctcaccaacatttataattttttgtctttttgataatagccatctgacaggtgtaaagtgatagctcattgcagttttaatttgcattttttgatgattagtaatgttgagaattttttcatatatctcttggccagttgcatgtcttctttggaaaaatgtctattcagttcctttgcccattttttaattgggatttttggtttcttgctattgagttgtttgaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 33 - DNA, 340 bp, synthetic construct
  source 1..340: mol_type = other DNA; note = Super Sequence, SS*
taaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacc

SEQ ID NO: 34 - DNA, 6326 bp, synthetic construct
  source 1..6326: mol_type = other DNA; note = pGL2-CAG-SecNLuc-2A-eGFP-WPRE-bGlobin polyA
cccgggaggtaccgagctcttacgcgtgctagcctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacattggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 35 - DNA, 1626 bp, synthetic construct
  source 1..1626: mol_type = other DNA; note = CAG [E1X3+CBA promoter+intron]
gggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcacgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 36 - DNA, 1559 bp, synthetic construct
  source 1..1559: mol_type = other DNA; note = CAG [E2+CBA promoter+intron]
tgggactttccactagacatgacacagcaatctgatatgcttgcgtgagaagaggattcatatcctgggactttccacagattttaccggaagttgttagatgcttgcgtgagaagatctaacatgacacagcaatccttagtgggactttccaagtatgtggggcggggagtatacatgacacagcaattgatcattaccggaagtttataggtgggactttccagacctatgcttgcgtgagaagaaaggtctgggactttccagtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 37 - DNA, 826 bp, synthetic construct
  source 1..826: mol_type = other DNA; note = CAG [E1X3+CBA promoter+UTR1]
gggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcacgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtc

SEQ ID NO: 38 - DNA, 759 bp, synthetic construct
  source 1..759: mol_type = other DNA; note = CAG [E2 (U100)+CBA promoter+UTR1]
tgggactttccactagacatgacacagcaatctgatatgcttgcgtgagaagaggattcatatcctgggactttccacagattttaccggaagttgttagatgcttgcgtgagaagatctaacatgacacagcaatccttagtgggactttccaagtatgtggggcggggagtatacatgacacagcaattgatcattaccggaagtttataggtgggactttccagacctatgcttgcgtgagaagaaaggtctgggactttccagtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtc

SEQ ID NO: 39 - DNA, 808 bp, synthetic construct
  source 1..808: mol_type = other DNA; note = CMV enhancer-EF1-UTR1
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtc

SEQ ID NO: 40 - DNA, 7772 bp, synthetic construct
  source 1..7772: mol_type = other DNA; note = 40 4-1 pGL2-SS*-CAG [CMV enhancer+CBA Promoter+intron]-SecNLuc-2A-eGFP-WPRE-3'UTR(108 to 120 polyA)-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 41 - DNA, 7725 bp, synthetic construct
  source 1..7725: mol_type = other DNA; note = 4-2 pGL2-SS*-CAG [E1 X3+CBA promoter+introne]-SecNLuc-2A-eGFP-WPRE-3'UTR(108 to 120 polyA)-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcacgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 42 - DNA, 7658 bp, synthetic construct
  source 1..7658: mol_type = other DNA; note = 4-3 pGL2-SS*-CAG [E2(U100)+CBA promoter+introne]-SecNLuc-2A-eGFP-WPRE-3'UTR(108 to 120 polyA)-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgactgggactttccactagacatgacacagcaatctgatatgcttgcgtgagaagaggattcatatcctgggactttccacagattttaccggaagttgttagatgcttgcgtgagaagatctaacatgacacagcaatccttagtgggactttccaagtatgtggggcggggagtatacatgacacagcaattgatcattaccggaagtttataggtgggactttccagacctatgcttgcgtgagaagaaaggtctgggactttccagtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 43 - DNA, 6850 bp, synthetic construct
  source 1..6850: mol_type = other DNA; note = 4-4 pGL2-SS*-CAG [E1 X3+CBA promoter+UTR1]-SecNLuc-2A-eGFP-WPRE-3'UTR(108 to 120 polyA)-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcacgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 44 - DNA, 6783 bp, synthetic construct
  source 1..6783: mol_type = other DNA; note = 4-5-pGL2-SS*-CAG [E2 (U100)+CBA promoter+UTR1]-SecNLuc-2A-eGFP-WPRE-3'UTR (108 to 120 polyA)-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgactgggactttccactagacatgacacagcaatctgatatgcttgcgtgagaagaggattcatatcctgggactttccacagattttaccggaagttgttagatgcttgcgtgagaagatctaacatgacacagcaatccttagtgggactttccaagtatgtggggcggggagtatacatgacacagcaattgatcattaccggaagtttataggtgggactttccagacctatgcttgcgtgagaagaaaggtctgggactttccagtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 45 - DNA, 6832 bp, synthetic construct
  source 1..6832: mol_type = other DNA; note = 4-6-pGL2-SS*-CMV enhancer-EF1-UTR1-SecNLuc-2A-eGFP-WPRE-3'UTR(108 to 120 polyA)-SS*
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgggtcgacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgccttctccctcctgtgagtttggtaagtcgacgggccgggcctgggccgggtccgggccgggtcgttggatccccactacagcccgatactcaagcttgacgaattcgagtatccaaggtagtggactagtgtgacgctgctgacccctttctttcccttctgcaggttggtgtacagtagcttccaaattgattaattcgagcgaacgcgtcgccaccatgaactccttctccacaagcgccttcggtccagttgccttctccctgggcctgctcctggtgttgcctgctgccttccctgccccagtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggctagcgctactaacttcagcctgctgaagcaggctggagacgtggaggagaaccctggacctggaagcggagagggcagaggaagtctgctaacatgcggtgacgtcgaggagaatcctggacctggatccggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaactcggaaggacatggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 46 - DNA, 333 bp, synthetic construct
  source 1..333: mol_type = other DNA; note = 3 copies of Enhancer-1
gggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcacgggactttccggggcggggcacgtggtgcacgggactttccgtgcacgtgcacgggactttccgggactttccgggactttccgtgcaccacgtggggactttccgtgcac

SEQ ID NO: 47 - DNA, 1016 bp, synthetic construct
  source 1..1016: mol_type = other DNA; note = chimeric intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

(19) United States
(12) Patent Application Publication
(10) Pub. No.: US 2024/0229071 A1
(43) Pub. Date: Jul. 11, 2024

(54) EXPRESSION VECTORS, BACTERIAL SEQUENCE-FREE VECTORS, AND METHODS OF MAKING AND USING THE SAME

(71) Applicant: Mediphage Bioceuticals, Inc., Toronto (CA)

(72) Inventors: Roderick SLAVCEV, Waterloo (CA); Nafiseh NAFISSI, Toronto (CA)

(21) Appl. No.: 18/541,459

(22) Filed: Dec. 15, 2023

Related U.S. Application Data
(63) Continuation of application No. PCT/IB2022/055620, filed on Jun. 16, 2022.
(60) Provisional application No. 63/211,343, filed on Jun. 16, 2021.
(60) Provisional application No. 63/306,015, filed on Feb. 2, 2022.
(60) Provisional application No. 63/331,638, filed on Apr. 15, 2022.

(51) Int. Cl. C12N 15/86; C12N 9/22

(52) U.S. Cl. CPC C12N 15/86; C12N 9/22; C12N 2310/20; C12N 2710/16143; C12N 2710/16145; C12N 2800/30; C12N 2830/48

(57) ABSTRACT

The present disclosure provides expression vectors, bacterial sequence-free vectors, such as ministring DNA (msDNA), and methods of making the bacterial sequence-free vectors, including with vector production systems. The present disclosure also provides compositions comprising the vectors, and uses of the vectors and compositions.

### CROSS REFERENCE TO RELATED APPLICATIONS

This application is a continuation of PCT Application No. PCT/IB2022/055620, filed Jun. 16, 2022, which claims the priority benefit of U.S. Provisional Application Nos. 63/211,343, filed Jun. 16, 2021, 63/306,015, filed Feb. 2, 2022, and 63/331,638, filed Apr. 15, 2022, which are incorporated herein by reference in their entireties.

### REFERENCE TO SEQUENCE LISTING SUBMITTED ELECTRONICALLY

The content of the electronically submitted sequence listing (Name: 4471_0070003_SequenceListing_ST26; Size: 211,617 Bytes; and Date of Creation: Mar. 29, 2024) is herein incorporated by reference in its entirety.

### FIELD OF DISCLOSURE

The present disclosure provides expression vectors, bacterial sequence-free vectors, vector production systems for making the bacterial sequence-free vectors, and uses thereof.

### BACKGROUND

Gene therapy has significant therapeutic promise, but challenges remain in realizing its potential.

Most clinical trials have utilized viral delivery systems, such as adenoviral vectors, lentiviral vectors, and adeno-associated viral vectors. While progress has been made, viral systems vary in transduction and transgene expression efficiencies and concerns remain regarding undesirable effects such as inflammatory and immune responses or insertional mutagenesis. Moreover, production, purification, and storage of viral vectors is often costly, highly variable, and inefficient. See, e.g., Lingelbach, D., Drug Development & Delivery 20(5): 50-54 (2020); Wright, J. F., Gene Therapy 15:840-848 (2008).

Nonviral vectors also have been investigated as gene therapy delivery systems. While safer than their viral counterparts, the effectiveness of nonviral vectors can be limited, for example, by low transgene expression levels and durability of expression. See, e.g., Kay, M., Nature Reviews Genetics 12: 316-328 (2011).

There is a need for improved vectors such as those described herein.

### BRIEF SUMMARY

The present disclosure is directed to an expression vector comprising: (a) a backbone sequence, (b) a sequence comprising: (i) an expression cassette comprising a nucleic acid sequence of interest, (ii) a first target sequence for a first recombinase flanking the 5′ side of the expression cassette, (iii) a second target sequence for the first recombinase flanking the 3′ side of the expression cassette, and (iv) one or more additional target sequences for one or more additional recombinases integrated within the first and second target sequences in non-binding regions for the first recombinase, and (c) one or more of: (i) an endonuclease target sequence integrated within the first and/or second target sequences for the first recombinase in non-binding regions for the first recombinase and the one or more additional recombinases, wherein the endonuclease target sequence is between the backbone sequence and cleavage sites for the first recombinase and the one or more additional recombinases, (ii) a synthetic enhancer comprising a nucleic acid sequence at least about 90% identical to SEQ ID NO:12 integrated between the 3′ end of the first target sequence for the first recombinase and the 5′ end of another enhancer or a promoter in the expression cassette, (iii) a cytomegalovirus (CMV) enhancer integrated between the 3′ end of the first target sequence for the first recombinase and the 5′ end of a promoter in the expression cassette, (iv) a 5′ untranslated region (5′UTR) comprising an intron, wherein the 5′UTR is integrated in the expression cassette between a promoter and the nucleic acid sequence of interest, (v) a vertebrate chromatin insulator integrated in the expression cassette between the nucleic acid of interest and a polyadenylation signal, (vi) a woodchuck hepatitis virus post-transcriptional regulatory element (WPRE) integrated in the expression cassette between the nucleic acid of interest and a polyadenylation signal, (vii) a scaffold/matrix attachment region (S/MAR) integrated in the expression cassette between the nucleic acid of interest and a polyadenylation signal, or (viii) a DNA nuclear targeting sequence (DTS) integrated within the first and/or second target sequences for the first recombinase in non-binding regions for the first recombinase and the one or more additional recombinases, wherein the DTS is between the expression cassette and cleavage sites for the first recombinase and the one or more additional recombinases.

In some aspects, the expression vector comprises an endonuclease target sequence integrated within the first and/or second target sequences for the first recombinase in non-binding regions for the first recombinase and the one or more additional recombinases, wherein the endonuclease target sequence is between the backbone sequence and cleavage sites for the first recombinase and the one or more additional recombinases. In some aspects, the endonuclease target sequence is integrated within the first and second target sequences for the first recombinase. In some aspects, the endonuclease target sequence is for a homing endonuclease. In some aspects, the endonuclease target sequence is for I-AniI, I-CeuI, I-ChuI, I-CpaI, I-CpaII, I-CreI, I-DmoI, H-DreI, I-HmuI, I-HmuII, I-LlaI, I-MsoI, PI-PfuI, PI-PkoII, I-PorI, I-PpoI, PI-PspI, I-ScaI, I-SceI, PI-SceI, I-SceII, I-SecIII, I-SceIV, I-SceV, I-SceVI, I-SceVII, I-Ssp6803I, I-TevI, I-TevII, I-TevIII, PI-TliI, PI-TliII, I-Tsp061I, or I-Vdi141I. In some aspects, the endonuclease target sequence is for I-SceI. In some aspects, the endonuclease target sequence is for PI-SceI. In some aspects, the endonuclease target sequence is for a Cas endonuclease. In some aspects, the Cas endonuclease is Cas9.

In some aspects, the expression vector comprises a synthetic enhancer comprising a nucleic acid sequence at least about 90% identical to SEQ ID NO:12 integrated between the 3′ end of the first target sequence for the first recombinase and the 5′ end of another enhancer or a promoter in the expression cassette. In some aspects, the synthetic enhancer comprises multiple contiguous copies of a nucleic acid sequence at least about 90% identical to SEQ ID NO:12. In some aspects, the synthetic enhancer comprises a nucleic acid sequence at least about 90% identical to SEQ ID NO:46. In some aspects, the synthetic enhancer is integrated at the 5′ end of a chicken β-actin promoter. In some aspects, a chimeric intron comprising a nucleic acid sequence at least about 90% identical to SEQ ID NO:47 is integrated at the 3′ end of the chicken β-actin promoter and 5′ to the nucleic acid sequence of interest.

In some aspects, the expression vector comprises a CMV enhancer integrated between the 3′ end of the first target sequence for the first recombinase and the 5′ end of a promoter in the expression cassette. In some aspects, the CMV enhancer is integrated at the 3′ end of a synthetic enhancer comprising a nucleic acid sequence at least about 90% identical to SEQ ID NO:12 or SEQ ID NO:46. In some aspects, a CMV promoter is integrated at the 3′ end of the CMV enhancer and 5′ to the nucleic acid sequence of interest.

In some aspects, the expression vector comprises a nucleic acid sequence at least about 90% identical to SEQ ID NO:35, SEQ ID NO:36, SEQ ID NO:37, SEQ ID NO:38, or SEQ ID NO:39 integrated between the first target sequence for the first recombinase and the nucleic acid sequence of interest.

In some aspects, the expression vector comprises a 5′UTR comprising an intron, wherein the 5′UTR is integrated in the expression cassette between a promoter and the nucleic acid sequence of interest. In some aspects, the intron comprises a nucleic acid sequence at least about 90% identical to SEQ ID NO:1. In some aspects, the 5′UTR further comprises a non-coding sequence integrated within the intron. In some aspects, the 5′UTR comprises a non-coding sequence integrated between two of the nucleotides in the intron corresponding to any two nucleotides from positions 25 to 55 of SEQ ID NO:1. In some aspects, the non-coding sequence is an S/MAR. In some aspects, the S/MAR is MAR-5. In some aspects, the 5′UTR comprises a nucleic acid sequence at least about 90% identical SEQ ID NO:3. In some aspects, the 5′UTR comprises a nucleic acid sequence at least about 90% identical SEQ ID NO:5. In some aspects, the promoter is a chicken β-actin promoter. In some aspects, the promoter is a CMV promoter. In some aspects, the promoter is integrated at the 3′ end of a CMV enhancer. In some aspects, the CMV enhancer is integrated at the 3′ end of a synthetic enhancer comprising a nucleic acid sequence at least about 90% identical to SEQ ID NO: 12 or SEQ ID NO:46.

In some aspects, the expression vector comprises a polyadenylation signal that is integrated at the 3′ end of the nucleic acid sequence of interest. In some aspects, the polyadenylation signal comprises a nucleic acid sequence at least about 90% identical to SEQ ID NO:13, SEQ ID NO:14, or SEQ ID NO:15.

In some aspects, the expression vector comprises a vertebrate chromatin insulator integrated in the expression cassette between the nucleic acid of interest and a polyadenylation signal. In some aspects, the vertebrate chromatin insulator is 5′-HS4 chicken-β-globin insulator (cHS4). In some aspects, the polyadenylation signal comprises a nucleic acid sequence at least about 90% identical to SEQ ID NO:13, SEQ ID NO:14, or SEQ ID NO:15.

In some aspects, the expression vector comprises a WPRE integrated in the expression cassette between the nucleic acid of interest and a polyadenylation signal. In some aspects, the polyadenylation signal comprises a nucleic acid sequence at least about 90% identical to SEQ ID NO: 13, SEQ ID NO: 14, or SEQ ID NO: 15.

In some aspects, the expression vector comprises a S/MAR integrated in the expression cassette between the nucleic acid of interest and a polyadenylation signal. In some aspects, the S/MAR is MAR-5. In some aspects, the polyadenylation signal comprises a nucleic acid sequence at least about 90% identical to SEQ ID NO: 13, SEQ ID NO:14, or SEQ ID NO:15.

In some aspects, the expression vector comprises an enhancer sequence flanking each side of the first and second target sequences for the first recombinase. In some aspects, the expression vector comprises at least two enhancer sequences flanking each side of the first and second target sequences for the first recombinase. In some aspects, the enhancer sequence is a SV40 enhancer sequence.

In some aspects, the expression vector comprises a DTS integrated within the first and/or second target sequences for the first recombinase in non-binding regions for the first recombinase and the one or more additional recombinases, wherein the DTS is between the expression cassette and cleavage sites for the first recombinase and the one or more additional recombinases. In some aspects, the DTS is a SV40 enhancer sequence. In some aspects, the DTS is cell-specific.

In some aspects, the first and second target sequences and the one or more additional target sequences are selected from the group consisting of the PY54 pal site, the N15 telRL site, the loxP site, φK02 telRL site, the FRT site, the phiC31 attP site, and the λ attP site. In some aspects, the expression vector comprises each of the target sequences. In some aspects, the expression vector comprises the pal site and the telRL, loxP, and FRT recombinase target binding sequences integrated within the pal site. In some aspects, the first and second target sequences for the first recombinase each comprise the nucleic acid sequence of SEQ ID NO:33.

In some aspects, the expression vector is for producing a bacterial sequence-free vector. In some aspects, the bacterial sequence-free vector is a circular covalently closed vector. In some aspects, the bacterial sequence-free vector is a linear covalently closed vector.

The present disclosure is directed to a vector production system comprising recombinant cells encoding a recombinase under the control of an inducible promoter, wherein the recombinant cells comprise any of the above expression vectors, and wherein the recombinase targets the first and second target sequences for the first recombinase or one of the one or more additional target sequences for the one or more additional recombinases in the expression vector. In some aspects, the recombinase is TelN, Tel, Cre, or Flp.

In some aspects, the recombinant cells further encode an endonuclease under the control of an inducible promoter, wherein the endonuclease targets the endonuclease target sequence in an expression vector comprising the endonuclease target sequence. In some aspects, the endonuclease is a homing endonuclease. In some aspects, the homing endonuclease is I-AniI, I-CeuI, I-ChuI, I-CpaI, I-CpaII, I-CreI, I-DmoI, H-DreI, I-HmuI, I-HmuII, I-LlaI, I-MsoI, PI-PfuI, PI-PkoII, I-PorI, I-PpoI, PI-PspI, I-ScaI, I-SceI, PI-SceI, I-SceII, I-SecIII, I-SceIV, I-SceV, I-SceVI, I-SceVII, I-Ssp6803I, I-TevI, I-TevII, I-TevIII, PI-TliI, PI-TliII, I-Tsp061I, or I-Vdi141I. In some aspects, the endonuclease is I-SceI. In some aspects, the endonuclease is PI-SceI. In some aspects, the recombinant cells encode a nuclease genome editing system comprising the endonuclease. In some aspects, the nuclease genome editing system is a clustered regularly interspaced short palindromic repeats (CRISPR) nuclease system comprising a guide RNA and a Cas endonuclease. In some aspects, the Cas endonuclease is Cas9. In some aspects, the inducible promoter is thermally-regulated, chemically-regulated, IPTG regulated, glucose-regulated, arabinose inducible, T7 polymerase regulated, cold-shock inducible, pH inducible, or combinations thereof.

The present disclosure is directed to a method of producing a bacterial sequence-free vector comprising incubating any of the above vector production systems under suitable conditions for expression of the recombinase. In some aspects, the method further comprises incubating any of the above vector production systems that encode an endonuclease under suitable conditions for expression of the endonuclease. In some aspects, the method further comprises incubating any of the above vector production systems that encode a nuclease genome editing system under suitable conditions for expression of the nuclease genome editing system. In some aspects, the method further comprises harvesting the bacterial sequence-free vector.

The present disclosure is directed to a bacterial sequence-free vector produced by any of the above methods of producing a bacterial sequence-free vector.

The present disclosure is directed to a bacterial sequence-free vector comprising: (a) an expression cassette comprising a nucleic acid sequence of interest, and (b) one or more of: (i) a synthetic enhancer comprising a nucleic acid sequence at least about 90% identical to SEQ ID NO:12 located 5′ to another enhancer or a promoter in the expression cassette, (ii) a CMV enhancer located 5′ to a promoter in the expression cassette, (iii) a 5′UTR comprising an intron, wherein the 5′UTR is integrated in the expression cassette between a promoter and the nucleic acid sequence of interest, (iv) a vertebrate chromatin insulator integrated in the expression cassette between the nucleic acid of interest and a polyadenylation signal, (v) a WPRE integrated in the expression cassette between the nucleic acid of interest and a polyadenylation signal, (vi) a S/MAR integrated in the expression cassette between the nucleic acid of interest and a polyadenylation signal, or (vii) a DTS located 5′ to the expression cassette.

In some aspects, the bacterial sequence-free vector comprises a synthetic enhancer comprising a nucleic acid sequence at least about 90% identical to SEQ ID NO:12 located 5′ to another enhancer or a promoter in the expression cassette. In some aspects, the synthetic enhancer comprises multiple contiguous copies of a nucleic acid sequence at least about 90% identical to SEQ ID NO: 12 In some aspects, the synthetic enhancer comprises a nucleic acid sequence at least about 90% identical to SEQ ID NO:46. In some aspects, the synthetic enhancer is integrated at the 5′ end of a chicken β-actin promoter. In some aspects, a chimeric intron comprising a nucleic acid sequence at least about 90% identical to SEQ ID NO:47 is integrated at the 3′ end of the chicken β-actin promoter and 5′ to the nucleic acid sequence of interest.

In some aspects, the bacterial sequence-free vector comprises a CMV enhancer located 5′ to a promoter in the expression cassette. In some aspects, the CMV enhancer is integrated at the 3′ end of a synthetic enhancer comprising a nucleic acid sequence at least about 90% identical to SEQ ID NO:12 or SEQ ID NO:46. In some aspects, a CMV promoter is integrated at the 3′ end of the CMV enhancer and 5′ to the nucleic acid sequence of interest.

In some aspects, the bacterial sequence-free vector comprises a nucleic acid sequence at least about 90% identical to SEQ ID NO:35, SEQ ID NO:36, SEQ ID NO:37, SEQ ID NO:38, or SEQ ID NO:39 located 5′ to the nucleic acid sequence of interest.

In some aspects, the bacterial sequence-free vector comprises a 5′UTR comprising an intron, wherein the 5′UTR is integrated in the expression cassette between a promoter and the nucleic acid sequence of interest. In some aspects, the intron comprises a nucleic acid sequence at least about 90% identical to SEQ ID NO:1. In some aspects, the 5′UTR further comprises a non-coding sequence integrated within the intron. In some aspects, the 5′UTR further comprises a non-coding sequence integrated between two of the nucleotides in the intron corresponding to any two nucleotides from nucleotide positions 25 and 55 of SEQ ID NO: 1. In some aspects, the non-coding sequence is an S/MAR. In some aspects, the S/MAR is MAR-5. In some aspects, the 5′UTR comprises a nucleic acid sequence at least about 90% identical SEQ ID NO:3. In some aspects, the 5′UTR comprises a nucleic acid sequence at least about 90% identical SEQ ID NO:5. In some aspects, the promoter is a chicken β-actin promoter. In some aspects, the promoter is a CMV promoter. In some aspects, the promoter is integrated at the 3′ end of a CMV enhancer. In some aspects, the CMV enhancer is integrated at the 3′ end of a synthetic enhancer comprising a nucleic acid sequence at least about 90% identical to SEQ ID NO: 12 or SEQ ID NO:46.

In some aspects, the bacterial sequence-free vector comprises a polyadenylation signal that is integrated at the 3′ end of the nucleic acid sequence of interest. In some aspects, the polyadenylation signal comprises a nucleic acid sequence at least about 90% identical to SEQ ID NO:13, SEQ ID NO: 14, or SEQ ID NO:15.

In some aspects, the bacterial sequence-free vector comprises a vertebrate chromatin insulator integrated in the expression cassette between the nucleic acid of interest and a polyadenylation signal. In some aspects, the vertebrate chromatin insulator is cHS4. In some aspects, the polyadenylation signal comprises a nucleic acid sequence at least about 90% identical to SEQ ID NO: 13, SEQ ID NO: 14, or SEQ ID NO: 15.

In some aspects, the bacterial sequence-free vector comprises a WPRE integrated in the expression cassette between the nucleic acid of interest and a polyadenylation signal. In some aspects, the polyadenylation signal comprises a nucleic acid sequence at least about 90% identical to SEQ ID NO:13, SEQ ID NO:14, or SEQ ID NO:15.

In some aspects, the bacterial sequence-free vector comprises a S/MAR integrated in the expression cassette between the nucleic acid of interest and a polyadenylation signal. In some aspects, the S/MAR is MAR-5.

In some aspects, the bacterial sequence-free vector comprises an enhancer sequence flanking each side of the expression cassette. In some aspects, the bacterial sequence-free vector comprises at least two enhancer sequences flanking each side of the expression cassette. In some aspects, the enhancer sequence is a SV40 enhancer sequence.

In some aspects, the bacterial sequence-free vector comprises a DTS located 5′ to the expression cassette. In some aspects, the DTS is a SV40 enhancer sequence. In some aspects, the DTS is cell-specific.

In some aspects, the bacterial sequence-free vector is a circular covalently closed vector.

In some aspects, the bacterial sequence-free vector is a linear covalently closed vector.

The present disclosure is directed to a recombinant cell comprising any of the above expression vectors or any of the above bacterial sequence-free vectors.

The present disclosure is directed to a composition comprising any of the above expression vectors or any of the above bacterial sequence-free vectors. In some aspects, the composition further comprises a delivery agent. In some aspects, the delivery agent is a nanoparticle. In some aspects, the delivery agent comprises a targeting ligand. In some aspects, the composition is a pharmaceutical composition further comprising a pharmaceutically acceptable carrier.

The present disclosure is directed to a method of treating a disease or disorder in a subject in need thereof, comprising administering to the subject any of the above expression vectors, any of the above bacterial sequence-free vectors, or the above pharmaceutical composition.

The present disclosure is directed to a polynucleotide comprising a nucleic acid sequence at least about 90% identical to SEQ ID NO:1.

The present disclosure is directed to a polynucleotide comprising a nucleic acid sequence at least about 90% identical to SEQ ID NO:2.

The present disclosure is directed to a polynucleotide comprising a nucleic acid sequence at least about 90% identical to SEQ ID NO:3.

The present disclosure is directed to a polynucleotide comprising a nucleic acid sequence at least about 90% identical to SEQ ID NO:5.

The present disclosure is directed to a polynucleotide comprising a nucleic acid sequence at least about 90% identical to SEQ ID NO: 12.

The present disclosure is directed to a polynucleotide comprising a nucleic acid sequence at least about 90% identical to SEQ ID NO:46.

The present disclosure is directed to a polynucleotide comprising a nucleic acid sequence at least about 90% identical to SEQ ID NO:13.

The present disclosure is directed to a polynucleotide comprising a nucleic acid sequence at least about 90% identical to SEQ ID NO:14.

The present disclosure is directed to a polynucleotide comprising a nucleic acid sequence at least about 90% identical to SEQ ID NO:15.

In some aspects, any of the above polynucleotides comprising a nucleic acid sequence at least about 90% identical to any one of SEQ ID NOs: 13-15 further comprises 100 to 120 adenine nucleotides at the 3′ end of the nucleic acid sequence.

The present disclosure is directed to a polynucleotide comprising a nucleic acid sequence at least about 90% identical to SEQ ID NO:16.

The present disclosure is directed to a polynucleotide comprising a nucleic acid sequence at least about 90% identical to SEQ ID NO:17.

The present disclosure is directed to a polynucleotide comprising a nucleic acid sequence at least about 90% identical to SEQ ID NO:18.

The present disclosure is directed to a polynucleotide comprising a nucleic acid sequence at least about 90% identical to SEQ ID NO:35.

The present disclosure is directed to a polynucleotide comprising a nucleic acid sequence at least about 90% identical to SEQ ID NO:36.

The present disclosure is directed to a polynucleotide comprising a nucleic acid sequence at least about 90% identical to SEQ ID NO:37.

The present disclosure is directed to a polynucleotide comprising a nucleic acid sequence at least about 90% identical to SEQ ID NO:38.

The present disclosure is directed to a polynucleotide comprising a nucleic acid sequence at least about 90% identical to SEQ ID NO:39.

The present disclosure is directed to an expression vector comprising any of the above polynucleotides.

The present disclosure is directed to an expression vector comprising a polynucleotide comprising a nucleic acid sequence at least about 90% identical to any one of SEQ ID NOs:2, 3, or 5, and (i) a polynucleotide comprising a nucleic acid sequence at least about 90% identical to any one of SEQ ID NOs: 13-18, or (ii) a polynucleotide comprising a nucleic acid sequence at least about 90% identical to any one of SEQ ID NOs:13-15 and 100 to 120 adenine nucleotides at the 3′ end of the nucleic acid sequence.

The present disclosure is directed to a method of gene editing comprising inserting a nucleic acid sequence of interest from any of the above expression vectors, any of the bacterial sequence-free vectors, or any of the above pharmaceutical compositions into a target site for gene editing. In some aspects, the gene editing is by non-homologous end joining. In some aspects, the gene editing is by homology-directed repair.

### BRIEF DESCRIPTION OF THE DRAWINGS

FIG. 1 shows a vector map of the expression vector pGL2-SS*-CAG-SecNLuc-2A-eGFP-BGpA-SS*.

FIG. 2 shows a vector map of the expression vector pcDNA-CMV-5′UTR-SecNLuc-P2A-eGFP-bGHpA.

FIG. 3 shows photomicrographs evaluating fluorescence in HEK-293 cells via live imaging. (A) shows negative control cells exposed to lipofectamine without plasmid, (B) shows cells transfected with the expression vector of FIG. 1, (C) shows cells transfected with the expression vector of FIG. 2, and (D) shows positive control cells transfected with a parental expression vector, pGL2-SS*-CAG-eGFP-BGpA-SS* (PP-CAG-GFP), expressing eGFP under the control of a CAG promoter.

FIG. 4 shows a bar graph of the relative fluorescence intensities of cells transected according to FIG. 3(A)-(D). “pGL2-SecNLuc-eGFP” in FIGS. 4-5 indicates cells transfected with the pGL2-SS*-CAG-SecNLuc-2A-eGFP-BGpA-SS* expression vector of FIG. 1. “pcDNA-SecNLuc-eGFP” in FIGS. 4-5 indicates cells transfected with the pcDNA-CMV-5′UTR-SecNLuc-P2A-eGFP-bGHpA expression vector of FIG. 2.

FIG. 5 shows a bar graph of relative luciferase intensities in the media of cells transfected according to FIG. 3(A)-(C).

FIG. 6 shows a vector map of the expression vector pGL2-SS*-CAG-SecNLuc-2A-eGFP-WPRE-BGpA-SS*.

FIG. 7 shows a vector map of the expression vector pGL2-SS*-CMV-UTR1-SecNLuc-2A-eGFP-WPRE-BGpA-SS*.

FIG. 8 shows a vector map of the expression vector pGL2-SS*-CMV-UTR2-SecNLuc-2A-eGFP-WPRE-BGpA-SS*.

FIG. 9 shows a line graph of long-term luciferase activity as indicated by luminescence in Relative Luminometer Units (also termed Relative Light Units, RLU) in the media of HEK-293 cells at Days 2, 6, 10, 14, 17, 20, 27, and 34 after electroporation of cells with the expression vectors of FIG. 1 (pGL2-SecNLuc-eGFP), FIG. 6 (WPRE), FIG. 7 (5′UTR1+WPRE), and FIG. 8 (5′UTR2+WPRE) as compared to a negative control in which cells were electroporated with a puc57 plasmid lacking a mammalian expression cassette (Neg. Ctl. (no plasmid)). *=p<0.05, **=p<0.01, *p<0.001, and ****=p<0.0001.

FIG. 10 shows a bar graph of luciferase activity as indicated by luminescence in RLU in the media of transfected and negative control HEK-293 cells as described in FIG. 9 at passage numbers 1, 2, 3, and 5.

FIG. 11 shows a line graph of relative luciferase intensity in HEK-293 cells at passage numbers 1, 2, 3, 4, 5, 6, and 7, corresponding to Days 8, 15, 24, 31, 38, 45, and 52, respectively, after electroporation of the cells with the expression vectors of FIG. 7 (2nd gen pDNA (CMV+U1+W)), msDNA produced from the expression vector of FIG. 7 (2nd gen msDNA (CMV+U1+W)), or the expression vector of FIG. 2 containing a luciferase transgene (conventional pcDNA). **=p<0.01, ***=p<0.001, and ****=p<0.0001 as compared to the conventional pcDNA dataset.

FIG. 12 shows fluorescence in cells transfected with the expression vector of FIG. 1 or FIG. 7 as described in FIG. 9. (A) shows photomicrographs evaluating fluorescence in HEK-293 cells via live imaging at passage numbers 1, 2, 3, and 5. (B) shows a line graph of eGFP positive (GFP) cells observed in the field of view from three live fluorescent images at each passage number; non-significant (ns)=p>0.05. (C) shows a dot plot depicting Mean Fluorescence Intensity (MFI) for GFP′ cells measured from three live fluorescent images at passage number 5. The underlying bar graph for each construct shows the average MFI value from all measured GFP+ cells. ****=p<0.0001.

FIG. 13 shows a line graph of RLU/mg protein in plasma collected from wild-type mice at days 1, 3, 7, 10, 15, 22, 28, 42, and 56 after a single hydrodynamic tail vein injection of 50 μg of pcDNA-CMV-5′UTR-SecNLuc-P2A-eGFP-bGHpA (positive control, PSNLuc), pGL2-SS*-CAG-SecNLuc-2A-eGFP-BGpA-SS* (pCAGLuc), or pGL2-SS*-CMV-UTR1-SecNLuc-2A-eGFP-WPRE-BGpA-SS* (pGSNLuc-WPRE).

FIG. 14 shows a line graph of RLU/mg protein in plasma collected from wild-type mice at days 1, 3, 7, 10, 15, 22, 28, 42, and 56 after a single hydrodynamic tail vein injection of 50 μg of pcDNA-CMV-5′UTR-SecNLuc-P2A-eGFP-bGHpA (positive control, PSNLuc), pGL2-SS*-CAG-SecNLuc-2A-eGFP-BGpA-SS* (pCAGLuc), or pGL2-SS*-CAG-SecNLuc-2A-eGFP-WPRE-BGpA-SS* (pCAGLucWPRE).

FIG. 15 shows a line graph of RLU/mg protein in plasma collected from wild-type mice at days 1, 3, 7, 10, 15, 22, 28, 42, and 56 after a single hydrodynamic tail vein injection of 5 μg of pcDNA-CMV-5′UTR-SecNLuc-P2A-eGFP-bGHpA (positive control, pDNA CMV-U (no SSeq)), pGL2-SS*-CAG-SecNLuc-2A-eGFP-BGpA-SS* (SSeq pDNA CAG), or pGL2-SS*-CAG-SecNLuc-2A-eGFP-WPRE-BGpA-SS* (SSeq pDNA CAG-W).

FIG. 16 shows a line graph of RLU/mg protein in plasma collected from wild-type mice at days 1, 3, 7, 10, 15, 22, 28, 42, and 56 after a single hydrodynamic tail vein injection of 5 μg of pcDNA-CMV-5′UTR-SecNLuc-P2A-eGFP-bGHpA (positive control, No-SSeq pDNA CMV-U), pGL2-SS*-CAG-SecNLuc-2A-eGFP-WPRE-BGpA-SS* (2×SSeq pDNA CAG-W), or msDNA-CAG-SecNLuc-2A-eGFP-WPRE-BGpA (2×SSeq msDNA CAG-W).

FIGS. 17A, 17B, 17C, and 17D show bar graphs of GFP expression as determined by ELISA in liver from wild-type mice at day 56 after a single hydrodynamic tail vein injection of 5 μg of the vectors described in FIG. 16 as well as a negative control mouse with no injection of vector. (FIG. 17A) shows the GFP concentration in μg/mL. (FIG. 17B) shows μg of GFP normalized to μg of total protein. (FIG. 17C) shows μg of GFP normalized to g of total tissue. (FIG. 17D) shows expression levels of GFP relative to control. *P<0.05, **P<0.01, ***P<0.001, ****P<0.0001.

FIG. 18 shows a bar graph of cytoplasmic GFP protein concentration (pg/mL) in liver from wild-type mice at day 56 after a single hydrodynamic tail vein injection of 5 μg of vectors as described in FIG. 16 as well as a negative control mouse with no injection of vector.

FIG. 19 shows a bar graph of total flux in photons/second from in vivo whole body bioluminescence imaging after a single intravenous tail vein injection in mice with a lipid nanoparticle (LNP) carrier (Vehicle(control)) or lipoplex of the LNP and msDNA-CAG-SecNLuc-2A-eGFP-WPRE-BGpA (LNP-2G msDNA-CAG-SecretedNanoLuc), pGL2-SS*-CAG-SecNLuc-2A-eGFP-WPRE-BGpA-SS* (LNP-2G ppDNA-CAG-SecretedNanoLuc), msDNA-CMV-UTR1-SecNLuc-2A-eGFP-WPRE-BGpA (LNP-2G msDNA-CMV-SecretedNanoLuc), pGL2-SS*-CMV-UTR1-SecNLuc-2A-eGFP-WPRE-BGpA-SS* (LNP-2G ppDNA-CMV-SecretedNanoLuc), or pcDNA-CMV-5′UTR-SecNLuc-P2A-eGFP-bGHpA (LNP-Conv.pDNA-CMV-SecretedNanoLuc) on days 1, 3, 10, 30, 58, 92, 119, and 174 as indicated by the bar graphs from left to right for each injection. The bar graphs for the LNP-2G msDNA-CAG-SecretedNanoLuc injection are enclosed within the hashed lines.

FIG. 20 shows photomicrographs of green fluorescent protein (GFP) expression in sagittal brain sections from the cortex, thalamus, brainstem, and cerebellum from a mouse injected with msDNA-CAG-SecNLuc-2A-eGFP-WPRE-BGpA. White arrows indicate transgene expression. Nuclei are indicated by staining with diamidino-2-phenylindole (DAPI).

FIGS. 21-22 show photomicrographs of GFP expression in sagittal brain sections from the cortex and thalamus (FIG. 21) and from the cerebellum and brainstem (FIG. 22) from a mouse injected with msDNA-CAG-SecNLuc-2A-eGFP-WPRE-BGpA. Neurons are indicated with the neuronal marker NeuN.

FIGS. 23-24 show photomicrographs of GFP expression in sagittal brain sections from the cortex and thalamus (FIG. 23) and from the cerebellum and brainstem (FIG. 24) from a mouse injected with msDNA-CMV-UTR1-SecNLuc-2A-eGFP-WPRE-BGpA. Neurons are indicated with the neuronal marker NeuN.

FIGS. 25-26 show bar graphs of luminescence associated with luciferase expression in human T cells (Pan-T(TA+) cells, FIG. 25) or hepatocytes (Huh7 cells, FIG. 26) at 3 and 5 days after transfection with a lipoplex of a lipid nanoparticle carrier (LNP) and pcDNA-CMV-5′UTR-SecNLuc-P2A-eGFP-bGHpA (positive control, LNP-Conv.pDNA-CMV-SecretedNLuc), pGL2-SS*-CMV-UTR1-SecNLuc-2A-eGFP-WPRE-BGpA-SS* (LNP-ppDNA-CMV-SecretedNLuc), msDNA-CMV-UTR1-SecNLuc-2A-eGFP-WPRE-BGpA (LNP-msDNA-CMV-SecretedNLuc), pGL2-SS*-CAG-SecNLuc-2A-eGFP-WPRE-BGpA-SS* (LNP-ppDNA-CAG-SecretedNLuc), or msDNA-CAG-SecNLuc-2A-eGFP-WPRE-BGpA (LNP-msDNA-CAG-SecretedNLuc), a PBS control, or untreated cells.

FIGS. 27A, 27B, 27C, 28A, 28B, 28C, 29A, and 29B show fluorescence activated cell sorting (FACS) scatter plots of knock-in (KI) efficiencies (Q3) for a gene of interest (GOI) at 3 days (FIGS. 27A, 27B, and 27C), 7 days (FIGS. 28A, 28B, and 28C), and 15 days (FIGS. 29A and 29B) after transfection of a CRISPR gene editing system and either a conventional plasmid or msDNA carrying the GOI flanked by 5′ and 3′ homology arms (HDR-GOI-HDR). (FIGS. 27A and 28A) show a FACS scatter plot for control, wild-type (WT) induced pluripotent stem cells (iPSCs) without any HDR KI of the GOI. (FIGS. 27B and 28B) and (FIG. 29A) show a FACS scatter plot in iPSCs following HDR KI of the GOI using a conventional plasmid (Plasmid DNA HDR-GOI-HDR). (FIGS. 27C and 28C) in and (FIG. 29B) show a FACS scatter plot in iPSCs following HDR KI of the GOI using msDNA (msDNA HDR-GOI-HDR).

FIG. 30 shows a vector map of the expression vector SS*-CMV-UTR1-SecNLuc-2A-eGFP-3′UTR[2hBGpA-A120]-SS*.

FIG. 31 shows a vector map of the expression vector SS*-E1-CMV-UTR1-SecNLuc-2A-eGFP-3′UTR[2hBGpA-A120]-SS*.

FIG. 32 shows a vector map of the expression vector SS*-E1-CMV-UTR1-SecNLuc-2A-eGFP-WPRE-3′UTR[2hBGpA-A120]-SS*.

FIG. 33 shows a vector map of the expression vector SS*-UCOE-E1-CMV-UTR1-SecNLuc-2A-eGFP-WPRE-3′UTR[2hBGpA-A120]-SS*.

FIG. 34 shows a vector map of the expression vector SS*-E1-CMV-UTR1-SecNLuc-2A-eGFP-huMAR-3′UTR[2hBGpA-A120]-SS*.

FIG. 35 shows a vector map of the expression vector SS*-UCOE-E1-CMV-UTR1-SecNLuc-2A-eGFP-huMAR-3′UTR[2hBGpA-A120]-SS*.

FIG. 36 shows a vector map of the expression vector SS*-UCOE-E1-CMV-UTR1-SecNLuc-2A-eGFP-WPRE-3′UTR[2hBGpA-A120]-SS*.

FIG. 37 shows a vector map of the expression vector SS*-E1-CMV-UTR1-SecNLuc-2A-eGFP-huMAR-WPRE-3′UTR[2hBGpA-A120]-SS*.

FIG. 38 shows a vector map of the expression vector SS*-UCOE-E1-CMV-UTR1-SecNLuc-2A-eGFP-huMAR-WPRE-3′UTR[2hBGpA-A120]-SS*.

FIG. 39 shows a line graph of luciferase activity as indicated by luminescence in RLU in the media of HEK-293 cells at Days 2, 3, 7, 10, 14, 21, and 28 after electroporation of cells with the expression vectors of FIG. 2 (Conventional pDNA CMV-U), FIG. 30 (A: CMV-U1-3′UTR), FIG. 31 (B: E1-CMV-U1-3′UTR), and FIG. 32 (C: E1-CMV-U1-WPRE-3′UTR). *=p<0.05 and **=p<0.01.

FIG. 40 shows a line graph of relative luciferase intensity in HEK-293 cells at passage numbers 1, 2, 3, 4, and 5 after passaging every 7 days following electroporation of the cells at day 0 with the expression vectors described in FIG. 39. *=p<0.05 and **=p<0.01.

FIG. 41 shows a vector map of the expression vector pGL2-CAG-SecNLuc-2A-eGFP-WPRE-bGlobin poly A.

FIG. 42 shows a vector map of the expression vector 4-1 pGL2-SS*-CAG [CMV enhancer+CBA Promoter+intron]-SecNLuc-2A-eGFP-WPRE-3′UTR(108 to 120 polyA)-SS*.

FIG. 43 shows a vector map of the expression vector 4-2 pGL2-SS*-CAG [E1 X3+CBA promoter+introne]-SecNLuc-2A-eGFP-WPRE-3′UTR(108 to 120 polyA)-SS*.

FIG. 44 shows a vector map of the expression vector 4-3 pGL2-SS*-CAG [E2(U100)+CBA promoter+introne]-SecNLuc-2A-eGFP-WPRE-3′UTR(108 to 120 polyA)-SS*.

FIG. 45 shows a vector map of the expression vector 4-4 pGL2-SS*-CAG [E1 X3+CBA promoter+UTR1]-SecNLuc-2A-eGFP-WPRE-3′UTR(108 to 120 polyA)-SS*.

FIG. 46 shows a vector map of the expression vector 4-5-pGL2-SS*-CAG [E2 (U100)+CBA promoter+UTR1]-SecNLuc-2A-eGFP-WPRE-3′UTR (108 to 120 polyA)-SS*.

FIG. 47 shows a vector map of the expression vector 4-6-pGL2-SS*-CMV enhancer-EF1-UTR1-SecNLuc-2A-eGFP-WPRE-3′UTR(108 to 120 polyA)-SS*.

FIG. 48 shows a bar graph of luciferase activity as indicated by luminescence in RLU in the media of HEK-293 cells at 3 and 6 days after transfection with the expression vectors shown in FIG. 41 (Conventional no SSeq pDNA CAG-W) and FIGS. 42-47 (4-1 to 4-6, respectively).

FIG. 49 shows a diagram of an exemplary sequence for a self-restricting CRISPR gene editing system that contains flanking Super Sequences (SSeq), a synthetic enhancer (E1), a CMV promoter (PCMv), a synthetic 5′UTR containing an optimized internal intron with a tRNA-gRNA-PAM insertion (UTR-tRNA-gRNA-PAM-1), a Casβ2 gene, and a 3′UTR containing a human beta-globin polyadenylation signal and a gRNA-PAM insertion (HBg3′UTR-gRNA-PAM).

FIG. 50 shows a diagram of self-limiting Cas expression from the sequence of FIG. 52 during homology-directed repair (HDR) of chromosomal DNA with a therapeutic GOI flanked by homology arms.

FIG. 51 shows a diagram for two gene-editing scenarios with a self-restricting CRISPR gene editing system. In Scenario 1, a msDNA containing a human expression cassette (e.g., therapeutic GOI) is first transfected for transient expression followed by the gene editing system of FIG. 42 for HDR knock-in. In Scenario 2, HDR knock-in is mediated by a single msDNA containing both the self-restricting CRISPR gene editing system and the human expression cassette flanked by homology arms.

### DETAILED DESCRIPTION

The present disclosure provides expression vectors, bacterial sequence-free vectors (e.g., ministring DNA (msDNA)), vector production systems, methods of making the bacterial sequence-free vectors, compositions, and uses thereof.

All publications cited herein are hereby incorporated by reference in their entireties, including without limitation all journal articles, books, manuals, patent applications, and patents cited herein, to the same extent as if each individual publication was specifically and individually indicated to be incorporated by reference.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

The term “and/or” where used herein is to be taken as specific disclosure of each of the specified features or components with or without the other. Thus, the term “and/or” as used in a phrase such as “A and/or B” is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (i.e., +10%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range, or integer range is to be understood to include the value of any integer within the recited range and, when appropriate, fractions thereof (such as one tenth and one hundredth of an integer), unless otherwise indicated. Numeric ranges are inclusive of the numbers defining the range.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form.

Unless otherwise indicated, nucleotide sequences are written left to right in 5′ to 3′ orientation. Amino acid sequences are written left to right in amino to carboxy orientation.

The headings provided herein are not limitations of the various aspects of the disclosure, which can be had by reference to the specification as a whole. Accordingly, the terms defined immediately below are more fully defined by reference to the specification in its entirety.

“Amino acid” is a molecule having the structure wherein a central carbon atom (the alpha-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

“Protein” or “polypeptide” refers to any polymer of two or more individual amino acids (whether or not naturally occurring) linked via a peptide bond, and occurs when the carboxyl carbon atom of the carboxylic acid group bonded to the alpha-carbon of one amino acid (or amino acid residue) becomes covalently bound to the amino nitrogen atom of amino group bonded to the non alpha-carbon of an adjacent amino acid. The terms “protein” and “polypeptide” can be used interchangeably herein. Similarly, fragments of proteins and polypeptides are also within the scope of the disclosure and may be referred to herein as “proteins” or “polypeptides.” In one aspect of the disclosure, a polypeptide comprises a chimera of two or more parental peptide segments or proteins. The term “polypeptide” is also intended to refer to and encompass the products of post-translation modification (“PTM”) of the polypeptide, including without limitation disulfide bond formation, glycosylation, carbamylation, lipidation, acetylation, phosphorylation, amidation, derivatization by known protecting/blocking groups, proteolytic cleavage, modification by non-naturally occurring amino acids, or any other manipulation or modification, such as conjugation with a labeling component. A polypeptide can be derived from a natural biological source or produced by recombinant technology, but is not necessarily translated from a designated nucleic acid sequence. It can be generated in any manner, including by chemical synthesis. An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

Recombinant polypeptides (i.e., recombinant proteins) comprising two or more proteins as disclosed herein can be encoded by a single coding sequence that comprises polynucleotide sequences encoding each protein. Unless stated otherwise, the polynucleotide sequences encoding each protein are “in frame” such that translation of a single mRNA comprising the polynucleotide sequences results in a single polypeptide comprising each protein. Typically, the proteins in a recombinant polypeptide as described herein will be fused directly to one another or will be separated by a peptide linker. Various polynucleotide sequences encoding peptide linkers are known in the art and include, for example, self-cleaving peptides.

“Polynucleotide” or “nucleic acid” as used herein refers to a polymeric form of nucleotides. In some instances, a polynucleotide comprises a sequence that is either not immediately contiguous with the coding sequences or is immediately contiguous (on the 5′ end or on the 3′ end) with the coding sequences in the naturally occurring genome of the organism from which it is derived. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA) independent of other sequences. The nucleotides of the disclosure can be ribonucleotides, deoxyribonucleotides, or modified forms of either nucleotide. A polynucleotide as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. The term polynucleotide encompasses genomic DNA or RNA (depending upon the organism, i.e., RNA genome of viruses), as well as mRNA encoded by the genomic DNA, and cDNA. In certain aspects, a polynucleotide comprises a conventional phosphodiester bond or a non-conventional bond (e.g., an amide bond, such as found in peptide nucleic acids (PNA)). By “isolated” nucleic acid or polynucleotide is intended a nucleic acid molecule, e.g., DNA or RNA, which has been removed from its native environment. For example, a nucleic acid molecule comprising a polynucleotide encoding a recombinant polypeptide contained in a vector is considered “isolated” for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include polynucleotides and nucleic acids (e.g., nucleic acid molecules) produced synthetically.

As used herein, a “coding region” or “coding sequence” is a portion of a polynucleotide, which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino-terminus of the resultant polypeptide, and a translation stop codon at the 3′ terminus, encoding the carboxyl-terminus of the resulting polypeptide.

As used herein, an “expression cassette” comprises a nucleic acid sequence of interest (e.g., a nucleic acid sequence for expression of a polypeptide, DNA, or RNA) and an expression control region.

As used herein, a “transgene” can be used interchangeably with “gene of interest (GOI)” and refers to a portion of a polynucleotide that contains codons translatable into amino acids. Although a “stop codon” (TAG, TGA, or TAA) is typically not translated into an amino acid, it can be considered to be part of a transgene, but any flanking sequences, for example promoters, ribosome binding sites, transcriptional terminators, introns, and the like, are not part of the transgene. The boundaries of a transgene are typically determined by a start codon at the 5′ terminus, encoding the amino-terminus of the resultant polypeptide, and a translation stop codon at the 3′ terminus, encoding the carboxyl-terminus of the resulting polypeptide.

As used herein, the term “expression control region” refers to a transcription control element that is operably associated with a coding region to direct or control expression of the product encoded by the coding region, including, for example, cis-regulatory modules (CRMs), promoters (e.g., a tissue specific promoter and/or an inducible promoter), enhancers, operators, repressors, ribosome binding sites, translation leader sequences, introns, post-transcriptional elements, polyadenylation recognition sequences, RNA processing sites, effector binding sites, stem-loop structures, and transcription termination signals, miRNA binding sites, and combinations thereof. Expression control regions include nucleotide sequences located upstream (5′), within, or downstream (3′) of a nucleic acid sequence of interest, and which influence the transcription, RNA processing, stability, or translation of the associated nucleic acid sequence of interest. If a transgene is intended for expression in a eukaryotic cell, a polyadenylation signal and transcription termination sequence will usually be located 3′ to the transgene.

A coding region and a promoter are “operably associated” (i.e., “operably linked”) if induction of promoter function results in the transcription of mRNA comprising a coding region that encodes the product, and if the nature of the linkage between the promoter and the coding region does not interfere with the ability of the promoter to direct the expression of the product encoded by the coding region or interfere with the ability of the DNA template to be transcribed. Expression control regions include nucleotide sequences located upstream (5′ non-coding sequences), within, or downstream (3′ non-coding sequences) of a coding region, and which influence the transcription, RNA processing, stability, or translation of the associated coding region. If a coding region is intended for expression in a eukaryotic cell, a polyadenylation signal and transcription termination sequence will usually be located 3′ to the coding sequence.

As used herein, the terms “host cell” and “cell” can be used interchangeably and can refer to any type of cell or a population of cells, e.g., a primary cell, a cell in culture, or a cell from a cell line, that harbors or is capable of harboring a nucleic acid molecule (e.g., a recombinant nucleic acid molecule). Host cells can be a prokaryotic cell, or alternatively, the host cells can be eukaryotic, for example, fungal cells, such as yeast cells, and various animal cells, such as insect cells or mammalian cells.

“Culture,” “to culture” and “culturing,” as used herein, means to incubate cells under in vitro conditions that allow for cell growth or division or to maintain cells in a living state. “Cultured cells,” as used herein, means cells that are propagated in vitro.

A “subject” includes any human or nonhuman animal. The term “nonhuman animal” includes, but is not limited to, vertebrates such as mammals, avians, pets, farm animals, nonhuman primates, sheep, cows, goats, pigs, chickens, dogs, cats, and rodents such as mice, rats, and guinea pigs. In preferred aspects, the subject is a human. The terms, “subject” and “patient” are used interchangeably herein.

“Administering” refers to the physical introduction of a therapeutic agent to a subject, using any of the various methods and delivery systems known to those skilled in the art.

The terms “treat,” “treating,” “treatment,” or “therapy” of a subject as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis, such as vaccination).

The term “effective dose” “effective dosage,” or “effective amount” is defined as an amount of an agent sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Various aspects of the disclosure are described in further detail in the following subsections.

### II. Expression Vectors and Vector Production Systems for Producing Bacterial Sequence-Free Vectors

Bacterial sequence-free vectors and their production are described in U.S. Pat. Nos. 9,290,778 and 9,862,954; Nafissi and Slavcev, Microbial Cell Factories 11:154 (2012); and Nafissi et al., Nucleic Acids 3(6):e165 (2014), incorporated by reference herein in their entireties. These bacterial sequence-free vectors are produced from an expression vector (e.g., a plasmid) that contains specialized “Super Sequence” (“SS” or, alternatively, “SSeq”) sites comprising target sequences for recombinases flanking each side (i.e., the 5′ and 3′ sides) of an expression cassette containing a nucleic acid sequence(s) of interest. Specifically, each SS contains a target sequence for a first recombinase, with an additional target sequence for one or more additional recombinases integrated within non-binding regions for the first recombinase. When the expression vector is present in a recombinant cell that expresses an appropriate recombinase, a bacterial sequence-free vector containing the expression cassette is separated from the backbone DNA of the expression vector. To produce a circular covalently closed (CCC) bacterial sequence-free vector, the expression vector is placed into a recombinant cell expressing a recombinase such as Cre or Flp, for example, that acts through its target sequences in the SS. To produce a linear covalently closed (LCC) bacterial sequence-free vector, also referred to herein as a ministring DNA (msDNA), the expression vector is placed into a recombinant cell expressing a recombinase such as TelN or Tel, for example, that acts through its target sequences in the SS. The bacterial sequence-free vector resulting from the recombination can then be purified from the cells and used directly as a delivery vector. See U.S. Pat. Nos. 9,290,778 and 9,862,954, Nafissi and Slavcev, and Nafissi et al.

msDNA vectors with LCC ends are torsion-free and not subject to gyrase-directed negative supercoiling during their production in E. coli. Furthermore, due to its double stranded LCC topology, integration of msDNA into a cell's chromosome causes a chromosomal break, thereby eliminating the cell from the population. Thus, msDNA eliminates any risk of insertional mutagenesis, protecting patients who are administered the msDNA from potential genotoxicity and cancer (Nafissi et. al.).

The present disclosure provides improved production of bacterial sequence-free vectors and improved bacterial sequence-free vectors. In some aspects, production of the bacterial sequence-free vectors is improved by removal of contaminating expression vector sequences. In some aspects, the bacterial sequence-free vectors is improved through its capacity for establishment in cells (i.e., transfection efficiencies), improved transgene expression (e.g., mediated by a combination of enhanced transcription and translation), and improved expansion in cells (e.g., replication and partition of the vector to daughter cells).

In some aspects, the improvements disclosed herein can be adapted to CCC or LCC vectors produced according to other methods known in the art.

### A. Expression Vectors

Provided herein is an expression vector comprising: (a) a backbone sequence, (b) a sequence comprising: (i) an expression cassette comprising a nucleic acid sequence of interest, (ii) a first target sequence for a first recombinase flanking the 5′ side of the expression cassette, (iii) a second target sequence for the first recombinase flanking the 3′ side of the expression cassette, and (iv) one or more additional target sequences for one or more additional recombinases integrated within the first and second target sequences in non-binding regions for the first recombinase, and (c) one or more of: (i) an endonuclease target sequence integrated within the first and/or second target sequences for the first recombinase in non-binding regions for the first recombinase and the one or more additional recombinases, wherein the endonuclease target sequence is between the backbone sequence and cleavage sites for the first recombinase and the one or more additional recombinases, (ii) a synthetic enhancer comprising a nucleic acid sequence at least about 90% identical to SEQ ID NO: 12 integrated between the 3′ end of the first target sequence for the first recombinase and the 5′ end of another enhancer or a promoter in the expression cassette, (iii) a cytomegalovirus (CMV) enhancer integrated between the 3′ end of the first target sequence for the first recombinase and the 5′ end of a promoter in the expression cassette, (iv) a 5′ untranslated region (5′UTR) comprising an intron, wherein the 5′UTR is integrated in the expression cassette between a promoter and the nucleic acid sequence of interest, (v) a vertebrate chromatin insulator integrated in the expression cassette between the nucleic acid of interest and a polyadenylation signal, (vi) a woodchuck hepatitis virus post-transcriptional regulatory element (WPRE) integrated in the expression cassette between the nucleic acid of interest and a polyadenylation signal, (vii) a scaffold/matrix attachment region (S/MAR) integrated in the expression cassette between the nucleic acid of interest and a polyadenylation signal, or (viii) a DNA nuclear targeting sequence (DTS) integrated within the first and/or second target sequences for the first recombinase in non-binding regions for the first recombinase and the one or more additional recombinases, wherein the DTS is between the expression cassette and cleavage sites for the first recombinase and the one or more additional recombinases.

A “backbone” sequence as referred to herein is the sequence of the expression vector outside of the sequence of the expression cassette and the flanking SS sites comprising the first and second target sequences of the first recombinase. The backbone sequence can include, for example, sequences for amplification and antibiotic selection of the expression vector in a host cell (e.g., E. coli) as described herein.

“Non-binding” regions for a recombinase are regions within the target sequence for the first recombinase that are not acted upon by a recombinase as described herein (e.g., not bound and/or cleaved by the recombinase).

A “cleavage site” for a recombinase is the site at which a recombinase initiates a double-strand break or single-stranded nick in the DNA associated with recombination.

In some aspects, the expression vector comprises an endonuclease target sequence integrated within the first and/or second target sequences for the first recombinase in non-binding regions for the first recombinase and the one or more additional recombinases, wherein the endonuclease target sequence is between the backbone sequence and cleavage sites for the first recombinase and the one or more additional recombinases. In some aspects, the endonuclease target sequence is integrated within the first target sequence for the first recombinase. In some aspects, the endonuclease target sequence is integrated within the second target sequence for the first recombinase. In some aspects, the endonuclease target sequence is integrated within the first and second target sequences for the first recombinase. In some aspects, the same endonuclease target sequence is integrated within the first and second target sequences for the first recombinase. In some aspects, the endonuclease target sequences integrated within the first and second target sequences for the first recombinase are for the same endonuclease. In some aspects, the endonuclease target sequence integrated within the first target sequence for the first recombinase is different from the endonuclease target sequence integrated within the second target sequence for the first recombinase. In some aspects, the endonuclease target sequence integrated within the first target sequence for the first recombinase is for a different endonuclease than the endonuclease target sequence integrated within the second target sequence for the first recombinase.

The location of the endonuclease target sequence between the backbone sequence and cleavage sites for the recombinases in the expression vector ensures that the endonuclease target sequence remains associated with the backbone sequence, and not the bacterial sequence-free vector, following recombination as described herein. Thus, following recombination, sequences containing backbone sequence and the endonuclease target site can be removed from a preparation containing bacterial sequence-free vector by exposure to an endonuclease, reducing or avoiding the need for purification steps to remove backbone sequences in methods of producing the bacterial sequence-free vector. In some aspects, the endonuclease is expressed following recombination in a host cell of a vector production system as described herein, wherein the endonuclease cuts the DNA at the endonuclease target site, and the sequence containing the backbone sequence and the endonuclease target site is degraded by an exonuclease (e.g., exonuclease V).

In some aspects, the expression vector comprises an endonuclease target sequence for a homing endonuclease. In some aspects, the endonuclease target sequence is for I-AniI, I-CeuI, I-ChuI, I-CpaI, I-CpaII, I-CreI, I-DmoI, H-DreI, I-HmuI, I-HmuII, I-LlaI, I-MsoI, PI-PfuI, PI-PkoII, I-PorI, I-PpoI, PI-PspI, I-ScaI, I-SceI, PI-SceI, I-SceII, I-SecIII, I-SceIV, I-SceV, I-SceVI, I-SceVII, I-Ssp6803I, I-TevI, I-TevII, I-TevIII, PI-TliI, PI-TliII, I-Tsp061I, or I-Vdi141I. In some aspects, the endonuclease target sequence is for I-SceI. In some aspects, the endonuclease target sequence is for PI-SceI. Target sequences for homing endonucleases are well-known in the art.

In some aspects, the expression vector comprises an endonuclease target sequence for an endonuclease used in genome editing, including an endonuclease that is part of a nuclease genome editing system. In some aspects, the nuclease genome editing system is a Clustered Regularly Interspaced Short Palindromic Repeats-Cas (CRISPR-Cas) system, a Transcription Activator-Like Effector Nuclease (TALEN) system, a Zinc-Finger Nuclease (ZFN) system, or a meganuclease system.

In some aspects, the expression vector comprises an endonuclease target sequence for a Cas endonuclease. In some aspects, the Cas endonuclease is Cas9 (e.g., a Streptococcus pyogenes Cas 9 (SpCas9), a Staphylococcus aureus Cas9 (SaCas9), a Francisella novicida Cas9 (FnCas9), or a Neisseria meningitides Cas9 (NmCas9)), a Cas9 variant (e.g., Cas9β2, xCas9, SpCas9-NG, SpCas9-NRRH, SpCas9-NRCH, SpCas9-NRTH, SpG, SpRY), Cas3, Cas12 (e.g., Cas12a, Cas12b, Cas12c, Cas12d, or Cas12e), Cas13 (e.g., Cas13a, Cas13b, Cas13c, or Cas13d), or Cas14. In some aspects, an endonuclease target sequence for a Cas endonuclease as used herein is homologous to a guide RNA (gRNA) targeting sequence and includes a protospacer adjacent motif (PAM) recognized by a Cas endonuclease. Sequences homologous to gRNA targeting sequences with PAM sites can be routinely designed based on well-known CRISPR systems. The gRNA comprises a fusion of a targeting RNA (crRNA) sequence and a trans-activating RNA (tracrRNA) sequence, which interact and function to direct the Cas endonuclease to the endonuclease target site and catalyze cleavage.

In some aspects, the expression vector comprises a synthetic enhancer comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO: 12 integrated between the 3′ end of the first target sequence for the first recombinase and the 5′ end of another enhancer or a promoter in the expression cassette. In some aspects, the expression vector comprises a synthetic enhancer comprising the nucleic acid sequence of SEQ ID NO:12 integrated between the 3′ end of the first target sequence for the first recombinase and the 5′ end of another enhancer or a promoter in the expression cassette. In some aspects, the synthetic enhancer comprises multiple contiguous copies of the nucleic acid sequence, such as, for example, 1, 2, 3, 4, 5, or more contiguous copies. In some aspects, the synthetic enhancer comprises 3 contiguous copies of the nucleic acid sequence. In some aspects, the synthetic enhancer comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:46. In some aspects, the synthetic enhancer comprises the nucleic acid sequence of SEQ ID NO:46. In some aspects, the synthetic enhancer is integrated at the 5′ end of a chicken β-actin promoter. In some aspects, a chimeric intron comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:47 is integrated at the 3′ end of the chicken β-actin promoter and 5′ to the nucleic acid sequence of interest. In some aspects, a chimeric intron comprising the nucleic acid sequence of SEQ ID NO:47 is integrated at the 3′ end of the chicken β-actin promoter and 5′ to the nucleic acid sequence of interest.

In some aspects, the expression vector comprises a CMV enhancer integrated between the 3′ end of the first target sequence for the first recombinase and the 5′ end of a promoter in the expression cassette. In some aspects, the CMV enhancer is integrated at the 3′ end of a synthetic enhancer comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:12. In some aspects, the CMV enhancer is integrated at the 3′ end of a synthetic enhancer comprising the nucleic acid sequence of SEQ ID NO: 12. In some aspects, the CMV enhancer is integrated at the 3′ end of multiple contiguous copies of the synthetic enhancer, such as, for example, at the 3′ end of 1, 2, 3, 4, 5, or more contiguous copies of the synthetic enhancer. In some aspects, the CMV enhancer is integrated at the 3′ end of 3 contiguous copies of the synthetic enhancer. In some aspects, the CMV enhancer is integrated at the 3′ end of a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:46. In some aspects, the CMV enhancer is integrated at the 3′ end of the nucleic acid sequence of SEQ ID NO:46. In some aspects, a CMV promoter is integrated at the 3′ end of the CMV enhancer and 5′ to the nucleic acid sequence of interest.

In some aspects, the expression vector comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:35, SEQ ID NO:36, SEQ ID NO:37, SEQ ID NO:38, or SEQ ID NO:39 integrated between the first target sequence for the first recombinase and the nucleic acid sequence of interest. In some aspects, the expression vector comprises the nucleic acid sequence of SEQ ID NO:35, SEQ ID NO:36, SEQ ID NO:37, SEQ ID NO:38, or SEQ ID NO:39 integrated between the first target sequence for the first recombinase and the nucleic acid sequence of interest. In some aspects, a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:35, SEQ ID NO:36, SEQ ID NO:37, SEQ ID NO:38, or SEQ ID NO:39, or the nucleic acid sequence of SEQ ID NO:35, SEQ ID NO:36, SEQ ID NO:37, SEQ ID NO:38, or SEQ ID NO:39, comprises all regulatory elements in the expression cassette located 5′ to the nucleic acid sequence of interest.

In some aspects, the expression vector comprises a 5′UTR comprising an intron, wherein the 5′UTR (i.e., the 5′UTR comprising the intron) is integrated in the expression cassette between a promoter and the nucleic acid sequence of interest.

In some aspects, the 5′UTR is for improving transgene transcript splicing and translation from the expression vector or from a bacterial sequence-free vector produced from the expression vector as compared to the same expression vector or bacterial sequence-free vector, respectively, lacking the 5′UTR.

In some aspects, the intron comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:1. In some aspects, the intron comprises the nucleic acid sequence of SEQ ID NO:1.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:2, which is an optimized 5′UTR with an internal minimal intron, also referred to herein as “5′UTR1.” In some aspects, the 5′UTR comprises the nucleic acid sequence of SEQ ID NO:2.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:4. In some aspects, the 5′UTR comprises the nucleic acid sequence of SEQ ID NO:4.

In some aspects, the 5′UTR further comprises a non-coding sequence integrated within the intron.

In some aspects, the intron is at least about 90%, at least about 91%, at least about 92%, at least about 93%, at least about 94%, at least about 95%, at least about 96%, at least about 97%, at least about 98%, or at least about 99% identical to SEQ ID NO:1, or comprises SEQ ID NO: 1, and the non-coding sequence is integrated between two of the nucleotides in the intron corresponding to any two nucleotides from positions 25 to 55 of SEQ ID NO:1.

In some aspects, the non-coding sequence is non-prokaryotic and non-viral. In some aspects, the non-coding sequence is a eukaryotic sequence. In some aspects, the non-coding sequence comprises an intron, a ubiquitous chromatin opening element (UCOE), an S/MAR, an SV40 enhancer sequence (e.g., one or more than one SV40 enhancer sequences, such as two, three, four, five or more SV40 enhancer sequences), a vertebrate chromatin insulator (e.g., cHS4), a WPRE, or any combination thereof.

In some aspects, the non-coding sequence comprises an S/MAR. In some aspects, the S/MAR is MAR-5, provided herein as SEQ ID NO:9.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:3. In some aspects, the 5′UTR comprises SEQ ID NO:3.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:5. In some aspects, the 5′UTR comprises SEQ ID NO:5.

In some aspects, the 5′UTR is integrated in the expression cassette between a chicken β-actin promoter and the nucleic acid sequence of interest.

In some aspects, the 5′UTR is integrated in the expression cassette between a CMV promoter and the nucleic acid sequence of interest.

In some aspects, the 5′UTR is integrated in the expression cassette between a promoter and the nucleic acid sequence of interest, wherein the promoter is integrated at the 3′ end of a CMV enhancer. In some aspects, the CMV enhancer is integrated at the 3′ end of a synthetic enhancer comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:12. In some aspects, the CMV enhancer is integrated at the 3′ end of a synthetic enhancer comprising the nucleic acid sequence of SEQ ID NO: 12. In some aspects, the CMV enhancer is integrated at the 3′ end of multiple contiguous copies of the synthetic enhancer, such as, for example, at the 3′ end of 1, 2, 3, 4, 5, or more contiguous copies of the synthetic enhancer. In some aspects, the CMV enhancer is integrated at the 3′ end of 3 contiguous copies of the synthetic enhancer. In some aspects, the CMV enhancer is integrated at the 3′ end of a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:46. In some aspects, the CMV enhancer is integrated at the 3′ end of a nucleic acid sequence of SEQ ID NO:46.

In some aspects, the expression vector comprises a polyadenylation signal integrated at the 3′ end of the nucleic acid sequence of interest. In some aspects, the polyadenylation signal comprises a Xenopus laevis beta-globin polyadenylation signal, a human beta-globin polyadenylation signal, or a hybrid Xenopus laevis and human beta-globin polyadenylation signal. In some aspects, the polyadenylation signal comprises multiple copies of a Xenopus laevis beta-globin polyadenylation signal, a human beta-globin polyadenylation signal, or a hybrid Xenopus laevis and human beta-globin polyadenylation signal, such as, for example, 1, 2, 3, 4, or 5 copies. In some aspects, the polyadenylation signal comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO: 13, SEQ ID NO: 14, or SEQ ID NO:15. In some aspects, the polyadenylation signal comprises the nucleic acid sequence of SEQ ID NO:13, SEQ ID NO:14, or SEQ ID NO:15. In some aspects, a polyadenylic acid tail (i.e., poly(A) tail is located at the 3′ end of the polyadenylation signal. In some aspects, the poly(A) tail is 100, 101, 102, 103, 104, 105, 106, 107, 108, 109, 110, 111, 112, 113, 114, 115, 116, 117, 118, 119, 120, or more residues in length. In some aspects, the sequence comprising the polyadenylation signal and the poly(A) tail is at least about 90%, at least about 91%, at least about 92%, at least about 93%, at least about 94%, at least about 95%, at least about 96%, at least about 97%, at least about 98%, or at least about 99% identical to SEQ ID NO:16, SEQ ID NO:17, or SEQ ID NO:18. In some aspects, the sequence comprising the polyadenylation signal and the poly(A) tail comprises SEQ ID NO: 16, SEQ ID NO:17, or SEQ ID NO:18.

In some aspects, the expression vector comprises a vertebrate chromatin insulator in the expression cassette. In some aspects, the vertebrate chromatin insulator is 5′-HS4 chicken-β-globin insulator (cHS4). See, e.g., Benabdellah et al., PLOS ONE 9(1): e84268 (2014); Lu et al., FEBS Open Bio 10: 644-656 (2020); Hanawa et al., Mol. Ther. 17(4): 667-674 (2009); Walters et al., Mol. Cell. Biol. 19(5): 3714-3726 (1999). In some aspects, the vertebrate chromatin insulator is integrated in the expression cassette between the nucleic acid of interest and a polyadenylation signal as described herein. In some aspects, the vertebrate chromatin insulator is integrated within the intron of a 5′UTR as described herein.

In some aspects, the vertebrate chromatin insulator comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:8. In some aspects, the vertebrate chromatin insulator comprises SEQ ID NO:8.

In some aspects, the vertebrate chromatin insulator is for improving establishment (i.e., transfection efficiency) of the expression vector or a bacterial sequence-free vector produced from the expression vector as compared to the same expression vector or bacterial sequence-free vector, respectively, without the vertebrate chromatin insulator.

In some aspects, the expression vector comprises a WPRE in the expression cassette. See, e.g., Higashimoto et al., Gene Therapy 14: 1298-1304 (2007). In some aspects, the WPRE is integrated in the expression cassette between the nucleic acid of interest and a polyadenylation signal as described herein. In some aspects, the WPRE is integrated in the expression cassette at the 3′ end of a S/MAR as described herein and the 5′ end of a polyadenylation signal as described herein. In some aspects, the WPRE is integrated within the intron of a 5′UTR as described herein.

In some aspects, the WPRE comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:11. In some aspects, the WPRE comprises SEQ ID NO:11.

In some aspects, the WPRE improves expression of the transgene from the expression vector or the bacterial sequence-free vector produced from the expression vector as compared to the same expression vector or bacterial sequence-free vector, respectively, lacking the WPRE.

In some aspects, the expression vector comprises a S/MAR in the expression cassette. See, e.g., Martens et al., Mol. Cell. Biol. 22(8): 2598-2606 (2002); Narwade et al., Nucleic Acids Res. 47(14): 7247-7261 (2019). In some aspects, the S/MAR is integrated in the expression cassette between the nucleic acid of interest and a polyadenylation signal. In some aspects, the S/MAR is integrated in the expression cassette at the 3′ end of a nucleic acid sequence of interest and the 5′ end of a WPRE as described herein. In some aspects, the S/MAR is integrated within the intron of a 5′UTR as described herein.

In some aspects, the S/MAR is MAR-3, MAR-4, or MAR-5, which are fragments of human beta-interferon MAR. See, e.g., Wang et al., Mol. Biol. Cell 30: 2761-2770 (2019). In some aspects, the S/MAR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:9. In some aspects, the S/MAR comprises SEQ ID NO:9.

In some aspects, the S/MAR is human cytotoxic serine protease-B (CSP-B) MAR or CSP-C MAR. See, e.g., Hanson and Ley, Blood 79(3): 610-618 (1992); Klein et al., Tissue Antigens 35(5):220-228 (1990). In some aspects, the S/MAR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:10. In some aspects, the S/MAR comprises SEQ ID NO:10.

In some aspects, the S/MAR is for improving expression levels, stability, and/or durability (e.g., by episomal maintenance and replication, such as expansion and partition of the vector to daughter cells, and/or by preventing epigenetic silencing) of the expression vector or a bacterial sequence-free vector (produced from the expression vector as compared to the same expression vector or bacterial sequence-free vector, respectively, lacking the S/MAR.

In some aspects, the expression vector comprising any one of more of (c)(i)-(c)(vii) as described above (i.e., without a DTS) further comprises an enhancer sequence flanking each side of the first and second target sequences for the first recombinase. In some aspects, the enhancer sequence flanking each side of the first and second target sequences for the first recombinase is at least two enhancer sequences flanking each side of the first and second target sequences for the first recombinase. In some aspects, the enhancer sequence is a SV40 enhancer sequence.

In some aspects, the expression vector comprises a DTS. In some aspects, the DTS is integrated within the first and/or second target sequences for the first recombinase in non-binding regions for the first recombinase and the one or more additional recombinases, wherein the DTS is between the expression cassette and cleavage sites for the first recombinase and the one or more additional recombinases. In some aspects, the DTS is a SV40 enhancer sequence. In some aspects, the DTS is cell-specific. In some aspects, the DTS is specific for smooth muscle cells, embryonic stem cells, type II pneumonocytes, endothelial cells, or osteoblasts.

The location of the DTS between the expression cassette and cleavage sites for the recombinases in the expression vector ensures that the DTS remains associated with the bacterial sequence-free vector, and not the backbone sequence, following recombination as described herein.

In some aspects, the expression vector comprises a UCOE in the expression cassette. See, e.g., Müller-Kuller et al., Nucleic Acids Res. 43(3): 1577-1592 (2015); Skipper et al., BMC Biotechnol. 19:75 (2019); Rudina et al., bioRxiv, doi.org/10.1101/626713 (2019); Neville et al., Biotechnol. Adv. 35(5): 557-564 (2017). In some aspects, the UCOE is located between the 3′ end of the first target sequence for the first recombinase and the 5′ end of a promoter or any enhancer in the expression cassette. In some aspects, the UCOE is integrated within the intron of a 5′UTR as described herein.

In some aspects, the UCOE is A2UCOE. In some aspects, the UCOE comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:6. In some aspects, the UCOE is SEQ ID NO:6.

In some aspects, the UCOE is SRF-UCOE. See, e.g., International Publication No. WO2020223160. In some aspects, the UCOE comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:7. In some aspects, the UCOE is SEQ ID NO:7.

In some aspects, the UCOE improves expression of the transgene from the expression vector or a bacterial sequence-free vector produced from the expression vector as compared to the same expression vector or bacterial sequence-free vector, respectively, lacking the UCOE.

In some aspects, the expression vector comprises Enhancer-1 in the expression cassette. In some aspects, Enhancer-1 is integrated between the 3′ end of the first target sequence for the first recombinase and the 5′ end of a promoter or any other enhancer in the expression cassette. In some aspects, Enhancer-1 is integrated between the 3′ end of a UCOE and the 5′ end of a CMV enhancer. In some aspects, Enhancer-1 comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO: 12. In some aspects, Enhancer-1 is SEQ ID NO: 12.

In some aspects, the expression vector comprises a CMV, EF1, SV40, CAG, Rho, VDM2, HCR, or HLP promoter, or variant thereof, in the expression cassette. In some aspects, the expression vector comprises a CMV promoter variant in the expression cassette. See, e.g., International Publication No. WO2012099540; Xu et al., Bioengineered 10(1): 548-560, DOI: 10.1080/21655979.2019.1684863 (2019).

In some aspects, the expression vector comprises an EF1-alpha promoter in the expression cassette. In some aspects, the expression vector comprises a CMV enhancer and an EF1-alpha promoter in the expression cassette.

In some aspects, the expression vector comprises a 3′UTR in the expression cassette comprising two copies of a beta-globin polyadenylation signal. In some aspects, the 3′UTR is integrated between the nucleic acid sequence of interest and the 5′ end of the second target sequence for the first recombinase.

In some aspects, the 3′UTR comprises two copies of a Xenopus laevis beta-globin polyadenylation signal. In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:13. In some aspects, the 3′UTR is SEQ ID NO:13.

In some aspects, the 3′UTR comprises two copies of a human beta-globin polyadenylation signal. In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:14. In some aspects, the 3′UTR is SEQ ID NO:14.

In some aspects, the 3′UTR comprises one copy of a Xenopus laevis beta-globin polyadenylation signal and one copy of a human beta-globin polyadenylation signal. In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:15. In some aspects, the 3′UTR is SEQ ID NO:15.

In some aspects, the 3′UTR further comprises a poly(A) tail (i.e., at the 3′ end of the 3′UTR) comprising 100 to 120 adenine nucleotides, i.e., 100, 101, 102, 103, 104, 105, 106, 107, 108, 109, 110, 111, 112, 113, 114, 115, 116, 117, 118, 119, or 120 adenine nucleotides.

In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:16. In some aspects, the 3′UTR is SEQ ID NO:16.

In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:17. In some aspects, the 3′UTR is SEQ ID NO:17.

In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:18. In some aspects, the 3′UTR is SEQ ID NO:18.

The expression vector can contain any combination of the above modifications to the first and/or second target sequences and/or the expression cassette as described herein. In some aspects, the combination provides a synergistic effect.

In some aspects, the first and second target sequences for the first recombinase and the one or more additional target sequences for the one or more additional recombinases are selected from the group consisting of the PY54 pal site, the N15 telRL site, the loxP site, φK02 telRL site, the FRT site, the phiC31 attP site, and the λ attP site. In some aspects, the expression vector comprises each of the target sequences. In some aspects, the expression vector comprises the pal site and the telRL, loxP, and FRT recombinase target binding sequences integrated within the pal site. In some aspects, the first and second target sequences for the first recombinase each comprise the nucleic acid sequence of SEQ ID NO:33.

In some aspects, the nucleic acid sequence of interest in any of the expression cassettes described herein comprises a sequence encoding: a polypeptide, an RNA (messenger RNA (mRNA), micro-RNA (miRNA), small interfering RNA (siRNA), small hairpin RNA (shRNA), ribozyme, or antisense RNA), or a non-coding DNA (e.g., an antisense oligonucleotide). In some aspects, the nucleic acid sequence of interest is a genomic DNA sequence comprising introns and/or exons. In some aspects, the nucleic acid sequence of interest comprises a sequence encoding: an anti-cancer agent, a tumor suppressor, an apoptotic agent, an anti-angiogenesis agent, an enzyme, a cytotoxic agent, a suicide gene, a cytokine, an interferon, an interleukin, an immunomodulatory agent, an immunostimulatory agent, an immunoinhibitory agent, a chemokine, an antigen for stimulating an antigen-presenting cell, an antibody (e.g., a heavy chain and/or a light chain of an antibody, such as a monoclonal, chimeric, humanized, or human antibody, or an antigen-binding fragment thereof), a genome editing system or a portion thereof (e.g., CRISPR-Cas, TALEN, ZFN, or meganuclease systems or portions thereof, such as a Cas endonuclease or a gRNA), or an immunogenic agent (e.g., as a VLP and/or vaccine). In some aspects, the nucleic acid sequence of interest comprises sequences encoding polypeptides that are capable of forming a VLP when the nucleic acid sequence is expressed intracellularly.

Exemplary therapeutic targets and indications include: a gene associated with a monogenic disorder, including, for example, a liver, blood, or eye disorder, galactosidase alpha (GLA, e.g., for treating Fabry disease), sodium voltage-gated channel alpha subunit 1 (SCNIA, e.g., for treating dravet syndrome), ATP binding cassette subfamily A member 4 (ABCA4, e.g., for treating Stargardt disease), surfactant protein B (SP-B, e.g., for treating surfactant dysfunction disorder), surfactant protein C (SP-C, e.g., for treating surfactant dysfunction disorder), ATP-binding cassette sub-family A member 3 (ABCA3, e.g., for treating surfactant dysfunction disorder), solute carrier family 34 member 2 (SLC34A2, e.g., for treating pulmonary alveolar microlithiasis and/or testicular microlithiasis), cystic fibrosis transmembrane conductance regulator (CFTR, e.g., for treating cystic fibrosis), glutamate decarboxylase (GAD, e.g., GAD65 or GAD67, e.g., for treating Parkinson's disease), aspartoacylase gene (ASPA, also known as aminoacylase (AAC), e.g., for treating Canavan disease), aromatic L-amino acid decarboxylase (AADC, e.g., for treating Parkinson's disease and/or for treating AADC deficiency), neurturin (NRTN, e.g., for treating Parkinson's disease), glial cell line-derived neurotrophic factor (GDNF, e.g., for treating Parkinson's disease), nerve growth factor (NGF, e.g., for treating Alzheimer's disease), tripeptidyl peptidase I (TPP1, also known as ceroid lipofuscinosis neuronal-2 (CLN2), e.g., for treating Batten disease, e.g., CLN2 disease), arylsulfatase A (ARSA, e.g., for treating metachromatic leukodystrophy), N-sulphoglucosamine sulphohydrolase (SGSH, e.g., for treating Sanfilippo syndrome, Type A), Sulfatase-modifying factor 1 (SUMF1, e.g., for treating Sanfilippo syndrome, Type A), N-acetyl-alpha-glucosaminidase (NAGLU, e.g., for treating Sanfilippo syndrome, Type B), survival of motor neuron 1 (SMN1, e.g., for treating spinal muscular atrophy 1), retinal pigment epithelium-specific 65 kDa protein (RPE65, also known as retinoid isomerohydrolase, e.g., for treating Leber's congenital amaurosis), Rab escort protein 1 (REP1, e.g., for treating choroideremia), retinoschisin 1 (RS1, e.g., for treating X-linked juvenile retinoschisis), alpha-1 antitrypsin (AAT, e.g., for treating hereditary emphysema or AAT deficiency), minidystrophin (e.g., for treating Duchenne's muscular dystrophy), α-sarcoglycan (αSG, e.g., for treating Duchenne's muscular dystrophy or limb girdle muscular dystrophy type 2), β-sarcoglycan (BSG), γ-sarcoglycan (γSG, e.g., for treating limb girdle muscular dystrophy type 2), δ-sarcoglycan (γSG), ipoprotein lipase (LPL, e.g., for treating familial LPL deficiency), acid alpha-glucosidase (GAA, e.g., for treating Pompe disease), tumor necrosis factor receptor:Fc (TNFR:Fc, e.g., for treating arthritis, e.g., inflammatory arthritis), sarcoplasmic/endoplasmic reticulum Ca(2+)ATPase 2a (SERCA2a, e.g., for treating congestive heart failure), Factor VIII or Factor IX (FVIII or FIX, e.g., for treating hemophilia B), porphobilinogen deaminase gene (PBGD, e.g., for treating acute intermittent porphyria), soluble fms-like tyrosine kinase-1 (sFLT1, e.g., for treating age-related macular degeneration or cancer, e.g., ovarian cancer), a soluble chimeric vascular endothelial growth factor (VEGF) receptor comprising domains of VEGFR-1 and VEGF-R2 (e.g., for treating cancer, e.g., melanoma or colon cancer), soluble VEGFR3 (e.g., for treating cancer, e.g., endometrial cancer), a soluble VEGF-C decoy receptor (sVEGFR3-Fc, e.g., for treating cancer, e.g., melanoma, renal cell carcinoma, or prostate cancer), pigment epithelium-derived growth factor (PEDF, e.g., for treating cancer, e.g., Lewis lung carcinoma), a neutralizing monoclonal antibody against VEGFR2 (e.g., DC101, e.g., for treating cancer, e.g., melanoma or glioblastoma), endostatin (e.g., for treating cancer, e.g., bladder or pancreatic cancer), angiostatin (e.g., for treating cancer, e.g., liver cancer), both endostatin and angiostatin (i.e., as a bicistronic sequence, e.g., for treating cancer, e.g., ovarian or prostate cancer), an endostatin mutant (i.e., P1254A-endostatin, e.g., for treating cancer, e.g., ovarian cancer), antiangiogenic domain of TSP-1 (3TSR, e.g., for treating cancer, e.g., pancreatic cancer), tissue factor pathway inhibitor-2 (TFPI-2, e.g., for treating cancer, e.g., glioblastoma), a fragment of plasminogen (e.g., kringle 5, e.g., for treating cancer, e.g., ovarian cancer), plasminogen kringle 1-5 (e.g., for treating cancer, e.g., melanoma or lung cancer), siRNA against an unfolded protein response protein (UPR; e.g., IRE1α, XBP-1, or ATF6, e.g., for treating cancer, e.g., breast cancer), vasostatin (e.g., for treating cancer, e.g., lung cancer), herpes simplex virus type 1 thymidine kinase (HSV-TK, e.g., for treating cancer, e.g., breast cancer), sc39TK (e.g., for treating cancer, e.g., cervical cancer), diphtheria toxin A (DTA, e.g., for treating cancer, e.g., cervical cancer or myeloma), p53 upregulated modulator of apoptosis (PUMA, e.g., for treating cancer, e.g., cervical cancer or myeloma), tumor necrosis factor (TNF)-related apoptosis-inducing ligand (TRAIL, e.g., for treating cancer, e.g., lymphoma, hepatocellular carcinoma, head and neck squamous cell carcinoma (i.e., head and neck cancer), or glioblastoma), soluble TRAIL (e.g., for treating cancer, e.g., liver cancer or lung adenocarcinoma), IFN-β (e.g., for treating cancer, e.g., colorectal cancer, lung cancer, neuroblastoma, or glioblastoma multiforme), IFN-α (e.g., for treating cancer, e.g., metastatic melanoma), a CD-40 ligand (CD40L) or CD40L mutant (e.g., for treating cancer, e.g., lung cancer), melanoma differentiation-associated gene-7 and interleukin 24 (mda-7 and IL24, e.g., for treating cancer, e.g., Ehrlich ascites tumor), apoptotin and IL24 (e.g., for treating cancer, e.g., liver cancer), IL24 (e.g., for treating cancer, e.g., mixed-lineage leukemia (MLL)/AF4 positive acute lymphoblastic leukemia (ALL)), IL15 (e.g., for treating cancer, e.g., metastatic hepatocellular carcinoma), secondary lymphoid tissue chemokine (SLC, e.g., for treating cancer, e.g., liver cancer), Nk4 (the N-terminal hairpin and subsequent four kringle domains of hepatocyte growth factor (HGF), e.g., for treating cancer, e.g., metastatic Lewis lung carcinoma), tumor necrosis factor superfamily member 14 (TNFSF14, also known as LIGHT, e.g., for treating cancer, e.g., cervical cancer), Granulocyte-macrophage colony-stimulating factor (GM-CSF, e.g., for treating cancer), TNF-α (e.g., for treating cancer, e.g., glioma), a dominant negative mutant of survivin (e.g., C84A or T34A, e.g., for treating cancer, e.g., colon or gastric cancer), the C-terminal fragment of the human telomerase reverse transcriptase (hTERTC27, e.g., for treating cancer, e.g., glioblastoma multiforme), maspin (e.g., for treating cancer, e.g., prostate cancer), nm23H1 (e.g., for treating cancer, e.g., metastatic ovarian cancer), kringle 1 domain of human hepatocyte growth factor (HGFK1, e.g., for treating cancer, e.g., colorectal carcinoma), anti-calcitonin ribozyme (e.g., for treating cancer, e.g., prostate cancer), eukaryotic translation initiation factor 4E-binding protein 1 (4EBP1, e.g., for treating cancer, e.g., lung cancer), C-X-C motif chemokine receptor 2 (CXCR2) C-tail sequence (e.g., for treating cancer, e.g., pancreatic cancer), alpha-tocopherol-associated protein (TAP, e.g., for treating cancer, e.g., prostate cancer), trichosanthin (e.g., for treating cancer, e.g., hepatocellular carcinoma), decorin (e.g., for treating cancer, e.g., glioblastoma multiforme), cathelicidin (e.g., for treating cancer, e.g., colon cancer), Niemann-Pcik type C2 (NPC2, e.g., for treating cancer, e.g., hepatocellular carcinoma), Mullerian inhibiting substance (MIS, e.g., for treating cancer, e.g., ovarian cancer), p53 (e.g., for treating cancer, e.g., bronchoalveolar cancer), shRNA against highly expressed in cancer 1 (Hec1, e.g., for treating cancer, e.g., glioma), shRNA against Epstein-Barr virus latent membrane protein-1 (EBV LMP-1, e.g., for treating cancer, e.g., nasopharyngeal cancer), anti-sense RNA against human papilloma virus 16 E7 oncogene (HPV16-E7, e.g., for treating cancer, e.g., cervical cancer), shRNA against androgen receptor (AR, e.g., for treating cancer, e.g., prostate cancer), siRNA against Snail (also known as SNA1, e.g., for treating cancer, e.g., pancreatic cancer), siRNA against Slug (i.e., the protein product of SNAI2, e.g., for treating cancer, e.g., cholangiocarcinoma (liver cancer)), shRNA against Four and a half LIM-only protein 2 (FHL2, e.g., for treating cancer, e.g., colon cancer), miR-26a (e.g., for treating cancer, e.g., hepatocellular carcinoma), HPV 16 structural protein L1 (HPV16-L1, e.g., for treating cancer, e.g., cervical cancer), HPV 16 E5, E6, and E7 oncogenes (HPV16 E5/E6/E7, e.g., for treating cancer, e.g., cervical cancer), B-cell leukemia/lymphoma 1 (BLC1) idiotype (e.g., for treating cancer, e.g., B cell leukemia/lymphoma 1), EBV LMP1 and LMP2 fused to heat shock protein (EBV LMP2/1-hsp, e.g., for treating cancer, e.g., nasopharyngeal carcinoma), carcinoembryonic antigen (CEA, e.g., for treating cancer, e.g., colon cancer), soluble form of B and T lymphocyte attenuator in combination with a heat shock protein (BTLA and HSP70, e.g., for treating cancer, e.g., melanoma pulmonary metastasis), HPV16-L1/E7 (e.g., for treating cancer, e.g., cervical cancer), HPV16-L1 (e.g., for treating cancer, e.g., cervical cancer), an anti-EGFR antibody (e.g., 14D1, e.g., for treating cancer, e.g., vulvar carcinoma), an anti-death receptor 5 (DR5) antibody (e.g., adximab, e.g., for treating cancer, e.g., liver or colon cancer), an anti-Enolase 1 (ENOI1) antibody (e.g., for treating cancer, e.g., pancreatic ductal adenocarcinoma), an anti-VEGFA antibody (e.g., bevacizumab, e.g., for treating cancer, e.g., metastatic lung cancer or ovarian cancer), the Mucin 1 (MUC1) antigen (e.g., for treating cancer, e.g., gastric cancer), or an aquaporin (e.g., hAQP1, e.g., for treating irradiation induced parotid salivary hypofunction, i.e., xerostomia).

In some aspects, the nucleic acid sequence of interest is for use in gene editing (e.g., gene therapy, including treatment of a genetic deficiency, disorder, or disease).

In some aspects, the nucleic acid sequence of interest is for insertion into a target site for gene editing (i.e., a site within a DNA or RNA sequence that is the target of gene editing). A target site for gene editing includes any genetic element, such as any cis element. In some aspects, the target site for gene editing is located within an exon of a gene, an intron of a gene, or a regulatory element of a gene.

In some aspects, the gene editing comprises an endonuclease. In some aspects, the endonuclease is associated with a genome editing system. In some aspects, the endonuclease is, for example, a homing endonuclease, a site-specific nuclease, a structure-guided nuclease, or an RNA-guided nuclease (e.g., a transposon-encoded RNA-guided nuclease).

In some aspects, the gene editing comprises a genome editing system that produces a double-strand break within the target site for gene editing. In some aspects, the genome editing system is a CRISPR-Cas, TALEN, ZFN, or meganuclease gene editing system.

In some aspects, the nucleic acid sequence of interest is inserted into the target site for gene editing by non-homologous end joining at the double-strand break. In some aspects, the double-strand break is produced by a CRISPR-Cas system. In some aspects, an expression vector as described herein comprises a Cas endonuclease target sequence (i.e., a sequence homologous to a gRNA targeting sequence) located between the first and second target sequences for the first recombinase and the nucleic acid sequence of interest (i.e., between the 5′ Super Sequence and the nucleic acid sequence of interest and between the 3′ Super Sequence and the nucleic acid sequence of interest), wherein the target site for gene editing (e.g., a target site in a chromosome) comprises the same Cas endonuclease target sequence. For example, processing of the Cas endonuclease target sequences flanking the nucleic acid sequence in a bacterial sequence-free vector (e.g., msDNA) produced from the expression vector results in removal of the Super Sequences, rendering a linear covalently closed bacterial sequence-free vector such as msDNA to instead be linear and open-ended, with reactive ends that are amenable to non-homologous end-joining events.

In some aspects, the nucleic acid sequence of interest is inserted into the target site for gene editing by homology-directed repair, which occurs through recombination between sequences flanking the double-strand break and homologous sequences associated with the nucleic acid sequence of interest.

In some aspects, the nucleic acid sequence of interest has sufficient homology with sequences flanking the double-strand break to support homology-directed repair.

In some aspects, the nucleic acid sequence of interest is flanked by 5′ and 3′ homology arms (i.e., sequences that have sufficient homology with sequences flanking the double-strand break to mediate homology-directed repair).

In some aspects, sufficient homology to mediate homology-directed repair comprises at least about 70%, at least about 75%, at least about 80%, at least about 85%, at least about 90%, at least about 95%, or about 100% homology between the nucleic acid sequence of interest and the sequences flanking the double-strand break or between homology arms flanking the nucleic acid sequence of interest and the sequences flanking the double-strand break. In some aspects, a sequence flanking the double-strand break is within about 100 bases, about 90 bases, about 80 bases, about 70 bases, about 60 bases, about 50 bases, about 45 bases, about 40 bases, about 35 bases, about 30 bases, about 25 bases, about 20 bases, about 15 bases, about 10 bases, or about 5 bases of the double-strand break, or immediately flanks the double-strand break.

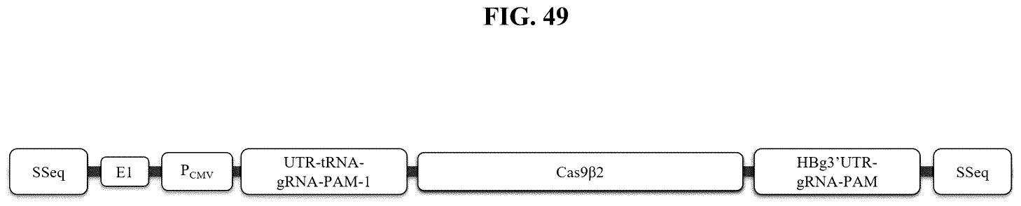

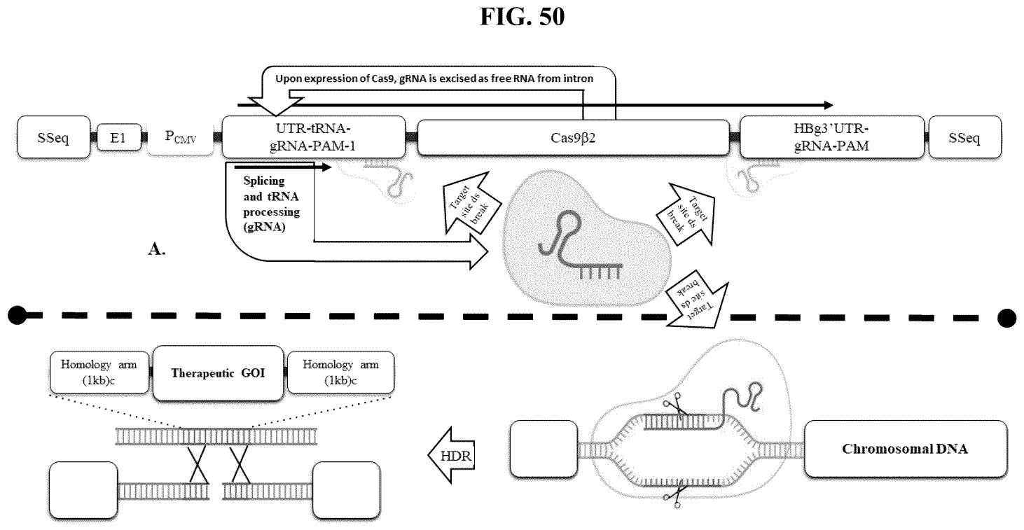

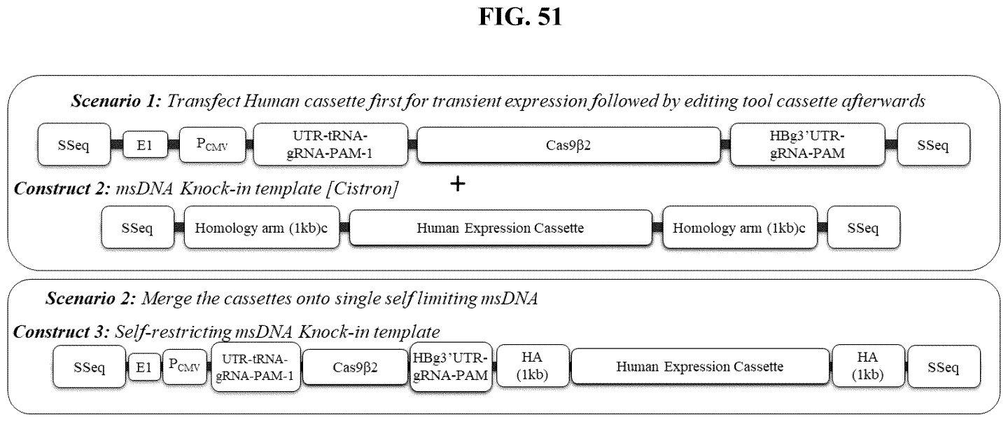

In some aspects, the homology-directed repair is by a CRISPR-Cas system. In some aspects, an expression vector as described herein comprises the CRISPR-Cas system. In some aspects, the expression vector comprises a tRNA-gRNA polycistron flanking each side of a sequence encoding a Cas endonuclease (e.g., an immunosilenced Cas9-B2). An exemplary aspect is shown in FIG. 49. In some aspects, the expression vector comprises a 5′UTR (e.g., 5′UTR1) as described herein comprising the tRNA-gRNA polycistron in an intron. In some aspects, the expression vector comprises a chimeric intron as described herein comprising the tRNA-gRNA polycistron. In some aspects, an EF1-alpha promoter as described herein comprises the tRNA-gRNA polycistron in an inherent intron. In some aspects, a polyadenylation signal or 3′UTR as described herein comprises a tRNA-gRNA polycistron. For example, upon expression of a Cas endonuclease from the vector (i.e., from the expression vector or bacterial sequence-free vector (e.g., msDNA)) comprising the flanking tRNA-gRNA polycistrons, the gRNA is excised as free RNA and targets the Cas endonuclease to the target site for gene editing (e.g., a target site in a chromosome) as well as the flanking gRNA sites on the vector. This results in self-restriction of the Cas endonuclease from the vector, which limits further expression of the Cas endonuclease. A schematic of this process is shown in FIG. 50, which also shows mediation of homology-directed repair with a nucleic acid of interest (i.e., gene of interest, GOI) flanked by homology arms on a separate vector. In some aspects, an expression vector as described herein comprises the nucleic acid sequence of interest flanked by homology arms as shown, for example, in Scenario 1 of FIG. 51. In some aspects, a nucleic acid sequence of interest and a self-restricting CRISPR-Cas system as described herein are located on a single expression vector as described herein as shown in Scenario 2 of FIG. 51. In the latter aspects, the sequences comprising the self-restricting CRISPR-Cas system are located 5′ to the sequence comprising the nucleic acid sequence of interest flanked by homology arms.

In some aspects, the nucleic acid sequence of interest is homologous to the target site for gene editing and comprises one or more nucleotide insertions, deletions, inversions, or rearrangements as compared to the target site. In some aspects, the nucleic acid of interest is a genomic sequence, a coding region, an exon, an intron, or any portion thereof that replaces a homologous sequence at the target site.

In some aspects, the nucleic acid sequence of interest is non-homologous to the target site for gene editing.

In some aspects, the nucleic acid sequence of interest restores a missing function, corrects an abnormal function, or provides an additional function associated with the target site for gene editing.

In some aspects, the nucleic acid sequence of interest is for knockout of gene expression associated with a target site for gene editing (i.e., gene silencing).

In some aspects, the nucleic acid sequence of interest is for in vivo gene editing.

In some aspects, the nucleic acid sequence of interest is for in vitro gene editing.

In some aspects, the nucleic acid sequence of interest is for ex vivo gene editing (e.g., cell therapy, such as chimeric antigen receptor (CAR) T cell therapy).

In some aspects, the gene editing comprises an epigenetic modification, and an expression vector as described herein comprises an epigenetic effector molecule as the nucleic acid of interest. In some aspects, the epigenetic effector molecule mediates, for example, acetylation or deacetylation, methylation or demethylation, or phosphorylation or dephosphorylation. In some aspects, the epigenetic effector molecule inhibits acetylation or deacetylation, methylation or demethylation, or phosphorylation or dephosphorylation. In some aspects, the epigenetic modification is a histone modification. In some aspects, the histone modification is histone acetylation and the nucleic acid of interest is a histone acetyltransferase. In some aspects, the histone modification is histone deacetylation and the nucleic acid of interest is a histone deacetylase. In some aspects, the epigenetic modification is a DNA modification. In some aspects, the DNA modification is DNA methylation and the nucleic acid of interest is a DNA methylase. In some aspects, the DNA modification is DNA demethylation and the nucleic acid of interest is a DNA demethylase. In some aspects, the epigenetic effector molecule is fused to a targeting molecule, such as a DNA-binding molecule to target the effector to a location on the chromosome.

In some aspects, the expression cassette is polygenic, i.e., the expression cassette comprises two or more nucleic acid sequences of interest encoding two or more polypeptides, respectively.

In some aspects, the expression cassette comprises a single open reading frame comprising a nucleic acid sequence encoding a self-cleaving peptide between each nucleic acid sequence encoding a polypeptide, such that the translation product of the expression cassette is cleaved intracellularly into two or more polypeptides. In some aspects, the self-cleaving peptide is a 2A self-cleaving peptide. In some aspects, the 2A self-cleaving peptide is P2A from porcine teschovirus-1. In some aspects, the 2A self-cleaving peptide is T2A from thosea asigna virus 2A. In some aspects, the self-cleaving peptide comprises any one or more of 2A, P2A, and T2A. In some aspects, the self-cleaving peptide comprises P2A and T2A.

In some aspects, the expression cassette further comprises a nucleic acid sequence encoding a marker for gene expression. In some aspects, the marker for gene expression is a fluorescent reporter gene, such as green fluorescent protein (GFP, e.g., enhanced GFP (eGFP)), red fluorescent protein (RFP), yellow fluorescent protein (YFP), or near-infrared fluorescent protein (iRFP); a bioluminescent reporter genes such as luciferase (e.g., nanoluciferase, i.e., NanoLuc® (NLuc), England et al., Bioconjug. Chem. 27(5):1175-1187 (2016), Promega Corporation); a selectable antibiotic marker; or LacZ. In some aspects, the expression cassette comprises a nucleic acid sequence encoding a self-cleaving peptide between the nucleic acid sequence encoding a marker for gene expression and any other nucleic acid sequence encoding a polypeptide.

The expression cassette can contain any expression control region known to those of skill in the art operably linked to the nucleic acid sequence(s) of interest. In some aspects, the expression control region is a promoter, enhancer, operator, repressor, ribosome binding site, translation leader sequence, intron, polyadenylation recognition sequence, RNA processing site, effector binding site, stem-loop structure, transcription termination signal, or a combination thereof.

In some aspects, the expression vector is for producing a bacterial sequence-free vector. In some aspects, the bacterial sequence-free vector is a circular covalently closed vector. In some aspects, the bacterial sequence-free vector is a linear covalently closed vector.

### B. Vector Production Systems

Provided herein is a vector production system comprising recombinant cells encoding a recombinase under the control of an inducible promoter, wherein the recombinant cells comprise an expression vector as described herein that contains first and second target sequences for a first recombinase and one or more additional target sequences for one or more additional recombinases, and wherein the recombinase targets the first and second target sequences for the first recombinase or one of the one or more additional target sequences for the one or more additional recombinases.

Suitable host cells for use in the vector production system include microbial cells, for example, bacterial cells such as E. coli cells, and yeast cells such as S. cerevisiae. Mammalian host cells can also be used, including Chinese hamster ovary (CHO) cells (e.g., the K1 lineage (ATCC CCL 61) or the Pro5 variant (ATCC CRL 1281)); fibroblast-like cells derived from SV40-transformed African Green monkey kidney of the CV-1 lineage (ATCC CCL 70), of the COS-1 lineage (ATCC CRL 1650), or of the COS-7 lineage (ATCC CRL 1651; murine L-cells; murine 3T3 cells (ATCC CRL 1658); murine C127 cells; human embryonic kidney cells of the 293 lineage (ATCC CRL 1573); human carcinoma cells including those of the HeLa lineage (ATCC CCL 2); and neuroblastoma cells of the lines IMR-32 (ATCC CCL 127), SK-N-MC (ATCC HTB 10), or SK-N-SH (ATCC HTB 11).

Suitable recombinases catalyze DNA exchange at a target sequence for a recombinase as described herein including, but not limited to, TelN, Tel, Tel (gp26 K02 phage), Cre, Flp, phiC31, Int, and other lambdoid phage integrases, e.g. phi 80, HK022 and HP1 recombinases. In some aspects, the recombinase is TelN, Tel, Cre, or Flp.

In some aspects, the recombinant cells further encode an endonuclease under the control of an inducible promoter, wherein the endonuclease targets an endonuclease target sequence in the expression vector.

Suitable endonucleases cleave polynucleotides at the endonuclease target sequence. In some aspects, the endonuclease is a homing endonuclease. In some aspects, the homing endonuclease is I-AniI, I-CeuI, I-ChuI, I-CpaI, I-CpaII, I-CreI, I-DmoI, H-DreI, I-HmuI, I-HmuII, I-LlaI, I-MsoI, PI-PfuI, PI-PkoII, I-PorI, I-PpoI, PI-PspI, I-ScaI, I-SceI, PI-SceI, I-SceII, I-SecIII, I-SceIV, I-SceV, I-SceVI, I-SceVII, I-Ssp6803I, I-TevI, I-TevII, I-TevIII, PI-TliI, PI-TliII, I-Tsp061I, or I-Vdi141I. In some aspects, the endonuclease is I-SceI. In some aspects, the endonuclease is PI-SceI. In some aspects, the recombinant cells encode a nuclease genome editing system comprising the endonuclease. In some aspects, the genome editing system is a CRISPR-Cas, a TALEN, a ZFN, or a meganuclease system. In some aspects, the nuclease genome editing system is a Class 1 or a Class 2 CRISPR-Cas system. In some aspects, the nuclease genome editing system is Type I, II, III, IV, V, or VI CRISPR-Cas system. In some aspects, the Cas endonuclease in the CRISPR-Cas system is Cas9 (e.g., a SpCas9, a SaCas9, a FnCas9, or a NmCas9), a Cas9 variant (e.g., CasB9, xCas9, SpCas9-NG, SpCas9-NRRH, SpCas9-NRCH, SpCas9-NRTH, SpG, SpRY), Cas3, Cas12 (e.g., Cas12a, Cas12b, Cas12c, Cas12d, or Cas12e), Cas13 (e.g., Cas13a, Cas13b, Cas13c, or Cas13d), or Cas14.

Recombinant host cells encoding a recombinase, or a recombinase and an endonuclease, are prepared using well-known techniques. For example, a nucleic acid sequence encoding a selected recombinase or endonuclease is introduced into the cell using a suitable vector under appropriate conditions for cell transformation. The recombinant host cells can be transformed via an expression vector, or by integration of a recombinase-encoding and/or endonuclease-encoding nucleic acid sequence into the host cell genome. In aspects where the endonuclease is associated with a nuclease genome editing system, the host cell can be designed to encode all of the components of the nuclease genome editing system, either by transformation of the host cell with one or more expression vectors comprising all of the components, by integration of all of the components into the host cell genome, or by a mixture of transformation and integration of the components. In some aspects, the host cell encodes a Cas or Cas-like endonuclease and a gRNA.

Expression of the recombinase or endonuclease, including an endonuclease of a nuclease genome editing system, is under the control of an inducible promoter, i.e., a promoter which is activated under a particular physical or chemical condition or stimulus. In some aspects, the inducible promoter is thermally-regulated, chemically-regulated, IPTG regulated, glucose-regulated, arabinose inducible, T7 polymerase regulated, cold-shock inducible, pH inducible, or combinations thereof.

Provided herein is a recombinant cell comprising an expression vector as described herein that contains first and second target sequences for a first recombinase and one or more additional target sequences for one or more additional recombinases. In some aspects, the recombinant cell encodes the first recombinase and/or one or more of the one or more recombinases as described herein. In some aspects, the recombinant cell encodes one or more endonucleases as described herein. In some aspects, the recombinant cell encodes a nuclease genome editing system as described herein.

Provided herein is a method of producing a bacterial sequence-free vector comprising incubating a vector production system as described herein under suitable conditions for expression of the recombinase. In some aspects, the method further comprises incubating the vector production system under suitable conditions for expression of an endonuclease encoded by the recombinant cells. In some aspects, the method further comprises incubating the vector production system under suitable conditions for expression of a nuclease genome editing system encoded by the recombinant cells. In some aspects, the method further comprises harvesting the bacterial sequence-free vector.

Provided herein is a bacterial sequence-free vector produced by a method of producing a bacterial sequence-free vector as described herein.

### III. Bacterial Sequence-Free Vectors

Provided herein is a bacterial sequence-free vector comprising: (a) an expression cassette comprising a nucleic acid sequence of interest, and (b) one or more of: (i) a synthetic enhancer comprising a nucleic acid sequence at least about 90% identical to SEQ ID NO:12 located 5′ to another enhancer or a promoter in the expression cassette, (ii) a CMV enhancer located 5′ to a promoter in the expression cassette, (iii) a 5′UTR comprising an intron, wherein the 5′UTR is integrated in the expression cassette between a promoter and the nucleic acid sequence of interest, (iv) a vertebrate chromatin insulator integrated in the expression cassette between the nucleic acid of interest and a polyadenylation signal, (v) a WPRE integrated in the expression cassette between the nucleic acid of interest and a polyadenylation signal, (vi) a S/MAR integrated in the expression cassette between the nucleic acid of interest and a polyadenylation signal, or (vii) a DTS located 5′ to the expression cassette.

In some aspects, the bacterial sequence-free vector comprises a synthetic enhancer comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:12 located 5′ to another enhancer or a promoter in the expression cassette. In some aspects, the bacterial sequence-free vector comprises a synthetic enhancer comprising the nucleic acid sequence of SEQ ID NO: 12 located 5′ to another enhancer or a promoter in the expression cassette. In some aspects, the synthetic enhancer comprises multiple contiguous copies of the nucleic acid sequence, such as, for example, 1, 2, 3, 4, 5, or more contiguous copies. In some aspects, the synthetic enhancer comprises 3 contiguous copies of the nucleic acid sequence. In some aspects, the synthetic enhancer comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:46. In some aspects, the synthetic enhancer comprises the nucleic acid sequence of SEQ ID NO:46. In some aspects, the synthetic enhancer is integrated at the 5′ end of a chicken β-actin promoter. In some aspects, a chimeric intron comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:47 is integrated at the 3′ end of the chicken β-actin promoter and 5′ to the nucleic acid sequence of interest. In some aspects, a chimeric intron comprising the nucleic acid sequence of SEQ ID NO:47 is integrated at the 3′ end of the chicken β-actin promoter and 5′ to the nucleic acid sequence of interest.

In some aspects, the bacterial sequence-free vector comprises a CMV enhancer located 5′ to a promoter in the expression cassette. In some aspects, the CMV enhancer is integrated at the 3′ end of a synthetic enhancer comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:12. In some aspects, the CMV enhancer is integrated at the 3′ end of a synthetic enhancer comprising the nucleic acid sequence of SEQ ID NO:12. In some aspects, the CMV enhancer is integrated at the 3′ end of multiple contiguous copies of the synthetic enhancer, such as, for example, at the 3′ end of 1, 2, 3, 4, 5, or more contiguous copies of the synthetic enhancer. In some aspects, the CMV enhancer is integrated at the 3′ end of 3 contiguous copies of the synthetic enhancer. In some aspects, the CMV enhancer is integrated at the 3′ end of a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:46. In some aspects, the CMV enhancer is integrated at the 3′ end of the nucleic acid sequence of SEQ ID NO:46. In some aspects, a CMV promoter is integrated at the 3′ end of the CMV enhancer and 5′ to the nucleic acid sequence of interest.

In some aspects, the bacterial sequence-free vector comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:35, SEQ ID NO:36, SEQ ID NO:37, SEQ ID NO:38, or SEQ ID NO:39 located 5′ to the nucleic acid sequence of interest. In some aspects, the bacterial sequence-free vector comprises the nucleic acid sequence of SEQ ID NO:35, SEQ ID NO:36, SEQ ID NO:37, SEQ ID NO:38, or SEQ ID NO:39 located 5′ to the nucleic acid sequence of interest. In some aspects, a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:35, SEQ ID NO:36, SEQ ID NO:37, SEQ ID NO:38, or SEQ ID NO:39, or the nucleic acid sequence of SEQ ID NO:35, SEQ ID NO:36, SEQ ID NO:37, SEQ ID NO:38, or SEQ ID NO:39, comprises all regulatory elements in the expression cassette located 5′ to the nucleic acid sequence of interest.

In some aspects, the bacterial sequence-free vector comprises a 5′UTR comprising an intron, wherein the 5′UTR (i.e., the 5′UTR comprising the intron) is integrated in the expression cassette between a promoter and the nucleic acid sequence of interest.

In some aspects, the 5′UTR is for improving transgene transcript splicing and translation from the bacterial sequence-free vector as compared to the same bacterial sequence-free vector lacking the 5′UTR.

In some aspects, the intron comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:1. In some aspects, the intron comprises the nucleic acid sequence of SEQ ID NO:1.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:2. In some aspects, the 5′UTR comprises the nucleic acid sequence of SEQ ID NO:2.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:4. In some aspects, the 5′UTR comprises the nucleic acid sequence of SEQ ID NO:4.

In some aspects, the 5′UTR further comprises a non-coding sequence integrated within the intron.

In some aspects, the intron is at least about 90%, at least about 91%, at least about 92%, at least about 93%, at least about 94%, at least about 95%, at least about 96%, at least about 97%, at least about 98%, or at least about 99% identical to SEQ ID NO:1, or comprises SEQ ID NO: 1, and the non-coding sequence is integrated between two of the nucleotides in the intron corresponding to any two nucleotides from positions 25 to 55 of SEQ ID NO:1.

In some aspects, the non-coding sequence is non-prokaryotic and non-viral. In some aspects, the non-coding sequence is eukaryotic. In some aspects, the non-coding sequence comprises an intron, a UCOE, a S/MAR, a SV40 enhancer sequence (e.g., one or more than one SV40 enhancer sequences, such as two, three, four, five or more SV40 enhancer sequences), a vertebrate chromatin insulator (e.g., cHS4), a WPRE, or any combination thereof.

In some aspects, the non-coding sequence comprises an S/MAR. In some aspects, the S/MAR is MAR-5, provided herein as SEQ ID NO:9.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:3. In some aspects, the 5′UTR comprises SEQ ID NO:3.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:5. In some aspects, the 5′UTR comprises SEQ ID NO:5.

In some aspects, the 5′UTR is integrated in the expression cassette between a chicken β-actin promoter and the nucleic acid sequence of interest.

In some aspects, the 5′UTR is integrated in the expression cassette between a CMV promoter and the nucleic acid sequence of interest.

In some aspects, the 5′UTR is integrated in the expression cassette between a promoter and the nucleic acid sequence of interest, wherein the promoter is integrated at the 3′ end of a CMV enhancer. In some aspects, the CMV enhancer is integrated at the 3′ end of a synthetic enhancer comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:12. In some aspects, the CMV enhancer is integrated at the 3′ end of a synthetic enhancer comprising the nucleic acid sequence of SEQ ID NO: 12. In some aspects, the CMV enhancer is integrated at the 3′ end of multiple contiguous copies of the synthetic enhancer, such as, for example, at the 3′ end of 1, 2, 3, 4, 5, or more contiguous copies of the synthetic enhancer. In some aspects, the CMV enhancer is integrated at the 3′ end of 3 contiguous copies of the synthetic enhancer. In some aspects, the CMV enhancer is integrated at the 3′ end of a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:46. In some aspects, the CMV enhancer is integrated at the 3′ end of a nucleic acid sequence of SEQ ID NO:46.

In some aspects, the bacterial sequence-free vector comprises a polyadenylation signal integrated at the 3′ end of the nucleic acid sequence of interest. In some aspects, the polyadenylation signal comprises a Xenopus laevis beta-globin polyadenylation signal, a human beta-globin polyadenylation signal, or a hybrid Xenopus laevis and human beta-globin polyadenylation signal. In some aspects, the polyadenylation signal comprises multiple copies of a Xenopus laevis beta-globin polyadenylation signal, a human beta-globin polyadenylation signal, or a hybrid Xenopus laevis and human beta-globin polyadenylation signal, such as, for example, 1, 2, 3, 4, or 5 copies. In some aspects, the polyadenylation signal comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:13, SEQ ID NO: 14, or SEQ ID NO:15. In some aspects, the polyadenylation signal comprises the nucleic acid sequence of SEQ ID NO: 13, SEQ ID NO:14, or SEQ ID NO:15. In some aspects, a polyadenylic acid tail (i.e., poly(A) tail is located at the 3′ end of the polyadenylation signal. In some aspects, the poly(A) tail is 100, 101, 102, 103, 104, 105, 106, 107, 108, 109, 110, 111, 112, 113, 114, 115, 116, 117, 118, 119, 120, or more residues in length. In some aspects, the sequence comprising the polyadenylation signal and the poly(A) tail is at least about 90%, at least about 91%, at least about 92%, at least about 93%, at least about 94%, at least about 95%, at least about 96%, at least about 97%, at least about 98%, or at least about 99% identical to SEQ ID NO:16, SEQ ID NO:17, or SEQ ID NO:18. In some aspects, the sequence comprising the polyadenylation signal and the poly(A) tail comprises SEQ ID NO:16, SEQ ID NO:17, or SEQ ID NO:18.

In some aspects, the bacterial sequence-free vector comprises a vertebrate chromatin insulator in the expression cassette. In some aspects, the vertebrate chromatin insulator is cHS4. In some aspects, the vertebrate chromatin insulator is integrated in the expression cassette between the nucleic acid of interest and a polyadenylation signal as described herein. In some aspects, the vertebrate chromatin insulator is integrated within the intron of a 5′UTR as described herein.

In some aspects, the vertebrate chromatin insulator comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:8. In some aspects, the vertebrate chromatin insulator comprises SEQ ID NO:8.

In some aspects, the vertebrate chromatin insulator is for improving establishment (i.e., transfection efficiency) of a bacterial sequence-free vector as compared to the same bacterial sequence-free vector without the vertebrate chromatin insulator.

In some aspects, the bacterial sequence-free vector comprises a WPRE in the expression cassette. In some aspects, the WPRE is integrated in the expression cassette between the nucleic acid of interest and a polyadenylation signal as described herein. In some aspects, the WPRE is integrated in the expression cassette at the 3′ end of a S/MAR as described herein and the 5′ end of a polyadenylation signal as described herein. In some aspects, the WPRE is integrated within the intron of a 5′UTR as described herein.

In some aspects, the WPRE comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:11. In some aspects, the WPRE comprises SEQ ID NO:11.

In some aspects, the WPRE improves expression of the transgene from the bacterial sequence-free vector as compared to the same bacterial sequence-free vector lacking the WPRE.

In some aspects, the bacterial sequence-free vector comprises an S/MAR in the expression cassette. In some aspects, the S/MAR is integrated in the expression cassette between the nucleic acid of interest and a polyadenylation signal. In some aspects, the S/MAR is integrated in the expression cassette at the 3′ end of a nucleic acid sequence of interest and the 5′ end of a WPRE as described herein. In some aspects, the S/MAR is integrated within the intron of a 5′UTR as described herein.

In some aspects, the S/MAR is MAR-3, MAR-4, or MAR-5. In some aspects, the S/MAR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:9. In some aspects, the S/MAR comprises SEQ ID NO:9.

In some aspects, the S/MAR is human CSP-B MAR or CSP-C MAR. In some aspects, the S/MAR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:10. In some aspects, the S/MAR comprises SEQ ID NO:10.

In some aspects, the S/MAR is for improving expression levels, stability, and/or durability of the bacterial sequence-free vector (e.g., by episomal maintenance and replication, such as expansion and partition of the vector to daughter cells, and/or by preventing epigenetic silencing) as compared to the same bacterial sequence-free vector lacking the S/MAR.

In some aspects, the bacterial sequence-free vector comprising any one of more of (b)(i)-(b)(v) as described above (i.e., without a DTS) further comprises an enhancer sequence flanking each side of the expression cassette. In some aspects, the enhancer sequence flanking each side of the expression cassette is at least two enhancer sequences flanking each side of the expression cassette. In some aspects, the enhancer sequence is a SV40 enhancer sequence.

In some aspects, the bacterial sequence-free vector comprises a DTS. In some aspects, the DTS is located 5′ to the expression cassette. In some aspects, the DTS is a SV40 enhancer sequence. In some aspects, the DTS is cell-specific. In some aspects, the DTS is specific for smooth muscle cells, embryonic stem cells, type II pneumonocytes, endothelial cells, or osteoblasts.

In some aspects, a bacterial sequence-free vector as described herein further comprises a UCOE in the expression cassette. In some aspects, the UCOE is located 5′ to the promoter or any enhancer in the expression cassette. In some aspects, the UCOE is integrated within the intron of a 5′UTR as described herein.

In some aspects, the UCOE is A2UCOE. In some aspects, the UCOE comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:6. In some aspects, the UCOE is SEQ ID NO:6.

In some aspects, the UCOE is SRF-UCOE. In some aspects, the UCOE comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:7. In some aspects, the UCOE is SEQ ID NO:7.

In some aspects, the UCOE improves expression of the transgene from the bacterial sequence-free vector as compared to the same bacterial sequence-free vector lacking the UCOE.

In some aspects, the bacterial sequence-free vector comprises Enhancer-1 in the expression cassette. In some aspects, Enhancer-1 is integrated 5′ to the promoter or any other enhancer in the expression cassette. In some aspects, Enhancer-1 is integrated between the 3′ end of a UCOE and the 5′ end of a CMV enhancer. In some aspects, Enhancer-1 comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:12. In some aspects, Enhancer-1 is SEQ ID NO: 12.

In some aspects, the bacterial sequence-free vector comprises a CMV, EF1, SV40, CAG, Rho, VDM2, HCR, or HLP promoter, or variant thereof, in the expression cassette. In some aspects, the bacterial sequence-free vector comprises a CMV promoter variant in the expression cassette.

In some aspects, the bacterial sequence-free vector comprises an EF1-alpha promoter in the expression cassette. In some aspects, the bacterial sequence-free vector comprises a CMV enhancer and an EF1-alpha promoter in the expression cassette.

In some aspects, the bacterial sequence-free vector comprises a 3′UTR in the expression cassette comprising two copies of a beta-globin polyadenylation signal. In some aspects, the 3′UTR is integrated 3′ to the nucleic acid sequence of interest.

In some aspects, the 3′UTR comprises two copies of a Xenopus laevis beta-globin polyadenylation signal. In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO: 13. In some aspects, the 3′UTR is SEQ ID NO:13.

In some aspects, the 3′UTR comprises two copies of a human beta-globin polyadenylation signal. In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO: 14. In some aspects, the 3′UTR is SEQ ID NO:14.

In some aspects, the 3′UTR comprises one copy of a Xenopus laevis beta-globin polyadenylation signal and one copy of a human beta-globin polyadenylation signal. In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:15. In some aspects, the 3′UTR is SEQ ID NO:15.

In some aspects, the 3′UTR further comprises a poly(A) tail comprising 100 to 120 adenine nucleotides, i.e., 100, 101, 102, 103, 104, 105, 106, 107, 108, 109, 110, 111, 112, 113, 114, 115, 116, 117, 118, 119, or 120 adenine nucleotides.

In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:16. In some aspects, the 3′UTR is SEQ ID NO:16.

In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:17. In some aspects, the 3′UTR is SEQ ID NO:17.

In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:18. In some aspects, the 3′UTR is SEQ ID NO:18.

The nucleic acid sequence of interest of a bacterial sequence-free vector as described herein includes any of the nucleic acid sequences described herein with respect to the expression vectors for producing the bacterial sequence-free vectors.

In some aspects, a bacterial sequence-free vector as described herein comprises a Cas endonuclease target sequence (i.e., a sequence homologous to a gRNA targeting sequence) located 5′ and 3′ to the nucleic acid sequence of interest, wherein a target site for gene editing (e.g., a target site in a chromosome) comprises the same Cas endonuclease target sequence.

In some aspects, a bacterial sequence-free vector as described herein comprises a CRISPR-Cas system. In some aspects, the bacterial sequence-free vector comprises a tRNA-gRNA polycistron flanking each side of a sequence encoding a Cas endonuclease (e.g., an immunosilenced Cas9-B2). In some aspects, the bacterial sequence-free vector comprises a 5′UTR (e.g., 5′UTR1) as described herein comprising the tRNA-gRNA polycistron in an intron. In some aspects, the bacterial sequence-free vector comprises a chimeric intron as described herein comprising the tRNA-gRNA polycistron. In some aspects, an EF1-alpha promoter as described herein comprises the tRNA-gRNA polycistron in an inherent intron. In some aspects, a polyadenylation signal or 3′UTR as described herein comprises a tRNA-gRNA polycistron. In some aspects, a nucleic acid sequence of interest and a self-restricting CRISPR-Cas system as described herein are located on a single bacterial sequence-free vector as described herein. In the latter aspects, the sequences comprising the self-restricting CRISPR-Cas system are located 5′ to the sequence comprising the nucleic acid sequence of interest flanked by homology arms.

A bacterial sequence-free vector as described herein can contain any combination of the above modifications. In some aspects, the combination provides a synergistic effect.

In some aspects, the bacterial sequence-free vector is a circular covalently closed vector.

In some aspects, the bacterial sequence-free vector is a linear covalently closed vector.

Provided herein is a recombinant cell comprising a bacterial sequence-free vector as disclosed herein.

### IV. Other Expression Vectors

Improvements and modifications described above can also be applied to other expression vectors such as, but not limited to, expression vectors that are utilized for direct gene expression rather than production of bacterial sequence-free vectors. In some aspects, the nucleic acid sequences described herein are provided as DNA sequences, and the expression vectors are DNA expression vectors. In some aspects, the nucleic acid sequences described herein are provided as RNA sequences, and the expression vectors are RNA expression vectors. RNA sequences can correspond to the DNA sequence provided as any SEQ ID NO herein or can correspond to the DNA sequence that is complementary to the DNA sequence provided as any SEQ ID NO herein.

Provided herein is a polynucleotide comprising any combination of nucleic acid sequences as described herein.

Provided herein is a polynucleotide comprising a nucleic acid sequence of: an intron, a 5′UTR comprising an intron, and/or a 3′UTR as described herein.

Provided herein is a polynucleotide comprising a nucleic acid sequence at least about 90%, at least about 91%, at least about 92%, at least about 93%, at least about 94%, at least about 95%, at least about 96%, at least about 97%, at least about 98%, or at least about 99% identical to any one of SEQ ID NOs: 1, 2, 3, 5, 13, 14, 15, 16, 17, or 18. In some aspects, the polynucleotide comprises 100 to 120 adenine nucleotides at the 3′ end of the nucleic acid sequence. In some aspects, the polynucleotide comprises a nucleic acid sequence at least about 90%, at least about 91%, at least about 92%, at least about 93%, at least about 94%, at least about 95%, at least about 96%, at least about 97%, at least about 98%, or at least about 99% identical to any one of SEQ ID NOs:13, 14, or 15, and 100 to 120 adenine nucleotides at the 3′ end of the nucleic acid sequence. In some aspects, the polynucleotide comprises the nucleic acid sequence of any one of SEQ ID NOs: 1, 2, 3, 5, 13, 14, 15, 16, 17, or 18.

Provided herein is an expression vector comprising one or more of the polynucleotides described herein. In some aspects, the expression vector comprises a polynucleotide comprising a nucleic acid sequence at least about 90%, at least about 91%, at least about 92%, at least about 93%, at least about 94%, at least about 95%, at least about 96%, at least about 97%, at least about 98%, or at least about 99% identical to any one of SEQ ID NOs: 1, 2, 3, 5, 13, 14, 15, 16, 17, or 18. In some aspects, the polynucleotide comprises 100 to 120 adenine nucleotides at the 3′ end of the nucleic acid sequence. In some aspects, the polynucleotide comprises a nucleic acid sequence at least about 90%, at least about 91%, at least about 92%, at least about 93%, at least about 94%, at least about 95%, at least about 96%, at least about 97%, at least about 98%, or at least about 99% identical to any one of SEQ ID NOs: 13, 14, or 15, and 100 to 120 adenine nucleotides at the 3′ end of the nucleic acid sequence. In some aspects, the expression vector comprises a polynucleotide comprising the nucleic acid sequence of any one of SEQ ID NOs: 1, 2, 3, 5, 13, 14, 15, 16, 17, or 18. In some aspects, the expression vector comprises a polynucleotide comprising the nucleic acid sequence of any one of SEQ ID NOs: 2, 3, or 5, and (a) a polynucleotide comprising the nucleic acid sequence of any one of SEQ ID NOs: 13, 14, 15, 16, 17, or 18, or (b) a polynucleotide comprising the nucleic acid sequence of any one of SEQ ID NOs: 13, 14, or 15 and 100 to 120 adenine nucleotides at the 3′ end of the nucleic acid sequence.

Provided herein is an expression vector comprising: a 5′UTR comprising an intron, wherein the 5′UTR is integrated in the expression cassette between a promoter and a nucleic acid sequence of interest, and/or a 3′UTR comprising two copies of a beta-globin polyadenylation signal integrated in the expression cassette 3′ to the nucleic acid sequence of interest.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:2. In some aspects, the 5′UTR comprises the nucleic acid sequence of SEQ ID NO:2.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:4. In some aspects, the 5′UTR comprises the nucleic acid sequence of SEQ ID NO:4.

In some aspects, the 5′UTR further comprises a non-coding sequence integrated within the intron.

In some aspects, the intron is at least about 90%, at least about 91%, at least about 92%, at least about 93%, at least about 94%, at least about 95%, at least about 96%, at least about 97%, at least about 98%, or at least about 99% identical to SEQ ID NO:1, or comprises SEQ ID NO:1, and the non-coding sequence is integrated between two of the nucleotides in the intron corresponding to any two nucleotides from positions 25 to 55 of SEQ ID NO:1.

In some aspects, the non-coding sequence is non-prokaryotic and non-viral. In some aspects, the non-coding sequence is a eukaryotic sequence. In some aspects, the non-coding sequence comprises an intron, a UCOE, an S/MAR, an SV40 enhancer sequence (e.g., one or more than one SV40 enhancer sequences, such as two, three, four, five or more SV40 enhancer sequences), a vertebrate chromatin insulator (e.g., cHS4), a WPRE, or any combination thereof.

In some aspects, the non-coding sequence is an S/MAR. In some aspects, the S/MAR is MAR-5, provided herein as SEQ ID NO:9.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:3. In some aspects, the 5′UTR comprises SEQ ID NO:3.

In some aspects, the 5′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:5. In some aspects, the 5′UTR comprises SEQ ID NO:5.

In some aspects, the 3′UTR comprises two copies of a Xenopus laevis beta-globin polyadenylation signal. In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:13. In some aspects, the 3′UTR is SEQ ID NO:13.

In some aspects, the 3′UTR comprises two copies of a human beta-globin polyadenylation signal. In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:14. In some aspects, the 3′UTR is SEQ ID NO:14.

In some aspects, the 3′UTR comprises one copy of a Xenopus laevis beta-globin polyadenylation signal and one copy of a human beta-globin polyadenylation signal. In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO: 15. In some aspects, the 3′UTR is SEQ ID NO:15.

In some aspects, the 3′UTR further comprises a poly(A) tail comprising 100 to 120 adenine nucleotides, i.e., 100, 101, 102, 103, 104, 105, 106, 107, 108, 109, 110, 111, 112, 113, 114, 115, 116, 117, 118, 119, or 120 adenine nucleotides.

In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:16. In some aspects, the 3′UTR is SEQ ID NO:16.

In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:17. In some aspects, the 3′UTR is SEQ ID NO:17.

In some aspects, the 3′UTR comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:18. In some aspects, the 3′UTR is SEQ ID NO:18.

Provided herein is an expression vector comprising a synthetic enhancer comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:12. In some aspects, the expression vector comprises a synthetic enhancer comprising the nucleic acid sequence of SEQ ID NO: 12. In some aspects, the synthetic enhancer comprises multiple contiguous copies of the nucleic acid sequence, such as, for example, 1, 2, 3, 4, 5, or more contiguous copies. In some aspects, the synthetic enhancer comprises 3 contiguous copies of the nucleic acid sequence. In some aspects, the synthetic enhancer comprises a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:46. In some aspects, the synthetic enhancer comprises the nucleic acid sequence of SEQ ID NO:46.

Provided herein is an expression vector comprising a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:35, SEQ ID NO:36, SEQ ID NO:37, SEQ ID NO:38, or SEQ ID NO:39. In some aspects, the expression vector comprises the nucleic acid sequence of SEQ ID NO:35, SEQ ID NO:36, SEQ ID NO:37, SEQ ID NO:38, or SEQ ID NO:39. In some aspects, a nucleic acid sequence at least about 90%, at least about 91%, at least about 92%, at least about 93%, at least about 94%, at least about 95%, at least about 96%, at least about 97%, at least about 98%, or at least about 99% identical to SEQ ID NO:35, SEQ ID NO:36, SEQ ID NO:37, SEQ ID NO:38, or SEQ ID NO:39, or the nucleic acid sequence of SEQ ID NO:35, SEQ ID NO:36, SEQ ID NO:37, SEQ ID NO:38, or SEQ ID NO:39, comprises all regulatory elements in an expression cassette located 5′ to a nucleic acid sequence of interest in the expression vector.

### V. Compositions

Provided herein is a composition comprising an expression vector or bacterial sequence-free vector as described herein.

A variety of methods are known in the art and are suitable for introduction of nucleic acids into a cell. Examples include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG), and the like), or cell fusion.

Nanoparticle carriers such as liposomes, micelles, and polymeric nanoparticles have been investigated for improving bioavailability and pharmacokinetic properties of therapeutics via various mechanisms, for example, the enhanced permeability and retention (EPR) effect.

Further improvement can be achieved by conjugation of targeting ligands onto nanoparticles to achieve selective delivery to a target cell. For example, receptor-targeted nanoparticle delivery has been shown to improve therapeutic responses both in vitro and in vivo. Targeting ligands that have been investigated include folate, transferrin, antibodies, peptides, and aptamers. Additionally, multiple functionalities can be incorporated into the design of nanoparticles, e.g., to enable imaging and to trigger intracellular drug release.

In some aspects, the composition further comprises a delivery agent. In some aspects, the delivery agent is a nanoparticle. In some aspects, the delivery agent is selected from the group consisting of liposomes, non-lipid polymeric molecules, endosomes, and any combination thereof.

In some aspects, the delivery agent (e.g., a nanoparticle) comprises a targeting ligand.

In some aspects, the composition further comprises a physiologically acceptable carrier, excipient, or stabilizer. See, e.g., Remington: The Science and Practice of Pharmacy, 22^{nd} ed. (2013). Acceptable carriers, excipients, or stabilizers can include those that are nontoxic to a subject. In some aspects, the composition or one or more components of the composition are sterile. A sterile component can be prepared, for example, by filtration (e.g., by a sterile filtration membrane) or by irradiation (e.g., by gamma irradiation).

In some aspects, the composition comprising an expression vector or bacterial sequence-free vector as described herein is a pharmaceutical composition further comprising a pharmaceutically acceptable carrier.

An excipient of the present invention can be described as a “pharmaceutically acceptable” excipient when added to a pharmaceutical composition, meaning that the excipient is a compound, material, composition, salt, and/or dosage form which is, within the scope of sound medical judgment, suitable for contact with the tissues of human beings and animals without excessive toxicity, irritation, allergic response, or other problematic complications over the desired duration of contact commensurate with a reasonable benefit/risk ratio. In some aspects, the term “pharmaceutically acceptable” means approved by a regulatory agency of the Federal or a state government or listed in the U.S. Pharmacopeia or other generally recognized international pharmacopeia for use in animals, and more particularly in humans. Various excipients can be used. In some aspects, the excipient can be, but is not limited to, an alkaline agent, a stabilizer, an antioxidant, an adhesion agent, a separating agent, a coating agent, an exterior phase component, a controlled-release component, a solvent, a surfactant, a humectant, a buffering agent, a filler, an emollient, or combinations thereof. Excipients in addition to those discussed herein can include excipients listed in, though not limited to, Remington: The Science and Practice of Pharmacy, 22^{nd} ed. (2013). Inclusion of an excipient in a particular classification herein (e.g., “solvent”) is intended to illustrate rather than limit the role of the excipient. A particular excipient can fall within multiple classifications.

A pharmaceutical composition of the disclosure is formulated to be compatible with its intended route of administration. Exemplary routes of administration include enteral, topical, parenteral, oral, pulmonary, intranasal, intravenous, epidermal, transdermal, subcutaneous, intramuscular, or intraperitoneal administration, or inhalation. “Parenteral administration” as used herein means modes of administration other than enteral and topical administration, usually by injection or infusion, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrapleural, and intrasternal injection and infusion, as well as in vivo electroporation. In some aspects, the formulation is administered via a non-parenteral route, in some aspects, orally. Other non-parenteral routes include a topical, epidermal, or mucosal route of administration, for example, intranasally, vaginally, rectally, sublingually or topically.

In some aspects, the pharmaceutical composition is lyophilized.

### VI. Therapeutic Uses and Methods

Provided herein is a method of treating a disease or disorder in a subject in need thereof, comprising administering an expression vector, bacterial sequence-free vector, or pharmaceutical composition as described herein to the subject.

The expression vector, bacterial sequence-free vector, or composition can be administered to a subject by any route of administration that is effective for treating the disease or disorder.

In some aspects, the administering is by enteral, topical, parenteral, oral, pulmonary, intranasal, intravenous, epidermal, transdermal, subcutaneous, intramuscular, intrathecal, or intraperitoneal administration, inhalation, or cerebrospinal fluid (CSF)-based delivery via intracerebroventricular (ICV) injection, cisterna magna administration (ICM), or lumbar intrathecal puncture (LIT).

In some aspects, the administering is by parenteral or non-parenteral administration.

In some aspects, the parenteral administration is by injection or infusion.

In some aspects, the parenteral administration is by intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, retroorbital, intracerebroventricular, subarachnoid, intraspinal, epidural, intrapleural, or intrasternal injection or infusion, or by in vivo electroporation, nucleofection, microbubble, or ultrasound.

In some aspects, the non-parenteral administration is oral, topical, epidermal, mucosal, intranasal, vaginal, rectal, or sublingual.

In some aspects, the administering is by oral, pulmonary, intranasal, intravenous, epidermal, transdermal, subcutaneous, intramuscular, or intraperitoneal administration, or by inhalation.

In some aspects, the administering is by oral, nasal, or pulmonary administration. In some aspects, the administering is by nasal administration.

Administering can be performed, for example, once, a plurality of times, and/or over one or more extended periods. In some aspects, the administering is one time, two times (e.g., a first administration followed by a second administration about 1, about 2, about 3, about 4 or more weeks later), once about every week, once about every month, once about every 2 months, once about every 3 months, once about every 4 months, once about every 6 months, once about every year, or once about every decade.

Provided herein is a method of gene editing comprising inserting a nucleic acid sequence of interest from an expression vector, bacterial sequence-free vector, or pharmaceutical composition as described herein into a target site for gene editing.

In some aspects, the inserting is by non-homologous end joining.

In some aspects, the inserting is by homology directed repair. In some aspects, the nucleic acid sequence of interest is flanked by 5′ and 3′ homology arms as described herein.

In some aspects, the nucleic acid sequence of interest is homologous to the target site for gene editing and comprises one or more nucleotide insertions, deletions, inversions, or rearrangements as compared to the target site.

In some aspects, the nucleic acid sequence of interest is non-homologous to the target site for gene editing.

In some aspects, the nucleic acid sequence of interest restores a missing function, corrects an abnormal function, or provides an additional function associated with the target site for gene editing.

In some aspects, the nucleic acid sequence of interest is for knockout of gene expression associated with the target site for gene editing.

In some aspects, the method of gene editing is a method of treating a disease or disorder in a subject in need thereof.

In some aspects, the nucleic acid sequence of interest is for in vivo gene editing.

In some aspects, the nucleic acid sequence of interest is for in vitro gene editing.

In some aspects, the nucleic acid sequence of interest is for ex vivo gene editing (e.g., cell therapy, such as CAR T cell therapy).

In some aspects, the method is an in vitro method. In some aspects, the in vitro method further comprises administering the expression vector, bacterial sequence-free vector, or pharmaceutical composition to cells (e.g., for in vitro or ex vivo gene editing). In some aspects, the in vitro method further comprises administering an endonuclease for gene editing, or a genome editing system or components thereof (e.g., Cas endonuclease and gRNA for a CRISPR-Cas system) to the cells. In some aspects, the genome editing system is a CRISPR-Cas, TALEN, ZFN, or meganuclease gene editing system.

In some aspects, the method is an in vivo method. In some aspects, the in vivo method further comprises administering the expression vector, bacterial sequence-free vector, or pharmaceutical composition to a subject. In some aspects, the in vivo method further comprises administering an endonuclease for gene editing, or a genome editing system or components thereof (e.g., Cas endonuclease and gRNA for a CRISPR-Cas system) to the subject. In some aspects, the genome editing system is a CRISPR-Cas, TALEN, ZFN, or meganuclease gene editing system.

The endonuclease for gene editing, or the genome editing system or components thereof, can be administered by any methods described herein or as known in the art for administering nucleic acid sequences and/or polypeptides to cells or subjects, including through electroporation or vectors as applicable to the administration. For example, in aspects comprising a CRISPR-Cas system, RNA encoding Cas and/or gRNA can be administered, Cas and/or gRNA can be directly administered, bacterial sequence-free vectors or expression vectors as described herein can be administered that encode Cas and/or gRNA, or any other suitable vector known in the art can be administered that encode Cas and/or gRNA.

In some aspects, the nucleic acid of interest is provided in a linear covalently closed bacterial sequence-free vector (i.e., msDNA) as described herein. In some aspects, use of the linear covalently closed bacterial sequence-free vector in gene editing avoids any undesired non-homologous end joining because the ends of the bacterial sequence-free vector are closed and non-reactive with double strand breaks. In some aspects, use of the linear covalently closed bacterial sequence-free vector in gene editing enhances homology-directed repair. In some aspects, the recombination rate for homology-directed repair is higher when the nucleic acid sequence of interest is provided by a linear covalently closed bacterial sequence-free vector as described herein than when the nucleic acid sequence of interest is provided by a circular supercoiled vector.

The following examples are offered by way of illustration and not by way of limitation.

### EXAMPLES

### Example 1—Expression Vectors Containing a Chimeric Intron or a 5′UTR

### A. Expression Vectors

A polygenic expression vector was prepared by replacing the eGFP coding sequence of a parent ministring expression vector (Mediphage Bioceuticals, Inc., Toronto, CA, U.S. Pat. Nos. 9,290,778 and 9,862,954), pGL2-SS*-CAG-eGFP-BGpA-SS*, with an expression cassette encoding enhanced green fluorescent protein (eGFP) and the NanoLuc® luciferase reporter modified with a secretion sequence for extracellular expression (NLuc, Promega Corporation) between the two specialized Super Sequence (SS*) sites of the parent vector.

The expression cassette of the parent vector and the polygenic vector contained a CAG promoter, which is a synthetic promoter that includes a cytomegalovirus (CMV) enhancer, a promoter from chicken β-actin, and a chimeric intron.

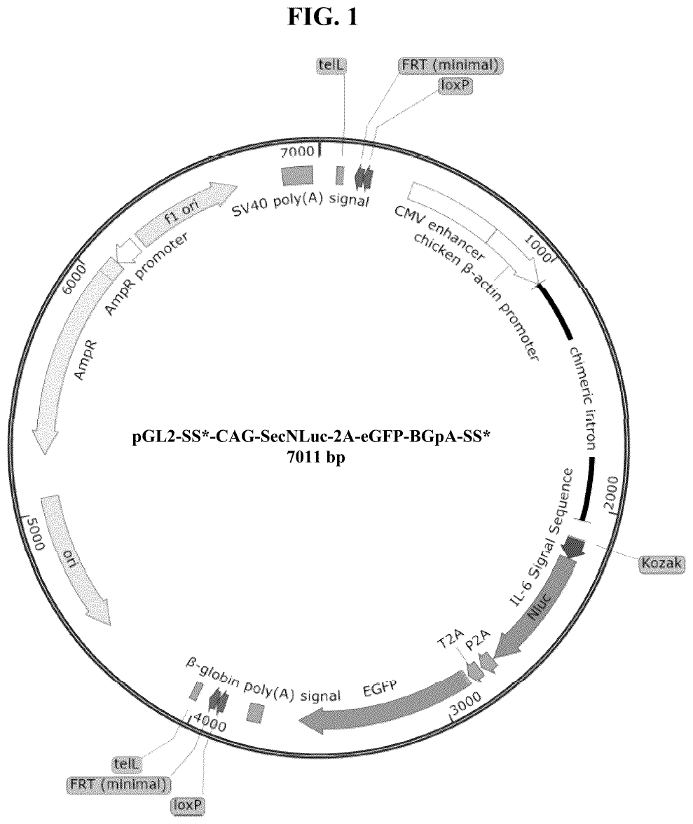

A map of the polygenic expression vector is shown in FIG. 1 (pGL2-SS*-CAG-SecNLuc-2A-eGFP-BGpA-SS*), which contains a specialized Super Sequence site (SS*) having recombinase target sequences (telL, FRT (minimal), and loxP) flanking a polygenic expression cassette containing the CAG promoter, sequences encoding enhanced green fluorescent protein (eGFP) and secreted nano-luciferase (SecNLuc) connected by P2A and T2A self-cleaving peptides (SecNLuc-2A-eGFP), and a rabbit beta-globin polyadenylation signal (BGpA). The nucleic acid sequence for the vector is provided as SEQ ID NO: 19.

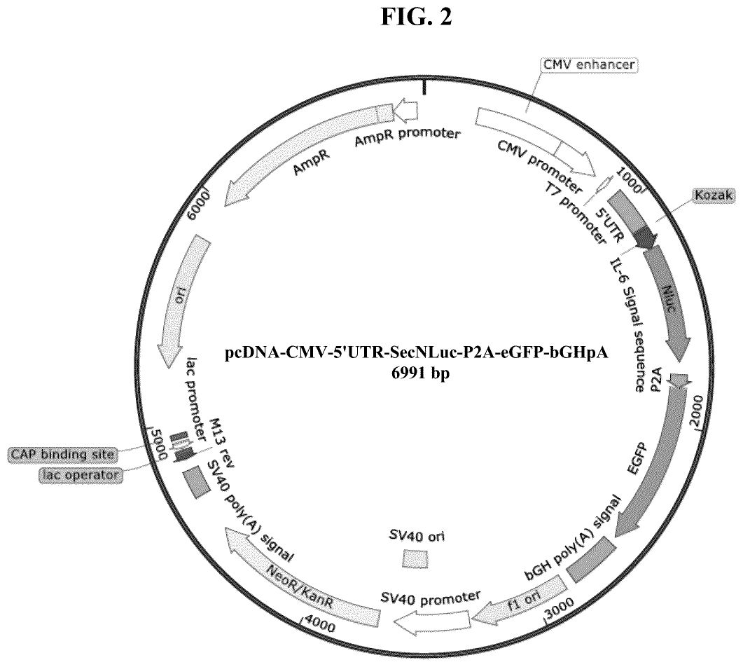

A second polygenic expression vector was prepared by cloning the same eGFP and Nluc sequences along with a 5′UTR into the pcDNA3.1 vector (Thermo Fisher Scientific). A map of the expression vector is shown in FIG. 2 (vector pcDNA-CMV-5′UTR-SecNLuc-P2A-eGFP-bGHpA), which contains a polygenic expression cassette containing the CMV enhancer/promoter, sequences encoding eGFP and SecNLuc connected by a P2A self-cleaving peptide (SecNLuc-P2A-eGFP), and a bovine growth hormone polyadenylation signal (bGHpA). The nucleic acid sequence for the vector is provided as SEQ ID NO:20.

### B. Transfection of HEK293 Cells

Adherent human embryonic kidney 293 (HEK293) cells were seeded in a 24-well plate at 1×10⁵ cells/well.

A complex of expression vector (1 μg) and lipofectamine (3 μL) was prepared and incubated using standard operating procedures for each of (1) pGL2-SS*-CAG-SecNLuc-2A-eGFP-BGpA-SS*, (2) pcDNA-CMV-5′UTR-SecNLuc-P2A-eGFP-bGHpA, and (3) pGL2-SS*-CAG-eGFP-BGpA-SS*.

The three complexes were used to separately transfect HEK293 cells via electroporation in individual wells, which were then incubated for 48 hours. HEK293 cells in other wells were treated with 3 μL lipofectamine containing no plasmid as a negative control.

Cells were evaluated for cytoplasmic GFP expression and luciferase expression 48 hours after transfection.

### C. Cytoplasmic GFP Expression

Cytoplasmic GFP expression was used as a measure of transfection efficiency and gene expression levels by the polygenic expression vectors. Expression was evaluated by fluorescent microscopy, and mean GFP expression/intensity of the experimental expression vectors (pGL2-SS*-CAG-SecNLuc-2A-eGFP-BGpA-SS* and pcDNA-CMV-5′UTR-SecNLuc-P2A-eGFP-bGHpA) was measured relative to the negative control (cells treated with lipofectamine and no plasmid) and the positive control (pGL2-SS*-CAG-eGFP-BGpA-SS*), also referred to herein as parental plasmid CAG-GFP, i.e., PP-CAG-GFP).

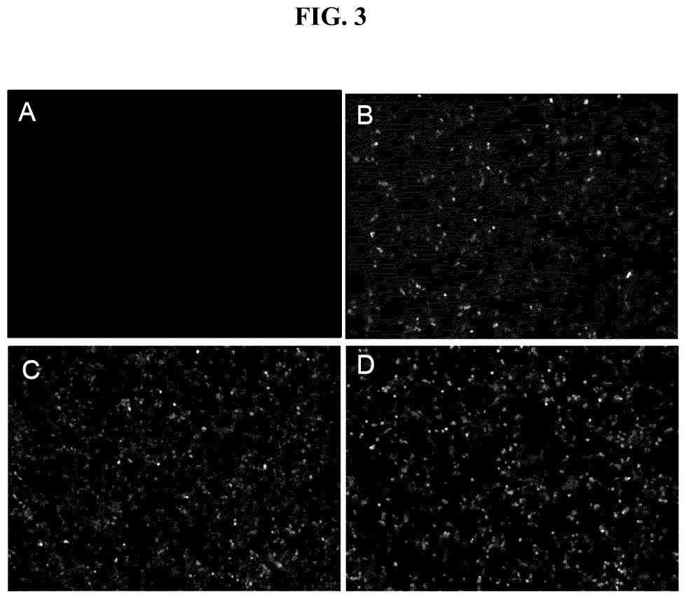

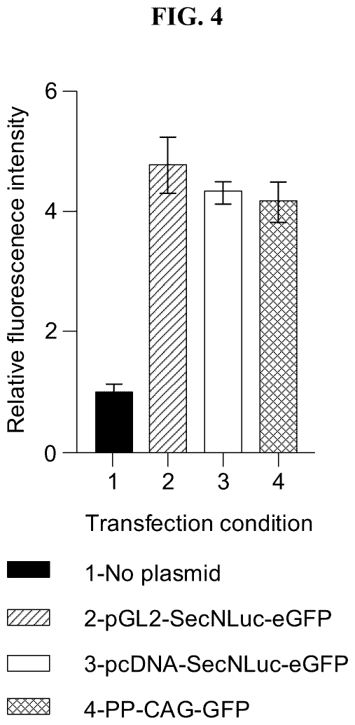

Live imaging of fluorescent cells under auto exposure mode showed that the experimental expression vectors produced GFP, with the chimeric intron of pGL2-SS*-CAG-SecNLuc-2A-eGFP-BGpA-SS*and the 5′UTR of pcDNA-CMV-5′UTR-SecNLuc-P2A-eGFP-bGHpA having similar expression. See FIGS. 3 and 4. Polygenic expression by the experimental expression vectors did not impact GFP expression based on mean relative fluorescence intensities as compared to the positive control. Id. The mean fluorescence intensity in cells transfected with the experimental expression vectors was at least 3-fold higher than negative control cells. See FIG. 4.

### D. Luciferase Expression

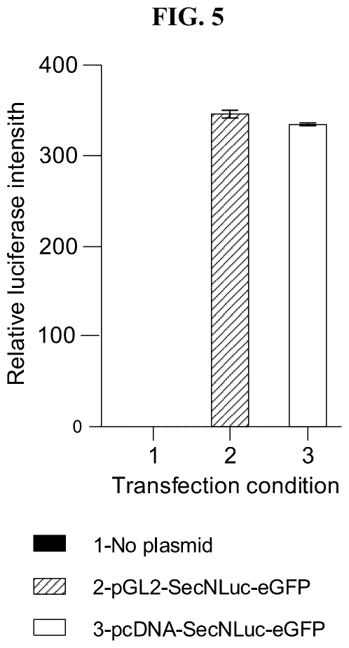

Luciferase expression was evaluated by measuring the intensity of secreted luciferase in the media of transfected cells and negative control cells using the Nano-Glo® Luciferase Assay System (Promega) according to manufacturer protocols. Both experimental expression vectors expressed luciferase. See FIG. 5. The mean relative luciferase intensity in the media of cells transfected with the experimental expression vectors was at least 300-fold higher than in the media of negative control cells. Id.

### Example 2—Expression Vectors Containing WPRE and Engineered 5′UTRs

### A. Expression Vectors

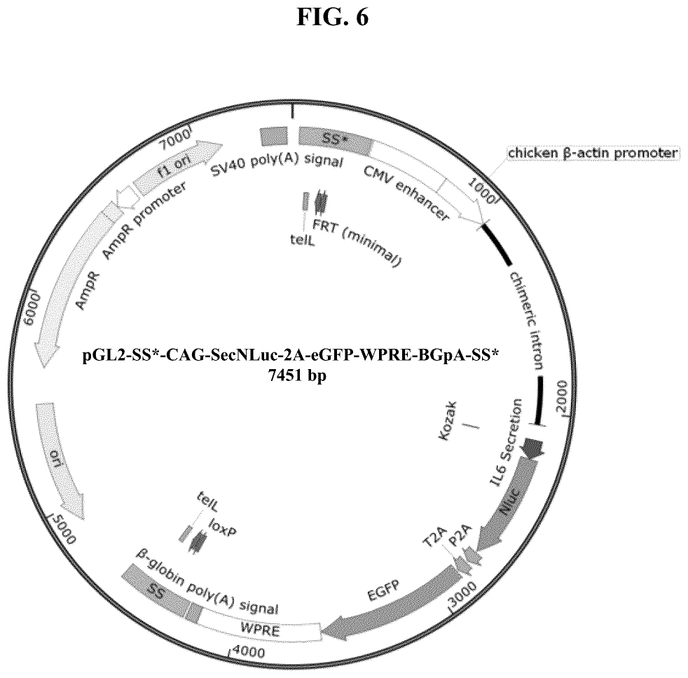

A polygenic expression vector was prepared by cloning a woodchuck hepatitis virus post-transcriptional regulatory element (WPRE) between the sequence encoding eGFP and BGpA in the expression vector of FIG. 1. The map of the resultant expression vector is shown in FIG. 6 (pGL2-SS*-CAG-SecNLuc-2A-eGFP-WPRE-BGpA-SS*). The nucleic acid sequence for the vector is provided as SEQ ID NO:21.

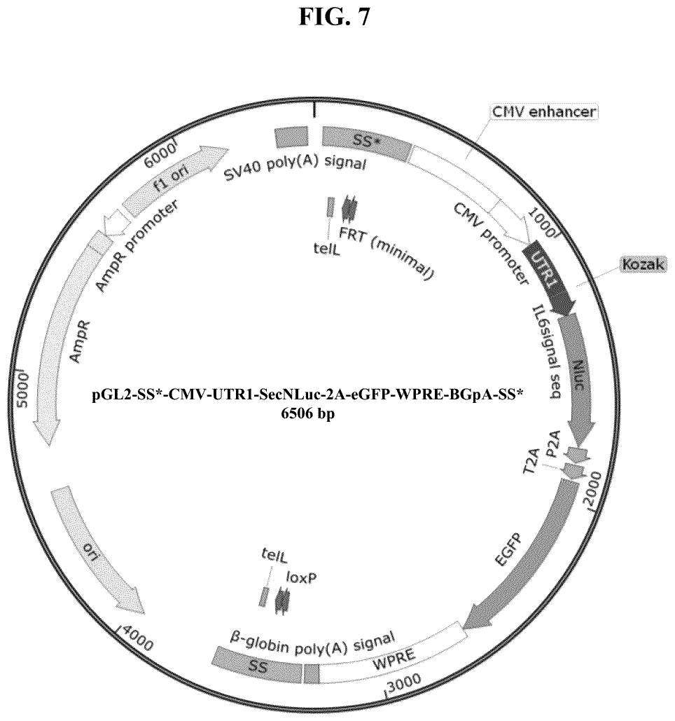

Another polygenic expression vector was prepared that contains a CMV enhancer/promoter and an engineered 5′UTR containing an internal minimal intron sequence (i.e., 5′UTR1, SEQ ID NO:2) in place of the CAG promoter in FIG. 6. The map of the resultant expression vector is shown in FIG. 7 (pGL2-SS*-CMV-UTR1-SecNLuc-2A-eGFP-WPRE-BGpA-SS*). The nucleic acid sequence for the vector is provided as SEQ ID NO:22.

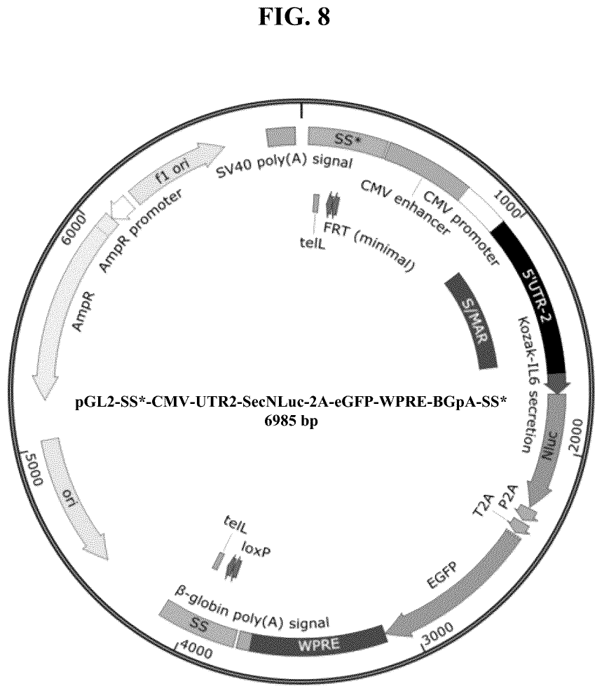

A further polygenic expression vector was prepared that contains a CMV enhancer/promoter and an engineered 5′UTR containing an intron with an integrated MAR-5 (i.e., 5′UTR2, SEQ ID NO:5) in place of the CAG promoter in FIG. 6. The map of the resultant expression vector is shown in FIG. 8 (pGL2-SS*-CMV-UTR2-SecNLuc-2A-eGFP-WPRE-BGpA-SS*). The nucleic acid sequence for the vector is provided as SEQ ID NO:23.

### B. Luciferase Expression Levels and Durability

Adherent HEK293 cells were detached, resolved in electroporation media, and counted at 1×10⁶ cells/tube.

The expression vector (1 μg) was prepared and incubated with cells using standard operating procedures for each of (1) pGL2-SS*-CAG-SecNLuc-2A-eGFP-BGpA-SS* (see Example 1), (2) pGL2-SS*-CAG-SecNLuc-2A-eGFP-WPRE-BGpA-SS*, (3) pGL2-SS*-CMV-UTR1-SecNLuc-2A-eGFP-WPRE-BGpA-SS*, and (4) pGL2-SS*-CMV-UTR2-SecNLuc-2A-eGFP-WPRE-BGpA-SS*.

HEK293 cells electroporated with puc57 plasmid lacking a mammalian expression cassette served as a negative control.

After electroporation, HEK293 cells were seeded and adhered to wells at 3×10⁵ cells/well.

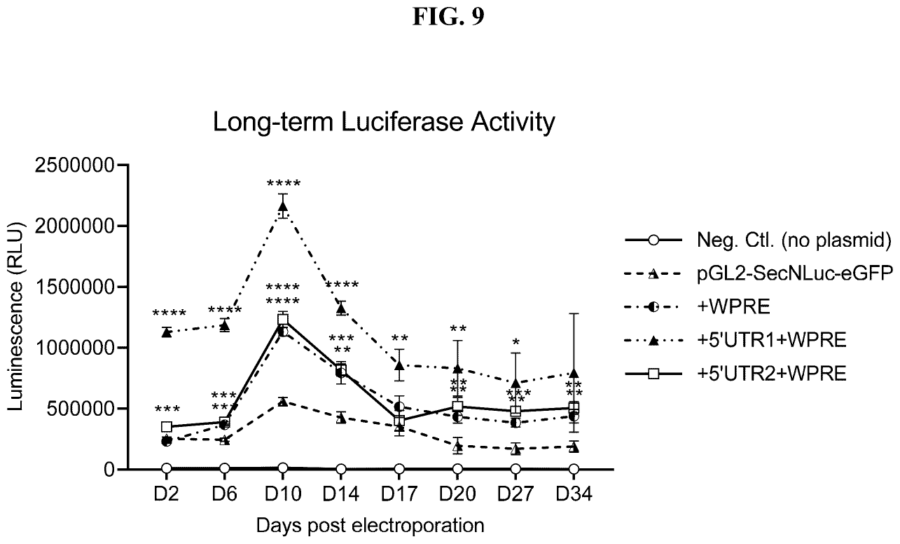

On days 2, 6, 10, 14, 17, 20, 27, and 34 after electroporation, luciferase expression was evaluated by measuring the intensity of secreted luciferase in 20 μL of cell culture media in triplicate for each of the four transfections and the negative control using the Nano-Glo® Luciferase Assay System (Promega) according to manufacturer protocols. Luciferase activity was measured using a BioTek® plate reader and displayed in Relative Luminometer Units (RLU). Statistical analysis of luciferase activity was performed by Student's T-test. See FIG. 9, showing expression levels in media from cells transfected with pGL2-SS*-CAG-SecNLuc-2A-eGFP-BGpA-SS* (pGL2-SecNLuc-eGFP), pGL2-SS*-CAG-SecNLuc-2A-eGFP-WPRE-BGpA-SS* (WPRE), pGL2-SS*-CMV-UTR1-SecNLuc-2A-eGFP-WPRE-BGpA-SS* (5′UTR1+WPRE), or pGL2-SS*-CMV-UTR2-SecNLuc-2A-eGFP-WPRE-BGpA-SS* (5′UTR2+WPRE) as compared to the negative control (Neg. Ctl. (no plasmid)). *=p<0.05, **=p<0.01, ***=p<0.001 and ****=p<0.0001.

Luciferase expression was detected throughout the duration of the experiment from cells transfected with any of the four expression vectors. pGL2-SS*-CAG-SecNLuc-2A-eGFP-WPRE-BGpA-SS*, pGL2-SS*-CMV-UTR1-SecNLuc-2A-eGFP-WPRE-BGpA-SS*, and pGL2-SS*-CMV-UTR2-SecNLuc-2A-eGFP-WPRE-BGpA-SS* all showed significantly higher luciferase expression as compared to pGL2-SS*-CAG-SecNLuc-2A-eGFP-BGpA-SS*, with pGL2-SS*-CMV-UTR1-SecNLuc-2A-eGFP-WPRE-BGpA-SS* showing the highest enhancement of expression.

### C. Vector Expansion to Daughter Cells and Luciferase Expression

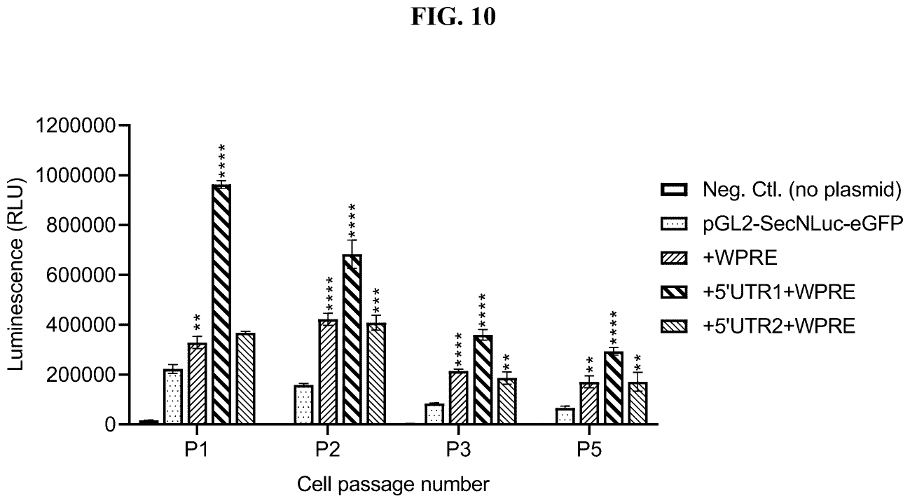

HEK293 cells were transfected with the four expression vectors or the puc57 plasmid as a negative control, as described in part B of this example. Cells were passaged weekly for five passages. At the time of cell passaging, cells were re-seeded at ⅙ of the original cell density for passage numbers 1-3, and 1/10 of the original cell density for passage numbers 4-5. For each cell passage, secreted luciferase expression was measured 6-8 days after cell re-seeding as described in part B of this example. See FIG. 10, showing expression levels in media from cells transfected with the vectors as compared to the negative control at each passage number. Statistical analysis and p values were as noted in part B of this example.

Luciferase expression was detected from cells transfected with any of the four expression vectors at each passage number, showing that the vectors were passed down to daughter cells with durable expression of luciferase. pGL2-SS*-CAG-SecNLuc-2A-eGFP-WPRE-BGpA-SS*, pGL2-SS*-CMV-UTR1-SecNLuc-2A-eGFP-WPRE-BGpA-SS*, and pGL2-SS*-CMV-UTR2-SecNLuc-2A-eGFP-WPRE-BGpA-SS* all showed significantly higher luciferase expression at each passage number as compared to pGL2-SS*-CAG-SecNLuc-2A-eGFP-BGpA-SS*, with pGL2-SS*-CMV-UTR1-SecNLuc-2A-eGFP-WPRE-BGpA-SS* showing the highest enhancement of expression.

In a subsequent study, msDNA expansion to daughter cells with durable expression of luciferase was also observed.

Briefly, msDNA was produced from pGL2-SS*-CMV-UTR1-SecNLuc-2A-eGFP-WPRE-BGpA-SS* in an inducible E. coli vector production system using methods described herein and in U.S. Pat. Nos. 9,290,778 and 9,862,954. Separate complexes with lipofectamine were prepared with (1) the msDNA (i.e., msDNA-CMV-UTR1-SecNLuc-2A-eGFP-WPRE-BGpA) (2) the parental plasmid (i.e., pGL2-SS*-CMV-UTR1-SecNLuc-2A-eGFP-WPRE-BGpA-SS*), and (3) a conventional plasmid with a luciferase expression cassette (i.e., pcDNA-CMV-5′UTR-SecNLuc-P2A-eGFP-bGHpA). HEK293 cells were separately transfected with the vectors via electroporation in individual wells for a total of 0.25 pmol vector/well. Cells were passaged 7 times, with a 10-fold cell dilution at each passage. Relative luciferase intensity was determined on days 8, 15, 24, 31, 38, 45, and 52 for passage numbers 1, 2, 3, 4, 5, 6, and 7, respectively.

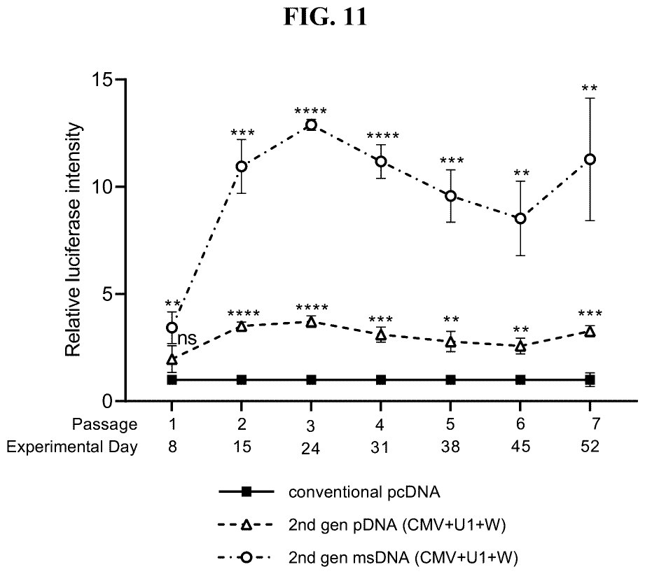

As shown in FIG. 11, pGL2-SS*-CMV-UTR1-SecNLuc-2A-eGFP-WPRE-BGpA-SS* (i.e., pDNA (CMV+U1+W)) and msDNA-CMV-UTR1-SecNLuc-2A-eGFP-WPRE-BGpA (i.e., msDNA (CMV+U1+W) demonstrated durable transgene expression at much higher levels across all passage numbers than pcDNA-CMV-5′UTR-SecNLuc-P2A-eGFP-bGHpA (i.e., conventional plasmid containing no supersequence (conventional pcDNA)). **=p<0.01, ***=p<0.001 and ****=p<0.0001 as compared to the conventional plasmid dataset.

D. Vector Expansion to Daughter Cells and eGFP Expression

Cells transfected with pGL2-SS*-CAG-SecNLuc-2A-eGFP-BGpA-SS* (pGL2-SecNLuc-eGFP) or pGL2-SS*-CMV-UTR1-SecNLuc-2A-eGFP-WPRE-BGpA-SS* (5′UTR1+WPRE) and passaged according to part C of this example were analyzed for eGFP expression.

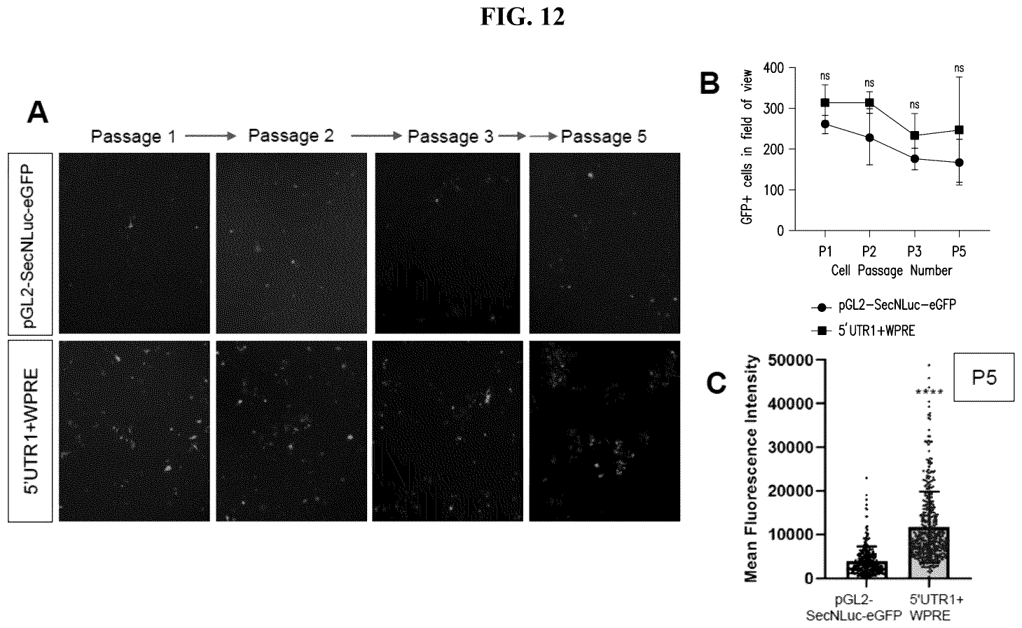

Imaging was performed 6 to 8 days after cell passaging for each passage number. Live cell imaging was performed using a BioTek® Cytation™ 5 plate reader. See FIG. 12A, showing representative photomicrographs of fluorescence in HEK-293 cells at passage numbers 1, 2, 3, and 5. eGFP expression was detected from cells transfected with either of the expression vectors at each passage number, showing that the vectors were passed down to daughter cells with durable expression of eGFP. pGL2-SS*-CMV-UTR1-SecNLuc-2A-eGFP-WPRE-BGpA-SS* showed a stronger fluorescent signal at each passage number as compared to pGL2-SS*-CAG-SecNLuc-2A-eGFP-BGpA-SS*, indicating a higher transfection efficiency.

One representative image was taken from each triplicate well for each expression vector, and eGFP expressing cells (GFP) were quantified by manual cell counting using ImageJ computer software. Statistical analysis of GFP′ cells was performed using a Student's t-test. See FIG. 12B, showing a line graph of GFP′ cells observed in the field of view from the triplicate live fluorescent images at each passage number (non-significant (ns)=p>0.05 due to variance in the triplicate images).

ImageJ software was used to manually select each GFP′ cell and measure the Mean Fluorescence Intensity (MFI) for each cell based on pixel intensity. To calculate the final MFI value for each cell, the following formula was used (Final MFI=Cell MFI−Background MFI). MFI measurements were obtained for at least 50 cells from each of the 3 images taken for each treatment group. All MFI measurements were then pooled and used to generate a dot plot. Statistical analysis was performed using a Student's t-test. See FIG. 12C, showing a dot plot of MFI at passage number 5, n=257 cells for pGL2-SS*-CAG-SecNLuc-2A-eGFP-BGpA-SS* and n=414 cells for pGL2-SS*-CMV-UTR1-SecNLuc-2A-eGFP-WPRE-BGpA-SS*, ****=p<0.0001. The underlying bar graph in FIG. 12C shows the average MFI value for all measured GFP′ cells. The MFI of pGL2-SS*-CMV-UTR1-SecNLuc-2A-eGFP-WPRE-BGpA-SS* was measured to be roughly 3-fold higher than pGL2-SS*-CAG-SecNLuc-2A-eGFP-BGpA-SS*.

### Example 4—Nonviral Delivery with msDNA in Animal Models

Studies were conducted to assess targeted delivery of msDNA to the liver, retina, and brain. For each target tissue, different routes of administration (ROAs), doses, dosing regimens, and delivery techniques were evaluated. Secreted luciferase expression kinetics, cytoplasmic eGFP expression levels, and transfection efficiency (TE) were evaluated. In addition, tolerability to the msDNA was evaluated after single or multiple injections by physiological assessment, tissue morphology analysis, plasma cytokine assay, and liver toxicity analysis.

Across all delivery techniques, msDNA showed strong efficacy and tolerability profiles in the brain and liver tissues via multiple intracerebroventricular (ICV) or hydrodynamic injections (HDI) and intravenous (IV) injections, respectively. Adult mice treated with msDNA showed sustained secreted luciferase levels (>10⁸ RLU/mg protein) after a single IV injection. The msDNA showed durable (>100 days) expression in the liver tissue after a single IV injection. Significant biodistribution to deep tissue regions was also demonstrated, with 80% to 97% TE in brainstem, cerebellum, cortex, and thalamus. The triple ICV injections with the nanocarrier-msDNA complex did not show any side effects.

### A. Liver

1. Expression of Luciferase from a Single High Dose, 2 mg/kg (50 μg), Hydrodynamic Injection of Carrier-Free Naked Plasmid

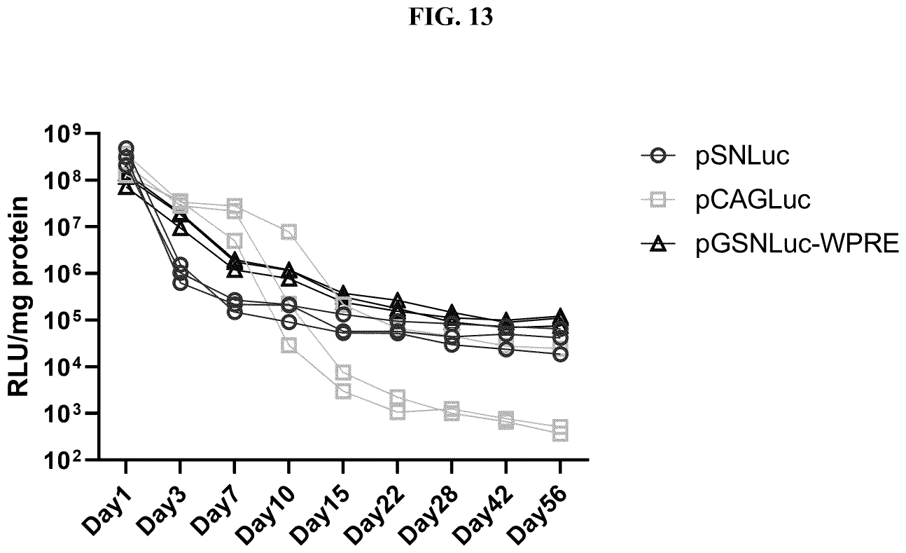

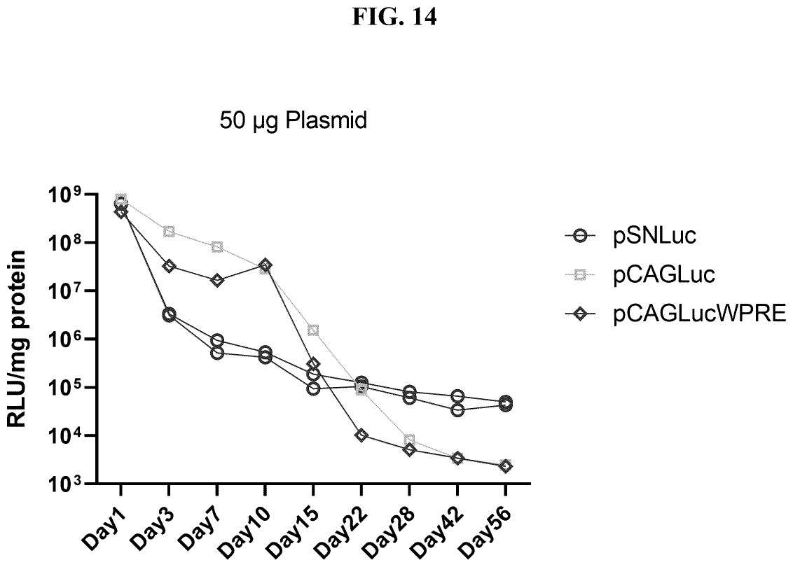

C57BL/6J male wild-type adult 8-12 weeks old mice were administered a single high dose of 2 mg/kg (50 μg) of carrier-free pcDNA-CMV-5′UTR-SecNLuc-P2A-eGFP-bGHpA (positive control with no supersequence), pGL2-SS*-CAG-SecNLuc-2A-eGFP-BGpA-SS*, pGL2-SS*-CMV-UTR1-SecNLuc-2A-eGFP-WPRE-BGpA-SS*, or pGL2-SS*-CAG-SecNLuc-2A-eGFP-WPRE-BGpA-SS* by hydrodynamic injection (HDI) via the tail vein. The plasma of the treated mice was collected on days 1, 3, 7, 10, 15, 22, 28, 42, and 56 after HDI to examine luciferase gene expression. On day 1 post-vector administration, all mice exhibited high levels of luciferase expression (108-109 RLU per mg of plasma protein). On day-7, the pCAGLuc and the pCAGLuc-WPRE treated mice produced 107-108 RLU/mg of plasma protein, but the pGSNLuc-WPRE treated mice yielded lower luciferase levels (˜106 RLU/mg protein). After 8-weeks post-vector administration, all mice exhibited low levels of luciferase expression (around 105 RLU/mg protein). The rapid drop of luciferase levels may have resulted from humoral or cell-mediated immune responses induced in the plasmid treated mice. See FIGS. 13-14.

2. Expression of Luciferase from a Single Low Dose, 0.2 mg/kg (5 μg), Hydrodynamic Injection of Carrier-Free Naked Plasmid

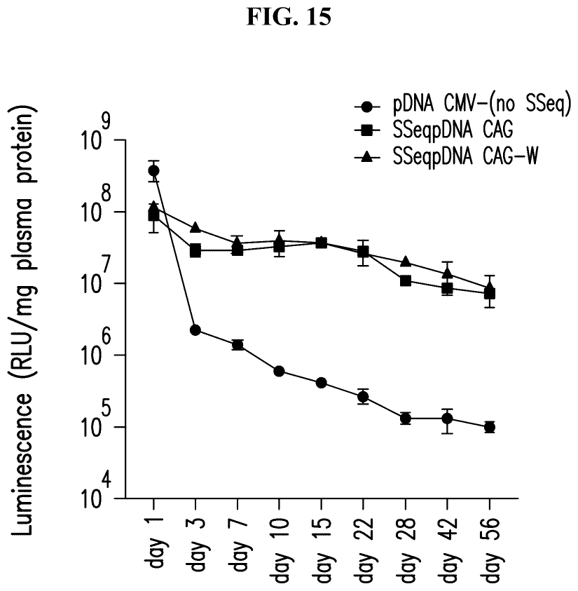

To test dose response of plasmid DNA following nonviral gene delivery in animal models, C57BL/6J male wild-type adult 8-12 weeks old mice were administered a single low dose of 0.2 mg/kg (5 μg) of carrier-free pcDNA-CMV-5′UTR-SecNLuc-P2A-eGFP-bGHpA (positive control with no supersequence, 2 mice), pGL2-SS*-CAG-SecNLuc-2A-eGFP-BGpA-SS* (2 mice), or pGL2-SS*-CAG-SecNLuc-2A-eGFP-WPRE-BGpA-SS* (2 mice) by HDI via the tail vein. An additional 2 mice were not injected and served as a negative control. The plasma of the mice was collected on days 1, 3, 7, 10, 15, 22, 28, 42, and 56 after HDI to examine luciferase gene expression. The mice treated with pGL2-SS*-CAG-SecNLuc-2A-eGFP-BGpA-SS* and pGL2-SS*-CAG-SecNLuc-2A-eGFP-WPRE-BGpA-SS* showed sustained high levels of luciferase expression (107-108 RLU/mg protein) more than 8 weeks post-vector administration and more than 100-fold higher expression than the conventional control plasmid having an isogenic expression cassette but with no supersequence (SS). See FIG. 15.

In vivo whole body bioluminescence imaging (BLI) with IVIS was conducted by injecting a 1:5 dilution of fluorofurimazine (FFz) intraperitoneally 24 hours after HDI of the vectors. The BLI was shown to correlate with the level of luciferase in the plasma samples (data not shown).

3. Expression of Luciferase from a Single Low Dose, 0.2 mg/kg (5 μg), Hydrodynamic Injection of Carrier-Free Naked msDNA

msDNAs were produced from pGL2-SS*-CAG-SecNLuc-2A-eGFP-WPRE-BGpA-SS* and pGL2-SS*-CMV-UTR1-SecNLuc-2A-eGFP-WPRE-BGpA-SS* in an inducible E. coli vector production system using methods described herein and in U.S. Pat. Nos. 9,290,778 and 9,862,954.

C57BL/6J male wild-type adult 8-12 weeks old mice were administered a single low dose of 0.2 mg/kg (5 μg) of carrier-free pcDNA-CMV-5′UTR-SecNLuc-P2A-eGFP-bGHpA (positive control, 5 mice), msDNA-CAG-SecNLuc-2A-eGFP-WPRE-BGpA (5 mice), or msDNA-CMV-UTR1-SecNLuc-2A-eGFP-WPRE-BGpA (5 mice) by hydrodynamic injection (HDI) via the tail vein. An additional 2 mice were not injected and served as a negative control. The plasma of the treated mice was collected on days 1, 3, 7, 10, 15, 22, 28, 42, and 56 after HDI to examine luciferase gene expression.

Similarly to plasmid treated mice, the msDNA-CAG-SecNLuc-2A-eGFP-WPRE-BGpA treated mice produced sustained high levels of luciferase expression (107-108 RLU/mg protein) more than 8 weeks post-vector administration, whereas the luciferase expression in msDNA-CMV-UTR1-SecNLuc-2A-eGFP-WPRE-BGpA treated mice dropped to low levels (˜106 RLU/mg protein) in less than one month. The rapid drop of luciferase expression in msDNA-CMV-UTR1-SecNLuc-2A-eGFP-WPRE-BGpA treated mice was likely due to silencing of the CMV promoter in hepatocytes.

Luciferase gene expression was confirmed via whole body live imaging with IVIS.

Table 1, below, provides data from individual mice on days 1, 7, and 28 for luciferase expression in plasma samples (RLU/mg protein) and as detected by BLI (photons/see).

TABLE 1

| | Day 1 | Day 7 | Day 28 |
|---|---|---|---|
| | RLU/mg | RLU/mg | RLU/mg |
| Vector | photons/sec | photons/sec | photons/sec |
| pcDNA-CMV- | 3.92 × 10⁸ | 4.38 × 10⁶ | 2.18 × 10⁶ |
| 5′UTR-SecNLuc- | 670 | 7.9 | 11.6 |
| P2A-eGFP- | | | |
| bGHpA | | | |
| msDNA-CMV- | 2.49 × 10⁸ | 5.54 × 10⁶ | 8.30 × 10⁵ |
| UTR1-SecNLuc- | 505 | 18 | 4.81 |
| 2A-eGFP-WPRE- | | | |
| BGpA | | | |
| msDNA-CAG- | 1.53 × 10⁸ | 3.76 × 10⁷ | 2.55 × 10⁷ |
| SecNLuc-2A- | 325 | 188 | 178 |
| eGFP-WPRE- | | | |
| BGpA | | | |

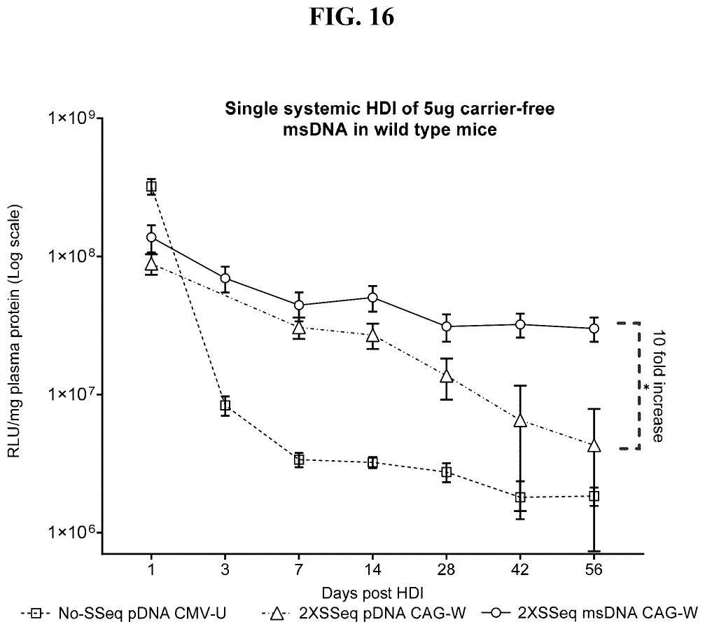

As shown in FIG. 16, mice treated with msDNA-CAG-SecNLuc-2A-eGFP-WPRE-BGpA demonstrated a 10-fold increase in luciferase expression as compared to the parental plasmid, pGL2-SS*-CAG-SecNLuc-2A-eGFP-WPRE-BGpA-SS* at day 56 after HDI.

The data show that nonviral delivery with msDNA in mice was highly efficient and the resulting gene expression was stable for more than two months.

4. Expression of eGFP from a Single Low Dose, 0.2 mg/kg (5 μg), Hydrodynamic Injection of Carrier-Free Naked msDNA

Intracellular cytoplasmic eGFP expression levels were evaluated by ELISA. Briefly, liver samples were collected from mice at 56 days after HDI with the single low dose of 0.2 mg/kg (5 μg) of the vectors as described in part 3 and homogenized for protein extraction. Total protein concentrations were determined from the liver lysates. GFP protein levels were then analyzed by ELISA.

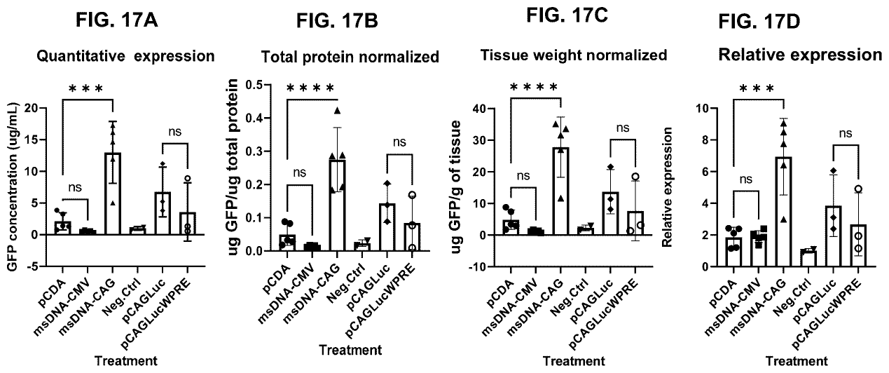

As evident by comparing the data in FIG. 17 to the data for luciferase, cytoplasmic GFP expression levels directly correlated to secretion levels of luciferase from the same constructs.

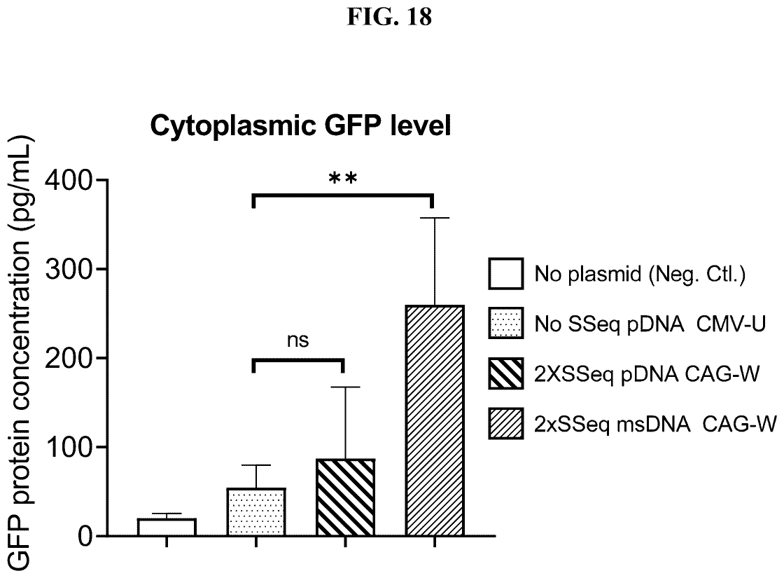

As shown in FIG. 18, single HDI tail-vein injection of 5 μg of carrier-free msDNA-CAG-SecNLuc-2A-eGFP-WPRE-BgpA showed strong and durable cytoplasmic eGFP expression in the liver tissue for a minimum of 56 days after HDI.

5. Expression of msDNA in Liver after Low Dose Single Intravenous Injection and Tolerability Profile

C57BL/6J male wild-type adult 8-12 weeks old mice were administered 0.3 mg/kg pcDNA-CMV-5′UTR-SecNLuc-P2A-eGFP-bGHpA (positive control with no supersequence), msDNA-CMV-UTR1-SecNLuc-2A-eGFP-WPRE-BGpA, pGL2-SS*-CMV-UTR1-SecNLuc-2A-eGFP-WPRE-BGpA-SS*, msDNA-CAG-SecNLuc-2A-eGFP-WPRE-BGpA, or pGL2-SS*-CAG-SecNLuc-2A-eGFP-WPRE-BGpA-SS* lipoplexed with a lipid nanoparticle carrier through a single intravenous tail vein injection. The carrier also served as a negative vehicle control.

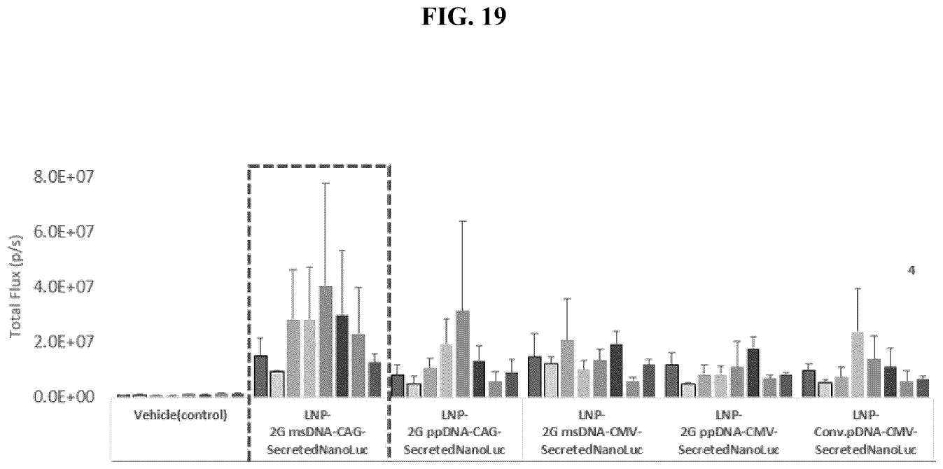

In vivo whole body bioluminescence imaging (BLI) with IVIS was conducted as described above on days 1, 3, 10, 30, 58, 92, 119, and 174 after the single IV injection of the vectors. As shown in FIG. 19, the msDNA constructs outperform the precursor and conventional plasmids and show high and sustained luciferase secretion.

Serum alanine aminotransferase (ALT) level, liver cytotoxicity, and cytokine responses also were evaluated following injection of the vectors. Precursor plasmid and msDNA containing the CAG promoter showed a higher tolerability profile compared to constructs containing the CMV promoter. However, msDNA containing the CMV promoter showed dramatically lower cytokine and liver toxicity responses compared to the CMV precursor parent and the conventional plasmid. See Table 2, below, showing cytokine concentrations (pg/mL) and enzyme concentrations (U/L) of liver function markers at 4 hours and 14 days after injection.

TABLE 2

| | | DNA Dose | Group Average - Concentration (pg/ml) | | | | | |
|---|---|---|---|---|---|---|---|---|
| | | (mg/kg) | IFN-α | IFN-γ | IL-1β | IL-6 | L-12 p70 | IP-10 |
| 4 h | Vehicle (Control) | — | 40.89 | 1.39 | 5.55 | 29.5 | 97.68 | 112.77 |
| | LNP-2G ppDNA- | 0.3 | 27506.75 | 22697.04 | 491.49 | 329619.3 | 610.06 | 195642.5 |
| | CMV- | | | | | | | |
| | SecretedNanoLuc | | | | | | | |
| | LNP-2G msDNA- | 0.3 | 11175.01 | 1195.49 | 27.66 | 74947.2 | 308.78 | 101806.5 |
| | CMV- | | | | | | | |
| | SecretedNanoLuc | | | | | | | |
| | LNP-2G msDNA- | 0.3 | 10946.45 | 1025.52 | 30.9 | 64315.92 | 271.29 | 120918.1 |
| | CAG- | | | | | | | |
| | SecretedNanoLuc | | | | | | | |
| DAY | Vehicle(control) | — | 35.79 | 1.25 | 4.63 | 22.38 | 106.9 | 53.45 |
| 14 | LNP-2G ppDNA- | 0.3 | 26.71 | 1.53 | 4.44 | 24.38 | 89.53 | 98.53 |
| | CMV- | | | | | | | |
| | SecretedNanoLuc | | | | | | | |
| | LNP-2G msDNA- | 0.3 | 34.58 | 2.18 | 4.7 | 24.19 | 98.62 | 84.34 |
| | CMV- | | | | | | | |
| | SecretedNanoLuc | | | | | | | |
| | LNP-2G msDNA- | 0.3 | 46.42 | 1.46 | 4.98 | 26.34 | 105.96 | 95.04 |
| | CAG- | | | | | | | |
| | SecretedNanoLuc | | | | | | | |

| | | | | | | Group Average - | | |
|---|---|---|---|---|---|---|---|---|
| | | Group Average - Concentration (pg/ml) | | | | Concentration (U/L) | | |
| | | MCP-1 | MIP-1α | MIP-1β | TNF-α | AST | ALT | GLDH |
| 4 h | Vehicle (Control) | 7.38 | 1.47 | 53.28 | 9 | 54.8 | 32.3 | 14.9 |
| | LNP-2G ppDNA- | 41208.92 | 1529.52 | 40716.23 | 10770.27 | 617 | 276.3 | 165.9 |
| | CMV- | | | | | | | |
| | SecretedNanoLuc | | | | | | | |
| | LNP-2G msDNA- | 25085.67 | 734.63 | 19778.17 | 1864.67 | 149.3 | 54.1 | 31.3 |
| | CMV- | | | | | | | |
| | SecretedNanoLuc | | | | | | | |
| | LNP-2G msDNA- | 18144.14 | 568.99 | 19377.7 | 1296.14 | 158.3 | 46.1 | 37.4 |
| | CAG- | | | | | | | |
| | SecretedNanoLuc | | | | | | | |
| DAY | Vehicle(control) | 4.85 | 0.74 | 35.82 | 9.14 | 49.6 | 22.5 | 8.5 |
| 14 | LNP-2G ppDNA- | 6.2 | 0.98 | 37.21 | 14.05 | 65.9 | 24.6 | 10.4 |
| | CMV- | | | | | | | |
| | SecretedNanoLuc | | | | | | | |
| | LNP-2G msDNA- | 6.46 | 1.28 | 50.41 | 11.1 | 73.9 | 35.5 | 19.3 |
| | CMV- | | | | | | | |
| | SecretedNanoLuc | | | | | | | |
| | LNP-2G msDNA- | 6.34 | 1.84 | 46.84 | 13.63 | 67.4 | 26. | 10.3 |
| | CAG- | | | | | | | |
| | SecretedNanoLuc | | | | | | | |

### B. Brain

msDNAs were produced from pGL2-SS*-CAG-SecNLuc-2A-eGFP-WPRE-BGpA-SS* and pGL2-SS*-CMV-UTR1-SecNLuc-2A-eGFP-WPRE-BGpA-SS* in an inducible E. coli vector production system using methods described herein and in U.S. Pat. Nos. 9,290,778 and 9,862,954.

Adult wild type mice were administered msDNA-CAG-SecNLuc-2A-eGFP-WPRE-BGpA formulated with a nanocarrier (3 mice) or msDNA-CMV-UTR1-SecNLuc-2A-eGFP-WPRE-BGpA formulated with a nanocarrier (3 mice) by three intracerebroventricular (ICV) injections of 1 μg DNA each via implanted cannula on days 0, 14, and 28 after implantation. Animals were euthanized on day 42 after implantation, and sagittal brain sections were collected from the cortex, thalamus, brainstem, and cerebellum.

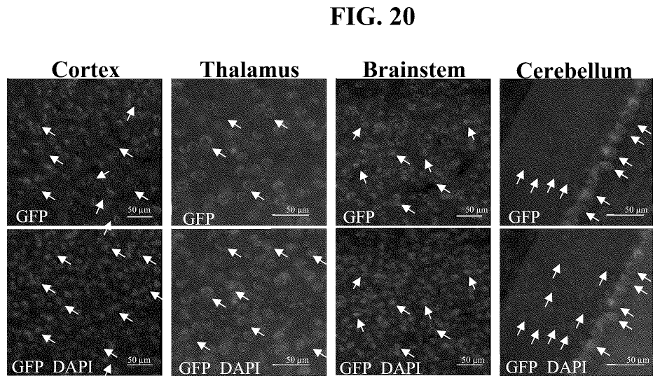

FIG. 20 shows cortex, thalamus, brainstem and cerebellum sections from Mouse #1 of the treatment group injected with msDNA-CAG-SecNLuc-2A-eGFP-WPRE-BGpA. Transfection efficiencies for msDNA in the sections were determined to be 81.9%, 73.0%, 69.2%, and 96.0% in the cortex, thalamus, brainstem, and cerebellum (Purkinje cells), respectively. Transfection efficiencies were calculated as the percentage of cells positive for both GFP and DAPI among all DAPI-positive cells.

Comparisons between GFP expression in the cortex, thalamus, and brainstem sections from Mouse #1 of the treatment group injected with msDNA-CAG-SecNLuc-2A-eGFP-WPRE-BGpA and a mouse injected with control plasmid, pcDNA-CMV-5′UTR-SecNLuc-P2A-eGFP-bGHpA, showed that transfection efficiencies and resultant GFP expression were higher with msDNA versus the conventional plasmid (data not shown).

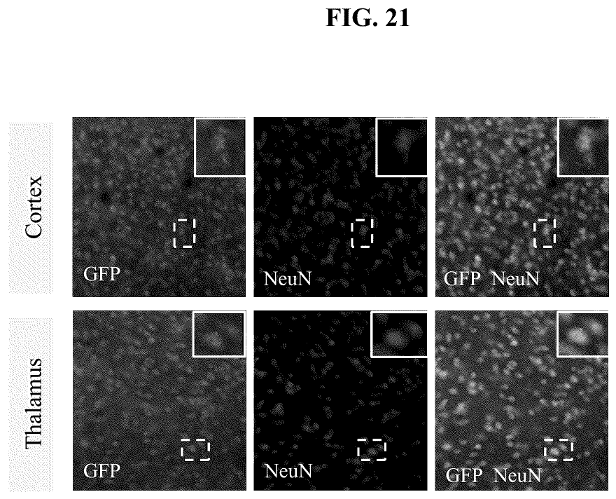

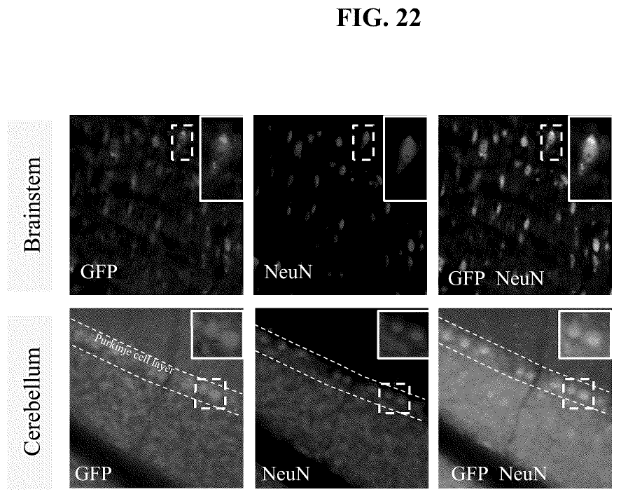

FIG. 21 shows sections from the cortex and thalamus and FIG. 22 shows sections from the brainstem and cerebellum from Mouse #2 of the treatment group injected with msDNA-CAG-SecNLuc-2A-eGFP-WPRE-BGpA. Neurons were marked with the neuronal marker NeuN and transfected cells were shown to express GFP. Transfection efficiencies were determined to be 99.6%, 98.8%, 98.5%, and 80.8% in the cortex, thalamus, brainstem, and the cerebellum (Purkinje cells), respectively. Transfection efficiencies were calculated as the percentage of cells positive for both GFP and NeuN among all NeuN-positive cells.

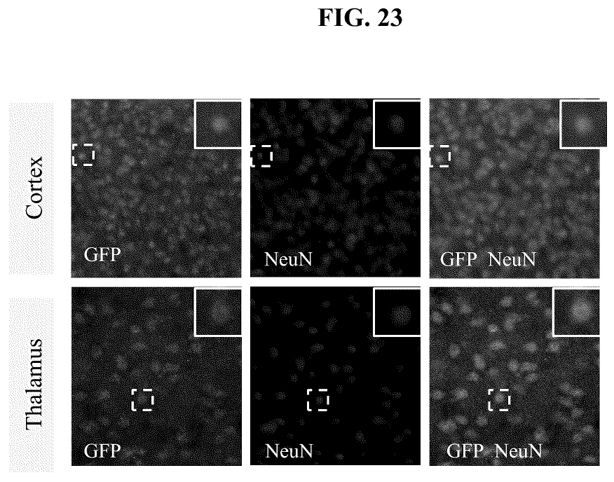

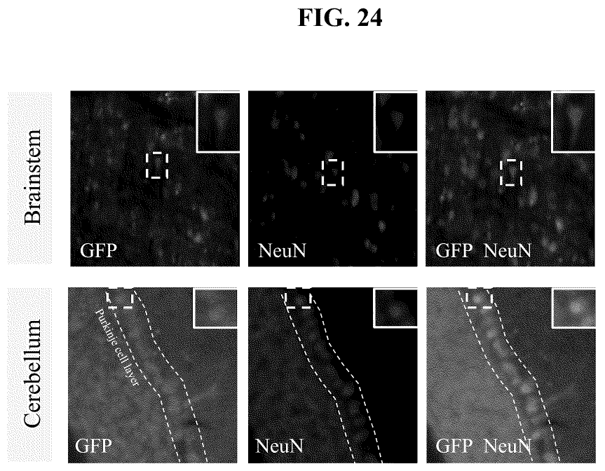

FIG. 23 shows sections from the cortex and thalamus and FIG. 24 shows sections from the brainstem and cerebellum from Mouse #1 of the treatment group injected with msDNA-CMV-UTR1-SecNLuc-2A-eGFP-WPRE-BGpA. Neurons were marked with the neuronal marker NeuN and transfected cells were shown to express GFP. Transfection efficiencies were determined to be 91.1%, 88.8%, 73.7%, and 92.1% in the cortex, thalamus, brainstem, and the cerebellum (Purkinje cells), respectively. Transfection efficiencies were calculated as the percentage of cells positive for both GFP and NeuN among all NeuN-positive cells.

Table 3 below summarizes the transfection efficiencies discussed above for mice injected with msDNA-CAG-SecNLuc-2A-eGFP-WPRE-BGpA (“CAG-WPRE”) or msDNA-CMV-UTR1-SecNLuc-2A-eGFP-WPRE-BGpA (“CMV-WPRE”).

TABLE 3

| | | Brain Region Transfection Efficiency (%) | | | |
|---|---|---|---|---|---|
| | | | | | Cerebellum |
| Treatment | | | | | (Purkinje |
| Group | Animal | Cortex | Thalamus | Brainstem | cells) |
| CAG-WPRE | Mouse #1 | 81.9 | 73.0 | 69.2 | 96.0 |
| CAG-WPRE | Mouse #2 | 99.6 | 98.8 | 98.5 | 80.8 |
| CMV-WPRE | Mouse #1 | 99.1 | 88.8 | 73.7 | 92.1 |

Repeated ICV injections via implanted cannula resulted in good overall tissue integrity with no signs of cytotoxicity or neurodegeneration.

The data show that msDNA was redosable and resulted in high transfection efficiencies, biodistribution, and transgene expression in multiple brain regions with no morphological adverse effects.

### Example 5—Efficacy and Safety in Human Cells

pcDNA-CMV-5′UTR-SecNLuc-P2A-eGFP-bGHpA (positive control), msDNA-CMV-UTR1-SecNLuc-2A-eGFP-WPRE-BGpA, pGL2-SS*-CMV-UTR1-SecNLuc-2A-eGFP-WPRE-BGpA-SS*, msDNA-CAG-SecNLuc-2A-eGFP-WPRE-BGpA, or pGL2-SS*-CAG-SecNLuc-2A-eGFP-WPRE-BGpA-SS* were each lipoplexed with a lipid nanoparticle carrier.

Human T cells (Pan-T(TA+)) and hepatocytes (Huh7) were transfected by 0.3 μg/mL or 1 μg/mL doses of the lipoplexed vectors.

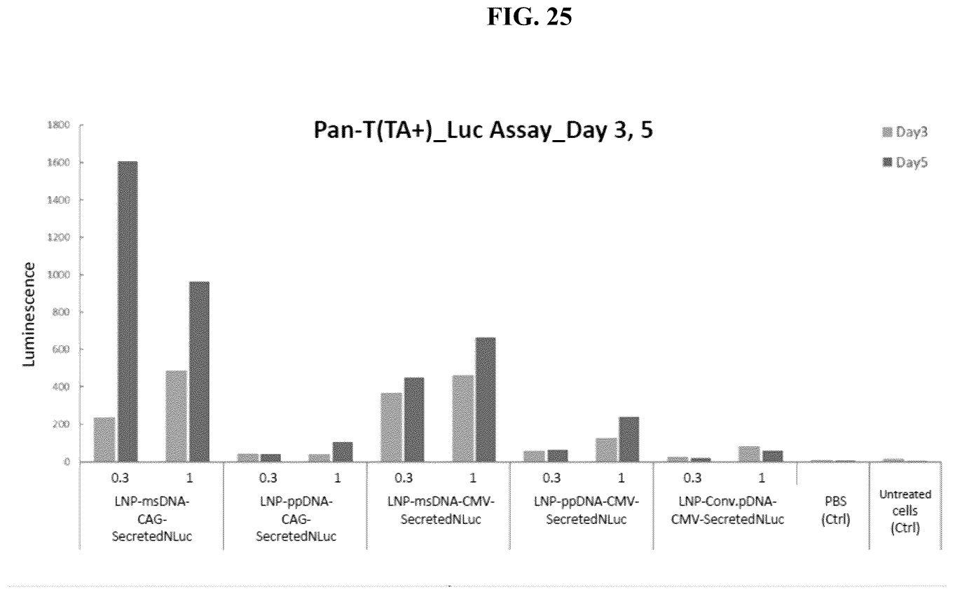

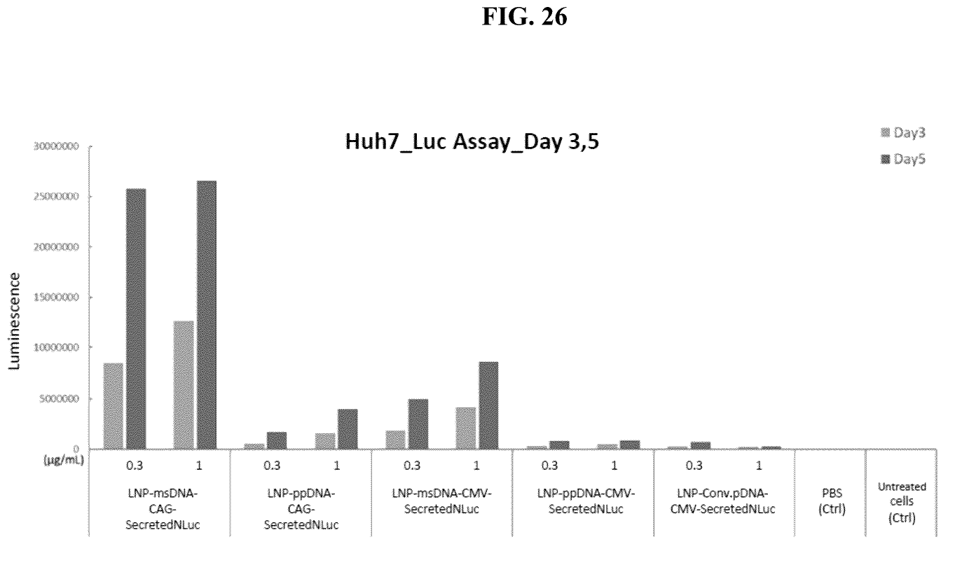

The lipoplexed msDNA vectors showed high expression in both cell types on days 3 and 5 after transfection as compared to the parental and conventional plasmids. See FIGS. 25-26.

Lipoplexed msDNA was also well-tolerated in human peripheral blood mononuclear cells (PBMCs) ex vivo. In particular, msDNA showed significantly lower cytokine profile levels in human PBMCs compared to conventional plasmid (data not shown).

### Example 6—Homology Directed Repair with msDNA

Studies were conducted to assess homology directed repair mediated by msDNA as compared to conventional plasmid DNA.

A conventional plasmid was produced with an expression cassette containing a gene of interest (GOI) flanked by 5′ and 3′ homology arms (Plasmid DNA HDR-GOI-HDR).

An msDNA expression vector was produced with the same HDR-GOI-HDR sequence as used in the conventional plasmid flanked by two Super Sequence sites. msDNA containing the HDR-GOI-HDR (msDNA HDR-GOI-HDR) was then produced in an inducible E. coli vector production system using methods described herein and in U.S. Pat. Nos. 9,290,778 and 9,862,954.

Induced pluripotent stem cells (iPSCs) were transfected with equal molarities of either Plasmid DNA HDR-GOI-HDR or msDNA HDR-GOI-HDR along with a CRISPR gene editing system to mediate homology directed repair knock-in (HDR KI) of the GOI.

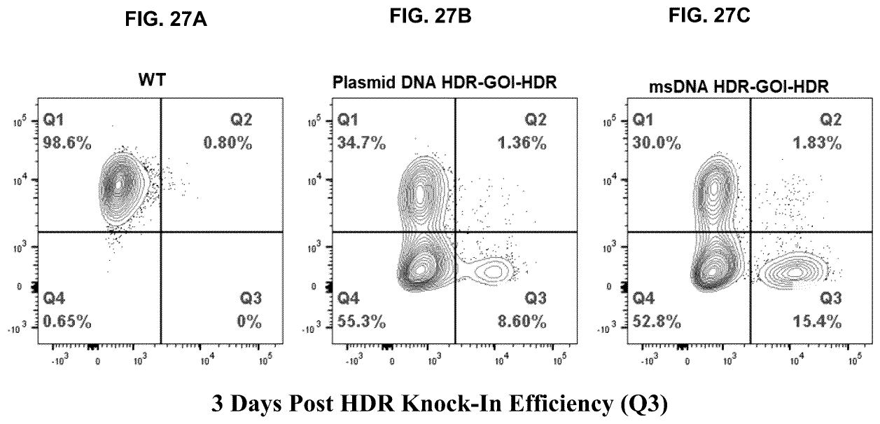

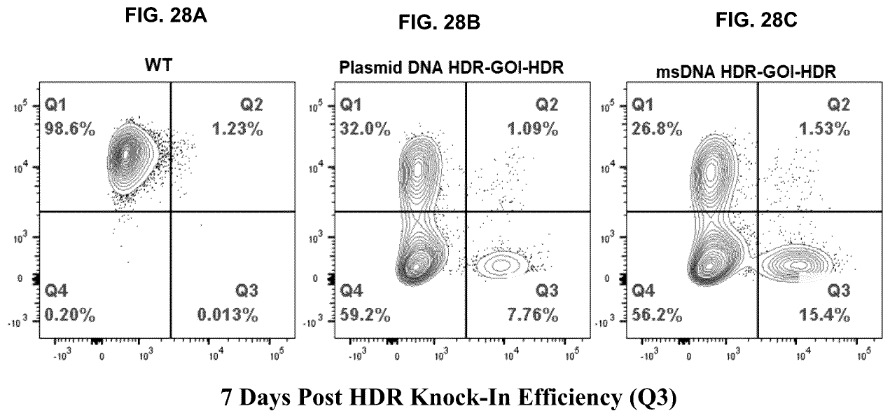

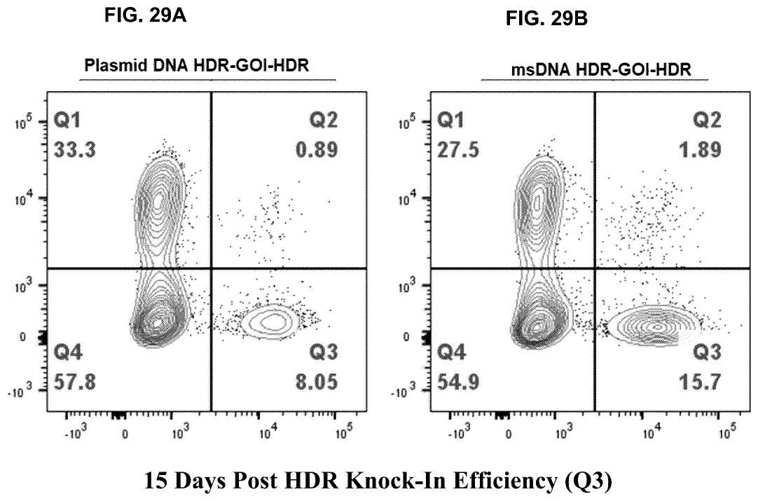

Homology directed repair knock-in (HDR KI) efficiencies of the GOI was evaluated with fluorescence activated cell sorting (FACS) by counting the total number of integrated healthy iPSCs that expressed the GOI on their surface relative to the total number of transfected cells on days 3, 7, and 15 after transfection. As shown in Q3 of FIGS. 27B, 28B, and 29A, the HDR KI efficiencies were 8.60%, 7.76%, and 8.05%, respectively, for the conventional plasmid at 3, 7, and 15 days after transfection, respectively. As shown in Q3 of FIGS. 27C, 28C, and 29B, higher HDR KI efficiencies of 15.4%, 15.4%, and 15.7%, respectively, were observed for msDNA at 3, 7, and 15 days after transfection, respectively.

### Example 7—Expression Vectors Containing Regulatory Sequence Modifications

### A. Expression Vectors

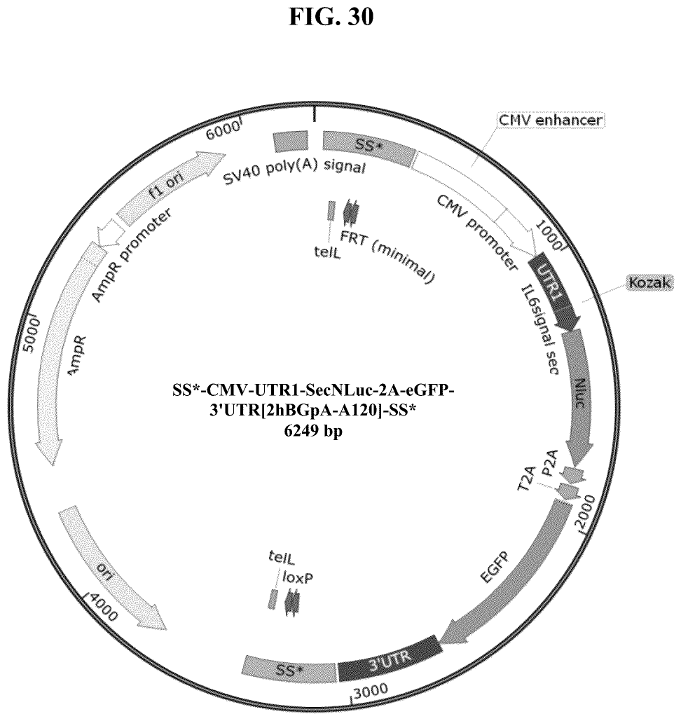

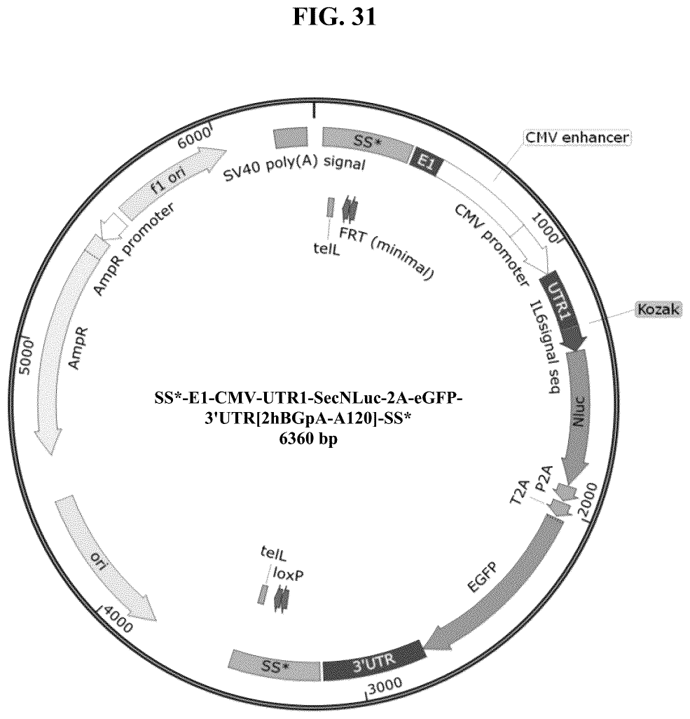

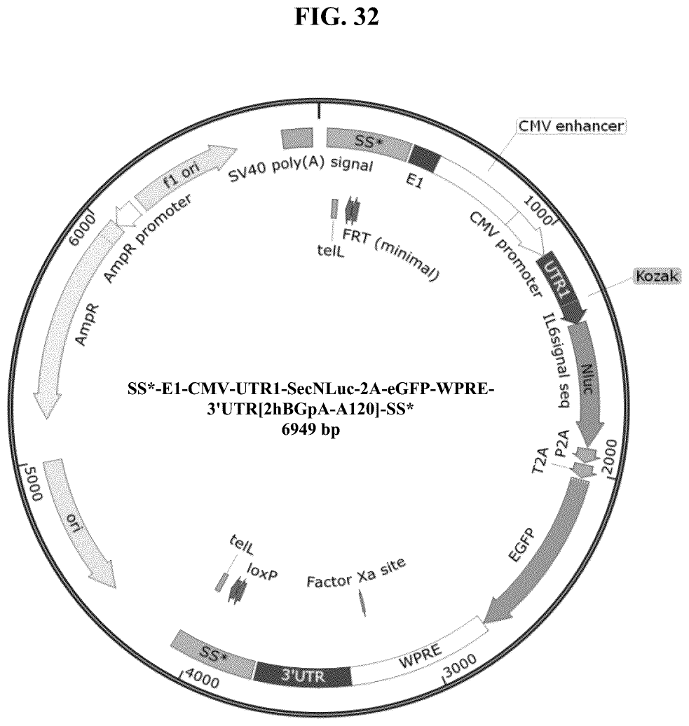

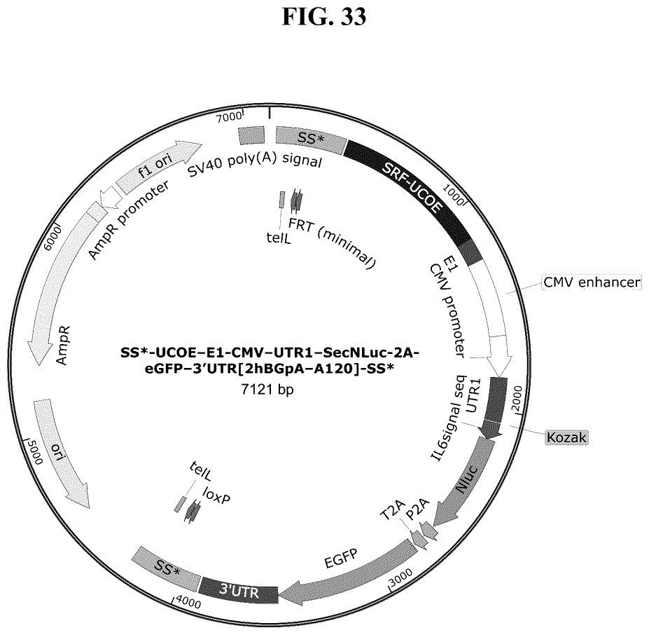

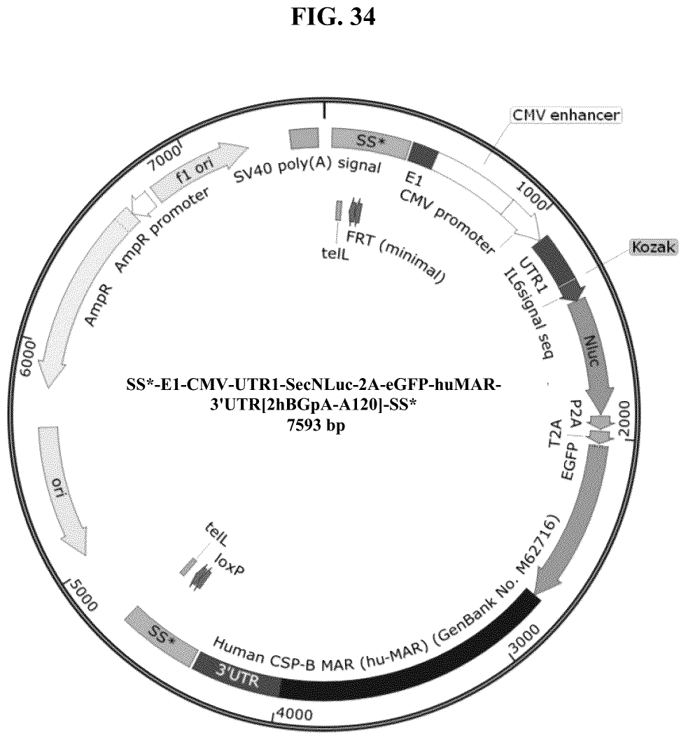

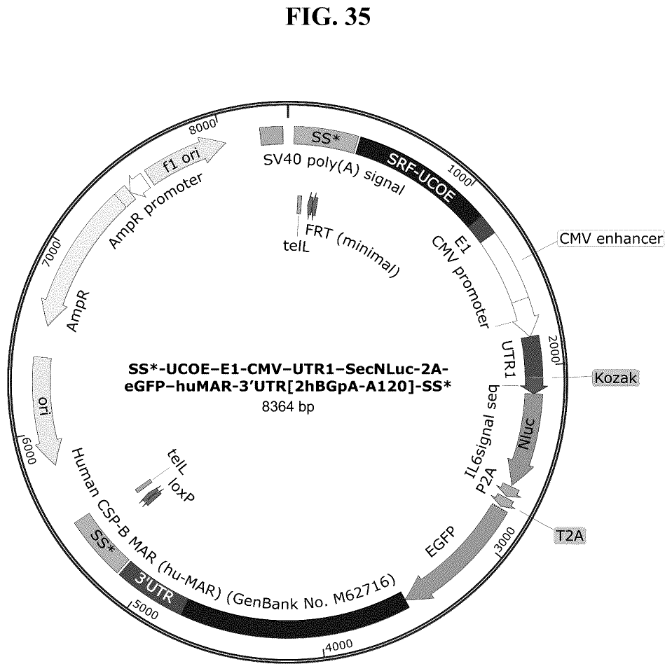

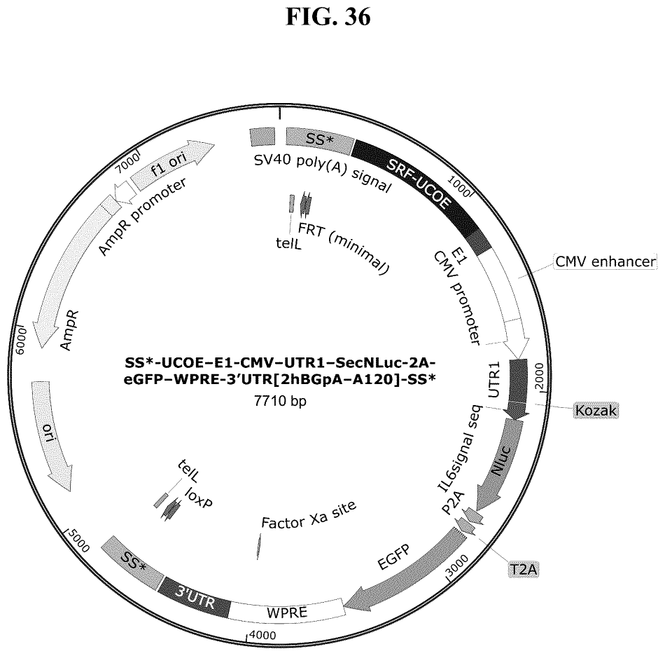

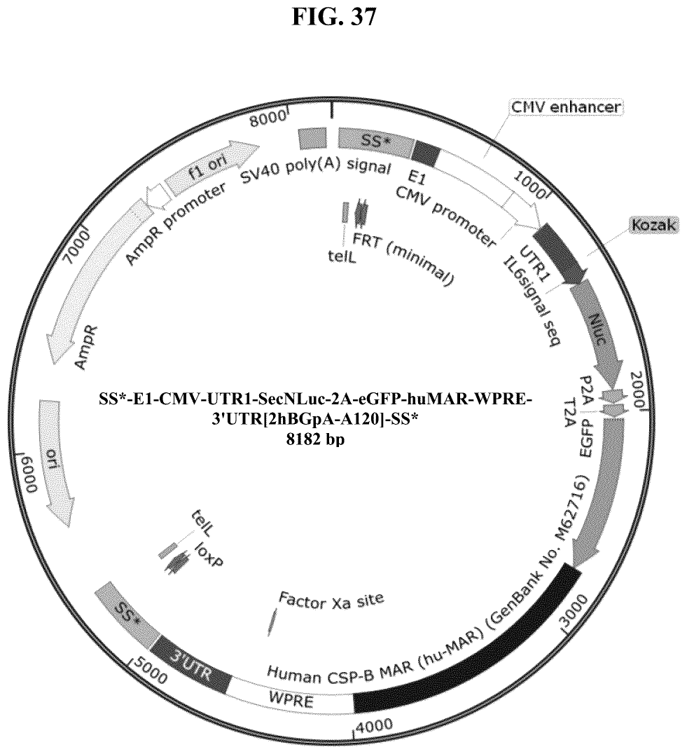

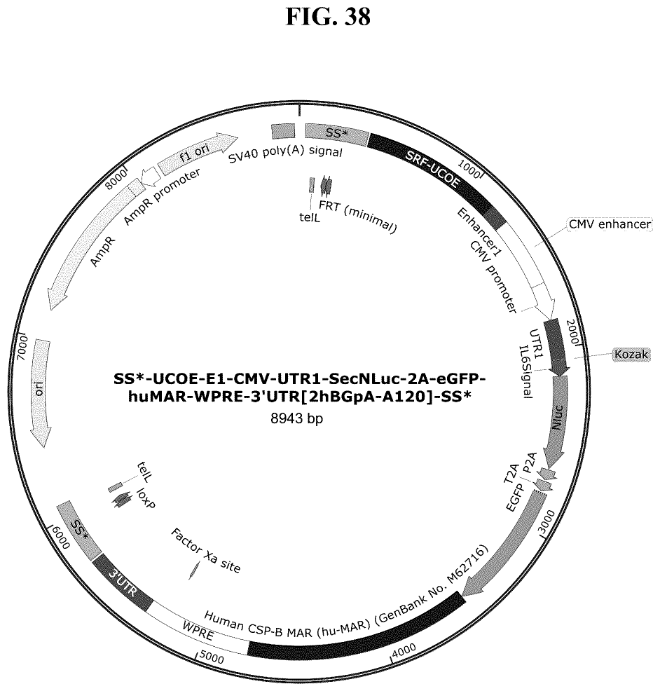

Expression vectors containing two Super Sequence sites, a CMV enhancer/promoter, an engineered 5′UTR containing an internal minimal intron sequence, and a polygenic expression cassette encoding eGFP and Nluc as described in Examples 1 and 2 were also designed to contain a 3′UTR containing two copies of a human beta-globin polyadenylation signal and 120 adenine nucleotides (i.e., 2huBGpA-A120, SEQ ID NO: 17) and one or more of: (1) a synthetic enhancer (i.e., Enhancer-1 (E1), SEQ ID NO: 12) located at the 5′ end of the CMV enhancer, (2) a WPRE located at the 5′ end of the 3′UTR, (3) a SRF-UCOE located at the 3′ end of the 5′ Super Sequence; and (4) a human CSP-B MAR (huMAR) located at the 3′ end of eGFP. Maps of the designed vectors are shown in FIGS. 30-38. FIG. 30 shows a map of the expression vector containing the 3′UTR (SS*-CMV-UTR1-SecNLuc-2A-eGFP-3′UTR[2hBGpA-A120]-SS*, SEQ ID NO: 24). FIG. 31 shows a map of the expression vector containing the E1 and 3′UTR (SS*-E1-CMV-UTR1-SecNLuc-2A-eGFP-3′UTR[2hBGpA-A120]-SS*, SEQ ID NO: 25). FIG. 32 shows a map of the expression vector containing the E1, WPRE, and 3′UTR (SS*-E1-CMV-UTR1-SecNLuc-2A-eGFP-WPRE-3′UTR[2hBGpA-A120]-SS*, SEQ ID NO: 26). FIG. 33 shows a map of the expression vector containing the UCOE, E1, WPRE, and 3′UTR (SS*-UCOE-E1-CMV-UTR1-SecNLuc-2A-eGFP-WPRE-3′UTR[2hBGpA-A120]-SS*, SEQ ID NO: 27). FIG. 34 shows a map of the expression vector containing the E1, huMAR, and 3′UTR (SS*-E1-CMV-UTR1-SecNLuc-2A-eGFP-huMAR-3′UTR[2hBGpA-A120]-SS*, SEQ ID NO: 28). FIG. 35 shows a map of the expression vector containing the UCOE, E1, huMAR, and 3′UTR (SS*-UCOE-E1-CMV-UTR1-SecNLuc-2A-eGFP-huMAR-3′UTR[2hBGpA-A120]-SS*, SEQ ID NO: 29). FIG. 36 shows a map of the expression vector containing the UCOE, E1, WPRE, and 3′UTR (SS*-UCOE-E1-CMV-UTR1-SecNLuc-2A-eGFP-WPRE-3′UTR[2hBGpA-A120]-SS*, SEQ ID NO: 30). FIG. 37 shows a map of the expression vector containing the E1, huMAR, WPRE, and 3′UTR (SS*-E1-CMV-UTR1-SecNLuc-2A-eGFP-huMAR-WPRE-3′UTR[2hBGpA-A120]-SS*, SEQ ID NO: 31). FIG. 38 shows a map of the expression vector containing the UCOE, E1, huMAR, WPRE, and 3′UTR (SS*-UCOE-E1-CMV-UTR1-SecNLuc-2A-eGFP-huMAR-WPRE-3′UTR[2hBGpA-A120]-SS*, SEQ ID NO: 32).

### B. Luciferase Expression Levels

HEK293 cells were separately transfected with (1) a conventional plasmid, pcDNA-CMV-5′UTR-SecNLuc-P2A-eGFP-bGHpA as shown in FIG. 2, (2) SS*-CMV-UTR1-SecNLuc-2A-eGFP-3′UTR[2hBGpA-A120]-SS*, (3) S*-E1-CMV-UTR1-SecNLuc-2A-eGFP-3′UTR[2hBGpA-A120]-SS*, and (4) SS*-E1-CMV-UTR1-SecNLuc-2A-eGFP-WPRE-3′UTR[2hBGpA-A120]-SS* using standard operating procedures.

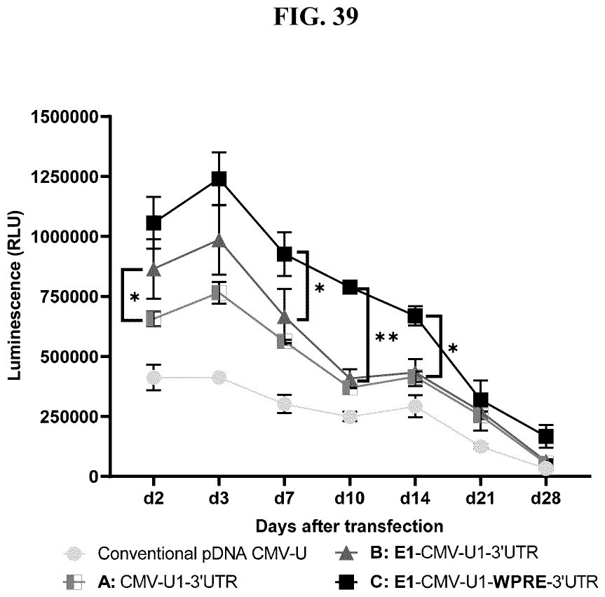

On days 2, 3, 7, 10, 14, 21, and 28 after electroporation, luciferase expression was evaluated by measuring the intensity of secreted luciferase from the media of cultured cells as described in Example 2B. See FIG. 39, showing expression levels in media from cells transfected with pcDNA-CMV-5′UTR-SecNLuc-P2A-eGFP-bGHpA (Conventional pDNA CMV-U), SS*-CMV-UTR1-SecNLuc-2A-eGFP-3′UTR[2hBGpA-A120]-SS* (A: CMV-U1-3′UTR), SS*-E1-CMV-UTR1-SecNLuc-2A-eGFP-3′UTR[2hBGpA-A120]-SS* (B: E1-CMV-U1-3′UTR), and SS*-E1-CMV-UTR1-SecNLuc-2A-eGFP-WPRE-3′UTR[2hBGpA-A120]-SS* (C: E1-CMV-U1-WPRE-3′UTR).

As shown in FIG. 39, luciferase expression was increased and durable in msDNA expression vectors containing the 3′UTR as compared to the conventional plasmid having an identical promoter and polygenic expression cassette. Expression was further increased with addition of E1 (A vs B) and WPRE (B vs C) genetic elements to the msDNA expression vectors, and the additive effects of the E1 and WPRE genetic elements resulted in the highest luciferase expression associated with construct C. Statistical analysis was performed using a t-test. *=p<0.05 and **=p<0.01.

### C. Vector Expansion to Daughter Cells and Luciferase Expression

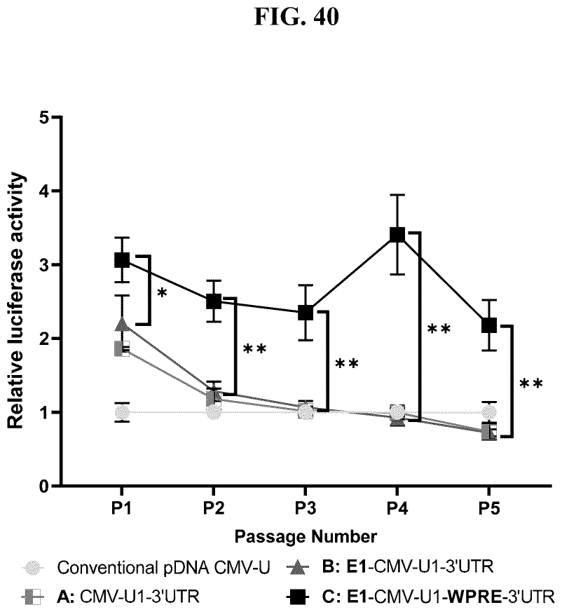

HEK293 cells were transfected with the four expression vectors described in part B of this example. Cells were passaged every 7 days for five passages. At the time of cell passaging, cells were re-seeded at 1/10 of the original cell density. For each cell passage, secreted luciferase expression was measured as described in Example 2B. See FIG. 40, showing expression levels in media from cells transfected with the msDNA expression vectors as compared to the conventional plasmid. Statistical analysis and p values were as noted in part B of this example.

Luciferase expression was detected from cells transfected with any of the msDNA expression vectors at each passage number, showing that the vectors were passed down to daughter cells with durable expression of luciferase.

As shown in FIG. 40, the additive effects of the E1 and WPRE genetic elements resulted in the highest luciferase expression associated with construct C at each passage number, and the most durable expression following multiple passages.

### Example 8—Expression Vectors Containing Synthetic Promoter Sequences

### A. Expression Vectors

Five synthetic promoter sequences were produced: (1) CAG [E1×3+CBA promoter+intron] (SEQ ID NO: 35), containing three copies of the synthetic enhancer E1 (i.e., 3 copies of SEQ ID NO: 12), a chicken β-actin promoter, and chimeric intron, (2) CAG [E2+CBA promoter+intron] (SEQ ID NO: 36), containing E2 (U100), a chicken β-actin promoter, and chimeric intron, (3) CAG [E1×3+CBA promoter+UTR1] (SEQ ID NO: 37), containing three copies of the synthetic enhancer E1, a chicken β-actin promoter, and 5′UTR1 (i.e., SEQ ID NO: 2), (4) CAG [E2 (U100)+CBA promoter+UTR1] (SEQ ID NO: 38), containing E2 (U100), a chicken β-actin promoter, and 5′UTR1, and (5) CMV enhancer-EF1-UTR1 (SEQ ID NO: 39), containing a CMV enhancer, an EF1a short promoter, and 5′UTR1.

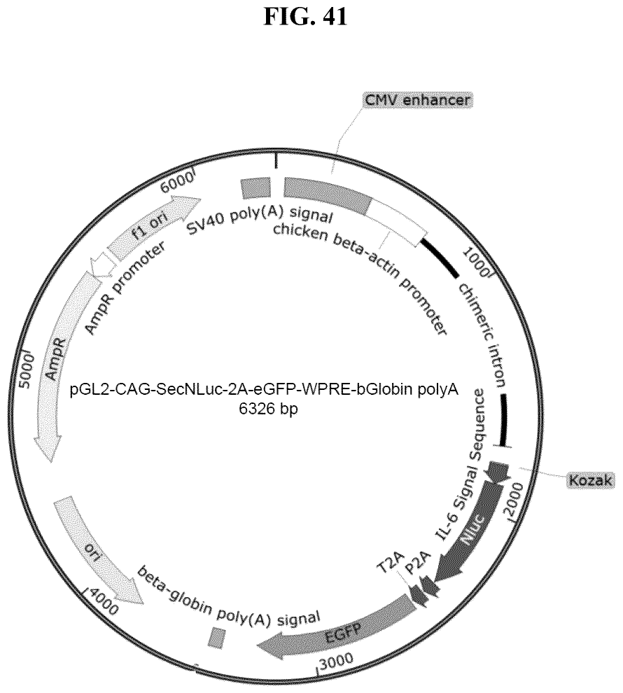

A conventional plasmid was produced containing a CMV enhancer, a chicken β-actin promoter, and chimeric intron and a polygenic expression cassette encoding eGFP and Nluc as described in Examples 1 and 2. A map of the conventional plasmid is shown in FIG. 41 (pGL2-CAG-SecNLuc-2A-eGFP-WPRE-bGlobin polyA, SEQ ID NO: 34).

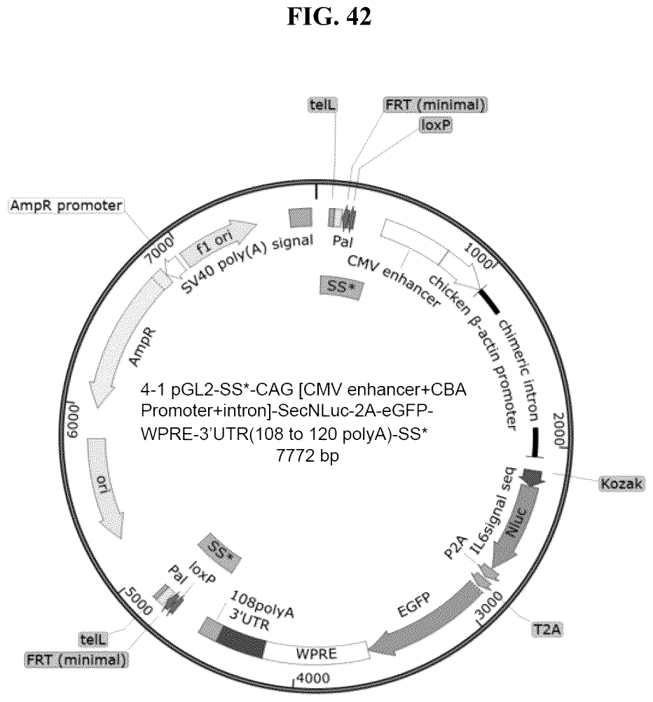

An msDNA expression vector was produced containing two Super Sequences sites, a CMV enhancer, a chicken β-actin promoter, chimeric intron, a polygenic expression cassette encoding eGFP and Nluc, WPRE, and 3′UTR. A map of the vector is shown in FIG. 42 (4-1 pGL2-SS*-CAG [CMV enhancer+CBA Promoter+intron]-SecNLuc-2A-eGFP-WPRE-3′UTR(108 to 120 polyA)-SS*, SEQ ID NO: 40).

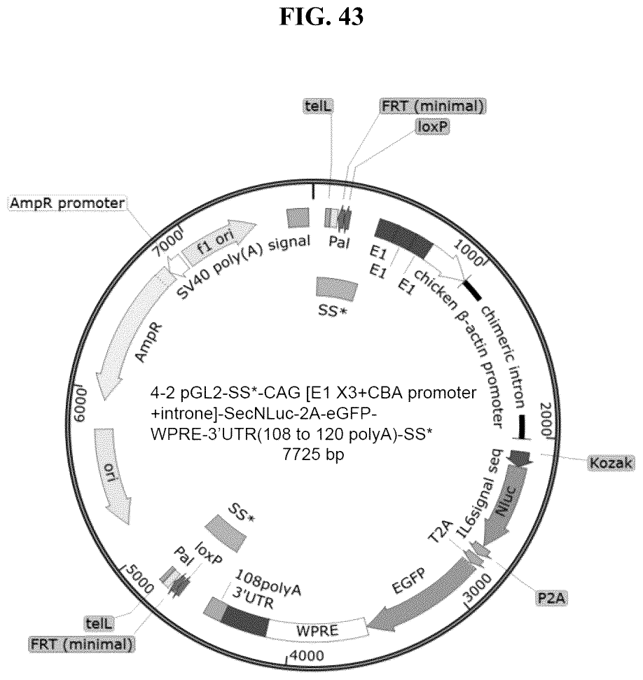

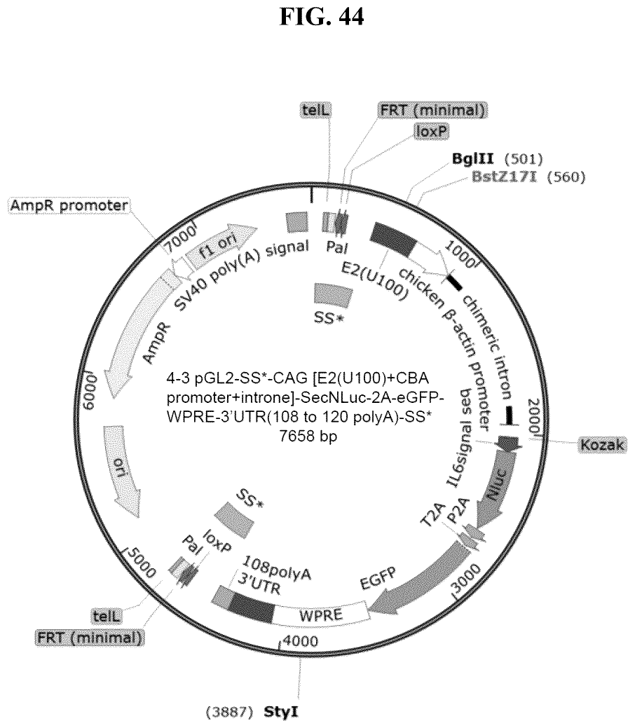

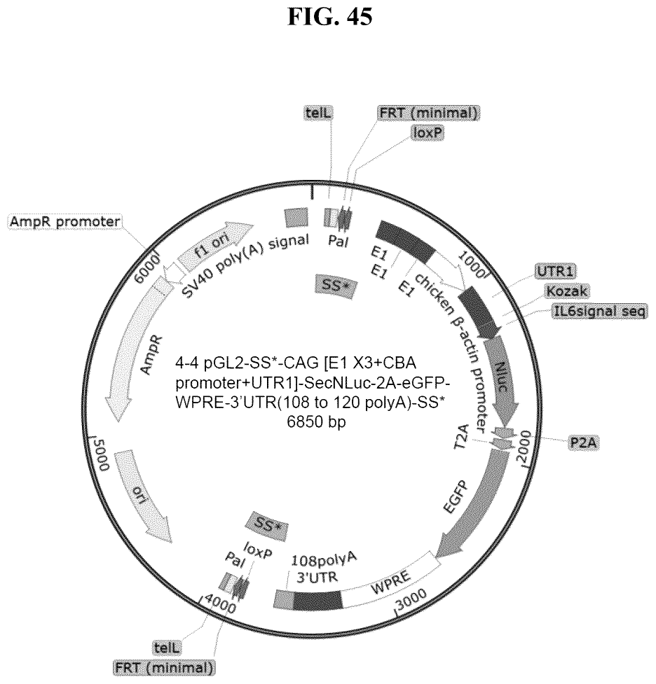

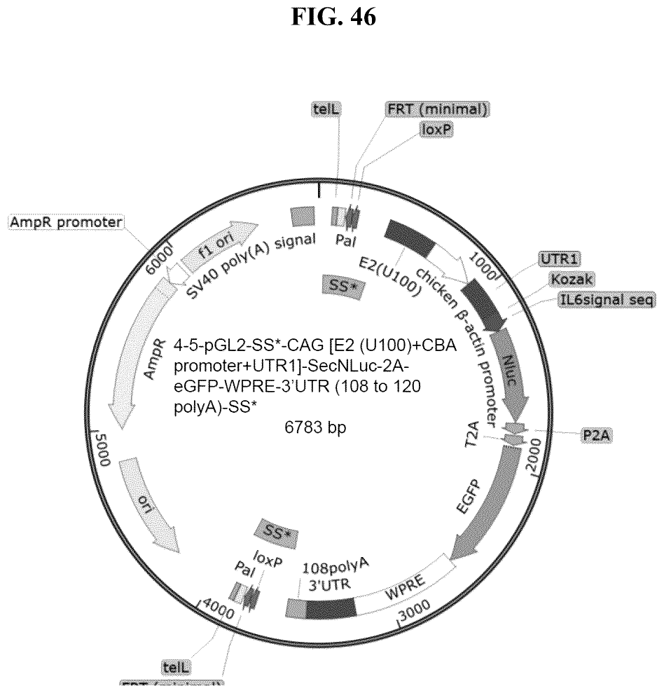

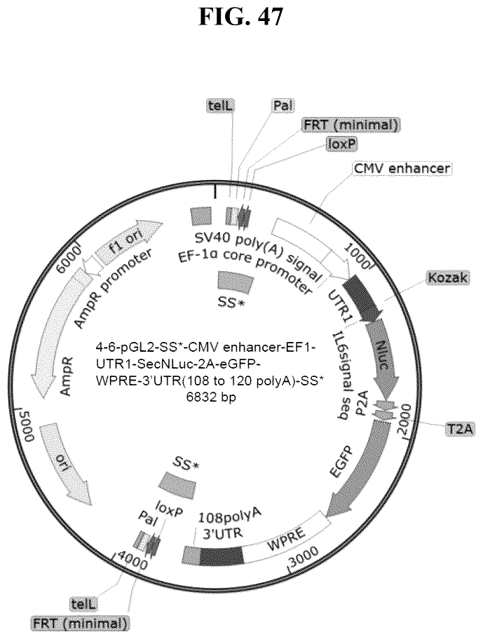

Five msDNA expression vectors were produced containing two Super Sequences sites, a polygenic expression cassette encoding eGFP and Nluc, WPRE, and 3′UTR along with one of synthetic promoters (1)-(5) as described above, with respective vector maps shown in FIG. 43 (4-2 pGL2-SS*-CAG [E1 X3+CBA promoter+intron]-SecNLuc-2A-eGFP-WPRE-3′UTR(108 to 120 polyA)-SS*, SEQ ID NO: 41), FIG. 44 (4-3 pGL2-SS*-CAG [E2(U100)+CBA promoter+intron]-SecNLuc-2A-eGFP-WPRE-3′UTR(108 to 120 polyA)-SS*, SEQ ID NO: 42), FIG. 45 (4-4 pGL2-SS*-CAG [E1 X3+CBA promoter+UTR1]-SecNLuc-2A-eGFP-WPRE-3′UTR(108 to 120 polyA)-SS*, SEQ ID NO: 43), FIG. 46 (4-5-pGL2-SS*-CAG [E2 (U100)+CBA promoter+UTR1]-SecNLuc-2A-eGFP-WPRE-3′UTR (108 to 120 polyA)-SS*, SEQ ID NO: 44), and FIG. 47 (4-6-pGL2-SS*-CMV enhancer-EF1-UTR1-SecNLuc-2A-eGFP-WPRE-3′UTR(108 to 120 polyA)-SS*, SEQ ID NO: 45).

### B. Luciferase Expression Levels

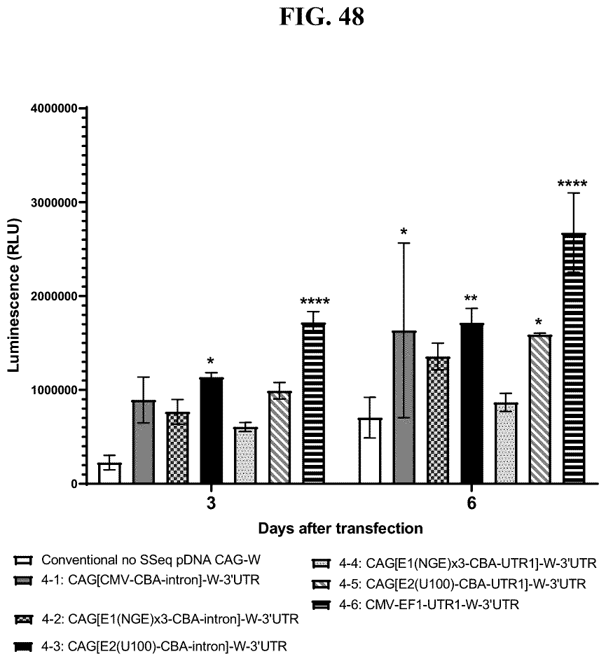

HEK293 cells were seeded in a 24-well plate at 1×10⁵ cells/well and separately transfected with a complex of lipofectamine and the vectors described in part A of this example at 0.25 pmol DNA/well. Secreted luciferase expression was measured as described in Example 2B at 3 and 6 days after transfection. See FIG. 48, showing expression levels in media from cells transfected with the msDNA expression vectors as compared to the conventional plasmid. Statistical analysis was performed using two-way Anova analysis relative to the conventional plasmid. *=p<0.05, **=p<0.01, and **** p<0.0001.

Luciferase expression levels were higher for all msDNA expression vectors as compared to the conventional plasmid. The highest expression was observed with 4-6-pGL2-SS*-CMV enhancer-EF1-UTR1-SecNLuc-2A-eGFP-WPRE-3′UTR(108 to 120 polyA (4-6: CMV-EF1-UTR1-W-3′UTR), which contains the EF-1 promoter element in combination with the CMV enhancer and 5′UTR1.

### Example 9—SS and Expression Cassette Modifications

The impact of modifications to the Super Sequence (SS) and the expression cassettes of the expression vectors as described in the present disclosure will be evaluated in terms of transfection efficiencies, expression of nucleic acid sequences of interest (including reporter genes, such as polygenic GFP and luciferase expression cassettes as described in Examples 1 and 2), and durability/expansion of the vectors in dividing cells (including rapid and slow dividing cells). Modifications to the SS also will be evaluated for restriction enzyme activity on these sites.

Modifications will include individual modifications and combinations such as, but not limited to, an endonuclease target sequence integrated in non-binding regions for the recombinases in the SS between the vector backbone and the cleavage sites for the recombinases, a CAG promoter integrated between the 3′ end of the first target sequence for the first recombinase (i.e., the 3′ end of the 5′ SS) and 5′ to the promoter in the expression cassette, a CMV enhancer integrated between the 3′ end of the first target sequence for the first recombinase (i.e., the 3′ end of the 5′ SS) and 5′ to the promoter in the expression cassette, an Enhancer-1 sequence located 5′ to a CMV enhancer and/or 3′ to a UCOE, a CMV, EF1, SV40, CAG, Rho, VDM2, HCR, or HLP promoter or variant thereof, a CMV promoter variant, an EF1-alpha promoter, a synthetic promoter, a 5′UTR comprising an intron integrated in the expression cassette between a promoter and nucleic acid sequence of interest with or without non-coding sequences integrated within the intron (e.g., a 5′UTR comprising the nucleic acid sequence of any one of SEQ ID NOs:2-5), a vertebrate chromatin insulator integrated in the expression cassette between the nucleic acid of interest and a polyadenylation signal, a woodchuck hepatitis virus post-transcriptional regulatory element integrated in the expression cassette between the nucleic acid of interest and a polyadenylation signal, a scaffold/matrix attachment region integrated in the expression cassette between the nucleic acid of interest and a polyadenylation signal, an ubiquitous chromatin opening element located 5′ to the promoter in the expression cassette (e.g., at the 3′ of the 5′ SS and prior to other sequences in the expression cassette), a 3′UTR integrated in the expression cassette between the nucleic acid of interest and the 3′ SS, such as directly following a stop codon (e.g., a 3′UTR comprising the nucleic acid sequence of any one of SEQ ID NOs: 13-16), and/or a poly(A) tail (e.g., as the 3′ end of a 3′UTR) comprising 100 to 120 adenine nucleotides.

### Sequences

artificial intron
SEQ ID NO: 1
gtaagtcgacgggccgggcctgggccgggtccgggccgggtcgttggatccccactacagcccga
tactcaagcttgacgaattcgagtatccaaggtagtggactagtgtgacgctgctgacccctttc
tttcccttctgcag
5′UTR1
SEQ ID NO: 2
ctgccttctccctcctgtgagtttggtaagtcgacgggccgggcctgggccgggtccgggccggg
tcgttggatccccactacagcccgatactcaagcttgacgaattcgagtatccaaggtagtggac
tagtgtgacgctgctgacccctttctttcccttctgcaggttggtgtacagtagcttcca
5′UTR1 with MAR-5 insertion
SEQ ID NO: 3
ctgccttctccctcctgtgagtttggtaagtcgacgggccgggcctgggccgggtccgggccggg
tatccatagctgattggtctaaaatgagatacatcaacgctcctccatgttttttgttttctttt
taaatgaaaaactttattttttaagaggagtttcaggttcatagcaaaattgagaggaaggtaca
ttcaagctgaggaagttttcctctattcctagtttactgagagattgcatcatgaatgggtgtta
aattttgtcaaatgctttttctgtgtctatcaatatgaccatgtgattttcttctttaacctgtt
gatgggacaaattacgttaattgattttcaaacgttgaaccacccttacatatctggaataaatt
ctacttggttgtggtgtatattttttgatacattcttggattctttttgctaatattttgttgaa
aatgtttgtatctttgttcatgagagatattggtctgttgttttcttttcttgtaatgtcatttt
ctagttccggtattaaggtaatgctggcctagttgaatgatttaggaagtattccctctgcttct
gtcttctgaaagagattgtagaaagttgatacaatttttttttctttaaatatcttgatagtcgt
tggatccccactacagcccgatactcaagcttgacgaattcgagtatccaaggtagtggactagt
gtgacgctgctgacccctttctttcccttctgcaggttggtgtacagtagcttcca
5′UTR
SEQ ID NO: 4
attgggatcttcacacagcaggtaaggttgcgggccgggcctgggccgggtccgggccgggccgc
actgacccctggtgttgctttttttttttaggccgcaagctgaagcgtgtcc
5′UTR2 (5′UTR of SEQ ID NO: 4 with MAR-5 insertion)
SEQ ID NO: 5
attgggatcttcacacagcaggtaaggttgcgggccgggcctgggccgggtccgggccgggtatc
catagctgattggtctaaaatgagatacatcaacgctcctccatgttttttgttttctttttaaa
tgaaaaactttattttttaagaggagtttcaggttcatagcaaaattgagaggaaggtacattca
agctgaggaagttttcctctattcctagtttactgagagattgcatcatgaatgggtgttaaatt
ttgtcaaatgctttttctgtgtctatcaatatgaccatgtgattttcttctttaacctgttgatg
ggacaaattacgttaattgattttcaaacgttgaaccacccttacatatctggaataaattctac
ttggttgtggtgtatattttttgatacattcttggattctttttgctaatattttgttgaaaatg
tttgtatctttgttcatgagagatattggtctgttgttttcttttcttgtaatgtcattttctag
ttccggtattaaggtaatgctggcctagttgaatgatttaggaagtattccctctgcttctgtct
tctgaaagagattgtagaaagttgatacaatttttttttctttaaatatcttgatagccgcactg
acccctggtgttgctttttttttttaggccgcaagctgaagcgtgtcc
A2UCOE element
SEQ ID NO: 6
gcggccgcacgcgtggccctccgcgcctacagctcaagccacatccgaagggggagggagccggg
agctgcgcgcggggccgccggggggaggggtggcaccgcccacgccgggcggccacgaagggcgg
ggcagcgggcgcgcgcgcggcggggggaggggccggcgccgcgcccgctgggaattggggcccta
gggggagggcggaggcgccgacgaccgcggcacttaccgttcgcggcgtggcgcccggtggtccc
caaggggagggaagggggaggcggggcgaggacagtgaccggagtctcctcagcggtggcttttc
tgcttggcagcctcagcggctggcgccaaaaccggactccgcccacttcctcgcccgccggtgcg
agggtgtggaatcctccagacgctgggggagggggagttgggagcttaaaaactagtaccccttt
gggaccactttcagcagcgaactctcctgtacaccaggggtcagttccacagacgcgggccaggg
gtgggtcattgcggcgtgaacaataatttgactagaagttgattcgggtgtttccggaaggggcc
gagtcaatccgccgagttggggcacggaaaacaaaaagggaaggctactaagatttttctggcgg
gggttatcattggcgtaactgcagggaccacctcccgggttgagggggctggatctccaggctgc
ggattaagcccctcccgtcggcgttaatttcaaactgcgcgacgtttctcacctgccttcgccaa
ggcaggggccgggaccctattccaagaggtagtaactagcaggactctagccttccgcaattcat
tgagcgcatttacggaagtaacgtcgggtactgtctctggccgcaagggtgggaggagtacgcat
ttggcgtaaggtggggcgtagagccttcccgccattggcggcggatagggcgtttacgcgacggc
ctgacgtagcggaagacgccttagtgggggggaaggttctagaaaagcggcggcagcggctctag
cggcagtagcagcagcgccgggtcccgtgcggaggtgctcctcgcagagttgtttctccagcagc
ggcagttctcactacagcgccaggacgagtccggttcgtgttcgtccgcggagatctctctcatc
tcgctcggctgcgggaaatcgggctgaagcgactgagtccgcgatggaggtaacgggtttgaaat
caatgagttattgaaaagggcatggcgaggccgttggcgcctcagtggaagtcggccagccgcct
ccgtgggagagaggcaggaaatcggaccaattcagtagcagtggggcttaaggtttatgaacggg
gtcttgagcggaggcctgagcgtacaaacagcttccccaccctcagcctcccggcgccatttccc
ttcactgggggtgggggatggggagctttcacatggcggacgctgccccgctggggtgaaagtgg
ggcgcggaggcgggacttcttattccctttctaaagcacgctgcttcgggggccacggcgtctcc
tcggacggccgggcgcgcc
SRF-UCOE
SEQ ID NO: 7
gcacacgaccacaattccactgaaagcattttaatacggaacttgtcactcccagggagcctccg
ctcagccggcagttggttcatttcaatccccacgacaacccttcaaagtgcagggcagacagcag
gtggctctgcccaggcgcctggatcacagcccggcctgcagccctcacctgggcgcggggagacc
ctgaggacgctcctccaggcggcgctggccggggcctgcggacacggacgggcgggctgagctcc
gggacccctccccgcgccccgcaccccgcaccccgcaccccgcaccccgcacccggcgctcaccc
gtcccagccccgccgcccgcagccccagctgcaacgcagccaccgccgccatcgcacccggcccc
gcgggcgcttccgggacgcaggaggcatctgcatccggggcgccgctgagtcccgcccagagccc
cgcccccggctccaggttctgcgagcggcttccgccgggctgctccgcgggcgcgtcggccatga
gcgagttgccgggcgacgtgcgggcgtttctgcgggagcacccgagcctgcggctccagacggac
gcccgcaaggttcgcagcgcgggaggggaacggagtggcggagaagggcgcagttgggatgaggg
gctgaggggagggcagggga
gaggagagggcaggggagaggggagaggggagagcaggagagaggggaaggcaggggagagggcg
cggcgggatcaggggaggagagggaa
cHS4 insulator
SEQ ID NO: 8
ggggagctcacggggacagcccccccccaaagcccccagggatgtaattacgtccctcccccgct
agggggcagcagcgaccgcccggggctccgctccggtccggcgctccccccgcatcccgagccgg
cagcgtgcggggacagcccgggcacggggaaggtggcacgggatcgctttcctctgaacgcttct
cgctgctctttgagcctgcagacacctgggggatacggggaaaaggggagctcacggggacagcc
cccccccaaagcccccagggatgtaattacgtccctcccccgctagggggcagcagcgaccgccc
ggggctccgctccggtccggcgctccccccgcatcccgagccggcagcgtgcggggacagcccgg
gcacggggaaggtggcacgggatcgctttcctctgaacgcttctcgctgctctttgagcctgcag
acacctgggggatacggggaaaa
MAR-5
SEQ ID NO: 9
tatccatagctgattggtctaaaatgagatacatcaacgctcctccatgttttttgttttctttt
taaatgaaaaactttattttttaagaggagtttcaggttcatagcaaaattgagaggaaggtaca
ttcaagctgaggaagttttcctctattcctagtttactgagagattgcatcatgaatgggtgtta
aattttgtcaaatgctttttctgtgtctatcaatatgaccatgtgattttcttctttaacctgtt
gatgggacaaattacgttaattgattttcaaacgttgaaccacccttacatatctggaataaatt
ctacttggttgtggtgtatattttttgatacattcttggattctttttgctaatattttgttgaa
aatgtttgtatctttgttcatgagagatattggtctgttgttttcttttcttgtaatgtcatttt
ctagttccggtattaaggtaatgctggcctagttgaatgatttaggaagtattccctctgcttct
gtcttctgaaagagattgtagaaagttgatacaatttttttttctttaaatatcttgatag
human CSP-B MAR (huMAR)
SEQ ID NO: 10
ggatcccattctccttgatgtactaatttttctttaaaagtgataataatagctcccatttagaa
tttttaaataacacaacaaatgtaaagtaactaatgtgtcctctggatcatggtaagtaatgaat
aaatttaactccctttaccttctccctttgctattttttccatgctaggatttatacatttttaa
aaaactaaatctgctatcaaatgacagctttaaatttactttttaaaatttgttattgtatatat
ttatggggtataaagtgatgttatgatatatatatacacaatgtacactgattaaatcaagccaa
ttaacattttatcatctcaaatacttaacattttttgtagtgagaacatttgaaatttactttta
gcaatttcaaaacatacattattattattaactatagtcaccatgatgtaccatagatctttaaa
aacttattcttcctgcctaactgaaactttgtactctttgactaacatcttttcattcccccact
tcccagcctctggtaatcaccattacacactctgcttctatgagttcaattgctttagactccac
gtaataaatgagatcatgcagcatttggctttctgtgcctggcttatccttgcttagcatggtgt
cttacaggttcatccatgttgcaacaaataacagaatctcattctttgttaaggctgaatactat
tccattgggtatatataccacattttccttatccattaatccactgatggacccttaggttgttg
attccatatattggctattgtaaatagtgcagcaatgaacatgagagtgcaactatctcttcaat
gtactgatttcgaatccttcggatctatctcagaagtgagattgcaggatcatataattctactt
ttagtcttttgaggagctccatacagctttccatatggccatactaattacattctcatcaacag
tgtacaatggtttccttttctccacatcctcaccaacatttataattttttgtctttttgataat
agccatctgacaggtgtaaagtgatagctcattgcagttttaatttgcattttttgatgattagt
aatgttgagaattttttcatatatctcttggccagttgcatgtcttctttggaaaaatgtctatt
cagttcctttgcccattttttaattgggatttttggtttcttgctattgagttgtttgaattc
WPRE
SEQ ID NO: 11
Tcgacaatcaacctctggattacaaaatttgtgaaagattgactggtattcttaactatgttgct
ccttttacgctatgtggatacgctgctttaatgcctttgtatcatgctattgcttcccgtatggc
tttcattttctcctccttgtataaatcctggttgctgtctctttatgaggagttgtggcccgttg
tcaggcaacgtggcgtggtgtgcactgtgtttgctgacgcaacccccactggttggggcattgcc
accacctgtcagctcctttccgggactttcgctttccccctccctattgccacggcggaactcat
cgccgcctgccttgcccgctgctggacaggggctcggctgttgggcactgacaattccgtggtgt
tgtcggggaagctgacgtcctttccatggctgctcgcctgtgttgccacctggattctgcgcggg
acgtccttctgctacgtcccttcggccctcaatccagcggaccttccttcccgcggcctgctgcc
ggctctgcggcctcttccgcgtcttcgccttcgccctcagacgagtcggatctccctttgggccg
cctccccgcctg
Enhancer-1
SEQ ID NO: 12
gggactttccggggcggggcacgtggtgcacgggactttccgtgcacgtgcacgggactttccgg
gactttccgggactttccgtgcaccacgtggggactttccgtgcac
2 copies of Xenopus leavis beta-globin polyadenylation signal
(2xlBGpA)
SEQ ID NO: 13
aaccagcctcaagaacacccgaatggagtctctaagctacataataccaacttacactttacaaa
atgttgtcccccaaaatgtagccattcgtatctgctcctaataaaaagaaagtttcttcacaacc
agcctcaagaacacccgaatggagtctctaagctacataataccaacttacactttacaaaatgt
tgtcccccaaaatgtagccattcgtatctgctcctaataaaaagaaagtttcttcac
2 copies of human beta-globin polyadenylation signal (2huBGpA)
SEQ ID NO: 14
gctcgctttcttgctgtccaatttctattaaaggttcctttgttccctaagtccaactactaaac
tgggggatattatgaagggccttgagcatctggattctgcctaataaaaaacatttattttcatt
gcaagctcgctttcttgctgtccaatttctattaaaggttcctttgttccctaagtccaactact
aaactgggggatattatgaagggccttgagcatctggattctgcctaataaaaaacatttatttt
cattgcaa
hybrid Xenopus leavis and human beta-globin polyadenylation
signal (xlhuBGpA)
SEQ ID NO: 15
aaccagcctcaagaacacccgaatggagtctctaagctacataataccaacttacactttacaaa
atgttgtcccccaaaatgtagccattcgtatctgctcctaataaaaagaaagtttcttcacgctc
gctttcttgctgtccaatttctattaaaggttcctttgttccctaagtccaactactaaactggg
ggatattatgaagggccttgagcatctggattctgcctaataaaaaacatttattttcattgcaa
2xlBGpA-A120
SEQ ID NO: 16
aaccagcctcaagaacacccgaatggagtctctaagctacataataccaacttacactttacaaa
atgttgtcccccaaaatgtagccattcgtatctgctcctaataaaaagaaagtttcttcacaacc
agcctcaagaacacccgaatggagtctctaagctacataataccaacttacactttacaaaatgt
tgtcccccaaaatgtagccattcgtatctgctcctaataaaaagaaagtttcttcacaaaaaaaa
aaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaa
2huBGpA-A120
SEQ ID NO: 17
gctcgctttcttgctgtccaatttctattaaaggttcctttgttccctaagtccaactactaaac
tgggggatattatgaagggccttgagcatctggattctgcctaataaaaaacatttattttcatt
gcaagctcgctttcttgctgtccaatttctattaaaggttcctttgttccctaagtccaactact
aaactgggggatattatgaagggccttgagcatctggattctgcctaataaaaaacatttatttt
cattgcaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaa
xlhuBGpA-A120
SEQ ID NO: 18
aaccagcctcaagaacacccgaatggagtctctaagctacataataccaacttacactttacaaa
atgttgtcccccaaaatgtagccattcgtatctgctcctaataaaaagaaagtttcttcacgctc
gctttcttgctgtccaatttctattaaaggttcctttgttccctaagtccaactactaaactggg
ggatattatgaagggccttgagcatctggattctgcctaataaaaaacatttattttcattgcaa
aaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaa
pGL2-SS*-CAG-SecNLuc-2A-eGFP-BGpA-SS*
SEQ ID NO: 19
ccgggaggtaccgagctcttacgcgtgctagaattaaagtaacccaatcagcacacaattgccat
tatacgcgcgtataatggactattgtgtgctgataaacctatttcagcatactacgcgcgtagta
tgctgaaataggtgactagaagttcctatactttctagagaataggaacttcataacttcgtata
atgtatgctatacgaagttatgggttactttaatttggttgctgactaattgagatgcatgcttt
gcatacttctgcctgctggggagcctggggactttccacacctggttgctgactaattgagatgc
atgctttgcatacttctgcctgctggggagcctggggactttccacacccctgggtcgacattga
ttattgactagttattaatagtaatcaattacggggtcattagttcatagcccatatatggagtt
ccgcgttacataacttacggtaaatggcccgcctggctgaccgcccaacgacccccgcccattga
cgtcaataatgacgtatgttcccatagtaacgccaatagggactttccattgacgtcaatgggtg
gagtatttacggtaaactgcccacttggcagtacatcaagtgtatcatatgccaagtacgccccc
tattgacgtcaatgacggtaaatggcccgcctggcattatgcccagtacatgaccttatgggact
ttcctacttggcagtacatctacgtattagtcatcgctattaccatggtcgaggtgagccccacg
ttctgcttcactctccccatctcccccccctccccacccccaattttgtatttatttatttttta
attattttgtgcagcgatgggggcggggggggggggggcgcgcgccaggcggggcggggcggggc
gaggggcggggcggggcgaggcggaaaggtgcggcggcagccaatcagagcggcgcgctccgaaa
gtttccttttatggcgaggcggcggcggcggcggccctataaaaagcgaagcgcgcggcgggcgg
gagtcgctgcgttgccttcgccccgtgccccgctccgcgccgcctcgcgccgcccgccccggctc
tgactgaccgcgttactcccacaggtgagcgggcgggacggcccttctcctccgggctgtaatta
gcgcttggtttaatgacggctcgtttcttttctgtggctgcgtgaaagccttaaagggctccggg
agggccctttgtgcgggggggagcggctcggggggtgcgtgcgtgtgtgtgtgcgtggggagcgc
cgcgtgcggctccgcgctgcccggcggctgtgagcgctgcgggcgcggcgcggggctttgtgcgc
tccgcagtgtgcgcgaggggagcgcggccgggggcggtgccccgcggtgcggggggggctgcgag
gggaacaaaggctgcgtgcggggtgtgtgcgtgggggggtgagcagggggtgtgggcgcggcggt
cgggctgtaacccccccctgcacccccctccccgagttgctgagcacggcccggcttcgggtgcg
gggctccgtacggggcgtggcgcggggctcgccgtgccgggcggggggtggcggcaggtgggggt
gccgggcggggcggggccgcctcgggccggggagggctcgggggaggggcgcggcggcccccgga
gcgccggcggctgtcgaggcgcggcgagccgcagccattgccttttatggtaatcgtgcgagagg
gcgcagggacttcctttgtcccaaatctgtgcggagccgaaatctgggaggcgccgccgcacccc
ctctagcgggcgcggggcgaagcggtgcggcgccggcaggaaggaaatgggcggggagggccttc
gtgcgtcgccgcgccgccgtccccttctccctctccagcctcggggctgtccgcggggggacggc
tgccttcgggggggacggggcagggcggggttcggcttctggcgtgtgaccggcggctctagagc
ctctgctaaccatgttcatgccttcttctttttcctacagctcctgggcaacgtgctggttattg
tgctgtctcatcattttggcaaagaattgattaattcgagcgaacgcgtcgccaccatgaactcc
ttctccacaagcgccttcggtccagttgccttctccctgggcctgctcctggtgttgcctgctgc
cttccctgccccagtcttcacactcgaagatttcgttggggactggcgacagacagccggctaca
acctggaccaagtccttgaacagggaggtgtgtccagtttgtttcagaatctcggggtgtccgta
actccgatccaaaggattgtcctgagcggtgaaaatgggctgaagatcgacatccatgtcatcat
cccgtatgaaggtctgagcggcgaccaaatgggccagatcgaaaaaatttttaaggtggtgtacc
ctgtggatgatcatcactttaaggtgatcctgcactatggcacactggtaatcgacggggttacg
ccgaacatgatcgactatttcggacggccgtatgaaggcatcgccgtgttcgacggcaaaaagat
cactgtaacagggaccctgtggaacggcaacaaaattatcgacgagcgcctgatcaaccccgacg
gctccctgctgttccgagtaaccatcaacggagtgaccggctggcggctgtgcgaacgcattctg
gcggctagcgctactaacttcagcctgctgaagcaggctggagacgtggaggagaaccctggacc
tggaagcggagagggcagaggaagtctgctaacatgcggtgacgtcgaggagaatcctggacctg
gatccggaatggtgagcaagggcgaggagctgttcaccggggtggtgcccatcctggtcgagctg
gacggcgacgtaaacggccacaagttcagcgtgtccggcgagggcgagggcgatgccacctacgg
caagctgaccctgaagttcatctgcaccaccggcaagctgcccgtgccctggcccaccctcgtga
ccaccctgacctacggcgtgcagtgcttcagccgctaccccgaccacatgaagcagcacgacttc
ttcaagtccgccatgcccgaaggctacgtccaggagcgcaccatcttcttcaaggacgacggcaa
ctacaagacccgcgccgaggtgaagttcgagggcgacaccctggtgaaccgcatcgagctgaagg
gcatcgacttcaaggaggacggcaacatcctggggcacaagctggagtacaactacaacagccac
aacgtctatatcatggccgacaagcagaagaacggcatcaaggtgaacttcaagatccgccacaa
catcgaggacggcagcgtgcagctcgccgaccactaccagcagaacacccccatcggcgacggcc
ccgtgctgctgcccgacaaccactacctgagcacccagtccgccctgagcaaagaccccaacgag
aagcgcgatcacatggtcctgctggagttcgtgaccgccgccgggatcactctcggcatggacga
gctgtacaagtaagcggccgcactcctcaggtgcaggctgcctatcagaaggtggtggctggtgt
ggccaatgccctggctcacaaataccactgagatctttttccctctgccaaaaattatggggaca
tcatgaagccccttgagcatctgacttctggctaataaaggaaatttattttcattgcaatagtg
tgttggaattttttgtgtctctcactcggaaggacatggtgtggaaagtccccaggctccccagc
aggcagaagtatgcaaagcatgcatctcaattagtcagcaaccaaattaaagtaacccataactt
cgtatagcatacattatacgaagttatgaagttcctattctctagaaagtataggaacttctagt
cacctatttcagcatactacgcgcgtagtatgctgaaataggtttatcagcacacaatagtccat
tatacgcgcgtataatggcaattgtgtgctgattgggttactttaattggtgtggaaagtcccca
ggctccccagcaggcagaagtatgcaaagcatgcatctcaattagtcagcaaccatggatccgtc
gaccgatgcccttgagagccttcaacccagtcagctccttccggtgggcgcggggcatgactatc
gtcgccgcacttatgactgtcttctttatcatgcaactcgtaggacaggtgccggcagcgctctt
ccgcttcctcgctcactgactcgctgcgctcggtcgttcggctgcggcgagcggtatcagctcac
tcaaaggcggtaatacggttatccacagaatcaggggataacgcaggaaagaacatgtgagcaaa
aggccagcaaaaggccaggaaccgtaaaaaggccgcgttgctggcgtttttccataggctccgcc
cccctgacgagcatcacaaaaatcgacgctcaagtcagaggtggcgaaacccgacaggactataa
agataccaggcgtttccccctggaagctccctcgtgcgctctcctgttccgaccctgccgcttac
cggatacctgtccgcctttctcccttcgggaagcgtggcgctttctcatagctcacgctgtaggt
atctcagttcggtgtaggtcgttcgctccaagctgggctgtgtgcacgaaccccccgttcagccc
gaccgctgcgccttatccggtaactatcgtcttgagtccaacccggtaagacacgacttatcgcc
actggcagcagccactggtaacaggattagcagagcgaggtatgtaggcggtgctacagagttct
tgaagtggtggcctaactacggctacactagaagaacagtatttggtatctgcgctctgctgaag
ccagttaccttcggaaaaagagttggtagctcttgatccggcaaacaaaccaccgctggtagcgg
tggtttttttgtttgcaagcagcagattacgcgcagaaaaaaaggatctcaagaagatcctttga
tcttttctacggggtctgacgctcagtggaacgaaaactcacgttaagggattttggtcatgaga
ttatcaaaaaggatcttcacctagatccttttaaattaaaaatgaagttttaaatcaatctaaag
tatatatgagtaaacttggtctgacagttaccaatgcttaatcagtgaggcacctatctcagcga
tctgtctatttcgttcatccatagttgcctgactccccgtcgtgtagataactacgatacgggag
ggcttaccatctggccccagtgctgcaatgataccgcgagacccacgctcaccggctccagattt
atcagcaataaaccagccagccggaagggccgagcgcagaagtggtcctgcaactttatccgcct
ccatccagtctattaattgttgccgggaagctagagtaagtagttcgccagttaatagtttgcgc
aacgttgttgccattgctacaggcatcgtggtgtcacgctcgtcgtttggtatggcttcattcag
ctccggttcccaacgatcaaggcgagttacatgatcccccatgttgtgcaaaaaagcggttagct
ccttcggtcctccgatcgttgtcagaagtaagttggccgcagtgttatcactcatggttatggca
gcactgcataattctcttactgtcatgccatccgtaagatgcttttctgtgactggtgagtactc
aaccaagtcattctgagaatagtgtatgcggcgaccgagttgctcttgcccggcgtcaatacggg
ataataccgcgccacatagcagaactttaaaagtgctcatcattggaaaacgttcttcggggcga
aaactctcaaggatcttaccgctgttgagatccagttcgatgtaacccactcgtgcacccaactg
atcttcagcatcttttactttcaccagcgtttctgggtgagcaaaaacaggaaggcaaaatgccg
caaaaaagggaataagggcgacacggaaatgttgaatactcatactcttcctttttcaatattat
tgaagcatttatcagggttattgtctcatgagcggatacatatttgaatgtatttagaaaaataa
acaaataggggttccgcgcacatttccccgaaaagtgccacctgacgcgccctgtagcggcgcat
taagcgcggcgggtgtggtggttacgcgcagcgtgaccgctacacttgccagcgccctagcgccc
gctcctttcgctttcttcccttcctttctcgccacgttcgccggctttccccgtcaagctctaaa
tcgggggctccctttagggttccgatttagtgctttacggcacctcgaccccaaaaaacttgatt
agggtgatggttcacgtagtgggccatcgccctgatagacggtttttcgccctttgacgttggag
tccacgttctttaatagtggactcttgttccaaactggaacaacactcaaccctatctcggtcta
ttcttttgatttataagggattttgccgatttcggcctattggttaaaaaatgagctgatttaac
aaaaatttaacgcgaattttaacaaaatattaacgcttacaatttgccattcgccattcaggctg
cgcaactgttgggaagggcgatcggtgcgggcctcttcgctattacgccagcccaagctaccatg
ataagtaagtaatattaaggtacgtggaggttttacttgctttaaaaaacctcccacacctcccc
ctgaacctgaaacataaaatgaatgcaattgttgttgttaacttgtttattgcagcttataatgg
ttacaaataaagcaatagcatcacaaatttcacaaataaagcatttttttcactgcattctagtt
gtggtttgtccaaactcatcaatgtatcttatggtactgtaactgagctaacataa
pcDNA-CMV-5′UTR-SecNLuc-P2A-eGFP-bGHpA
SEQ ID NO: 20
gacggatcgggagatctcccgatcccctatggtgcactctcagtacaatctgctctgatgccgc
atagttaagccagtatctgctccctgcttgtgtgttggaggtcgctgagtagtgcgcgagcaaa
atttaagctacaacaaggcaaggcttgaccgacaattgcatgaagaatctgcttagggttaggc
gttttgcgctgcttcgcgatgtacgggccagatatacgcgttgacattgattattgactagtta
ttaatagtaatcaattacggggtcattagttcatagcccatatatggagttccgcgttacataa
cttacggtaaatggcccgcctggctgaccgcccaacgacccccgcccattgacgtcaataatga
cgtatgttcccatagtaacgccaatagggactttccattgacgtcaatgggtggagtatttacg
gtaaactgcccacttggcagtacatcaagtgtatcatatgccaagtacgccccctattgacgtc
aatgacggtaaatggcccgcctggcattatgcccagtacatgaccttatgggactttcctactt
ggcagtacatctacgtattagtcatcgctattaccatggtgatgcggttttggcagtacatcaa
tgggcgtggatagcggtttgactcacggggatttccaagtctccaccccattgacgtcaatggg
agtttgttttggcaccaaaatcaacgggactttccaaaatgtcgtaacaactccgccccattga
cgcaaatgggcggtaggcgtgtacggtgggaggtctatataagcagagctctctggctaactag
agaacccactgcttactggcttatcgaaattaatacgactcactatagggagacccaagctggc
tagcgtttaaacttaagcttggtaccgagctcggatccctgccttctccctcctgtgagtttgg
taagtcactgactgtctatgcctgggaaagggtgggcaggagatggggcagtgcaggaaaagtg
gcactatgaaccctgcagccctaggaatgcatctagacaattgtactaaccttcttctctttcc
tctcctgacaggttggtgtacagtagcttccactcctgccaccatgaactccttctccacaagc
gccttcggtccagttgccttctccctgggcctgctcctggtgttgcctgctgccttccctgccc
cagtcttcacactcgaagatttcgttggggactggcgacagacagccggctacaacctggacca
agtccttgaacagggaggtgtgtccagtttgtttcagaatctcggggtgtccgtaactccgatc
caaaggattgtcctgagcggtgaaaatgggctgaagatcgacatccatgtcatcatcccgtatg
aaggtctgagcggcgaccaaatgggccagatcgaaaaaatttttaaggtggtgtaccctgtgga
tgatcatcactttaaggtgatcctgcactatggcacactggtaatcgacggggttacgccgaac
atgatcgactatttcggacggccgtatgaaggcatcgccgtgttcgacggcaaaaagatcactg
taacagggaccctgtggaacggcaacaaaattatcgacgagcgcctgatcaaccccgacggctc
cctgctgttccgagtaaccatcaacggagtgaccggctggcggctgtgcgaacgcattctggcg
gaattctgcagatatccagcacagtggcggccgctcgagtctagaggaagcggagctactaact
tcagcctgctgaagcaggctggagacgtggaggagaaccctggacctatgagcaagggcgagga
gctgttcaccggggtggtgcccatcctggtcgagctggacggcgacgtaaacggccacaagttc
agcgtgtccggcgagggcgagggcgatgccacctacggcaagctgaccctgaagttcatctgca
ccaccggcaagctgcccgtgccctggcccaccctcgtgaccaccctgacctacggcgtgcagtg
cttcagccgctaccccgaccacatgaagcagcacgacttcttcaagtccgccatgcccgaaggc
tacgtccaggagcgcaccatcttcttcaaggacgacggcaactacaagacccgcgccgaggtga
agttcgagggcgacaccctggtgaaccgcatcgagctgaagggcatcgacttcaaggaggacgg
caacatcctggggcacaagctggagtacaactacaacagccacaacgtctatatcatggccgac
aagcagaagaacggcatcaaggtgaacttcaagatccgccacaacatcgaggacggcagcgtgc
agctcgccgaccactaccagcagaacacccccatcggcgacggccccgtgctgctgcccgacaa
ccactacctgagcacccagtccgccctgagcaaagaccccaacgagaagcgcgatcacatggtc
ctgctggagttcgtgaccgccgccgggatcactcacggcatggacgagctgtacaagtaagggc
ccgtttaaacccgctgatcagcctcgactgtgccttctagttgccagccatctgttgtttgccc
ctcccccgtgccttccttgaccctggaaggtgccactcccactgtcctttcctaataaaatgag
gaaattgcatcgcattgtctgagtaggtgtcattctattctggggggtggggtggggcaggaca
gcaagggggaggattgggaagacaatagcaggcatgctggggatgcggtgggctctatggcttc
tgaggcggaaagaaccagctggggctctagggggtatccccacgcgccctgtagcggcgcatta
agcgcggcgggtgtggtggttacgcgcagcgtgaccgctacacttgccagcgccctagcgcccg
ctcctttcgctttcttcccttcctttctcgccacgttcgccggctttccccgtcaagctctaaa
tcgggggctccctttagggttccgatttagtgctttacggcacctcgaccccaaaaaacttgat
tagggtgatggttcacgtagtgggccatcgccctgatagacggtttttcgccctttgacgttgg
agtccacgttctttaatagtggactcttgttccaaactggaacaacactcaaccctatctcggt
ctattcttttgatttataagggattttgccgatttcggcctattggttaaaaaatgagctgatt
taacaaaaatttaacgcgaattaattctgtggaatgtgtgtcagttagggtgtggaaagtcccc
aggctccccagcaggcagaagtatgcaaagcatgcatctcaattagtcagcaaccaggtgtgga
aagtccccaggctccccagcaggcagaagtatgcaaagcatgcatctcaattagtcagcaacca
tagtcccgcccctaactccgcccatcccgcccctaactccgcccagttccgcccattctccgcc
ccatggctgactaattttttttatttatgcagaggccgaggccgcctctgcctctgagctattc
cagaagtagtgaggaggcttttttggaggcctaggcttttgcaaaaagctcccgggagcttgta
tatccattttcggatctgatcaagagacaggatgaggatcgtttcgcatgattgaacaagatgg
attgcacgcaggttctccggccgcttgggtggagaggctattcggctatgactgggcacaacag
acaatcggctgctctgatgccgccgtgttccggctgtcagcgcaggggcgcccggttctttttg
tcaagaccgacctgtccggtgccctgaatgaactgcaggacgaggcagcgcggctatcgtggct
ggccacgacgggcgttccttgcgcagctgtgctcgacgttgtcactgaagcgggaagggactgg
ctgctattgggcgaagtgccggggcaggatctcctgtcatctcaccttgctcctgccgagaaag
tatccatcatggctgatgcaatgcggcggctgcatacgcttgatccggctacctgcccattcga
ccaccaagcgaaacatcgcatcgagcgagcacgtactcggatggaagccggtcttgtcgatcag
gatgatctggacgaagagcatcaggggctcgcgccagccgaactgttcgccaggctcaaggcgc
gcatgcccgacggcgaggatctcgtcgtgacccatggcgatgcctgcttgccgaatatcatggt
ggaaaatggccgcttttctggattcatcgactgtggccggctgggtgtggcggaccgctatcag
gacatagcgttggctacccgtgatattgctgaagagcttggcggcgaatgggctgaccgcttcc
tcgtgctttacggtatcgccgctcccgattcgcagcgcatcgccttctatcgccttcttgacga
gttcttctgagcgggactctggggttcgaaatgaccgaccaagcgacgcccaacctgccatcac
gagatttcgattccaccgccgccttctatgaaaggttgggcttcggaatcgttttccgggacgc
cggctggatgatcctccagcgcggggatctcatgctggagttcttcgcccaccccaacttgttt
attgcagcttataatggttacaaataaagcaatagcatcacaaatttcacaaataaagcatttt
tttcactgcattctagttgtggtttgtccaaactcatcaatgtatcttatcatgtctgtatacc
gtcgacctctagctagagcttggcgtaatcatggtcatagctgtttcctgtgtgaaattgttat
ccgctcacaattccacacaacatacgagccggaagcataaagtgtaaagcctggggtgcctaat
gagtgagctaactcacattaattgcgttgcgctcactgcccgctttccagtcgggaaacctgtc
gtgccagctgcattaatgaatcggccaacgcgcggggagaggcggtttgcgtattgggcgctct
tccgcttcctcgctcactgactcgctgcgctcggtcgttcggctgcggcgagcggtatcagctc
actcaaaggcggtaatacggttatccacagaatcaggggataacgcaggaaagaacatgtgagc
aaaaggccagcaaaaggccaggaaccgtaaaaaggccgcgttgctggcgtttttccataggctc
cgcccccctgacgagcatcacaaaaatcgacgctcaagtcagaggtggcgaaacccgacaggac
tataaagataccaggcgtttccccctggaagctccctcgtgcgctctcctgttccgaccctgcc
gcttaccggatacctgtccgcctttctcccttcgggaagcgtggcgctttctcatagctcacgc
tgtaggtatctcagttcggtgtaggtcgttcgctccaagctgggctgtgtgcacgaaccccccg
ttcagcccgaccgctgcgccttatccggtaactatcgtcttgagtccaacccggtaagacacga
cttatcgccactggcagcagccactggtaacaggattagcagagcgaggtatgtaggcggtgct
acagagttcttgaagtggtggcctaactacggctacactagaagaacagtatttggtatctgcg
ctctgctgaagccagttaccttcggaaaaagagttggtagctcttgatccggcaaacaaaccac
cgctggtagcggtggtttttttgtttgcaagcagcagattacgcgcagaaaaaaaggatctcaa
gaagatcctttgatcttttctacggggtctgacgctcagtggaacgaaaactcacgttaaggga
ttttggtcatgagattatcaaaaaggatcttcacctagatccttttaaattaaaaatgaagttt
taaatcaatctaaagtatatatgagtaaacttggtctgacagttaccaatgcttaatcagtgag
gcacctatctcagcgatctgtctatttcgttcatccatagttgcctgactccccgtcgtgtaga
taactacgatacgggagggcttaccatctggccccagtgctgcaatgataccgcgagacccacg
ctcaccggctccagatttatcagcaataaaccagccagccggaagggccgagcgcagaagtggt
cctgcaactttatccgcctccatccagtctattaattgttgccgggaagctagagtaagtagtt
cgccagttaatagtttgcgcaacgttgttgccattgctacaggcatcgtggtgtcacgctcgtc
gtttggtatggcttcattcagctccggttcccaacgatcaaggcgagttacatgatcccccatg
ttgtgcaaaaaagcggttagctccttcggtcctccgatcgttgtcagaagtaagttggccgcag
tgttatcactcatggttatggcagcactgcataattctcttactgtcatgccatccgtaagatg
cttttctgtgactggtgagtactcaaccaagtcattctgagaatagtgtatgcggcgaccgagt
tgctcttgcccggcgtcaatacgggataataccgcgccacatagcagaactttaaaagtgctca
tcattggaaaacgttcttcggggcgaaaactctcaaggatcttaccgctgttgagatccagttc
gatgtaacccactcgtgcacccaactgatcttcagcatcttttactttcaccagcgtttctggg
tgagcaaaaacaggaaggcaaaatgccgcaaaaaagggaataagggcgacacggaaatgttgaa
tactcatactcttcctttttcaatattattgaagcatttatcagggttattgtctcatgagcgg
atacatatttgaatgtatttagaaaaataaacaaataggggttccgcgcacatttccccgaaaa
gtgccacctgacgtc
pGL2-SS*-CAG-SecNLuc-2A-eGFP-WPRE-BGpA-SS*
SEQ ID NO: 21
taaagtaacccaatcagcacacaattgccattatacgcgcgtataatggactattgtgtgctga
taaacctatttcagcatactacgcgcgtagtatgctgaaataggtgactagaagttcctatact
ttctagagaataggaacttcataacttcgtataatgtatgctatacgaagttatgggttacttt
aatttggttgctgactaattgagatgcatgctttgcatacttctgcctgctggggagcctgggg
actttccacacctggttgctgactaattgagatgcatgctttgcatacttctgcctgctgggga
gcctggggactttccacacccctgggtcgacattgattattgactagttattaatagtaatcaa
ttacggggtcattagttcatagcccatatatggagttccgcgttacataacttacggtaaatgg
cccgcctggctgaccgcccaacgacccccgcccattgacgtcaataatgacgtatgttcccata
gtaacgccaatagggactttccattgacgtcaatgggtggagtatttacggtaaactgcccact
tggcagtacatcaagtgtatcatatgccaagtacgccccctattgacgtcaatgacggtaaatg
gcccgcctggcattatgcccagtacatgaccttatgggactttcctacttggcagtacatctac
gtattagtcatcgctattaccatggtcgaggtgagccccacgttctgcttcactctccccatct
cccccccctccccacccccaattttgtatttatttattttttaattattttgtgcagcgatggg
ggcggggggggggggggcgcgcgccaggcggggcggggcggggcgaggggcggggcggggcgag
gcggaaaggtgcggcggcagccaatcagagcggcgcgctccgaaagtttccttttatggcgagg
cggcggcggcggcggccctataaaaagcgaagcgcgcggcgggcgggagtcgctgcgttgcctt
cgccccgtgccccgctccgcgccgcctcgcgccgcccgccccggctctgactgaccgcgttact
cccacaggtgagcgggcgggacggcccttctcctccgggctgtaattagcgcttggtttaatga
cggctcgtttcttttctgtggctgcgtgaaagccttaaagggctccgggagggccctttgtgcg
ggggggagcggctcggggggtgcgtgcgtgtgtgtgtgcgtggggagcgccgcgtgcggctccg
cgctgcccggcggctgtgagcgctgcgggcgcggcgcggggctttgtgcgctccgcagtgtgcg
cgaggggagcgcggccgggggcggtgccccgcggtgcggggggggctgcgaggggaacaaaggc
tgcgtgcggggtgtgtgcgtgggggggtgagcagggggtgtgggcgcggcggtcgggctgtaac
ccccccctgcacccccctccccgagttgctgagcacggcccggcttcgggtgcggggctccgta
cggggcgtggcgcggggctcgccgtgccgggcggggggtggcggcaggtgggggtgccgggcgg
ggcggggccgcctcgggccggggagggctcgggggaggggcgcggcggcccccggagcgccggc
ggctgtcgaggcgcggcgagccgcagccattgccttttatggtaatcgtgcgagagggcgcagg
gacttcctttgtcccaaatctgtgcggagccgaaatctgggaggcgccgccgcaccccctctag
cgggcgcggggcgaagcggtgcggcgccggcaggaaggaaatgggcggggagggccttcgtgcg
tcgccgcgccgccgtccccttctccctctccagcctcggggctgtccgcggggggacggctgcc
ttcgggggggacggggcagggcggggttcggcttctggcgtgtgaccggcggctctagagcctc
tgctaaccatgttcatgccttcttctttttcctacagctcctgggcaacgtgctggttattgtg
ctgtctcatcattttggcaaagaattgattaattcgagcgaacgcgtcgccaccatgaactcct
tctccacaagcgccttcggtccagttgccttctccctgggcctgctcctggtgttgcctgctgc
cttccctgccccagtcttcacactcgaagatttcgttggggactggcgacagacagccggctac
aacctggaccaagtccttgaacagggaggtgtgtccagtttgtttcagaatctcggggtgtccg
taactccgatccaaaggattgtcctgagcggtgaaaatgggctgaagatcgacatccatgtcat
catcccgtatgaaggtctgagcggcgaccaaatgggccagatcgaaaaaatttttaaggtggtg
taccctgtggatgatcatcactttaaggtgatcctgcactatggcacactggtaatcgacgggg
ttacgccgaacatgatcgactatttcggacggccgtatgaaggcatcgccgtgttcgacggcaa
aaagatcactgtaacagggaccctgtggaacggcaacaaaattatcgacgagcgcctgatcaac
cccgacggctccctgctgttccgagtaaccatcaacggagtgaccggctggcggctgtgcgaac
gcattctggcggctagcgctactaacttcagcctgctgaagcaggctggagacgtggaggagaa
ccctggacctggaagcggagagggcagaggaagtctgctaacatgcggtgacgtcgaggagaat
cctggacctggatccggaatggtgagcaagggcgaggagctgttcaccggggtggtgcccatcc
tggtcgagctggacggcgacgtaaacggccacaagttcagcgtgtccggcgagggcgagggcga
tgccacctacggcaagctgaccctgaagttcatctgcaccaccggcaagctgcccgtgccctgg
cccaccctcgtgaccaccctgacctacggcgtgcagtgcttcagccgctaccccgaccacatga
agcagcacgacttcttcaagtccgccatgcccgaaggctacgtccaggagcgcaccatcttctt
caaggacgacggcaactacaagacccgcgccgaggtgaagttcgagggcgacaccctggtgaac
cgcatcgagctgaagggcatcgacttcaaggaggacggcaacatcctggggcacaagctggagt
acaactacaacagccacaacgtctatatcatggccgacaagcagaagaacggcatcaaggtgaa
cttcaagatccgccacaacatcgaggacggcagcgtgcagctcgccgaccactaccagcagaac
acccccatcggcgacggccccgtgctgctgcccgacaaccactacctgagcacccagtccgccc
tgagcaaagaccccaacgagaagcgcgatcacatggtcctgctggagttcgtgaccgccgccgg
gatcactctcggcatggacgagctgtacaagtaaaatcaacctctggattacaaaatttgtgaa
agattgactggtattcttaactatgttgctccttttacgctatgtggatacgctgctttaatgc
ctttgtatcatgctattgcttcccgtatggctttcattttctcctccttgtataaatcctggtt
gctgtctctttatgaggagttgtggcccgttgtcaggcaacgtggcgtggtgtgcactgtgttt
gctgacgcaacccccactggttggggcattgccaccacctgtcagctcctttccgggactttcg
ctttccccctccctattgccacggcggaactcatcgccgcctgccttgcccgctgctggacagg
ggctcggctgttgggcactgacaattccgtggtgttgtcggggaaatcatcgtcctttccttgg
ctgctcgcctgtgttgccacctggattctgcgcgggacgtccttctgctacgtcccttcggccc
tcaatccagcggaccttccttcccgcggcctgctgccggctctgcggcctcttccgcgtcttcg
ccttcgccctcagacgagtcggatctccctttgggccgcctccccgcaataaaggaaatttatt
ttcattgcaatagtgtgttggaattttttgtgtctctcactcggaaggacatggtgtggaaagt
ccccaggctccccagcaggcagaagtatgcaaagcatgcatctcaattagtcagcaaccaaatt
aaagtaacccataacttcgtatagcatacattatacgaagttatgaagttcctattctctagaa
agtataggaacttctagtcacctatttcagcatactacgcgcgtagtatgctgaaataggttta
tcagcacacaatagtccattatacgcgcgtataatggcaattgtgtgctgattgggttacttta
attggtgtggaaagtccccaggctccccagcaggcagaagtatgcaaagcatgcatctcaatta
gtcagcaacca
pGL2-SS*-CMV-UTR1-SecNLuc-2A-eGFP-WPRE-BGpA-SS*
SEQ ID NO: 22
taaagtaacccaatcagcacacaattgccattatacgcgcgtataatggactattgtgtgctga
taaacctatttcagcatactacgcgcgtagtatgctgaaataggtgactagaagttcctatact
ttctagagaataggaacttcataacttcgtataatgtatgctatacgaagttatgggttacttt
aatttggttgctgactaattgagatgcatgctttgcatacttctgcctgctggggagcctgggg
actttccacacctggttgctgactaattgagatgcatgctttgcatacttctgcctgctgggga
gcctggggactttccacacccctgggtcgacgacattgattattgactagttattaatagtaat
caattacggggtcattagttcatagcccatatatggagttccgcgttacataacttacggtaaa
tggcccgcctggctgaccgcccaacgacccccgcccattgacgtcaataatgacgtatgttccc
atagtaacgccaatagggactttccattgacgtcaatgggtggagtatttacggtaaactgccc
acttggcagtacatcaagtgtatcatatgccaagtacgccccctattgacgtcaatgacggtaa
atggcccgcctggcattatgcccagtacatgaccttatgggactttcctacttggcagtacatc
tacgtattagtcatcgctattaccatggtgatgcggttttggcagtacatcaatgggcgtggat
agcggtttgactcacggggatttccaagtctccaccccattgacgtcaatgggagtttgttttg
gcaccaaaatcaacgggactttccaaaatgtcgtaacaactccgccccattgacgcaaatgggc
ggtaggcgtgtacggtgggaggtctatataagcagagctctgccttctccctcctgtgagtttg
gtaagtcgacgggccgggcctgggccgggtccgggccgggtcgttggatccccactacagcccg
atactcaagcttgacgaattcgagtatccaaggtagtggactagtgtgacgctgctgacccctt
tctttcccttctgcaggttggtgtacagtagcttccaaattgattaattcgagcgaacgcgtcg
ccaccatgaactccttctccacaagcgccttcggtccagttgccttctccctgggcctgctcct
ggtgttgcctgctgccttccctgccccagtcttcacactcgaagatttcgttggggactggcga
cagacagccggctacaacctggaccaagtccttgaacagggaggtgtgtccagtttgtttcaga
atctcggggtgtccgtaactccgatccaaaggattgtcctgagcggtgaaaatgggctgaagat
cgacatccatgtcatcatcccgtatgaaggtctgagcggcgaccaaatgggccagatcgaaaaa
atttttaaggtggtgtaccctgtggatgatcatcactttaaggtgatcctgcactatggcacac
tggtaatcgacggggttacgccgaacatgatcgactatttcggacggccgtatgaaggcatcgc
cgtgttcgacggcaaaaagatcactgtaacagggaccctgtggaacggcaacaaaattatcgac
gagcgcctgatcaaccccgacggctccctgctgttccgagtaaccatcaacggagtgaccggct
ggcggctgtgcgaacgcattctggcggctagcgctactaacttcagcctgctgaagcaggctgg
agacgtggaggagaaccctggacctggaagcggagagggcagaggaagtctgctaacatgcggt
gacgtcgaggagaatcctggacctggatccggaatggtgagcaagggcgaggagctgttcaccg
gggtggtgcccatcctggtcgagctggacggcgacgtaaacggccacaagttcagcgtgtccgg
cgagggcgagggcgatgccacctacggcaagctgaccctgaagttcatctgcaccaccggcaag
ctgcccgtgccctggcccaccctcgtgaccaccctgacctacggcgtgcagtgcttcagccgct
accccgaccacatgaagcagcacgacttcttcaagtccgccatgcccgaaggctacgtccagga
gcgcaccatcttcttcaaggacgacggcaactacaagacccgcgccgaggtgaagttcgagggc
gacaccctggtgaaccgcatcgagctgaagggcatcgacttcaaggaggacggcaacatcctgg
ggcacaagctggagtacaactacaacagccacaacgtctatatcatggccgacaagcagaagaa
cggcatcaaggtgaacttcaagatccgccacaacatcgaggacggcagcgtgcagctcgccgac
cactaccagcagaacacccccatcggcgacggccccgtgctgctgcccgacaaccactacctga
gcacccagtccgccctgagcaaagaccccaacgagaagcgcgatcacatggtcctgctggagtt
cgtgaccgccgccgggatcactctcggcatggacgagctgtacaagtaaaatcaacctctggat
tacaaaatttgtgaaagattgactggtattcttaactatgttgctccttttacgctatgtggat
acgctgctttaatgcctttgtatcatgctattgcttcccgtatggctttcattttctcctcctt
gtataaatcctggttgctgtctctttatgaggagttgtggcccgttgtcaggcaacgtggcgtg
gtgtgcactgtgtttgctgacgcaacccccactggttggggcattgccaccacctgtcagctcc
tttccgggactttcgctttccccctccctattgccacggcggaactcatcgccgcctgccttgc
ccgctgctggacaggggctcggctgttgggcactgacaattccgtggtgttgtcggggaaatca
tcgtcctttccttggctgctcgcctgtgttgccacctggattctgcgcgggacgtccttctgct
acgtcccttcggccctcaatccagcggaccttccttcccgcggcctgctgccggctctgcggcc
tcttccgcgtcttcgccttcgccctcagacgagtcggatctccctttgggccgcctccccgcaa
taaaggaaatttattttcattgcaatagtgtgttggaattttttgtgtctctcactcggaagga
catggtgtggaaagtccccaggctccccagcaggcagaagtatgcaaagcatgcatctcaatta
gtcagcaaccaaattaaagtaacccataacttcgtatagcatacattatacgaagttatgaagt
tcctattctctagaaagtataggaacttctagtcacctatttcagcatactacgcgcgtagtat
gctgaaataggtttatcagcacacaatagtccattatacgcgcgtataatggcaattgtgtgct
gattgggttactttaattggtgtggaaagtccccaggctccccagcaggcagaagtatgcaaag
catgcatctcaattagtcagcaacca
pGL2-SS*-CMV-UTR2-SecNLuc-2A-eGFP-WPRE-BGpA-SS*
SEQ ID NO: 23
aattaaagtaacccaatcagcacacaattgccattatacgcgcgtataatggactattgtgtgc
tgataaacctatttcagcatactacgcgcgtagtatgctgaaataggtgactagaagttcctat
actttctagagaataggaacttcataacttcgtataatgtatgctatacgaagttatgggttac
tttaatttggttgctgactaattgagatgcatgctttgcatacttctgcctgctggggagcctg
gggactttccacacctggttgctgactaattgagatgcatgctttgcatacttctgcctgctgg
ggagcctggggactttccacacccctgggtcgacattgattattgactagttattaatagtaat
caattacggggtcattagttcatagcccatatatggagttccgcgttacataacttacggtaaa
tggcccgcctggctgaccgcccaacgacccccgcccattgacgtcaataatgacgtatgttccc
atagtaacgccaatagggactttccattgacgtcaatgggtggagtatttacggtaaactgccc
acttggcagtacatcaagtgtatcatatgccaagtacgccccctattgacgtcaatgacggtaa
atggcccgcctggcattatgcccagtacatgaccttatgggactttcctacttggcagtacatc
tacgtattagtcatcgctattaccatggtgatgcggttttggcagtacatcaatgggcgtggat
agcggtttgactcacggggatttccaagtctccaccccattgacgtcaatgggagtttgttttg
gcaccaaaatcaacgggactttccaaaatgtcgtaacaactccgccccattgacgcaaatgggc
ggtaggcgtgtacggtgggaggtctatataagcagagctattgggatcttcacacagcaggtaa
ggttgcgggccgggcctgggccgggtccgggccgggtatccatagctgattggtctaaaatgag
atacatcaacgctcctccatgttttttgttttctttttaaatgaaaaactttattttttaagag
gagtttcaggttcatagcaaaattgagaggaaggtacattcaagctgaggaagttttcctctat
tcctagtttactgagagattgcatcatgaatgggtgttaaattttgtcaaatgctttttctgtg
tctatcaatatgaccatgtgattttcttctttaacctgttgatgggacaaattacgttaattga
ttttcaaacgttgaaccacccttacatatctggaataaattctacttggttgtggtgtatattt
tttgatacattcttggattctttttgctaatattttgttgaaaatgtttgtatctttgttcatg
agagatattggtctgttgttttcttttcttgtaatgtcattttctagttccggtattaaggtaa
tgctggcctagttgaatgatttaggaagtattccctctgcttctgtcttctgaaagagattgta
gaaagttgatacaatttttttttctttaaatatcttgatagccgcactgacccctggtgttgct
ttttttttttaggccgcaagctgaagcgtgtccgccaccatgaactccttctccacaagcgcct
tcggtccagttgccttctccctgggcctgctcctggtgttgcctgctgccttccctgccccagt
cttcacactcgaagatttcgttggggactggcgacagacagccggctacaacctggaccaagtc
cttgaacagggaggtgtgtccagtttgtttcagaatctcggggtgtccgtaactccgatccaaa
ggattgtcctgagcggtgaaaatgggctgaagatcgacatccatgtcatcatcccgtatgaagg
tctgagcggcgaccaaatgggccagatcgaaaaaatttttaaggtggtgtaccctgtggatgat
catcactttaaggtgatcctgcactatggcacactggtaatcgacggggttacgccgaacatga
tcgactatttcggacggccgtatgaaggcatcgccgtgttcgacggcaaaaagatcactgtaac
agggaccctgtggaacggcaacaaaattatcgacgagcgcctgatcaaccccgacggctccctg
ctgttccgagtaaccatcaacggagtgaccggctggcggctgtgcgaacgcattctggcggcta
gcgctactaacttcagcctgctgaagcaggctggagacgtggaggagaaccctggacctggaag
cggagagggcagaggaagtctgctaacatgcggtgacgtcgaggagaatcctggacctggatcc
ggaatggtgagcaagggcgaggagctgttcaccggggtggtgcccatcctggtcgagctggacg
gcgacgtaaacggccacaagttcagcgtgtccggcgagggcgagggcgatgccacctacggcaa
gctgaccctgaagttcatctgcaccaccggcaagctgcccgtgccctggcccaccctcgtgacc
accctgacctacggcgtgcagtgcttcagccgctaccccgaccacatgaagcagcacgacttct
tcaagtccgccatgcccgaaggctacgtccaggagcgcaccatcttcttcaaggacgacggcaa
ctacaagacccgcgccgaggtgaagttcgagggcgacaccctggtgaaccgcatcgagctgaag
ggcatcgacttcaaggaggacggcaacatcctggggcacaagctggagtacaactacaacagcc
acaacgtctatatcatggccgacaagcagaagaacggcatcaaggtgaacttcaagatccgcca
caacatcgaggacggcagcgtgcagctcgccgaccactaccagcagaacacccccatcggcgac
ggccccgtgctgctgcccgacaaccactacctgagcacccagtccgccctgagcaaagacccca
acgagaagcgcgatcacatggtcctgctggagttcgtgaccgccgccgggatcactctcggcat
ggacgagctgtacaagtaaaatcaacctctggattacaaaatttgtgaaagattgactggtatt
cttaactatgttgctccttttacgctatgtggatacgctgctttaatgcctttgtatcatgcta
ttgcttcccgtatggctttcattttctcctccttgtataaatcctggttgctgtctctttatga
ggagttgtggcccgttgtcaggcaacgtggcgtggtgtgcactgtgtttgctgacgcaaccccc
actggttggggcattgccaccacctgtcagctcctttccgggactttcgctttccccctcccta
ttgccacggcggaactcatcgccgcctgccttgcccgctgctggacaggggctcggctgttggg
cactgacaattccgtggtgttgtcggggaaatcatcgtcctttccttggctgctcgcctgtgtt
gccacctggattctgcgcgggacgtccttctgctacgtcccttcggccctcaatccagcggacc
ttccttcccgcggcctgctgccggctctgcggcctcttccgcgtcttcgccttcgccctcagac
gagtcggatctccctttgggccgcctccccgcaataaaggaaatttattttcattgcaatagtg
tgttggaattttttgtgtctctcactcggaaggacatggtgtggaaagtccccaggctccccag
caggcagaagtatgcaaagcatgcatctcaattagtcagcaaccaaattaaagtaacccataac
ttcgtatagcatacattatacgaagttatgaagttcctattctctagaaagtataggaacttct
agtcacctatttcagcatactacgcgcgtagtatgctgaaataggtttatcagcacacaatagt
ccattatacgcgcgtataatggcaattgtgtgctgattgggttactttaattggtgtggaaagt
ccccaggctccccagcaggcagaagtatgcaaagcatgcatctcaattagtcagcaacca
SS*-CMV-UTR1-SecNLuc-2A-eGFP-3′UTR[2huBGpA-A120]-SS*
SEQ ID NO: 24
cccgggaggtaccgagctcttacgcgtgctagaattaaagtaacccaatcagcacacaattgcc
attatacgcgcgtataatggactattgtgtgctgataaacctatttcagcatactacgcgcgta
gtatgctgaaataggtgactagaagttcctatactttctagagaataggaacttcataacttcg
tataatgtatgctatacgaagttatgggttactttaatttggttgctgactaattgagatgcat
gctttgcatacttctgcctgctggggagcctggggactttccacacctggttgctgactaattg
agatgcatgctttgcatacttctgcctgctggggagcctggggactttccacacccctgggtcg
acgacattgattattgactagttattaatagtaatcaattacggggtcattagttcatagccca
tatatggagttccgcgttacataacttacggtaaatggcccgcctggctgaccgcccaacgacc
cccgcccattgacgtcaataatgacgtatgttcccatagtaacgccaatagggactttccattg
acgtcaatgggtggagtatttacggtaaactgcccacttggcagtacatcaagtgtatcatatg
ccaagtacgccccctattgacgtcaatgacggtaaatggcccgcctggcattatgcccagtaca
tgaccttatgggactttcctacttggcagtacatctacgtattagtcatcgctattaccatggt
gatgcggttttggcagtacatcaatgggcgtggatagcggtttgactcacggggatttccaagt
ctccaccccattgacgtcaatgggagtttgttttggcaccaaaatcaacgggactttccaaaat
gtcgtaacaactccgccccattgacgcaaatgggcggtaggcgtgtacggtgggaggtctatat
aagcagagctctgccttctccctcctgtgagtttggtaagtcgacgggccgggcctgggccggg
tccgggccgggtcgttggatccccactacagcccgatactcaagcttgacgaattcgagtatcc
aaggtagtggactagtgtgacgctgctgacccctttctttcccttctgcaggttggtgtacagt
agcttccaaattgattaattcgagcgaacgcgtcgccaccatgaactccttctccacaagcgcc
ttcggtccagttgccttctccctgggcctgctcctggtgttgcctgctgccttccctgccccag
tcttcacactcgaagatttcgttggggactggcgacagacagccggctacaacctggaccaagt
ccttgaacagggaggtgtgtccagtttgtttcagaatctcggggtgtccgtaactccgatccaa
aggattgtcctgagcggtgaaaatgggctgaagatcgacatccatgtcatcatcccgtatgaag
gtctgagcggcgaccaaatgggccagatcgaaaaaatttttaaggtggtgtaccctgtggatga
tcatcactttaaggtgatcctgcactatggcacactggtaatcgacggggttacgccgaacatg
atcgactatttcggacggccgtatgaaggcatcgccgtgttcgacggcaaaaagatcactgtaa
cagggaccctgtggaacggcaacaaaattatcgacgagcgcctgatcaaccccgacggctccct
gctgttccgagtaaccatcaacggagtgaccggctggcggctgtgcgaacgcattctggcggct
agcgctactaacttcagcctgctgaagcaggctggagacgtggaggagaaccctggacctggaa
gcggagagggcagaggaagtctgctaacatgcggtgacgtcgaggagaatcctggacctggatc
cggaatggtgagcaagggcgaggagctgttcaccggggtggtgcccatcctggtcgagctggac
ggcgacgtaaacggccacaagttcagcgtgtccggcgagggcgagggcgatgccacctacggca
agctgaccctgaagttcatctgcaccaccggcaagctgcccgtgccctggcccaccctcgtgac
caccctgacctacggcgtgcagtgcttcagccgctaccccgaccacatgaagcagcacgacttc
ttcaagtccgccatgcccgaaggctacgtccaggagcgcaccatcttcttcaaggacgacggca
actacaagacccgcgccgaggtgaagttcgagggcgacaccctggtgaaccgcatcgagctgaa
gggcatcgacttcaaggaggacggcaacatcctggggcacaagctggagtacaactacaacagc
cacaacgtctatatcatggccgacaagcagaagaacggcatcaaggtgaacttcaagatccgcc
acaacatcgaggacggcagcgtgcagctcgccgaccactaccagcagaacacccccatcggcga
cggccccgtgctgctgcccgacaaccactacctgagcacccagtccgccctgagcaaagacccc
aacgagaagcgcgatcacatggtcctgctggagttcgtgaccgccgccgggatcactctcggca
tggacgagctgtacaagtaagctcgctttcttgctgtccaatttctattaaaggttcctttgtt
ccctaagtccaactactaaactgggggatattatgaagggccttgagcatctggattctgccta
ataaaaaacatttattttcattgcaagctcgctttcttgctgtccaatttctattaaaggttcc
tttgttccctaagtccaactactaaactgggggatattatgaagggccttgagcatctggattc
tgcctaataaaaaacatttattttcattgcaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaa
aaaaaaaaaaaaaaaaaaaaaaaactcggaaggacatggtgtggaaagtccccaggctccccag
caggcagaagtatgcaaagcatgcatctcaattagtcagcaaccaggtgtggaaagtccccagg
ctccccagcaggcagaagtatgcaaagcatgcatctcaattagtcagcaaccaaattaaagtaa
cccataacttcgtatagcatacattatacgaagttatgaagttcctattctctagaaagtatag
gaacttctagtcacctatttcagcatactacgcgcgtagtatgctgaaataggtttatcagcac
acaatagtccattatacgcgcgtataatggcaattgtgtgctgattgggttactttaatttgga
tccgtcgaccgatgcccttgagagccttcaacccagtcagctccttccggtgggcgcggggcat
gactatcgtcgccgcacttatgactgtcttctttatcatgcaactcgtaggacaggtgccggca
gcgctcttccgcttcctcgctcactgactcgctgcgctcggtcgttcggctgcggcgagcggta
tcagctcactcaaaggcggtaatacggttatccacagaatcaggggataacgcaggaaagaaca
tgtgagcaaaaggccagcaaaaggccaggaaccgtaaaaaggccgcgttgctggcgtttttcca
taggctccgcccccctgacgagcatcacaaaaatcgacgctcaagtcagaggtggcgaaacccg
acaggactataaagataccaggcgtttccccctggaagctccctcgtgcgctctcctgttccga
ccctgccgcttaccggatacctgtccgcctttctcccttcgggaagcgtggcgctttctcatag
ctcacgctgtaggtatctcagttcggtgtaggtcgttcgctccaagctgggctgtgtgcacgaa
ccccccgttcagcccgaccgctgcgccttatccggtaactatcgtcttgagtccaacccggtaa
gacacgacttatcgccactggcagcagccactggtaacaggattagcagagcgaggtatgtagg
cggtgctacagagttcttgaagtggtggcctaactacggctacactagaagaacagtatttggt
atctgcgctctgctgaagccagttaccttcggaaaaagagttggtagctcttgatccggcaaac
aaaccaccgctggtagcggtggtttttttgtttgcaagcagcagattacgcgcagaaaaaaagg
atctcaagaagatcctttgatcttttctacggggtctgacgctcagtggaacgaaaactcacgt
taagggattttggtcatgagattatcaaaaaggatcttcacctagatccttttaaattaaaaat
gaagttttaaatcaatctaaagtatatatgagtaaacttggtctgacagttaccaatgcttaat
cagtgaggcacctatctcagcgatctgtctatttcgttcatccatagttgcctgactccccgtc
gtgtagataactacgatacgggagggcttaccatctggccccagtgctgcaatgataccgcgag
acccacgctcaccggctccagatttatcagcaataaaccagccagccggaagggccgagcgcag
aagtggtcctgcaactttatccgcctccatccagtctattaattgttgccgggaagctagagta
agtagttcgccagttaatagtttgcgcaacgttgttgccattgctacaggcatcgtggtgtcac
gctcgtcgtttggtatggcttcattcagctccggttcccaacgatcaaggcgagttacatgatc
ccccatgttgtgcaaaaaagcggttagctccttcggtcctccgatcgttgtcagaagtaagttg
gccgcagtgttatcactcatggttatggcagcactgcataattctcttactgtcatgccatccg
taagatgcttttctgtgactggtgagtactcaaccaagtcattctgagaatagtgtatgcggcg
accgagttgctcttgcccggcgtcaatacgggataataccgcgccacatagcagaactttaaaa
gtgctcatcattggaaaacgttcttcggggcgaaaactctcaaggatcttaccgctgttgagat
ccagttcgatgtaacccactcgtgcacccaactgatcttcagcatcttttactttcaccagcgt
ttctgggtgagcaaaaacaggaaggcaaaatgccgcaaaaaagggaataagggcgacacggaaa
tgttgaatactcatactcttcctttttcaatattattgaagcatttatcagggttattgtctca
tgagcggatacatatttgaatgtatttagaaaaataaacaaataggggttccgcgcacatttcc
ccgaaaagtgccacctgacgcgccctgtagcggcgcattaagcgcggcgggtgtggtggttacg
cgcagcgtgaccgctacacttgccagcgccctagcgcccgctcctttcgctttcttcccttcct
ttctcgccacgttcgccggctttccccgtcaagctctaaatcgggggctccctttagggttccg
atttagtgctttacggcacctcgaccccaaaaaacttgattagggtgatggttcacgtagtggg
ccatcgccctgatagacggtttttcgccctttgacgttggagtccacgttctttaatagtggac
tcttgttccaaactggaacaacactcaaccctatctcggtctattcttttgatttataagggat
tttgccgatttcggcctattggttaaaaaatgagctgatttaacaaaaatttaacgcgaatttt
aacaaaatattaacgcttacaatttgccattcgccattcaggctgcgcaactgttgggaagggc
gatcggtgcgggcctcttcgctattacgccagcccaagctaccatgataagtaagtaatattaa
ggtacgtggaggttttacttgctttaaaaaacctcccacacctccccctgaacctgaaacataa
aatgaatgcaattgttgttgttaacttgtttattgcagcttataatggttacaaataaagcaat
agcatcacaaatttcacaaataaagcatttttttcactgcattctagttgtggtttgtccaaac
tcatcaatgtatcttatggtactgtaactgagctaacataa
SS*-E1-CMV-UTR1-SecNLuc-2A-eGFP-3′UTR[2huBGpA-A120]-SS*
SEQ ID NO: 25
cccgggaggtaccgagctcttacgcgtgctagaattaaagtaacccaatcagcacacaattgcc
attatacgcgcgtataatggactattgtgtgctgataaacctatttcagcatactacgcgcgta
gtatgctgaaataggtgactagaagttcctatactttctagagaataggaacttcataacttcg
tataatgtatgctatacgaagttatgggttactttaatttggttgctgactaattgagatgcat
gctttgcatacttctgcctgctggggagcctggggactttccacacctggttgctgactaattg
agatgcatgctttgcatacttctgcctgctggggagcctggggactttccacacccctgggtcg
acgggactttccggggcggggcacgtggtgcacgggactttccgtgcacgtgcacgggactttc
cgggactttccgggactttccgtgcaccacgtggggactttccgtgcacgacattgattattga
ctagttattaatagtaatcaattacggggtcattagttcatagcccatatatggagttccgcgt
tacataacttacggtaaatggcccgcctggctgaccgcccaacgacccccgcccattgacgtca
ataatgacgtatgttcccatagtaacgccaatagggactttccattgacgtcaatgggtggagt
atttacggtaaactgcccacttggcagtacatcaagtgtatcatatgccaagtacgccccctat
tgacgtcaatgacggtaaatggcccgcctggcattatgcccagtacatgaccttatgggacttt
cctacttggcagtacatctacgtattagtcatcgctattaccatggtgatgcggttttggcagt
acatcaatgggcgtggatagcggtttgactcacggggatttccaagtctccaccccattgacgt
caatgggagtttgttttggcaccaaaatcaacgggactttccaaaatgtcgtaacaactccgcc
ccattgacgcaaatgggcggtaggcgtgtacggtgggaggtctatataagcagagctctgcctt
ctccctcctgtgagtttggtaagtcgacgggccgggcctgggccgggtccgggccgggtcgttg
gatccccactacagcccgatactcaagcttgacgaattcgagtatccaaggtagtggactagtg
tgacgctgctgacccctttctttcccttctgcaggttggtgtacagtagcttccaaattgatta
attcgagcgaacgcgtcgccaccatgaactccttctccacaagcgccttcggtccagttgcctt
ctccctgggcctgctcctggtgttgcctgctgccttccctgccccagtcttcacactcgaagat
ttcgttggggactggcgacagacagccggctacaacctggaccaagtccttgaacagggaggtg
tgtccagtttgtttcagaatctcggggtgtccgtaactccgatccaaaggattgtcctgagcgg
tgaaaatgggctgaagatcgacatccatgtcatcatcccgtatgaaggtctgagcggcgaccaa
atgggccagatcgaaaaaatttttaaggtggtgtaccctgtggatgatcatcactttaaggtga
tcctgcactatggcacactggtaatcgacggggttacgccgaacatgatcgactatttcggacg
gccgtatgaaggcatcgccgtgttcgacggcaaaaagatcactgtaacagggaccctgtggaac
ggcaacaaaattatcgacgagcgcctgatcaaccccgacggctccctgctgttccgagtaacca
tcaacggagtgaccggctggcggctgtgcgaacgcattctggcggctagcgctactaacttcag
cctgctgaagcaggctggagacgtggaggagaaccctggacctggaagcggagagggcagagga
agtctgctaacatgcggtgacgtcgaggagaatcctggacctggatccggaatggtgagcaagg
gcgaggagctgttcaccggggtggtgcccatcctggtcgagctggacggcgacgtaaacggcca
caagttcagcgtgtccggcgagggcgagggcgatgccacctacggcaagctgaccctgaagttc
atctgcaccaccggcaagctgcccgtgccctggcccaccctcgtgaccaccctgacctacggcg
tgcagtgcttcagccgctaccccgaccacatgaagcagcacgacttcttcaagtccgccatgcc
cgaaggctacgtccaggagcgcaccatcttcttcaaggacgacggcaactacaagacccgcgcc
gaggtgaagttcgagggcgacaccctggtgaaccgcatcgagctgaagggcatcgacttcaagg
aggacggcaacatcctggggcacaagctggagtacaactacaacagccacaacgtctatatcat
ggccgacaagcagaagaacggcatcaaggtgaacttcaagatccgccacaacatcgaggacggc
agcgtgcagctcgccgaccactaccagcagaacacccccatcggcgacggccccgtgctgctgc
ccgacaaccactacctgagcacccagtccgccctgagcaaagaccccaacgagaagcgcgatca
catggtcctgctggagttcgtgaccgccgccgggatcactctcggcatggacgagctgtacaag
taagctcgctttcttgctgtccaatttctattaaaggttcctttgttccctaagtccaactact
aaactgggggatattatgaagggccttgagcatctggattctgcctaataaaaaacatttattt
tcattgcaagctcgctttcttgctgtccaatttctattaaaggttcctttgttccctaagtcca
actactaaactgggggatattatgaagggccttgagcatctggattctgcctaataaaaaacat
ttattttcattgcaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaa
aaaaaaactcggaaggacatggtgtggaaagtccccaggctccccagcaggcagaagtatgcaa
agcatgcatctcaattagtcagcaaccaggtgtggaaagtccccaggctccccagcaggcagaa
gtatgcaaagcatgcatctcaattagtcagcaaccaaattaaagtaacccataacttcgtatag
catacattatacgaagttatgaagttcctattctctagaaagtataggaacttctagtcaccta
tttcagcatactacgcgcgtagtatgctgaaataggtttatcagcacacaatagtccattatac
gcgcgtataatggcaattgtgtgctgattgggttactttaatttggatccgtcgaccgatgccc
ttgagagccttcaacccagtcagctccttccggtgggcgcggggcatgactatcgtcgccgcac
ttatgactgtcttctttatcatgcaactcgtaggacaggtgccggcagcgctcttccgcttcct
cgctcactgactcgctgcgctcggtcgttcggctgcggcgagcggtatcagctcactcaaaggc
ggtaatacggttatccacagaatcaggggataacgcaggaaagaacatgtgagcaaaaggccag
caaaaggccaggaaccgtaaaaaggccgcgttgctggcgtttttccataggctccgcccccctg
acgagcatcacaaaaatcgacgctcaagtcagaggtggcgaaacccgacaggactataaagata
ccaggcgtttccccctggaagctccctcgtgcgctctcctgttccgaccctgccgcttaccgga
tacctgtccgcctttctcccttcgggaagcgtggcgctttctcatagctcacgctgtaggtatc
tcagttcggtgtaggtcgttcgctccaagctgggctgtgtgcacgaaccccccgttcagcccga
ccgctgcgccttatccggtaactatcgtcttgagtccaacccggtaagacacgacttatcgcca
ctggcagcagccactggtaacaggattagcagagcgaggtatgtaggcggtgctacagagttct
tgaagtggtggcctaactacggctacactagaagaacagtatttggtatctgcgctctgctgaa
gccagttaccttcggaaaaagagttggtagctcttgatccggcaaacaaaccaccgctggtagc
ggtggtttttttgtttgcaagcagcagattacgcgcagaaaaaaaggatctcaagaagatcctt
tgatcttttctacggggtctgacgctcagtggaacgaaaactcacgttaagggattttggtcat
gagattatcaaaaaggatcttcacctagatccttttaaattaaaaatgaagttttaaatcaatc
taaagtatatatgagtaaacttggtctgacagttaccaatgcttaatcagtgaggcacctatct
cagcgatctgtctatttcgttcatccatagttgcctgactccccgtcgtgtagataactacgat
acgggagggcttaccatctggccccagtgctgcaatgataccgcgagacccacgctcaccggct
ccagatttatcagcaataaaccagccagccggaagggccgagcgcagaagtggtcctgcaactt
tatccgcctccatccagtctattaattgttgccgggaagctagagtaagtagttcgccagttaa
tagtttgcgcaacgttgttgccattgctacaggcatcgtggtgtcacgctcgtcgtttggtatg
gcttcattcagctccggttcccaacgatcaaggcgagttacatgatcccccatgttgtgcaaaa
aagcggttagctccttcggtcctccgatcgttgtcagaagtaagttggccgcagtgttatcact
catggttatggcagcactgcataattctcttactgtcatgccatccgtaagatgcttttctgtg
actggtgagtactcaaccaagtcattctgagaatagtgtatgcggcgaccgagttgctcttgcc
cggcgtcaatacgggataataccgcgccacatagcagaactttaaaagtgctcatcattggaaa
acgttcttcggggcgaaaactctcaaggatcttaccgctgttgagatccagttcgatgtaaccc
actcgtgcacccaactgatcttcagcatcttttactttcaccagcgtttctgggtgagcaaaaa
caggaaggcaaaatgccgcaaaaaagggaataagggcgacacggaaatgttgaatactcatact
cttcctttttcaatattattgaagcatttatcagggttattgtctcatgagcggatacatattt
gaatgtatttagaaaaataaacaaataggggttccgcgcacatttccccgaaaagtgccacctg
acgcgccctgtagcggcgcattaagcgcggcgggtgtggtggttacgcgcagcgtgaccgctac
acttgccagcgccctagcgcccgctcctttcgctttcttcccttcctttctcgccacgttcgcc
ggctttccccgtcaagctctaaatcgggggctccctttagggttccgatttagtgctttacggc
acctcgaccccaaaaaacttgattagggtgatggttcacgtagtgggccatcgccctgatagac
ggtttttcgccctttgacgttggagtccacgttctttaatagtggactcttgttccaaactgga
acaacactcaaccctatctcggtctattcttttgatttataagggattttgccgatttcggcct
attggttaaaaaatgagctgatttaacaaaaatttaacgcgaattttaacaaaatattaacgct
tacaatttgccattcgccattcaggctgcgcaactgttgggaagggcgatcggtgcgggcctct
tcgctattacgccagcccaagctaccatgataagtaagtaatattaaggtacgtggaggtttta
cttgctttaaaaaacctcccacacctccccctgaacctgaaacataaaatgaatgcaattgttg
ttgttaacttgtttattgcagcttataatggttacaaataaagcaatagcatcacaaatttcac
aaataaagcatttttttcactgcattctagttgtggtttgtccaaactcatcaatgtatcttat
ggtactgtaactgagctaacataa
SS*-E1-CMV-UTR1-SecNLuc-2A-eGFP-WPRE-3′UTR[2huBGpA-A120]-SS*
SEQ ID NO: 26
cccgggaggtaccgagctcttacgcgtgctagaattaaagtaacccaatcagcacacaattgcc
attatacgcgcgtataatggactattgtgtgctgataaacctatttcagcatactacgcgcgta
gtatgctgaaataggtgactagaagttcctatactttctagagaataggaacttcataacttcg
tataatgtatgctatacgaagttatgggttactttaatttggttgctgactaattgagatgcat
gctttgcatacttctgcctgctggggagcctggggactttccacacctggttgctgactaattg
agatgcatgctttgcatacttctgcctgctggggagcctggggactttccacacccctgggtcg
acgggactttccggggcggggcacgtggtgcacgggactttccgtgcacgtgcacgggactttc
cgggactttccgggactttccgtgcaccacgtggggactttccgtgcacgacattgattattga
ctagttattaatagtaatcaattacggggtcattagttcatagcccatatatggagttccgcgt
tacataacttacggtaaatggcccgcctggctgaccgcccaacgacccccgcccattgacgtca
ataatgacgtatgttcccatagtaacgccaatagggactttccattgacgtcaatgggtggagt
atttacggtaaactgcccacttggcagtacatcaagtgtatcatatgccaagtacgccccctat
tgacgtcaatgacggtaaatggcccgcctggcattatgcccagtacatgaccttatgggacttt
cctacttggcagtacatctacgtattagtcatcgctattaccatggtgatgcggttttggcagt
acatcaatgggcgtggatagcggtttgactcacggggatttccaagtctccaccccattgacgt
caatgggagtttgttttggcaccaaaatcaacgggactttccaaaatgtcgtaacaactccgcc
ccattgacgcaaatgggcggtaggcgtgtacggtgggaggtctatataagcagagctctgcctt
ctccctcctgtgagtttggtaagtcgacgggccgggcctgggccgggtccgggccgggtcgttg
gatccccactacagcccgatactcaagcttgacgaattcgagtatccaaggtagtggactagtg
tgacgctgctgacccctttctttcccttctgcaggttggtgtacagtagcttccaaattgatta
attcgagcgaacgcgtcgccaccatgaactccttctccacaagcgccttcggtccagttgcctt
ctccctgggcctgctcctggtgttgcctgctgccttccctgccccagtcttcacactcgaagat
ttcgttggggactggcgacagacagccggctacaacctggaccaagtccttgaacagggaggtg
tgtccagtttgtttcagaatctcggggtgtccgtaactccgatccaaaggattgtcctgagcgg
tgaaaatgggctgaagatcgacatccatgtcatcatcccgtatgaaggtctgagcggcgaccaa
atgggccagatcgaaaaaatttttaaggtggtgtaccctgtggatgatcatcactttaaggtga
tcctgcactatggcacactggtaatcgacggggttacgccgaacatgatcgactatttcggacg
gccgtatgaaggcatcgccgtgttcgacggcaaaaagatcactgtaacagggaccctgtggaac
ggcaacaaaattatcgacgagcgcctgatcaaccccgacggctccctgctgttccgagtaacca
tcaacggagtgaccggctggcggctgtgcgaacgcattctggcggctagcgctactaacttcag
cctgctgaagcaggctggagacgtggaggagaaccctggacctggaagcggagagggcagagga
agtctgctaacatgcggtgacgtcgaggagaatcctggacctggatccggaatggtgagcaagg
gcgaggagctgttcaccggggtggtgcccatcctggtcgagctggacggcgacgtaaacggcca
caagttcagcgtgtccggcgagggcgagggcgatgccacctacggcaagctgaccctgaagttc
atctgcaccaccggcaagctgcccgtgccctggcccaccctcgtgaccaccctgacctacggcg
tgcagtgcttcagccgctaccccgaccacatgaagcagcacgacttcttcaagtccgccatgcc
cgaaggctacgtccaggagcgcaccatcttcttcaaggacgacggcaactacaagacccgcgcc
gaggtgaagttcgagggcgacaccctggtgaaccgcatcgagctgaagggcatcgacttcaagg
aggacggcaacatcctggggcacaagctggagtacaactacaacagccacaacgtctatatcat
ggccgacaagcagaagaacggcatcaaggtgaacttcaagatccgccacaacatcgaggacggc
agcgtgcagctcgccgaccactaccagcagaacacccccatcggcgacggccccgtgctgctgc
ccgacaaccactacctgagcacccagtccgccctgagcaaagaccccaacgagaagcgcgatca
catggtcctgctggagttcgtgaccgccgccgggatcactctcggcatggacgagctgtacaag
taaaatcaacctctggattacaaaatttgtgaaagattgactggtattcttaactatgttgctc
cttttacgctatgtggatacgctgctttaatgcctttgtatcatgctattgcttcccgtatggc
tttcattttctcctccttgtataaatcctggttgctgtctctttatgaggagttgtggcccgtt
gtcaggcaacgtggcgtggtgtgcactgtgtttgctgacgcaacccccactggttggggcattg
ccaccacctgtcagctcctttccgggactttcgctttccccctccctattgccacggcggaact
catcgccgcctgccttgcccgctgctggacaggggctcggctgttgggcactgacaattccgtg
gtgttgtcggggaaatcatcgtcctttccttggctgctcgcctgtgttgccacctggattctgc
gcgggacgtccttctgctacgtcccttcggccctcaatccagcggaccttccttcccgcggcct
gctgccggctctgcggcctcttccgcgtcttcgccttcgccctcagacgagtcggatctccctt
tgggccgcctccccgcgctcgctttcttgctgtccaatttctattaaaggttcctttgttccct
aagtccaactactaaactgggggatattatgaagggccttgagcatctggattctgcctaataa
aaaacatttattttcattgcaagctcgctttcttgctgtccaatttctattaaaggttcctttg
ttccctaagtccaactactaaactgggggatattatgaagggccttgagcatctggattctgcc
taataaaaaacatttattttcattgcaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaa
aaaaaaaaaaaaaaaaaaaactcggaaggacatggtgtggaaagtccccaggctccccagcagg
cagaagtatgcaaagcatgcatctcaattagtcagcaaccaggtgtggaaagtccccaggctcc
ccagcaggcagaagtatgcaaagcatgcatctcaattagtcagcaaccaaattaaagtaaccca
taacttcgtatagcatacattatacgaagttatgaagttcctattctctagaaagtataggaac
ttctagtcacctatttcagcatactacgcgcgtagtatgctgaaataggtttatcagcacacaa
tagtccattatacgcgcgtataatggcaattgtgtgctgattgggttactttaatttggatccg
tcgaccgatgcccttgagagccttcaacccagtcagctccttccggtgggcgcggggcatgact
atcgtcgccgcacttatgactgtcttctttatcatgcaactcgtaggacaggtgccggcagcgc
tcttccgcttcctcgctcactgactcgctgcgctcggtcgttcggctgcggcgagcggtatcag
ctcactcaaaggcggtaatacggttatccacagaatcaggggataacgcaggaaagaacatgtg
agcaaaaggccagcaaaaggccaggaaccgtaaaaaggccgcgttgctggcgtttttccatagg
ctccgcccccctgacgagcatcacaaaaatcgacgctcaagtcagaggtggcgaaacccgacag
gactataaagataccaggcgtttccccctggaagctccctcgtgcgctctcctgttccgaccct
gccgcttaccggatacctgtccgcctttctcccttcgggaagcgtggcgctttctcatagctca
cgctgtaggtatctcagttcggtgtaggtcgttcgctccaagctgggctgtgtgcacgaacccc
ccgttcagcccgaccgctgcgccttatccggtaactatcgtcttgagtccaacccggtaagaca
cgacttatcgccactggcagcagccactggtaacaggattagcagagcgaggtatgtaggcggt
gctacagagttcttgaagtggtggcctaactacggctacactagaagaacagtatttggtatct
gcgctctgctgaagccagttaccttcggaaaaagagttggtagctcttgatccggcaaacaaac
caccgctggtagcggtggtttttttgtttgcaagcagcagattacgcgcagaaaaaaaggatct
caagaagatcctttgatcttttctacggggtctgacgctcagtggaacgaaaactcacgttaag
ggattttggtcatgagattatcaaaaaggatcttcacctagatccttttaaattaaaaatgaag
ttttaaatcaatctaaagtatatatgagtaaacttggtctgacagttaccaatgcttaatcagt
gaggcacctatctcagcgatctgtctatttcgttcatccatagttgcctgactccccgtcgtgt
agataactacgatacgggagggcttaccatctggccccagtgctgcaatgataccgcgagaccc
acgctcaccggctccagatttatcagcaataaaccagccagccggaagggccgagcgcagaagt
ggtcctgcaactttatccgcctccatccagtctattaattgttgccgggaagctagagtaagta
gttcgccagttaatagtttgcgcaacgttgttgccattgctacaggcatcgtggtgtcacgctc
gtcgtttggtatggcttcattcagctccggttcccaacgatcaaggcgagttacatgatccccc
atgttgtgcaaaaaagcggttagctccttcggtcctccgatcgttgtcagaagtaagttggccg
cagtgttatcactcatggttatggcagcactgcataattctcttactgtcatgccatccgtaag
atgcttttctgtgactggtgagtactcaaccaagtcattctgagaatagtgtatgcggcgaccg
agttgctcttgcccggcgtcaatacgggataataccgcgccacatagcagaactttaaaagtgc
tcatcattggaaaacgttcttcggggcgaaaactctcaaggatcttaccgctgttgagatccag
ttcgatgtaacccactcgtgcacccaactgatcttcagcatcttttactttcaccagcgtttct
gggtgagcaaaaacaggaaggcaaaatgccgcaaaaaagggaataagggcgacacggaaatgtt
gaatactcatactcttcctttttcaatattattgaagcatttatcagggttattgtctcatgag
cggatacatatttgaatgtatttagaaaaataaacaaataggggttccgcgcacatttccccga
aaagtgccacctgacgcgccctgtagcggcgcattaagcgcggcgggtgtggtggttacgcgca
gcgtgaccgctacacttgccagcgccctagcgcccgctcctttcgctttcttcccttcctttct
cgccacgttcgccggctttccccgtcaagctctaaatcgggggctccctttagggttccgattt
agtgctttacggcacctcgaccccaaaaaacttgattagggtgatggttcacgtagtgggccat
cgccctgatagacggtttttcgccctttgacgttggagtccacgttctttaatagtggactctt
gttccaaactggaacaacactcaaccctatctcggtctattcttttgatttataagggattttg
ccgatttcggcctattggttaaaaaatgagctgatttaacaaaaatttaacgcgaattttaaca
aaatattaacgcttacaatttgccattcgccattcaggctgcgcaactgttgggaagggcgatc
ggtgcgggcctcttcgctattacgccagcccaagctaccatgataagtaagtaatattaaggta
cgtggaggttttacttgctttaaaaaacctcccacacctccccctgaacctgaaacataaaatg
aatgcaattgttgttgttaacttgtttattgcagcttataatggttacaaataaagcaatagca
tcacaaatttcacaaataaagcatttttttcactgcattctagttgtggtttgtccaaactcat
caatgtatcttatggtactgtaactgagctaacataa
SS*-UCOE-E1-CMV-UTR1-SecNLuc-2A-eGFP-WPRE-3′UTR[2huBGpA-
A120]-SS*
SEQ ID NO: 27
cccgggaggtaccgagctcttacgcgtgctagaattaaagtaacccaatcagcacacaattgcc
attatacgcgcgtataatggactattgtgtgctgataaacctatttcagcatactacgcgcgta
gtatgctgaaataggtgactagaagttcctatactttctagagaataggaacttcataacttcg
tataatgtatgctatacgaagttatgggttactttaatttggttgctgactaattgagatgcat
gctttgcatacttctgcctgctggggagcctggggactttccacacctggttgctgactaattg
agatgcatgctttgcatacttctgcctgctggggagcctggggactttccacacccctgggtcg
acgcacacgaccacaattccactgaaagcattttaatacggaacttgtcactcccagggagcct
ccgctcagccggcagttggttcatttcaatccccacgacaacccttcaaagtgcagggcagaca
gcaggtggctctgcccaggcgcctggatcacagcccggcctgcagccctcacctgggcgcgggg
agaccctgaggacgctcctccaggcggcgctggccggggcctgcggacacggacgggcgggctg
agctccgggacccctccccgcgccccgcaccccgcaccccgcaccccgcaccccgcacccggcg
ctcacccgtcccagccccgccgcccgcagccccagctgcaacgcagccaccgccgccatcgcac
ccggccccgcgggcgcttccgggacgcaggaggcatctgcatccggggcgccgctgagtcccgc
ccagagccccgcccccggctccaggttctgcgagcggcttccgccgggctgctccgcgggcgcg
tcggccatgagcgagttgccgggcgacgtgcgggcgtttctgcgggagcacccgagcctgcggc
tccagacggacgcccgcaaggttcgcagcgcgggaggggaacggagtggcggagaagggcgcag
ttgggatgaggggctgaggggagggcaggggagaggagagggcaggggagaggggagaggggag
agcaggagagaggggaaggcaggggagagggcgcggcgggatcaggggaggagagggaagggac
tttccggggcggggcacgtggtgcacgggactttccgtgcacgtgcacgggactttccgggact
ttccgggactttccgtgcaccacgtggggactttccgtgcacgacattgattattgactagtta
ttaatagtaatcaattacggggtcattagttcatagcccatatatggagttccgcgttacataa
cttacggtaaatggcccgcctggctgaccgcccaacgacccccgcccattgacgtcaataatga
cgtatgttcccatagtaacgccaatagggactttccattgacgtcaatgggtggagtatttacg
gtaaactgcccacttggcagtacatcaagtgtatcatatgccaagtacgccccctattgacgtc
aatgacggtaaatggcccgcctggcattatgcccagtacatgaccttatgggactttcctactt
ggcagtacatctacgtattagtcatcgctattaccatggtgatgcggttttggcagtacatcaa
tgggcgtggatagcggtttgactcacggggatttccaagtctccaccccattgacgtcaatggg
agtttgttttggcaccaaaatcaacgggactttccaaaatgtcgtaacaactccgccccattga
cgcaaatgggcggtaggcgtgtacggtgggaggtctatataagcagagctctgccttctccctc
ctgtgagtttggtaagtcgacgggccgggcctgggccgggtccgggccgggtcgttggatcccc
actacagcccgatactcaagcttgacgaattcgagtatccaaggtagtggactagtgtgacgct
gctgacccctttctttcccttctgcaggttggtgtacagtagcttccaaattgattaattcgag
cgaacgcgtcgccaccatgaactccttctccacaagcgccttcggtccagttgccttctccctg
ggcctgctcctggtgttgcctgctgccttccctgccccagtcttcacactcgaagatttcgttg
gggactggcgacagacagccggctacaacctggaccaagtccttgaacagggaggtgtgtccag
tttgtttcagaatctcggggtgtccgtaactccgatccaaaggattgtcctgagcggtgaaaat
gggctgaagatcgacatccatgtcatcatcccgtatgaaggtctgagcggcgaccaaatgggcc
agatcgaaaaaatttttaaggtggtgtaccctgtggatgatcatcactttaaggtgatcctgca
ctatggcacactggtaatcgacggggttacgccgaacatgatcgactatttcggacggccgtat
gaaggcatcgccgtgttcgacggcaaaaagatcactgtaacagggaccctgtggaacggcaaca
aaattatcgacgagcgcctgatcaaccccgacggctccctgctgttccgagtaaccatcaacgg
agtgaccggctggcggctgtgcgaacgcattctggcggctagcgctactaacttcagcctgctg
aagcaggctggagacgtggaggagaaccctggacctggaagcggagagggcagaggaagtctgc
taacatgcggtgacgtcgaggagaatcctggacctggatccggaatggtgagcaagggcgagga
gctgttcaccggggtggtgcccatcctggtcgagctggacggcgacgtaaacggccacaagttc
agcgtgtccggcgagggcgagggcgatgccacctacggcaagctgaccctgaagttcatctgca
ccaccggcaagctgcccgtgccctggcccaccctcgtgaccaccctgacctacggcgtgcagtg
cttcagccgctaccccgaccacatgaagcagcacgacttcttcaagtccgccatgcccgaaggc
tacgtccaggagcgcaccatcttcttcaaggacgacggcaactacaagacccgcgccgaggtga
agttcgagggcgacaccctggtgaaccgcatcgagctgaagggcatcgacttcaaggaggacgg
caacatcctggggcacaagctggagtacaactacaacagccacaacgtctatatcatggccgac
aagcagaagaacggcatcaaggtgaacttcaagatccgccacaacatcgaggacggcagcgtgc
agctcgccgaccactaccagcagaacacccccatcggcgacggccccgtgctgctgcccgacaa
ccactacctgagcacccagtccgccctgagcaaagaccccaacgagaagcgcgatcacatggtc
ctgctggagttcgtgaccgccgccgggatcactctcggcatggacgagctgtacaagtaagctc
gctttcttgctgtccaatttctattaaaggttcctttgttccctaagtccaactactaaactgg
gggatattatgaagggccttgagcatctggattctgcctaataaaaaacatttattttcattgc
aagctcgctttcttgctgtccaatttctattaaaggttcctttgttccctaagtccaactacta
aactgggggatattatgaagggccttgagcatctggattctgcctaataaaaaacatttatttt
cattgcaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaa
ctcggaaggacatggtgtggaaagtccccaggctccccagcaggcagaagtatgcaaagcatgc
atctcaattagtcagcaaccaggtgtggaaagtccccaggctccccagcaggcagaagtatgca
aagcatgcatctcaattagtcagcaaccaaattaaagtaacccataacttcgtatagcatacat
tatacgaagttatgaagttcctattctctagaaagtataggaacttctagtcacctatttcagc
atactacgcgcgtagtatgctgaaataggtttatcagcacacaatagtccattatacgcgcgta
taatggcaattgtgtgctgattgggttactttaatttggatccgtcgaccgatgcccttgagag
ccttcaacccagtcagctccttccggtgggcgcggggcatgactatcgtcgccgcacttatgac
tgtcttctttatcatgcaactcgtaggacaggtgccggcagcgctcttccgcttcctcgctcac
tgactcgctgcgctcggtcgttcggctgcggcgagcggtatcagctcactcaaaggcggtaata
cggttatccacagaatcaggggataacgcaggaaagaacatgtgagcaaaaggccagcaaaagg
ccaggaaccgtaaaaaggccgcgttgctggcgtttttccataggctccgcccccctgacgagca
tcacaaaaatcgacgctcaagtcagaggtggcgaaacccgacaggactataaagataccaggcg
tttccccctggaagctccctcgtgcgctctcctgttccgaccctgccgcttaccggatacctgt
ccgcctttctcccttcgggaagcgtggcgctttctcatagctcacgctgtaggtatctcagttc
ggtgtaggtcgttcgctccaagctgggctgtgtgcacgaaccccccgttcagcccgaccgctgc
gccttatccggtaactatcgtcttgagtccaacccggtaagacacgacttatcgccactggcag
cagccactggtaacaggattagcagagcgaggtatgtaggcggtgctacagagttcttgaagtg
gtggcctaactacggctacactagaagaacagtatttggtatctgcgctctgctgaagccagtt
accttcggaaaaagagttggtagctcttgatccggcaaacaaaccaccgctggtagcggtggtt
tttttgtttgcaagcagcagattacgcgcagaaaaaaaggatctcaagaagatcctttgatctt
ttctacggggtctgacgctcagtggaacgaaaactcacgttaagggattttggtcatgagatta
tcaaaaaggatcttcacctagatccttttaaattaaaaatgaagttttaaatcaatctaaagta
tatatgagtaaacttggtctgacagttaccaatgcttaatcagtgaggcacctatctcagcgat
ctgtctatttcgttcatccatagttgcctgactccccgtcgtgtagataactacgatacgggag
ggcttaccatctggccccagtgctgcaatgataccgcgagacccacgctcaccggctccagatt
tatcagcaataaaccagccagccggaagggccgagcgcagaagtggtcctgcaactttatccgc
ctccatccagtctattaattgttgccgggaagctagagtaagtagttcgccagttaatagtttg
cgcaacgttgttgccattgctacaggcatcgtggtgtcacgctcgtcgtttggtatggcttcat
tcagctccggttcccaacgatcaaggcgagttacatgatcccccatgttgtgcaaaaaagcggt
tagctccttcggtcctccgatcgttgtcagaagtaagttggccgcagtgttatcactcatggtt
atggcagcactgcataattctcttactgtcatgccatccgtaagatgcttttctgtgactggtg
agtactcaaccaagtcattctgagaatagtgtatgcggcgaccgagttgctcttgcccggcgtc
aatacgggataataccgcgccacatagcagaactttaaaagtgctcatcattggaaaacgttct
tcggggcgaaaactctcaaggatcttaccgctgttgagatccagttcgatgtaacccactcgtg
cacccaactgatcttcagcatcttttactttcaccagcgtttctgggtgagcaaaaacaggaag
gcaaaatgccgcaaaaaagggaataagggcgacacggaaatgttgaatactcatactcttcctt
tttcaatattattgaagcatttatcagggttattgtctcatgagcggatacatatttgaatgta
tttagaaaaataaacaaataggggttccgcgcacatttccccgaaaagtgccacctgacgcgcc
ctgtagcggcgcattaagcgcggcgggtgtggtggttacgcgcagcgtgaccgctacacttgcc
agcgccctagcgcccgctcctttcgctttcttcccttcctttctcgccacgttcgccggctttc
cccgtcaagctctaaatcgggggctccctttagggttccgatttagtgctttacggcacctcga
ccccaaaaaacttgattagggtgatggttcacgtagtgggccatcgccctgatagacggttttt
cgccctttgacgttggagtccacgttctttaatagtggactcttgttccaaactggaacaacac
tcaaccctatctcggtctattcttttgatttataagggattttgccgatttcggcctattggtt
aaaaaatgagctgatttaacaaaaatttaacgcgaattttaacaaaatattaacgcttacaatt
tgccattcgccattcaggctgcgcaactgttgggaagggcgatcggtgcgggcctcttcgctat
tacgccagcccaagctaccatgataagtaagtaatattaaggtacgtggaggttttacttgctt
taaaaaacctcccacacctccccctgaacctgaaacataaaatgaatgcaattgttgttgttaa
cttgtttattgcagcttataatggttacaaataaagcaatagcatcacaaatttcacaaataaa
gcatttttttcactgcattctagttgtggtttgtccaaactcatcaatgtatcttatggtactg
taactgagctaacataa
SS*-E1-CMV-UTR1-SecNLuc-2A-eGFP-huMAR-3′UTR[2huBGpA-A120]-
SS*
SEQ ID NO: 28
cccgggaggtaccgagctcttacgcgtgctagaattaaagtaacccaatcagcacacaattgcc
attatacgcgcgtataatggactattgtgtgctgataaacctatttcagcatactacgcgcgta
gtatgctgaaataggtgactagaagttcctatactttctagagaataggaacttcataacttcg
tataatgtatgctatacgaagttatgggttactttaatttggttgctgactaattgagatgcat
gctttgcatacttctgcctgctggggagcctggggactttccacacctggttgctgactaattg
agatgcatgctttgcatacttctgcctgctggggagcctggggactttccacacccctgggtcg
acgggactttccggggcggggcacgtggtgcacgggactttccgtgcacgtgcacgggactttc
cgggactttccgggactttccgtgcaccacgtggggactttccgtgcacgacattgattattga
ctagttattaatagtaatcaattacggggtcattagttcatagcccatatatggagttccgcgt
tacataacttacggtaaatggcccgcctggctgaccgcccaacgacccccgcccattgacgtca
ataatgacgtatgttcccatagtaacgccaatagggactttccattgacgtcaatgggtggagt
atttacggtaaactgcccacttggcagtacatcaagtgtatcatatgccaagtacgccccctat
tgacgtcaatgacggtaaatggcccgcctggcattatgcccagtacatgaccttatgggacttt
cctacttggcagtacatctacgtattagtcatcgctattaccatggtgatgcggttttggcagt
acatcaatgggcgtggatagcggtttgactcacggggatttccaagtctccaccccattgacgt
caatgggagtttgttttggcaccaaaatcaacgggactttccaaaatgtcgtaacaactccgcc
ccattgacgcaaatgggcggtaggcgtgtacggtgggaggtctatataagcagagctctgcctt
ctccctcctgtgagtttggtaagtcgacgggccgggcctgggccgggtccgggccgggtcgttg
gatccccactacagcccgatactcaagcttgacgaattcgagtatccaaggtagtggactagtg
tgacgctgctgacccctttctttcccttctgcaggttggtgtacagtagcttccaaattgatta
attcgagcgaacgcgtcgccaccatgaactccttctccacaagcgccttcggtccagttgcctt
ctccctgggcctgctcctggtgttgcctgctgccttccctgccccagtcttcacactcgaagat
ttcgttggggactggcgacagacagccggctacaacctggaccaagtccttgaacagggaggtg
tgtccagtttgtttcagaatctcggggtgtccgtaactccgatccaaaggattgtcctgagcgg
tgaaaatgggctgaagatcgacatccatgtcatcatcccgtatgaaggtctgagcggcgaccaa
atgggccagatcgaaaaaatttttaaggtggtgtaccctgtggatgatcatcactttaaggtga
tcctgcactatggcacactggtaatcgacggggttacgccgaacatgatcgactatttcggacg
gccgtatgaaggcatcgccgtgttcgacggcaaaaagatcactgtaacagggaccctgtggaac
ggcaacaaaattatcgacgagcgcctgatcaaccccgacggctccctgctgttccgagtaacca
tcaacggagtgaccggctggcggctgtgcgaacgcattctggcggctagcgctactaacttcag
cctgctgaagcaggctggagacgtggaggagaaccctggacctggaagcggagagggcagagga
agtctgctaacatgcggtgacgtcgaggagaatcctggacctggatccggaatggtgagcaagg
gcgaggagctgttcaccggggtggtgcccatcctggtcgagctggacggcgacgtaaacggcca
caagttcagcgtgtccggcgagggcgagggcgatgccacctacggcaagctgaccctgaagttc
atctgcaccaccggcaagctgcccgtgccctggcccaccctcgtgaccaccctgacctacggcg
tgcagtgcttcagccgctaccccgaccacatgaagcagcacgacttcttcaagtccgccatgcc
cgaaggctacgtccaggagcgcaccatcttcttcaaggacgacggcaactacaagacccgcgcc
gaggtgaagttcgagggcgacaccctggtgaaccgcatcgagctgaagggcatcgacttcaagg
aggacggcaacatcctggggcacaagctggagtacaactacaacagccacaacgtctatatcat
ggccgacaagcagaagaacggcatcaaggtgaacttcaagatccgccacaacatcgaggacggc
agcgtgcagctcgccgaccactaccagcagaacacccccatcggcgacggccccgtgctgctgc
ccgacaaccactacctgagcacccagtccgccctgagcaaagaccccaacgagaagcgcgatca
catggtcctgctggagttcgtgaccgccgccgggatcactctcggcatggacgagctgtacaag
taaggatcccattctccttgatgtactaatttttctttaaaagtgataataatagctcccattt
agaatttttaaataacacaacaaatgtaaagtaactaatgtgtcctctggatcatggtaagtaa
tgaataaatttaactccctttaccttctccctttgctattttttccatgctaggatttatacat
ttttaaaaaactaaatctgctatcaaatgacagctttaaatttactttttaaaatttgttattg
tatatatttatggggtataaagtgatgttatgatatatatatacacaatgtacactgattaaat
caagccaattaacattttatcatctcaaatacttaacattttttgtagtgagaacatttgaaat
ttacttttagcaatttcaaaacatacattattattattaactatagtcaccatgatgtaccata
gatctttaaaaacttattcttcctgcctaactgaaactttgtactctttgactaacatcttttc
attcccccacttcccagcctctggtaatcaccattacacactctgcttctatgagttcaattgc
tttagactccacgtaataaatgagatcatgcagcatttggctttctgtgcctggcttatccttg
cttagcatggtgtcttacaggttcatccatgttgcaacaaataacagaatctcattctttgtta
aggctgaatactattccattgggtatatataccacattttccttatccattaatccactgatgg
acccttaggttgttgattccatatattggctattgtaaatagtgcagcaatgaacatgagagtg
caactatctcttcaatgtactgatttcgaatccttcggatctatctcagaagtgagattgcagg
atcatataattctacttttagtcttttgaggagctccatacagctttccatatggccatactaa
ttacattctcatcaacagtgtacaatggtttccttttctccacatcctcaccaacatttataat
tttttgtctttttgataatagccatctgacaggtgtaaagtgatagctcattgcagttttaatt
tgcattttttgatgattagtaatgttgagaattttttcatatatctcttggccagttgcatgtc
ttctttggaaaaatgtctattcagttcctttgcccattttttaattgggatttttggtttcttg
ctattgagttgtttgaattcgctcgctttcttgctgtccaatttctattaaaggttcctttgtt
ccctaagtccaactactaaactgggggatattatgaagggccttgagcatctggattctgccta
ataaaaaacatttattttcattgcaagctcgctttcttgctgtccaatttctattaaaggttcc
tttgttccctaagtccaactactaaactgggggatattatgaagggccttgagcatctggattc
tgcctaataaaaaacatttattttcattgcaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaa
aaaaaaaaaaaaaaaaaaaaaaaactcggaaggacatggtgtggaaagtccccaggctccccag
caggcagaagtatgcaaagcatgcatctcaattagtcagcaaccaggtgtggaaagtccccagg
ctccccagcaggcagaagtatgcaaagcatgcatctcaattagtcagcaaccaaattaaagtaa
cccataacttcgtatagcatacattatacgaagttatgaagttcctattctctagaaagtatag
gaacttctagtcacctatttcagcatactacgcgcgtagtatgctgaaataggtttatcagcac
acaatagtccattatacgcgcgtataatggcaattgtgtgctgattgggttactttaatttgga
tccgtcgaccgatgcccttgagagccttcaacccagtcagctccttccggtgggcgcggggcat
gactatcgtcgccgcacttatgactgtcttctttatcatgcaactcgtaggacaggtgccggca
gcgctcttccgcttcctcgctcactgactcgctgcgctcggtcgttcggctgcggcgagcggta
tcagctcactcaaaggcggtaatacggttatccacagaatcaggggataacgcaggaaagaaca
tgtgagcaaaaggccagcaaaaggccaggaaccgtaaaaaggccgcgttgctggcgtttttcca
taggctccgcccccctgacgagcatcacaaaaatcgacgctcaagtcagaggtggcgaaacccg
acaggactataaagataccaggcgtttccccctggaagctccctcgtgcgctctcctgttccga
ccctgccgcttaccggatacctgtccgcctttctcccttcgggaagcgtggcgctttctcatag
ctcacgctgtaggtatctcagttcggtgtaggtcgttcgctccaagctgggctgtgtgcacgaa
ccccccgttcagcccgaccgctgcgccttatccggtaactatcgtcttgagtccaacccggtaa
gacacgacttatcgccactggcagcagccactggtaacaggattagcagagcgaggtatgtagg
cggtgctacagagttcttgaagtggtggcctaactacggctacactagaagaacagtatttggt
atctgcgctctgctgaagccagttaccttcggaaaaagagttggtagctcttgatccggcaaac
aaaccaccgctggtagcggtggtttttttgtttgcaagcagcagattacgcgcagaaaaaaagg
atctcaagaagatcctttgatcttttctacggggtctgacgctcagtggaacgaaaactcacgt
taagggattttggtcatgagattatcaaaaaggatcttcacctagatccttttaaattaaaaat
gaagttttaaatcaatctaaagtatatatgagtaaacttggtctgacagttaccaatgcttaat
cagtgaggcacctatctcagcgatctgtctatttcgttcatccatagttgcctgactccccgtc
gtgtagataactacgatacgggagggcttaccatctggccccagtgctgcaatgataccgcgag
acccacgctcaccggctccagatttatcagcaataaaccagccagccggaagggccgagcgcag
aagtggtcctgcaactttatccgcctccatccagtctattaattgttgccgggaagctagagta
agtagttcgccagttaatagtttgcgcaacgttgttgccattgctacaggcatcgtggtgtcac
gctcgtcgtttggtatggcttcattcagctccggttcccaacgatcaaggcgagttacatgatc
ccccatgttgtgcaaaaaagcggttagctccttcggtcctccgatcgttgtcagaagtaagttg
gccgcagtgttatcactcatggttatggcagcactgcataattctcttactgtcatgccatccg
taagatgcttttctgtgactggtgagtactcaaccaagtcattctgagaatagtgtatgcggcg
accgagttgctcttgcccggcgtcaatacgggataataccgcgccacatagcagaactttaaaa
gtgctcatcattggaaaacgttcttcggggcgaaaactctcaaggatcttaccgctgttgagat
ccagttcgatgtaacccactcgtgcacccaactgatcttcagcatcttttactttcaccagcgt
ttctgggtgagcaaaaacaggaaggcaaaatgccgcaaaaaagggaataagggcgacacggaaa
tgttgaatactcatactcttcctttttcaatattattgaagcatttatcagggttattgtctca
tgagcggatacatatttgaatgtatttagaaaaataaacaaataggggttccgcgcacatttcc
ccgaaaagtgccacctgacgcgccctgtagcggcgcattaagcgcggcgggtgtggtggttacg
cgcagcgtgaccgctacacttgccagcgccctagcgcccgctcctttcgctttcttcccttcct
ttctcgccacgttcgccggctttccccgtcaagctctaaatcgggggctccctttagggttccg
atttagtgctttacggcacctcgaccccaaaaaacttgattagggtgatggttcacgtagtggg
ccatcgccctgatagacggtttttcgccctttgacgttggagtccacgttctttaatagtggac
tcttgttccaaactggaacaacactcaaccctatctcggtctattcttttgatttataagggat
tttgccgatttcggcctattggttaaaaaatgagctgatttaacaaaaatttaacgcgaatttt
aacaaaatattaacgcttacaatttgccattcgccattcaggctgcgcaactgttgggaagggc
gatcggtgcgggcctcttcgctattacgccagcccaagctaccatgataagtaagtaatattaa
ggtacgtggaggttttacttgctttaaaaaacctcccacacctccccctgaacctgaaacataa
aatgaatgcaattgttgttgttaacttgtttattgcagcttataatggttacaaataaagcaat
agcatcacaaatttcacaaataaagcatttttttcactgcattctagttgtggtttgtccaaac
tcatcaatgtatcttatggtactgtaactgagctaacataa
SS*-UCOE-E1-CMV-UTR1-SecNLuc-2A-eGFP-huMAR-3′UTR[2huBGpA-
A120]-SS*
SEQ ID NO: 29
cccgggaggtaccgagctcttacgcgtgctagaattaaagtaacccaatcagcacacaattgcc
attatacgcgcgtataatggactattgtgtgctgataaacctatttcagcatactacgcgcgta
gtatgctgaaataggtgactagaagttcctatactttctagagaataggaacttcataacttcg
tataatgtatgctatacgaagttatgggttactttaatttggttgctgactaattgagatgcat
gctttgcatacttctgcctgctggggagcctggggactttccacacctggttgctgactaattg
agatgcatgctttgcatacttctgcctgctggggagcctggggactttccacacccctgggtcg
acgcacacgaccacaattccactgaaagcattttaatacggaacttgtcactcccagggagcct
ccgctcagccggcagttggttcatttcaatccccacgacaacccttcaaagtgcagggcagaca
gcaggtggctctgcccaggcgcctggatcacagcccggcctgcagccctcacctgggcgcgggg
agaccctgaggacgctcctccaggcggcgctggccggggcctgcggacacggacgggcgggctg
agctccgggacccctccccgcgccccgcaccccgcaccccgcaccccgcaccccgcacccggcg
ctcacccgtcccagccccgccgcccgcagccccagctgcaacgcagccaccgccgccatcgcac
ccggccccgcgggcgcttccgggacgcaggaggcatctgcatccggggcgccgctgagtcccgc
ccagagccccgcccccggctccaggttctgcgagcggcttccgccgggctgctccgcgggcgcg
tcggccatgagcgagttgccgggcgacgtgcgggcgtttctgcgggagcacccgagcctgcggc
tccagacggacgcccgcaaggttcgcagcgcgggaggggaacggagtggcggagaagggcgcag
ttgggatgaggggctgaggggagggcaggggagaggagagggcaggggagaggggagaggggag
agcaggagagaggggaaggcaggggagagggcgcggcgggatcaggggaggagagggaagggac
tttccggggcggggcacgtggtgcacgggactttccgtgcacgtgcacgggactttccgggact
ttccgggactttccgtgcaccacgtggggactttccgtgcacgacattgattattgactagtta
ttaatagtaatcaattacggggtcattagttcatagcccatatatggagttccgcgttacataa
cttacggtaaatggcccgcctggctgaccgcccaacgacccccgcccattgacgtcaataatga
cgtatgttcccatagtaacgccaatagggactttccattgacgtcaatgggtggagtatttacg
gtaaactgcccacttggcagtacatcaagtgtatcatatgccaagtacgccccctattgacgtc
aatgacggtaaatggcccgcctggcattatgcccagtacatgaccttatgggactttcctactt
ggcagtacatctacgtattagtcatcgctattaccatggtgatgcggttttggcagtacatcaa
tgggcgtggatagcggtttgactcacggggatttccaagtctccaccccattgacgtcaatggg
agtttgttttggcaccaaaatcaacgggactttccaaaatgtcgtaacaactccgccccattga
cgcaaatgggcggtaggcgtgtacggtgggaggtctatataagcagagctctgccttctccctc
ctgtgagtttggtaagtcgacgggccgggcctgggccgggtccgggccgggtcgttggatcccc
actacagcccgatactcaagcttgacgaattcgagtatccaaggtagtggactagtgtgacgct
gctgacccctttctttcccttctgcaggttggtgtacagtagcttccaaattgattaattcgag
cgaacgcgtcgccaccatgaactccttctccacaagcgccttcggtccagttgccttctccctg
ggcctgctcctggtgttgcctgctgccttccctgccccagtcttcacactcgaagatttcgttg
gggactggcgacagacagccggctacaacctggaccaagtccttgaacagggaggtgtgtccag
tttgtttcagaatctcggggtgtccgtaactccgatccaaaggattgtcctgagcggtgaaaat
gggctgaagatcgacatccatgtcatcatcccgtatgaaggtctgagcggcgaccaaatgggcc
agatcgaaaaaatttttaaggtggtgtaccctgtggatgatcatcactttaaggtgatcctgca
ctatggcacactggtaatcgacggggttacgccgaacatgatcgactatttcggacggccgtat
gaaggcatcgccgtgttcgacggcaaaaagatcactgtaacagggaccctgtggaacggcaaca
aaattatcgacgagcgcctgatcaaccccgacggctccctgctgttccgagtaaccatcaacgg
agtgaccggctggcggctgtgcgaacgcattctggcggctagcgctactaacttcagcctgctg
aagcaggctggagacgtggaggagaaccctggacctggaagcggagagggcagaggaagtctgc
taacatgcggtgacgtcgaggagaatcctggacctggatccggaatggtgagcaagggcgagga
gctgttcaccggggtggtgcccatcctggtcgagctggacggcgacgtaaacggccacaagttc
agcgtgtccggcgagggcgagggcgatgccacctacggcaagctgaccctgaagttcatctgca
ccaccggcaagctgcccgtgccctggcccaccctcgtgaccaccctgacctacggcgtgcagtg
cttcagccgctaccccgaccacatgaagcagcacgacttcttcaagtccgccatgcccgaaggc
tacgtccaggagcgcaccatcttcttcaaggacgacggcaactacaagacccgcgccgaggtga
agttcgagggcgacaccctggtgaaccgcatcgagctgaagggcatcgacttcaaggaggacgg
caacatcctggggcacaagctggagtacaactacaacagccacaacgtctatatcatggccgac
aagcagaagaacggcatcaaggtgaacttcaagatccgccacaacatcgaggacggcagcgtgc
agctcgccgaccactaccagcagaacacccccatcggcgacggccccgtgctgctgcccgacaa
ccactacctgagcacccagtccgccctgagcaaagaccccaacgagaagcgcgatcacatggtc
ctgctggagttcgtgaccgccgccgggatcactctcggcatggacgagctgtacaagtaaggat
cccattctccttgatgtactaatttttctttaaaagtgataataatagctcccatttagaattt
ttaaataacacaacaaatgtaaagtaactaatgtgtcctctggatcatggtaagtaatgaataa
atttaactccctttaccttctccctttgctattttttccatgctaggatttatacatttttaaa
aaactaaatctgctatcaaatgacagctttaaatttactttttaaaatttgttattgtatatat
ttatggggtataaagtgatgttatgatatatatatacacaatgtacactgattaaatcaagcca
attaacattttatcatctcaaatacttaacattttttgtagtgagaacatttgaaatttacttt
tagcaatttcaaaacatacattattattattaactatagtcaccatgatgtaccatagatcttt
aaaaacttattcttcctgcctaactgaaactttgtactctttgactaacatcttttcattcccc
cacttcccagcctctggtaatcaccattacacactctgcttctatgagttcaattgctttagac
tccacgtaataaatgagatcatgcagcatttggctttctgtgcctggcttatccttgcttagca
tggtgtcttacaggttcatccatgttgcaacaaataacagaatctcattctttgttaaggctga
atactattccattgggtatatataccacattttccttatccattaatccactgatggaccctta
ggttgttgattccatatattggctattgtaaatagtgcagcaatgaacatgagagtgcaactat
ctcttcaatgtactgatttcgaatccttcggatctatctcagaagtgagattgcaggatcatat
aattctacttttagtcttttgaggagctccatacagctttccatatggccatactaattacatt
ctcatcaacagtgtacaatggtttccttttctccacatcctcaccaacatttataattttttgt
ctttttgataatagccatctgacaggtgtaaagtgatagctcattgcagttttaatttgcattt
tttgatgattagtaatgttgagaattttttcatatatctcttggccagttgcatgtcttctttg
gaaaaatgtctattcagttcctttgcccattttttaattgggatttttggtttcttgctattga
gttgtttgaattcgctcgctttcttgctgtccaatttctattaaaggttcctttgttccctaag
tccaactactaaactgggggatattatgaagggccttgagcatctggattctgcctaataaaaa
acatttattttcattgcaagctcgctttcttgctgtccaatttctattaaaggttcctttgttc
cctaagtccaactactaaactgggggatattatgaagggccttgagcatctggattctgcctaa
taaaaaacatttattttcattgcaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaa
aaaaaaaaaaaaaaaaactcggaaggacatggtgtggaaagtccccaggctccccagcaggcag
aagtatgcaaagcatgcatctcaattagtcagcaaccaggtgtggaaagtccccaggctcccca
gcaggcagaagtatgcaaagcatgcatctcaattagtcagcaaccaaattaaagtaacccataa
cttcgtatagcatacattatacgaagttatgaagttcctattctctagaaagtataggaacttc
tagtcacctatttcagcatactacgcgcgtagtatgctgaaataggtttatcagcacacaatag
tccattatacgcgcgtataatggcaattgtgtgctgattgggttactttaatttggatccgtcg
accgatgcccttgagagccttcaacccagtcagctccttccggtgggcgcggggcatgactatc
gtcgccgcacttatgactgtcttctttatcatgcaactcgtaggacaggtgccggcagcgctct
tccgcttcctcgctcactgactcgctgcgctcggtcgttcggctgcggcgagcggtatcagctc
actcaaaggcggtaatacggttatccacagaatcaggggataacgcaggaaagaacatgtgagc
aaaaggccagcaaaaggccaggaaccgtaaaaaggccgcgttgctggcgtttttccataggctc
cgcccccctgacgagcatcacaaaaatcgacgctcaagtcagaggtggcgaaacccgacaggac
tataaagataccaggcgtttccccctggaagctccctcgtgcgctctcctgttccgaccctgcc
gcttaccggatacctgtccgcctttctcccttcgggaagcgtggcgctttctcatagctcacgc
tgtaggtatctcagttcggtgtaggtcgttcgctccaagctgggctgtgtgcacgaaccccccg
ttcagcccgaccgctgcgccttatccggtaactatcgtcttgagtccaacccggtaagacacga
cttatcgccactggcagcagccactggtaacaggattagcagagcgaggtatgtaggcggtgct
acagagttcttgaagtggtggcctaactacggctacactagaagaacagtatttggtatctgcg
ctctgctgaagccagttaccttcggaaaaagagttggtagctcttgatccggcaaacaaaccac
cgctggtagcggtggtttttttgtttgcaagcagcagattacgcgcagaaaaaaaggatctcaa
gaagatcctttgatcttttctacggggtctgacgctcagtggaacgaaaactcacgttaaggga
ttttggtcatgagattatcaaaaaggatcttcacctagatccttttaaattaaaaatgaagttt
taaatcaatctaaagtatatatgagtaaacttggtctgacagttaccaatgcttaatcagtgag
gcacctatctcagcgatctgtctatttcgttcatccatagttgcctgactccccgtcgtgtaga
taactacgatacgggagggcttaccatctggccccagtgctgcaatgataccgcgagacccacg
ctcaccggctccagatttatcagcaataaaccagccagccggaagggccgagcgcagaagtggt
cctgcaactttatccgcctccatccagtctattaattgttgccgggaagctagagtaagtagtt
cgccagttaatagtttgcgcaacgttgttgccattgctacaggcatcgtggtgtcacgctcgtc
gtttggtatggcttcattcagctccggttcccaacgatcaaggcgagttacatgatcccccatg
ttgtgcaaaaaagcggttagctccttcggtcctccgatcgttgtcagaagtaagttggccgcag
tgttatcactcatggttatggcagcactgcataattctcttactgtcatgccatccgtaagatg
cttttctgtgactggtgagtactcaaccaagtcattctgagaatagtgtatgcggcgaccgagt
tgctcttgcccggcgtcaatacgggataataccgcgccacatagcagaactttaaaagtgctca
tcattggaaaacgttcttcggggcgaaaactctcaaggatcttaccgctgttgagatccagttc
gatgtaacccactcgtgcacccaactgatcttcagcatcttttactttcaccagcgtttctggg
tgagcaaaaacaggaaggcaaaatgccgcaaaaaagggaataagggcgacacggaaatgttgaa
tactcatactcttcctttttcaatattattgaagcatttatcagggttattgtctcatgagcgg
atacatatttgaatgtatttagaaaaataaacaaataggggttccgcgcacatttccccgaaaa
gtgccacctgacgcgccctgtagcggcgcattaagcgcggcgggtgtggtggttacgcgcagcg
tgaccgctacacttgccagcgccctagcgcccgctcctttcgctttcttcccttcctttctcgc
cacgttcgccggctttccccgtcaagctctaaatcgggggctccctttagggttccgatttagt
gctttacggcacctcgaccccaaaaaacttgattagggtgatggttcacgtagtgggccatcgc
cctgatagacggtttttcgccctttgacgttggagtccacgttctttaatagtggactcttgtt
ccaaactggaacaacactcaaccctatctcggtctattcttttgatttataagggattttgccg
atttcggcctattggttaaaaaatgagctgatttaacaaaaatttaacgcgaattttaacaaaa
tattaacgcttacaatttgccattcgccattcaggctgcgcaactgttgggaagggcgatcggt
gcgggcctcttcgctattacgccagcccaagctaccatgataagtaagtaatattaaggtacgt
ggaggttttacttgctttaaaaaacctcccacacctccccctgaacctgaaacataaaatgaat
gcaattgttgttgttaacttgtttattgcagcttataatggttacaaataaagcaatagcatca
caaatttcacaaataaagcatttttttcactgcattctagttgtggtttgtccaaactcatcaa
tgtatcttatggtactgtaactgagctaacataa
SS*-UCOE-E1-CMV-UTR1-SecNLuc-2A-eGFP-WPRE-3′UTR[2huBGpA-
A120]-SS*
SEQ ID NO: 30
cccgggaggtaccgagctcttacgcgtgctagaattaaagtaacccaatcagcacacaattgcc
attatacgcgcgtataatggactattgtgtgctgataaacctatttcagcatactacgcgcgta
gtatgctgaaataggtgactagaagttcctatactttctagagaataggaacttcataacttcg
tataatgtatgctatacgaagttatgggttactttaatttggttgctgactaattgagatgcat
gctttgcatacttctgcctgctggggagcctggggactttccacacctggttgctgactaattg
agatgcatgctttgcatacttctgcctgctggggagcctggggactttccacacccctgggtcg
acgcacacgaccacaattccactgaaagcattttaatacggaacttgtcactcccagggagcct
ccgctcagccggcagttggttcatttcaatccccacgacaacccttcaaagtgcagggcagaca
gcaggtggctctgcccaggcgcctggatcacagcccggcctgcagccctcacctgggcgcgggg
agaccctgaggacgctcctccaggcggcgctggccggggcctgcggacacggacgggcgggctg
agctccgggacccctccccgcgccccgcaccccgcaccccgcaccccgcaccccgcacccggcg
ctcacccgtcccagccccgccgcccgcagccccagctgcaacgcagccaccgccgccatcgcac
ccggccccgcgggcgcttccgggacgcaggaggcatctgcatccggggcgccgctgagtcccgc
ccagagccccgcccccggctccaggttctgcgagcggcttccgccgggctgctccgcgggcgcg
tcggccatgagcgagttgccgggcgacgtgcgggcgtttctgcgggagcacccgagcctgcggc
tccagacggacgcccgcaaggttcgcagcgcgggaggggaacggagtggcggagaagggcgcag
ttgggatgaggggctgaggggagggcaggggagaggagagggcaggggagaggggagaggggag
agcaggagagaggggaaggcaggggagagggcgcggcgggatcaggggaggagagggaagggac
tttccggggcggggcacgtggtgcacgggactttccgtgcacgtgcacgggactttccgggact
ttccgggactttccgtgcaccacgtggggactttccgtgcacgacattgattattgactagtta
ttaatagtaatcaattacggggtcattagttcatagcccatatatggagttccgcgttacataa
cttacggtaaatggcccgcctggctgaccgcccaacgacccccgcccattgacgtcaataatga
cgtatgttcccatagtaacgccaatagggactttccattgacgtcaatgggtggagtatttacg
gtaaactgcccacttggcagtacatcaagtgtatcatatgccaagtacgccccctattgacgtc
aatgacggtaaatggcccgcctggcattatgcccagtacatgaccttatgggactttcctactt
ggcagtacatctacgtattagtcatcgctattaccatggtgatgcggttttggcagtacatcaa
tgggcgtggatagcggtttgactcacggggatttccaagtctccaccccattgacgtcaatggg
agtttgttttggcaccaaaatcaacgggactttccaaaatgtcgtaacaactccgccccattga
cgcaaatgggcggtaggcgtgtacggtgggaggtctatataagcagagctctgccttctccctc
ctgtgagtttggtaagtcgacgggccgggcctgggccgggtccgggccgggtcgttggatcccc
actacagcccgatactcaagcttgacgaattcgagtatccaaggtagtggactagtgtgacgct
gctgacccctttctttcccttctgcaggttggtgtacagtagcttccaaattgattaattcgag
cgaacgcgtcgccaccatgaactccttctccacaagcgccttcggtccagttgccttctccctg
ggcctgctcctggtgttgcctgctgccttccctgccccagtcttcacactcgaagatttcgttg
gggactggcgacagacagccggctacaacctggaccaagtccttgaacagggaggtgtgtccag
tttgtttcagaatctcggggtgtccgtaactccgatccaaaggattgtcctgagcggtgaaaat
gggctgaagatcgacatccatgtcatcatcccgtatgaaggtctgagcggcgaccaaatgggcc
agatcgaaaaaatttttaaggtggtgtaccctgtggatgatcatcactttaaggtgatcctgca
ctatggcacactggtaatcgacggggttacgccgaacatgatcgactatttcggacggccgtat
gaaggcatcgccgtgttcgacggcaaaaagatcactgtaacagggaccctgtggaacggcaaca
aaattatcgacgagcgcctgatcaaccccgacggctccctgctgttccgagtaaccatcaacgg
agtgaccggctggcggctgtgcgaacgcattctggcggctagcgctactaacttcagcctgctg
aagcaggctggagacgtggaggagaaccctggacctggaagcggagagggcagaggaagtctgc
taacatgcggtgacgtcgaggagaatcctggacctggatccggaatggtgagcaagggcgagga
gctgttcaccggggtggtgcccatcctggtcgagctggacggcgacgtaaacggccacaagttc
agcgtgtccggcgagggcgagggcgatgccacctacggcaagctgaccctgaagttcatctgca
ccaccggcaagctgcccgtgccctggcccaccctcgtgaccaccctgacctacggcgtgcagtg
cttcagccgctaccccgaccacatgaagcagcacgacttcttcaagtccgccatgcccgaaggc
tacgtccaggagcgcaccatcttcttcaaggacgacggcaactacaagacccgcgccgaggtga
agttcgagggcgacaccctggtgaaccgcatcgagctgaagggcatcgacttcaaggaggacgg
caacatcctggggcacaagctggagtacaactacaacagccacaacgtctatatcatggccgac
aagcagaagaacggcatcaaggtgaacttcaagatccgccacaacatcgaggacggcagcgtgc
agctcgccgaccactaccagcagaacacccccatcggcgacggccccgtgctgctgcccgacaa
ccactacctgagcacccagtccgccctgagcaaagaccccaacgagaagcgcgatcacatggtc
ctgctggagttcgtgaccgccgccgggatcactctcggcatggacgagctgtacaagtaaaatc
aacctctggattacaaaatttgtgaaagattgactggtattcttaactatgttgctccttttac
gctatgtggatacgctgctttaatgcctttgtatcatgctattgcttcccgtatggctttcatt
ttctcctccttgtataaatcctggttgctgtctctttatgaggagttgtggcccgttgtcaggc
aacgtggcgtggtgtgcactgtgtttgctgacgcaacccccactggttggggcattgccaccac
ctgtcagctcctttccgggactttcgctttccccctccctattgccacggcggaactcatcgcc
gcctgccttgcccgctgctggacaggggctcggctgttgggcactgacaattccgtggtgttgt
cggggaaatcatcgtcctttccttggctgctcgcctgtgttgccacctggattctgcgcgggac
gtccttctgctacgtcccttcggccctcaatccagcggaccttccttcccgcggcctgctgccg
gctctgcggcctcttccgcgtcttcgccttcgccctcagacgagtcggatctccctttgggccg
cctccccgcgctcgctttcttgctgtccaatttctattaaaggttcctttgttccctaagtcca
actactaaactgggggatattatgaagggccttgagcatctggattctgcctaataaaaaacat
ttattttcattgcaagctcgctttcttgctgtccaatttctattaaaggttcctttgttcccta
agtccaactactaaactgggggatattatgaagggccttgagcatctggattctgcctaataaa
aaacatttattttcattgcaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaa
aaaaaaaaaaaaactcggaaggacatggtgtggaaagtccccaggctccccagcaggcagaagt
atgcaaagcatgcatctcaattagtcagcaaccaggtgtggaaagtccccaggctccccagcag
gcagaagtatgcaaagcatgcatctcaattagtcagcaaccaaattaaagtaacccataacttc
gtatagcatacattatacgaagttatgaagttcctattctctagaaagtataggaacttctagt
cacctatttcagcatactacgcgcgtagtatgctgaaataggtttatcagcacacaatagtcca
ttatacgcgcgtataatggcaattgtgtgctgattgggttactttaatttggatccgtcgaccg
atgcccttgagagccttcaacccagtcagctccttccggtgggcgcggggcatgactatcgtcg
ccgcacttatgactgtcttctttatcatgcaactcgtaggacaggtgccggcagcgctcttccg
cttcctcgctcactgactcgctgcgctcggtcgttcggctgcggcgagcggtatcagctcactc
aaaggcggtaatacggttatccacagaatcaggggataacgcaggaaagaacatgtgagcaaaa
ggccagcaaaaggccaggaaccgtaaaaaggccgcgttgctggcgtttttccataggctccgcc
cccctgacgagcatcacaaaaatcgacgctcaagtcagaggtggcgaaacccgacaggactata
aagataccaggcgtttccccctggaagctccctcgtgcgctctcctgttccgaccctgccgctt
accggatacctgtccgcctttctcccttcgggaagcgtggcgctttctcatagctcacgctgta
ggtatctcagttcggtgtaggtcgttcgctccaagctgggctgtgtgcacgaaccccccgttca
gcccgaccgctgcgccttatccggtaactatcgtcttgagtccaacccggtaagacacgactta
tcgccactggcagcagccactggtaacaggattagcagagcgaggtatgtaggcggtgctacag
agttcttgaagtggtggcctaactacggctacactagaagaacagtatttggtatctgcgctct
gctgaagccagttaccttcggaaaaagagttggtagctcttgatccggcaaacaaaccaccgct
ggtagcggtggtttttttgtttgcaagcagcagattacgcgcagaaaaaaaggatctcaagaag
atcctttgatcttttctacggggtctgacgctcagtggaacgaaaactcacgttaagggatttt
ggtcatgagattatcaaaaaggatcttcacctagatccttttaaattaaaaatgaagttttaaa
tcaatctaaagtatatatgagtaaacttggtctgacagttaccaatgcttaatcagtgaggcac
ctatctcagcgatctgtctatttcgttcatccatagttgcctgactccccgtcgtgtagataac
tacgatacgggagggcttaccatctggccccagtgctgcaatgataccgcgagacccacgctca
ccggctccagatttatcagcaataaaccagccagccggaagggccgagcgcagaagtggtcctg
caactttatccgcctccatccagtctattaattgttgccgggaagctagagtaagtagttcgcc
agttaatagtttgcgcaacgttgttgccattgctacaggcatcgtggtgtcacgctcgtcgttt
ggtatggcttcattcagctccggttcccaacgatcaaggcgagttacatgatcccccatgttgt
gcaaaaaagcggttagctccttcggtcctccgatcgttgtcagaagtaagttggccgcagtgtt
atcactcatggttatggcagcactgcataattctcttactgtcatgccatccgtaagatgcttt
tctgtgactggtgagtactcaaccaagtcattctgagaatagtgtatgcggcgaccgagttgct
cttgcccggcgtcaatacgggataataccgcgccacatagcagaactttaaaagtgctcatcat
tggaaaacgttcttcggggcgaaaactctcaaggatcttaccgctgttgagatccagttcgatg
taacccactcgtgcacccaactgatcttcagcatcttttactttcaccagcgtttctgggtgag
caaaaacaggaaggcaaaatgccgcaaaaaagggaataagggcgacacggaaatgttgaatact
catactcttcctttttcaatattattgaagcatttatcagggttattgtctcatgagcggatac
atatttgaatgtatttagaaaaataaacaaataggggttccgcgcacatttccccgaaaagtgc
cacctgacgcgccctgtagcggcgcattaagcgcggcgggtgtggtggttacgcgcagcgtgac
cgctacacttgccagcgccctagcgcccgctcctttcgctttcttcccttcctttctcgccacg
ttcgccggctttccccgtcaagctctaaatcgggggctccctttagggttccgatttagtgctt
tacggcacctcgaccccaaaaaacttgattagggtgatggttcacgtagtgggccatcgccctg
atagacggtttttcgccctttgacgttggagtccacgttctttaatagtggactcttgttccaa
actggaacaacactcaaccctatctcggtctattcttttgatttataagggattttgccgattt
cggcctattggttaaaaaatgagctgatttaacaaaaatttaacgcgaattttaacaaaatatt
aacgcttacaatttgccattcgccattcaggctgcgcaactgttgggaagggcgatcggtgcgg
gcctcttcgctattacgccagcccaagctaccatgataagtaagtaatattaaggtacgtggag
gttttacttgctttaaaaaacctcccacacctccccctgaacctgaaacataaaatgaatgcaa
ttgttgttgttaacttgtttattgcagcttataatggttacaaataaagcaatagcatcacaaa
tttcacaaataaagcatttttttcactgcattctagttgtggtttgtccaaactcatcaatgta
tcttatggtactgtaactgagctaacataa
SS*-E1-CMV-UTR1-SecNLuc-2A-eGFP-MAR-WPRE-3′UTR[2huBGpA-
A120]-SS*
SEQ ID NO: 31
cccgggaggtaccgagctcttacgcgtgctagaattaaagtaacccaatcagcacacaattgcc
attatacgcgcgtataatggactattgtgtgctgataaacctatttcagcatactacgcgcgta
gtatgctgaaataggtgactagaagttcctatactttctagagaataggaacttcataacttcg
tataatgtatgctatacgaagttatgggttactttaatttggttgctgactaattgagatgcat
gctttgcatacttctgcctgctggggagcctggggactttccacacctggttgctgactaattg
agatgcatgctttgcatacttctgcctgctggggagcctggggactttccacacccctgggtcg
acgggactttccggggcggggcacgtggtgcacgggactttccgtgcacgtgcacgggactttc
cgggactttccgggactttccgtgcaccacgtggggactttccgtgcacgacattgattattga
ctagttattaatagtaatcaattacggggtcattagttcatagcccatatatggagttccgcgt
tacataacttacggtaaatggcccgcctggctgaccgcccaacgacccccgcccattgacgtca
ataatgacgtatgttcccatagtaacgccaatagggactttccattgacgtcaatgggtggagt
atttacggtaaactgcccacttggcagtacatcaagtgtatcatatgccaagtacgccccctat
tgacgtcaatgacggtaaatggcccgcctggcattatgcccagtacatgaccttatgggacttt
cctacttggcagtacatctacgtattagtcatcgctattaccatggtgatgcggttttggcagt
acatcaatgggcgtggatagcggtttgactcacggggatttccaagtctccaccccattgacgt
caatgggagtttgttttggcaccaaaatcaacgggactttccaaaatgtcgtaacaactccgcc
ccattgacgcaaatgggcggtaggcgtgtacggtgggaggtctatataagcagagctctgcctt
ctccctcctgtgagtttggtaagtcgacgggccgggcctgggccgggtccgggccgggtcgttg
gatccccactacagcccgatactcaagcttgacgaattcgagtatccaaggtagtggactagtg
tgacgctgctgacccctttctttcccttctgcaggttggtgtacagtagcttccaaattgatta
attcgagcgaacgcgtcgccaccatgaactccttctccacaagcgccttcggtccagttgcctt
ctccctgggcctgctcctggtgttgcctgctgccttccctgccccagtcttcacactcgaagat
ttcgttggggactggcgacagacagccggctacaacctggaccaagtccttgaacagggaggtg
tgtccagtttgtttcagaatctcggggtgtccgtaactccgatccaaaggattgtcctgagcgg
tgaaaatgggctgaagatcgacatccatgtcatcatcccgtatgaaggtctgagcggcgaccaa
atgggccagatcgaaaaaatttttaaggtggtgtaccctgtggatgatcatcactttaaggtga
tcctgcactatggcacactggtaatcgacggggttacgccgaacatgatcgactatttcggacg
gccgtatgaaggcatcgccgtgttcgacggcaaaaagatcactgtaacagggaccctgtggaac
ggcaacaaaattatcgacgagcgcctgatcaaccccgacggctccctgctgttccgagtaacca
tcaacggagtgaccggctggcggctgtgcgaacgcattctggcggctagcgctactaacttcag
cctgctgaagcaggctggagacgtggaggagaaccctggacctggaagcggagagggcagagga
agtctgctaacatgcggtgacgtcgaggagaatcctggacctggatccggaatggtgagcaagg
gcgaggagctgttcaccggggtggtgcccatcctggtcgagctggacggcgacgtaaacggcca
caagttcagcgtgtccggcgagggcgagggcgatgccacctacggcaagctgaccctgaagttc
atctgcaccaccggcaagctgcccgtgccctggcccaccctcgtgaccaccctgacctacggcg
tgcagtgcttcagccgctaccccgaccacatgaagcagcacgacttcttcaagtccgccatgcc
cgaaggctacgtccaggagcgcaccatcttcttcaaggacgacggcaactacaagacccgcgcc
gaggtgaagttcgagggcgacaccctggtgaaccgcatcgagctgaagggcatcgacttcaagg
aggacggcaacatcctggggcacaagctggagtacaactacaacagccacaacgtctatatcat
ggccgacaagcagaagaacggcatcaaggtgaacttcaagatccgccacaacatcgaggacggc
agcgtgcagctcgccgaccactaccagcagaacacccccatcggcgacggccccgtgctgctgc
ccgacaaccactacctgagcacccagtccgccctgagcaaagaccccaacgagaagcgcgatca
catggtcctgctggagttcgtgaccgccgccgggatcactctcggcatggacgagctgtacaag
taaggatcccattctccttgatgtactaatttttctttaaaagtgataataatagctcccattt
agaatttttaaataacacaacaaatgtaaagtaactaatgtgtcctctggatcatggtaagtaa
tgaataaatttaactccctttaccttctccctttgctattttttccatgctaggatttatacat
ttttaaaaaactaaatctgctatcaaatgacagctttaaatttactttttaaaatttgttattg
tatatatttatggggtataaagtgatgttatgatatatatatacacaatgtacactgattaaat
caagccaattaacattttatcatctcaaatacttaacattttttgtagtgagaacatttgaaat
ttacttttagcaatttcaaaacatacattattattattaactatagtcaccatgatgtaccata
gatctttaaaaacttattcttcctgcctaactgaaactttgtactctttgactaacatcttttc
attcccccacttcccagcctctggtaatcaccattacacactctgcttctatgagttcaattgc
tttagactccacgtaataaatgagatcatgcagcatttggctttctgtgcctggcttatccttg
cttagcatggtgtcttacaggttcatccatgttgcaacaaataacagaatctcattctttgtta
aggctgaatactattccattgggtatatataccacattttccttatccattaatccactgatgg
acccttaggttgttgattccatatattggctattgtaaatagtgcagcaatgaacatgagagtg
caactatctcttcaatgtactgatttcgaatccttcggatctatctcagaagtgagattgcagg
atcatataattctacttttagtcttttgaggagctccatacagctttccatatggccatactaa
ttacattctcatcaacagtgtacaatggtttccttttctccacatcctcaccaacatttataat
tttttgtctttttgataatagccatctgacaggtgtaaagtgatagctcattgcagttttaatt
tgcattttttgatgattagtaatgttgagaattttttcatatatctcttggccagttgcatgtc
ttctttggaaaaatgtctattcagttcctttgcccattttttaattgggatttttggtttcttg
ctattgagttgtttgaattcaatcaacctctggattacaaaatttgtgaaagattgactggtat
tcttaactatgttgctccttttacgctatgtggatacgctgctttaatgcctttgtatcatgct
attgcttcccgtatggctttcattttctcctccttgtataaatcctggttgctgtctctttatg
aggagttgtggcccgttgtcaggcaacgtggcgtggtgtgcactgtgtttgctgacgcaacccc
cactggttggggcattgccaccacctgtcagctcctttccgggactttcgctttccccctccct
attgccacggcggaactcatcgccgcctgccttgcccgctgctggacaggggctcggctgttgg
gcactgacaattccgtggtgttgtcggggaaatcatcgtcctttccttggctgctcgcctgtgt
tgccacctggattctgcgcgggacgtccttctgctacgtcccttcggccctcaatccagcggac
cttccttcccgcggcctgctgccggctctgcggcctcttccgcgtcttcgccttcgccctcaga
cgagtcggatctccctttgggccgcctccccgcgctcgctttcttgctgtccaatttctattaa
aggttcctttgttccctaagtccaactactaaactgggggatattatgaagggccttgagcatc
tggattctgcctaataaaaaacatttattttcattgcaagctcgctttcttgctgtccaatttc
tattaaaggttcctttgttccctaagtccaactactaaactgggggatattatgaagggccttg
agcatctggattctgcctaataaaaaacatttattttcattgcaaaaaaaaaaaaaaaaaaaaa
aaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaactcggaaggacatggtgtggaaagtcc
ccaggctccccagcaggcagaagtatgcaaagcatgcatctcaattagtcagcaaccaggtgtg
gaaagtccccaggctccccagcaggcagaagtatgcaaagcatgcatctcaattagtcagcaac
caaattaaagtaacccataacttcgtatagcatacattatacgaagttatgaagttcctattct
ctagaaagtataggaacttctagtcacctatttcagcatactacgcgcgtagtatgctgaaata
ggtttatcagcacacaatagtccattatacgcgcgtataatggcaattgtgtgctgattgggtt
actttaatttggatccgtcgaccgatgcccttgagagccttcaacccagtcagctccttccggt
gggcgcggggcatgactatcgtcgccgcacttatgactgtcttctttatcatgcaactcgtagg
acaggtgccggcagcgctcttccgcttcctcgctcactgactcgctgcgctcggtcgttcggct
gcggcgagcggtatcagctcactcaaaggcggtaatacggttatccacagaatcaggggataac
gcaggaaagaacatgtgagcaaaaggccagcaaaaggccaggaaccgtaaaaaggccgcgttgc
tggcgtttttccataggctccgcccccctgacgagcatcacaaaaatcgacgctcaagtcagag
gtggcgaaacccgacaggactataaagataccaggcgtttccccctggaagctccctcgtgcgc
tctcctgttccgaccctgccgcttaccggatacctgtccgcctttctcccttcgggaagcgtgg
cgctttctcatagctcacgctgtaggtatctcagttcggtgtaggtcgttcgctccaagctggg
ctgtgtgcacgaaccccccgttcagcccgaccgctgcgccttatccggtaactatcgtcttgag
tccaacccggtaagacacgacttatcgccactggcagcagccactggtaacaggattagcagag
cgaggtatgtaggcggtgctacagagttcttgaagtggtggcctaactacggctacactagaag
aacagtatttggtatctgcgctctgctgaagccagttaccttcggaaaaagagttggtagctct
tgatccggcaaacaaaccaccgctggtagcggtggtttttttgtttgcaagcagcagattacgc
gcagaaaaaaaggatctcaagaagatcctttgatcttttctacggggtctgacgctcagtggaa
cgaaaactcacgttaagggattttggtcatgagattatcaaaaaggatcttcacctagatcctt
ttaaattaaaaatgaagttttaaatcaatctaaagtatatatgagtaaacttggtctgacagtt
accaatgcttaatcagtgaggcacctatctcagcgatctgtctatttcgttcatccatagttgc
ctgactccccgtcgtgtagataactacgatacgggagggcttaccatctggccccagtgctgca
atgataccgcgagacccacgctcaccggctccagatttatcagcaataaaccagccagccggaa
gggccgagcgcagaagtggtcctgcaactttatccgcctccatccagtctattaattgttgccg
ggaagctagagtaagtagttcgccagttaatagtttgcgcaacgttgttgccattgctacaggc
atcgtggtgtcacgctcgtcgtttggtatggcttcattcagctccggttcccaacgatcaaggc
gagttacatgatcccccatgttgtgcaaaaaagcggttagctccttcggtcctccgatcgttgt
cagaagtaagttggccgcagtgttatcactcatggttatggcagcactgcataattctcttact
gtcatgccatccgtaagatgcttttctgtgactggtgagtactcaaccaagtcattctgagaat
agtgtatgcggcgaccgagttgctcttgcccggcgtcaatacgggataataccgcgccacatag
cagaactttaaaagtgctcatcattggaaaacgttcttcggggcgaaaactctcaaggatctta
ccgctgttgagatccagttcgatgtaacccactcgtgcacccaactgatcttcagcatctttta
ctttcaccagcgtttctgggtgagcaaaaacaggaaggcaaaatgccgcaaaaaagggaataag
ggcgacacggaaatgttgaatactcatactcttcctttttcaatattattgaagcatttatcag
ggttattgtctcatgagcggatacatatttgaatgtatttagaaaaataaacaaataggggttc
cgcgcacatttccccgaaaagtgccacctgacgcgccctgtagcggcgcattaagcgcggcggg
tgtggtggttacgcgcagcgtgaccgctacacttgccagcgccctagcgcccgctcctttcgct
ttcttcccttcctttctcgccacgttcgccggctttccccgtcaagctctaaatcgggggctcc
ctttagggttccgatttagtgctttacggcacctcgaccccaaaaaacttgattagggtgatgg
ttcacgtagtgggccatcgccctgatagacggtttttcgccctttgacgttggagtccacgttc
tttaatagtggactcttgttccaaactggaacaacactcaaccctatctcggtctattcttttg
atttataagggattttgccgatttcggcctattggttaaaaaatgagctgatttaacaaaaatt
taacgcgaattttaacaaaatattaacgcttacaatttgccattcgccattcaggctgcgcaac
tgttgggaagggcgatcggtgcgggcctcttcgctattacgccagcccaagctaccatgataag
taagtaatattaaggtacgtggaggttttacttgctttaaaaaacctcccacacctccccctga
acctgaaacataaaatgaatgcaattgttgttgttaacttgtttattgcagcttataatggtta
caaataaagcaatagcatcacaaatttcacaaataaagcatttttttcactgcattctagttgt
ggtttgtccaaactcatcaatgtatcttatggtactgtaactgagctaacataa
SS*-UCOE-E1-CMV-UTR1-SecNLuc-2A-eGFP-MAR-WPRE-
3′UTR[2huBGpA-A120]-SS*
SEQ ID NO: 32
cccgggaggtaccgagctcttacgcgtgctagaattaaagtaacccaatcagcacacaattgcc
attatacgcgcgtataatggactattgtgtgctgataaacctatttcagcatactacgcgcgta
gtatgctgaaataggtgactagaagttcctatactttctagagaataggaacttcataacttcg
tataatgtatgctatacgaagttatgggttactttaatttggttgctgactaattgagatgcat
gctttgcatacttctgcctgctggggagcctggggactttccacacctggttgctgactaattg
agatgcatgctttgcatacttctgcctgctggggagcctggggactttccacacccctgggtcg
acgcacacgaccacaattccactgaaagcattttaatacggaacttgtcactcccagggagcct
ccgctcagccggcagttggttcatttcaatccccacgacaacccttcaaagtgcagggcagaca
gcaggtggctctgcccaggcgcctggatcacagcccggcctgcagccctcacctgggcgcgggg
agaccctgaggacgctcctccaggcggcgctggccggggcctgcggacacggacgggcgggctg
agctccgggacccctccccgcgccccgcaccccgcaccccgcaccccgcaccccgcacccggcg
ctcacccgtcccagccccgccgcccgcagccccagctgcaacgcagccaccgccgccatcgcac
ccggccccgcgggcgcttccgggacgcaggaggcatctgcatccggggcgccgctgagtcccgc
ccagagccccgcccccggctccaggttctgcgagcggcttccgccgggctgctccgcgggcgcg
tcggccatgagcgagttgccgggcgacgtgcgggcgtttctgcgggagcacccgagcctgcggc
tccagacggacgcccgcaaggttcgcagcgcgggaggggaacggagtggcggagaagggcgcag
ttgggatgaggggctgaggggagggcaggggagaggagagggcaggggagaggggagaggggag
agcaggagagaggggaaggcaggggagagggcgcggcgggatcaggggaggagagggaagggac
tttccggggcggggcacgtggtgcacgggactttccgtgcacgtgcacgggactttccgggact
ttccgggactttccgtgcaccacgtggggactttccgtgcacgacattgattattgactagtta
ttaatagtaatcaattacggggtcattagttcatagcccatatatggagttccgcgttacataa
cttacggtaaatggcccgcctggctgaccgcccaacgacccccgcccattgacgtcaataatga
cgtatgttcccatagtaacgccaatagggactttccattgacgtcaatgggtggagtatttacg
gtaaactgcccacttggcagtacatcaagtgtatcatatgccaagtacgccccctattgacgtc
aatgacggtaaatggcccgcctggcattatgcccagtacatgaccttatgggactttcctactt
ggcagtacatctacgtattagtcatcgctattaccatggtgatgcggttttggcagtacatcaa
tgggcgtggatagcggtttgactcacggggatttccaagtctccaccccattgacgtcaatggg
agtttgttttggcaccaaaatcaacgggactttccaaaatgtcgtaacaactccgccccattga
cgcaaatgggcggtaggcgtgtacggtgggaggtctatataagcagagctctgccttctccctc
ctgtgagtttggtaagtcgacgggccgggcctgggccgggtccgggccgggtcgttggatcccc
actacagcccgatactcaagcttgacgaattcgagtatccaaggtagtggactagtgtgacgct
gctgacccctttctttcccttctgcaggttggtgtacagtagcttccaaattgattaattcgag
cgaacgcgtcgccaccatgaactccttctccacaagcgccttcggtccagttgccttctccctg
ggcctgctcctggtgttgcctgctgccttccctgccccagtcttcacactcgaagatttcgttg
gggactggcgacagacagccggctacaacctggaccaagtccttgaacagggaggtgtgtccag
tttgtttcagaatctcggggtgtccgtaactccgatccaaaggattgtcctgagcggtgaaaat
gggctgaagatcgacatccatgtcatcatcccgtatgaaggtctgagcggcgaccaaatgggcc
agatcgaaaaaatttttaaggtggtgtaccctgtggatgatcatcactttaaggtgatcctgca
ctatggcacactggtaatcgacggggttacgccgaacatgatcgactatttcggacggccgtat
gaaggcatcgccgtgttcgacggcaaaaagatcactgtaacagggaccctgtggaacggcaaca
aaattatcgacgagcgcctgatcaaccccgacggctccctgctgttccgagtaaccatcaacgg
agtgaccggctggcggctgtgcgaacgcattctggcggctagcgctactaacttcagcctgctg
aagcaggctggagacgtggaggagaaccctggacctggaagcggagagggcagaggaagtctgc
taacatgcggtgacgtcgaggagaatcctggacctggatccggaatggtgagcaagggcgagga
gctgttcaccggggtggtgcccatcctggtcgagctggacggcgacgtaaacggccacaagttc
agcgtgtccggcgagggcgagggcgatgccacctacggcaagctgaccctgaagttcatctgca
ccaccggcaagctgcccgtgccctggcccaccctcgtgaccaccctgacctacggcgtgcagtg
cttcagccgctaccccgaccacatgaagcagcacgacttcttcaagtccgccatgcccgaaggc
tacgtccaggagcgcaccatcttcttcaaggacgacggcaactacaagacccgcgccgaggtga
agttcgagggcgacaccctggtgaaccgcatcgagctgaagggcatcgacttcaaggaggacgg
caacatcctggggcacaagctggagtacaactacaacagccacaacgtctatatcatggccgac
aagcagaagaacggcatcaaggtgaacttcaagatccgccacaacatcgaggacggcagcgtgc
agctcgccgaccactaccagcagaacacccccatcggcgacggccccgtgctgctgcccgacaa
ccactacctgagcacccagtccgccctgagcaaagaccccaacgagaagcgcgatcacatggtc
ctgctggagttcgtgaccgccgccgggatcactctcggcatggacgagctgtacaagtaaggat
cccattctccttgatgtactaatttttctttaaaagtgataataatagctcccatttagaattt
ttaaataacacaacaaatgtaaagtaactaatgtgtcctctggatcatggtaagtaatgaataa
atttaactccctttaccttctccctttgctattttttccatgctaggatttatacatttttaaa
aaactaaatctgctatcaaatgacagctttaaatttactttttaaaatttgttattgtatatat
ttatggggtataaagtgatgttatgatatatatatacacaatgtacactgattaaatcaagcca
attaacattttatcatctcaaatacttaacattttttgtagtgagaacatttgaaatttacttt
tagcaatttcaaaacatacattattattattaactatagtcaccatgatgtaccatagatcttt
aaaaacttattcttcctgcctaactgaaactttgtactctttgactaacatcttttcattcccc
cacttcccagcctctggtaatcaccattacacactctgcttctatgagttcaattgctttagac
tccacgtaataaatgagatcatgcagcatttggctttctgtgcctggcttatccttgcttagca
tggtgtcttacaggttcatccatgttgcaacaaataacagaatctcattctttgttaaggctga
atactattccattgggtatatataccacattttccttatccattaatccactgatggaccctta
ggttgttgattccatatattggctattgtaaatagtgcagcaatgaacatgagagtgcaactat
ctcttcaatgtactgatttcgaatccttcggatctatctcagaagtgagattgcaggatcatat
aattctacttttagtcttttgaggagctccatacagctttccatatggccatactaattacatt
ctcatcaacagtgtacaatggtttccttttctccacatcctcaccaacatttataattttttgt
ctttttgataatagccatctgacaggtgtaaagtgatagctcattgcagttttaatttgcattt
tttgatgattagtaatgttgagaattttttcatatatctcttggccagttgcatgtcttctttg
gaaaaatgtctattcagttcctttgcccattttttaattgggatttttggtttcttgctattga
gttgtttgaattcaatcaacctctggattacaaaatttgtgaaagattgactggtattcttaac
tatgttgctccttttacgctatgtggatacgctgctttaatgcctttgtatcatgctattgctt
cccgtatggctttcattttctcctccttgtataaatcctggttgctgtctctttatgaggagtt
gtggcccgttgtcaggcaacgtggcgtggtgtgcactgtgtttgctgacgcaacccccactggt
tggggcattgccaccacctgtcagctcctttccgggactttcgctttccccctccctattgcca
cggcggaactcatcgccgcctgccttgcccgctgctggacaggggctcggctgttgggcactga
caattccgtggtgttgtcggggaaatcatcgtcctttccttggctgctcgcctgtgttgccacc
tggattctgcgcgggacgtccttctgctacgtcccttcggccctcaatccagcggaccttcctt
cccgcggcctgctgccggctctgcggcctcttccgcgtcttcgccttcgccctcagacgagtcg
gatctccctttgggccgcctccccgcgctcgctttcttgctgtccaatttctattaaaggttcc
tttgttccctaagtccaactactaaactgggggatattatgaagggccttgagcatctggattc
tgcctaataaaaaacatttattttcattgcaagctcgctttcttgctgtccaatttctattaaa
ggttcctttgttccctaagtccaactactaaactgggggatattatgaagggccttgagcatct
ggattctgcctaataaaaaacatttattttcattgcaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaactcggaaggacatggtgtggaaagtccccaggct
ccccagcaggcagaagtatgcaaagcatgcatctcaattagtcagcaaccaggtgtggaaagtc
cccaggctccccagcaggcagaagtatgcaaagcatgcatctcaattagtcagcaaccaaatta
aagtaacccataacttcgtatagcatacattatacgaagttatgaagttcctattctctagaaa
gtataggaacttctagtcacctatttcagcatactacgcgcgtagtatgctgaaataggtttat
cagcacacaatagtccattatacgcgcgtataatggcaattgtgtgctgattgggttactttaa
tttggatccgtcgaccgatgcccttgagagccttcaacccagtcagctccttccggtgggcgcg
gggcatgactatcgtcgccgcacttatgactgtcttctttatcatgcaactcgtaggacaggtg
ccggcagcgctcttccgcttcctcgctcactgactcgctgcgctcggtcgttcggctgcggcga
gcggtatcagctcactcaaaggcggtaatacggttatccacagaatcaggggataacgcaggaa
agaacatgtgagcaaaaggccagcaaaaggccaggaaccgtaaaaaggccgcgttgctggcgtt
tttccataggctccgcccccctgacgagcatcacaaaaatcgacgctcaagtcagaggtggcga
aacccgacaggactataaagataccaggcgtttccccctggaagctccctcgtgcgctctcctg
ttccgaccctgccgcttaccggatacctgtccgcctttctcccttcgggaagcgtggcgctttc
tcatagctcacgctgtaggtatctcagttcggtgtaggtcgttcgctccaagctgggctgtgtg
cacgaaccccccgttcagcccgaccgctgcgccttatccggtaactatcgtcttgagtccaacc
cggtaagacacgacttatcgccactggcagcagccactggtaacaggattagcagagcgaggta
tgtaggcggtgctacagagttcttgaagtggtggcctaactacggctacactagaagaacagta
tttggtatctgcgctctgctgaagccagttaccttcggaaaaagagttggtagctcttgatccg
gcaaacaaaccaccgctggtagcggtggtttttttgtttgcaagcagcagattacgcgcagaaa
aaaaggatctcaagaagatcctttgatcttttctacggggtctgacgctcagtggaacgaaaac
tcacgttaagggattttggtcatgagattatcaaaaaggatcttcacctagatccttttaaatt
aaaaatgaagttttaaatcaatctaaagtatatatgagtaaacttggtctgacagttaccaatg
cttaatcagtgaggcacctatctcagcgatctgtctatttcgttcatccatagttgcctgactc
cccgtcgtgtagataactacgatacgggagggcttaccatctggccccagtgctgcaatgatac
cgcgagacccacgctcaccggctccagatttatcagcaataaaccagccagccggaagggccga
gcgcagaagtggtcctgcaactttatccgcctccatccagtctattaattgttgccgggaagct
agagtaagtagttcgccagttaatagtttgcgcaacgttgttgccattgctacaggcatcgtgg
tgtcacgctcgtcgtttggtatggcttcattcagctccggttcccaacgatcaaggcgagttac
atgatcccccatgttgtgcaaaaaagcggttagctccttcggtcctccgatcgttgtcagaagt
aagttggccgcagtgttatcactcatggttatggcagcactgcataattctcttactgtcatgc
catccgtaagatgcttttctgtgactggtgagtactcaaccaagtcattctgagaatagtgtat
gcggcgaccgagttgctcttgcccggcgtcaatacgggataataccgcgccacatagcagaact
ttaaaagtgctcatcattggaaaacgttcttcggggcgaaaactctcaaggatcttaccgctgt
tgagatccagttcgatgtaacccactcgtgcacccaactgatcttcagcatcttttactttcac
cagcgtttctgggtgagcaaaaacaggaaggcaaaatgccgcaaaaaagggaataagggcgaca
cggaaatgttgaatactcatactcttcctttttcaatattattgaagcatttatcagggttatt
gtctcatgagcggatacatatttgaatgtatttagaaaaataaacaaataggggttccgcgcac
atttccccgaaaagtgccacctgacgcgccctgtagcggcgcattaagcgcggcgggtgtggtg
gttacgcgcagcgtgaccgctacacttgccagcgccctagcgcccgctcctttcgctttcttcc
cttcctttctcgccacgttcgccggctttccccgtcaagctctaaatcgggggctccctttagg
gttccgatttagtgctttacggcacctcgaccccaaaaaacttgattagggtgatggttcacgt
agtgggccatcgccctgatagacggtttttcgccctttgacgttggagtccacgttctttaata
gtggactcttgttccaaactggaacaacactcaaccctatctcggtctattcttttgatttata
agggattttgccgatttcggcctattggttaaaaaatgagctgatttaacaaaaatttaacgcg
aattttaacaaaatattaacgcttacaatttgccattcgccattcaggctgcgcaactgttggg
aagggcgatcggtgcgggcctcttcgctattacgccagcccaagctaccatgataagtaagtaa
tattaaggtacgtggaggttttacttgctttaaaaaacctcccacacctccccctgaacctgaa
acataaaatgaatgcaattgttgttgttaacttgtttattgcagcttataatggttacaaataa
agcaatagcatcacaaatttcacaaataaagcatttttttcactgcattctagttgtggtttgt
ccaaactcatcaatgtatcttatggtactgtaactgagctaacataa
(Super Sequence, SS*)
SEQ ID NO: 33
taaagtaacccaatcagcacacaattgccattatacgcgcgtataatggactattgtgtgctgat
aaacctatttcagcatactacgcgcgtagtatgctgaaataggtgactagaagttcctatacttt
ctagagaataggaacttcataacttcgtataatgtatgctatacgaagttatgggttactttaat
ttggttgctgactaattgagatgcatgctttgcatacttctgcctgctggggagcctggggactt
tccacacctggttgctgactaattgagatgcatgctttgcatacttctgcctgctggggagcctg
gggactttccacacc
pGL2-CAG-SecNLuc-2A-eGFP-WPRE-bGlobin polyA
SEQ ID NO: 34
cccgggaggtaccgagctcttacgcgtgctagcctgggtcgacattgattattgactagttatta
atagtaatcaattacggggtcattagttcatagcccatatatggagttccgcgttacataactta
cggtaaatggcccgcctggctgaccgcccaacgacccccgcccattgacgtcaataatgacgtat
gttcccatagtaacgccaatagggactttccattgacgtcaatgggtggagtatttacggtaaac
tgcccacttggcagtacatcaagtgtatcatatgccaagtacgccccctattgacgtcaatgacg
gtaaatggcccgcctggcattatgcccagtacatgaccttatgggactttcctacttggcagtac
atctacgtattagtcatcgctattaccatggtcgaggtgagccccacgttctgcttcactctccc
catctcccccccctccccacccccaattttgtatttatttattttttaattattttgtgcagcga
tgggggcggggggggggggggcgcgcgccaggcggggcggggcggggcgaggggcggggcggggc
gaggcggaaaggtgcggcggcagccaatcagagcggcgcgctccgaaagtttccttttatggcga
ggcggcggcggcggcggccctataaaaagcgaagcgcgcggcgggcgggagtcgctgcgttgcct
tcgccccgtgccccgctccgcgccgcctcgcgccgcccgccccggctctgactgaccgcgttact
cccacaggtgagcgggcgggacggcccttctcctccgggctgtaattagcgcttggtttaatgac
ggctcgtttcttttctgtggctgcgtgaaagccttaaagggctccgggagggccctttgtgcggg
ggggagcggctcggggggtgcgtgcgtgtgtgtgtgcgtggggagcgccgcgtgcggctccgcgc
tgcccggcggctgtgagcgctgcgggcgcggcgcggggctttgtgcgctccgcagtgtgcgcgag
gggagcgcggccgggggcggtgccccgcggtgcggggggggctgcgaggggaacaaaggctgcgt
gcggggtgtgtgcgtgggggggtgagcagggggtgtgggcgcggcggtcgggctgtaaccccccc
ctgcacccccctccccgagttgctgagcacggcccggcttcgggtgcggggctccgtacggggcg
tggcgcggggctcgccgtgccgggcggggggtggcggcaggtgggggtgccgggcggggcggggc
cgcctcgggccggggagggctcgggggaggggcgcggcggcccccggagcgccggcggctgtcga
ggcgcggcgagccgcagccattgccttttatggtaatcgtgcgagagggcgcagggacttccttt
gtcccaaatctgtgcggagccgaaatctgggaggcgccgccgcaccccctctagcgggcgcgggg
cgaagcggtgcggcgccggcaggaaggaaatgggcggggagggccttcgtgcgtcgccgcgccgc
cgtccccttctccctctccagcctcggggctgtccgcggggggacggctgccttcgggggggacg
gggcagggcggggttcggcttctggcgtgtgaccggcggctctagagcctctgctaaccatgttc
atgccttcttctttttcctacagctcctgggcaacgtgctggttattgtgctgtctcatcatttt
ggcaaagaattgattaattcgagcgaacgcgtcgccaccatgaactccttctccacaagcgcctt
cggtccagttgccttctccctgggcctgctcctggtgttgcctgctgccttccctgccccagtct
tcacactcgaagatttcgttggggactggcgacagacagccggctacaacctggaccaagtcctt
gaacagggaggtgtgtccagtttgtttcagaatctcggggtgtccgtaactccgatccaaaggat
tgtcctgagcggtgaaaatgggctgaagatcgacatccatgtcatcatcccgtatgaaggtctga
gcggcgaccaaatgggccagatcgaaaaaatttttaaggtggtgtaccctgtggatgatcatcac
tttaaggtgatcctgcactatggcacactggtaatcgacggggttacgccgaacatgatcgacta
tttcggacggccgtatgaaggcatcgccgtgttcgacggcaaaaagatcactgtaacagggaccc
tgtggaacggcaacaaaattatcgacgagcgcctgatcaaccccgacggctccctgctgttccga
gtaaccatcaacggagtgaccggctggcggctgtgcgaacgcattctggcggctagcgctactaa
cttcagcctgctgaagcaggctggagacgtggaggagaaccctggacctggaagcggagagggca
gaggaagtctgctaacatgcggtgacgtcgaggagaatcctggacctggatccggaatggtgagc
aagggcgaggagctgttcaccggggtggtgcccatcctggtcgagctggacggcgacgtaaacgg
ccacaagttcagcgtgtccggcgagggcgagggcgatgccacctacggcaagctgaccctgaagt
tcatctgcaccaccggcaagctgcccgtgccctggcccaccctcgtgaccaccctgacctacggc
gtgcagtgcttcagccgctaccccgaccacatgaagcagcacgacttcttcaagtccgccatgcc
cgaaggctacgtccaggagcgcaccatcttcttcaaggacgacggcaactacaagacccgcgccg
aggtgaagttcgagggcgacaccctggtgaaccgcatcgagctgaagggcatcgacttcaaggag
gacggcaacatcctggggcacaagctggagtacaactacaacagccacaacgtctatatcatggc
cgacaagcagaagaacggcatcaaggtgaacttcaagatccgccacaacatcgaggacggcagcg
tgcagctcgccgaccactaccagcagaacacccccatcggcgacggccccgtgctgctgcccgac
aaccactacctgagcacccagtccgccctgagcaaagaccccaacgagaagcgcgatcacatggt
cctgctggagttcgtgaccgccgccgggatcactctcggcatggacgagctgtacaagtaagcgg
ccgcactcctcaggtgcaggctgcctatcagaaggtggtggctggtgtggccaatgccctggctc
acaaataccactgagatctttttccctctgccaaaaattatggggacatcatgaagccccttgag
catctgacttctggctaataaaggaaatttattttcattgcaatagtgtgttggaattttttgtg
tctctcactcggaaggacattggatccgtcgaccgatgcccttgagagccttcaacccagtcagc
tccttccggtgggcgcggggcatgactatcgtcgccgcacttatgactgtcttctttatcatgca
actcgtaggacaggtgccggcagcgctcttccgcttcctcgctcactgactcgctgcgctcggtc
gttcggctgcggcgagcggtatcagctcactcaaaggcggtaatacggttatccacagaatcagg
ggataacgcaggaaagaacatgtgagcaaaaggccagcaaaaggccaggaaccgtaaaaaggccg
cgttgctggcgtttttccataggctccgcccccctgacgagcatcacaaaaatcgacgctcaagt
cagaggtggcgaaacccgacaggactataaagataccaggcgtttccccctggaagctccctcgt
gcgctctcctgttccgaccctgccgcttaccggatacctgtccgcctttctcccttcgggaagcg
tggcgctttctcatagctcacgctgtaggtatctcagttcggtgtaggtcgttcgctccaagctg
ggctgtgtgcacgaaccccccgttcagcccgaccgctgcgccttatccggtaactatcgtcttga
gtccaacccggtaagacacgacttatcgccactggcagcagccactggtaacaggattagcagag
cgaggtatgtaggcggtgctacagagttcttgaagtggtggcctaactacggctacactagaaga
acagtatttggtatctgcgctctgctgaagccagttaccttcggaaaaagagttggtagctcttg
atccggcaaacaaaccaccgctggtagcggtggtttttttgtttgcaagcagcagattacgcgca
gaaaaaaaggatctcaagaagatcctttgatcttttctacggggtctgacgctcagtggaacgaa
aactcacgttaagggattttggtcatgagattatcaaaaaggatcttcacctagatccttttaaa
ttaaaaatgaagttttaaatcaatctaaagtatatatgagtaaacttggtctgacagttaccaat
gcttaatcagtgaggcacctatctcagcgatctgtctatttcgttcatccatagttgcctgactc
cccgtcgtgtagataactacgatacgggagggcttaccatctggccccagtgctgcaatgatacc
gcgagacccacgctcaccggctccagatttatcagcaataaaccagccagccggaagggccgagc
gcagaagtggtcctgcaactttatccgcctccatccagtctattaattgttgccgggaagctaga
gtaagtagttcgccagttaatagtttgcgcaacgttgttgccattgctacaggcatcgtggtgtc
acgctcgtcgtttggtatggcttcattcagctccggttcccaacgatcaaggcgagttacatgat
cccccatgttgtgcaaaaaagcggttagctccttcggtcctccgatcgttgtcagaagtaagttg
gccgcagtgttatcactcatggttatggcagcactgcataattctcttactgtcatgccatccgt
aagatgcttttctgtgactggtgagtactcaaccaagtcattctgagaatagtgtatgcggcgac
cgagttgctcttgcccggcgtcaatacgggataataccgcgccacatagcagaactttaaaagtg
ctcatcattggaaaacgttcttcggggcgaaaactctcaaggatcttaccgctgttgagatccag
ttcgatgtaacccactcgtgcacccaactgatcttcagcatcttttactttcaccagcgtttctg
ggtgagcaaaaacaggaaggcaaaatgccgcaaaaaagggaataagggcgacacggaaatgttga
atactcatactcttcctttttcaatattattgaagcatttatcagggttattgtctcatgagcgg
atacatatttgaatgtatttagaaaaataaacaaataggggttccgcgcacatttccccgaaaag
tgccacctgacgcgccctgtagcggcgcattaagcgcggcgggtgtggtggttacgcgcagcgtg
accgctacacttgccagcgccctagcgcccgctcctttcgctttcttcccttcctttctcgccac
gttcgccggctttccccgtcaagctctaaatcgggggctccctttagggttccgatttagtgctt
tacggcacctcgaccccaaaaaacttgattagggtgatggttcacgtagtgggccatcgccctga
tagacggtttttcgccctttgacgttggagtccacgttctttaatagtggactcttgttccaaac
tggaacaacactcaaccctatctcggtctattcttttgatttataagggattttgccgatttcgg
cctattggttaaaaaatgagctgatttaacaaaaatttaacgcgaattttaacaaaatattaacg
cttacaatttgccattcgccattcaggctgcgcaactgttgggaagggcgatcggtgcgggcctc
ttcgctattacgccagcccaagctaccatgataagtaagtaatattaaggtacgtggaggtttta
cttgctttaaaaaacctcccacacctccccctgaacctgaaacataaaatgaatgcaattgttgt
tgttaacttgtttattgcagcttataatggttacaaataaagcaatagcatcacaaatttcacaa
ataaagcatttttttcactgcattctagttgtggtttgtccaaactcatcaatgtatcttatggt
actgtaactgagctaacataa
CAG [E1X3 + CBA promoter + intron]
SEQ ID NO: 35
gggactttccggggcggggcacgtggtgcacgggactttccgtgcacgtgcacgggactttccgg
gactttccgggactttccgtgcaccacgtggggactttccgtgcacgggactttccggggcgggg
cacgtggtgcacgggactttccgtgcacgtgcacgggactttccgggactttccgggactttccg
tgcaccacgtggggactttccgtgcacgggactttccggggcggggcacgtggtgcacgggactt
tccgtgcacgtgcacgggactttccgggactttccgggactttccgtgcaccacgtggggacttt
ccgtgcacgtcgaggtgagccccacgttctgcttcactctccccatctcccccccctccccaccc
ccaattttgtatttatttattttttaattattttgtgcagcgatgggggcggggggggggggggc
gcgcgccaggcggggcggggcggggcgaggggcggggcggggcgaggcggaaaggtgcggcggca
gccaatcagagcggcgcgctccgaaagtttccttttatggcgaggcggcggcggcggcggcccta
taaaaagcgaagcgcgcggcgggcgggagtcgctgcgttgccttcgccccgtgccccgctccgcg
ccgcctcgcgccgcccgccccggctctgactgaccgcgttactcccacaggtgagcgggcgggac
ggcccttctcctccgggctgtaattagcgcttggtttaatgacggctcgtttcttttctgtggct
gcgtgaaagccttaaagggctccgggagggccctttgtgcgggggggagcggctcggggggtgcg
tgcgtgtgtgtgtgcgtggggagcgccgcgtgcggctccgcgctgcccggcggctgtgagcgctg
cgggcgcggcgcggggctttgtgcgctccgcagtgtgcgcgaggggagcgcggccgggggcggtg
ccccgcggtgcggggggggctgcgaggggaacaaaggctgcgtgcggggtgtgtgcgtggggggg
tgagcagggggtgtgggcgcggcggtcgggctgtaacccccccctgcacccccctccccgagttg
ctgagcacggcccggcttcgggtgcggggctccgtacggggcgtggcgcggggctcgccgtgccg
ggcggggggtggcggcaggtgggggtgccgggcggggcggggccgcctcgggccggggagggctc
gggggaggggcgcggcggcccccggagcgccggcggctgtcgaggcgcggcgagccgcagccatt
gccttttatggtaatcgtgcgagagggcgcagggacttcctttgtcccaaatctgtgcggagccg
aaatctgggaggcgccgccgcaccccctctagcgggcgcggggcgaagcggtgcggcgccggcag
gaaggaaatgggcggggagggccttcgtgcgtcgccgcgccgccgtccccttctccctctccagc
ctcggggctgtccgcggggggacggctgccttcgggggggacggggcagggcggggttcggcttc
tggcgtgtgaccggcggctctagagcctctgctaaccatgttcatgccttcttctttttcctaca
g
CAG [E2 + CBA promoter + intron]
SEQ ID NO: 36
tgggactttccactagacatgacacagcaatctgatatgcttgcgtgagaagaggattcatatcc
tgggactttccacagattttaccggaagttgttagatgcttgcgtgagaagatctaacatgacac
agcaatccttagtgggactttccaagtatgtggggcggggagtatacatgacacagcaattgatc
attaccggaagtttataggtgggactttccagacctatgcttgcgtgagaagaaaggtctgggac
tttccagtcgaggtgagccccacgttctgcttcactctccccatctcccccccctccccaccccc
aattttgtatttatttattttttaattattttgtgcagcgatgggggcggggggggggggggcgc
gcgccaggcggggcggggcggggcgaggggcggggcggggcgaggcggaaaggtgcggcggcagc
caatcagagcggcgcgctccgaaagtttccttttatggcgaggcggcggcggcggcggccctata
aaaagcgaagcgcgcggcgggcgggagtcgctgcgttgccttcgccccgtgccccgctccgcgcc
gcctcgcgccgcccgccccggctctgactgaccgcgttactcccacaggtgagcgggcgggacgg
cccttctcctccgggctgtaattagcgcttggtttaatgacggctcgtttcttttctgtggctgc
gtgaaagccttaaagggctccgggagggccctttgtgcgggggggagcggctcggggggtgcgtg
cgtgtgtgtgtgcgtggggagcgccgcgtgcggctccgcgctgcccggcggctgtgagcgctgcg
ggcgcggcgcggggctttgtgcgctccgcagtgtgcgcgaggggagcgcggccgggggcggtgcc
ccgcggtgcggggggggctgcgaggggaacaaaggctgcgtgcggggtgtgtgcgtgggggggtg
agcagggggtgtgggcgcggcggtcgggctgtaacccccccctgcacccccctccccgagttgct
gagcacggcccggcttcgggtgcggggctccgtacggggcgtggcgcggggctcgccgtgccggg
cggggggtggcggcaggtgggggtgccgggcggggcggggccgcctcgggccggggagggctcgg
gggaggggcgcggcggcccccggagcgccggcggctgtcgaggcgcggcgagccgcagccattgc
cttttatggtaatcgtgcgagagggcgcagggacttcctttgtcccaaatctgtgcggagccgaa
atctgggaggcgccgccgcaccccctctagcgggcgcggggcgaagcggtgcggcgccggcagga
aggaaatgggcggggagggccttcgtgcgtcgccgcgccgccgtccccttctccctctccagcct
cggggctgtccgcggggggacggctgccttcgggggggacggggcagggcggggttcggcttctg
gcgtgtgaccggcggctctagagcctctgctaaccatgttcatgccttcttctttttcctacag
CAG [E1X3 + CBA promoter + UTR1]
SEQ ID NO: 37
gggactttccggggcggggcacgtggtgcacgggactttccgtgcacgtgcacgggactttccgg
gactttccgggactttccgtgcaccacgtggggactttccgtgcacgggactttccggggcgggg
cacgtggtgcacgggactttccgtgcacgtgcacgggactttccgggactttccgggactttccg
tgcaccacgtggggactttccgtgcacgggactttccggggcggggcacgtggtgcacgggactt
tccgtgcacgtgcacgggactttccgggactttccgggactttccgtgcaccacgtggggacttt
ccgtgcacgtcgaggtgagccccacgttctgcttcactctccccatctcccccccctccccaccc
ccaattttgtatttatttattttttaattattttgtgcagcgatgggggcggggggggggggggc
gcgcgccaggcggggcggggcggggcgaggggcggggcggggcgaggcggaaaggtgcggcggca
gccaatcagagcggcgcgctccgaaagtttccttttatggcgaggcggcggcggcggcggcccta
taaaaagcgaagcgcgcggcgggcgctgccttctccctcctgtgagtttggtaagtcgacgggcc
gggcctgggccgggtccgggccgggtcgttggatccccactacagcccgatactcaagcttgacg
aattcgagtatccaaggtagtggactagtgtgacgctgctgacccctttctttcccttctgcagg
ttggtgtacagtagcttccaaattgattaattcgagcgaacgcgtc
CAG [E2 (U100) + CBA promoter + UTR1]
SEQ ID NO: 38
Tgggactttccactagacatgacacagcaatctgatatgcttgcgtgagaagaggattcatatcc
tgggactttccacagattttaccggaagttgttagatgcttgcgtgagaagatctaacatgacac
agcaatccttagtgggactttccaagtatgtggggcggggagtatacatgacacagcaattgatc
attaccggaagtttataggtgggactttccagacctatgcttgcgtgagaagaaaggtctgggac
tttccagtcgaggtgagccccacgttctgcttcactctccccatctcccccccctccccaccccc
aattttgtatttatttattttttaattattttgtgcagcgatgggggcggggggggggggggcgc
gcgccaggcggggcggggcggggcgaggggcggggcggggcgaggcggaaaggtgcggcggcagc
caatcagagcggcgcgctccgaaagtttccttttatggcgaggcggcggcggcggcggccctata
aaaagcgaagcgcgcggcgggcgctgccttctccctcctgtgagtttggtaagtcgacgggccgg
gcctgggccgggtccgggccgggtcgttggatccccactacagcccgatactcaagcttgacgaa
ttcgagtatccaaggtagtggactagtgtgacgctgctgacccctttctttcccttctgcaggtt
ggtgtacagtagcttccaaattgattaattcgagcgaacgcgtc
CMV enhancer-EF1-UTR1
SEQ ID NO: 39
Gacattgattattgactagttattaatagtaatcaattacggggtcattagttcatagcccatat
atggagttccgcgttacataacttacggtaaatggcccgcctggctgaccgcccaacgacccccg
cccattgacgtcaataatgacgtatgttcccatagtaacgccaatagggactttccattgacgtc
aatgggtggagtatttacggtaaactgcccacttggcagtacatcaagtgtatcatatgccaagt
acgccccctattgacgtcaatgacggtaaatggcccgcctggcattatgcccagtacatgacctt
atgggactttcctacttggcagtacatctacgtattagtcatcgctattaccatggggcagagcg
cacatcgcccacagtccccgagaagttggggggaggggtcggcaattgaaccggtgcctagagaa
ggtggcgcggggtaaactgggaaagtgatgtcgtgtactggctccgcctttttcccgagggtggg
ggagaaccgtatataagtgcagtagtcgccgtgaacgttctttttcgcaacgggtttgccgccag
aacacagctgccttctccctcctgtgagtttggtaagtcgacgggccgggcctgggccgggtccg
ggccgggtcgttggatccccactacagcccgatactcaagcttgacgaattcgagtatccaaggt
agtggactagtgtgacgctgctgacccctttctttcccttctgcaggttggtgtacagtagcttc
caaattgattaattcgagcgaacgcgtc
4-1 pGL2-SS*-CAG [CMV enhancer + CBA Promoter + intron]-
SecNLuc-2A-eGFP-WPRE-3′UTR(108 to 120 polyA)-SS*
SEQ ID NO: 40
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacacccctgggtcgacgaca
ttgattattgactagttattaatagtaatcaattacggggtcattagttcatagcccatatatgg
agttccgcgttacataacttacggtaaatggcccgcctggctgaccgcccaacgacccccgccca
ttgacgtcaataatgacgtatgttcccatagtaacgccaatagggactttccattgacgtcaatg
ggtggagtatttacggtaaactgcccacttggcagtacatcaagtgtatcatatgccaagtacgc
cccctattgacgtcaatgacggtaaatggcccgcctggcattatgcccagtacatgaccttatgg
gactttcctacttggcagtacatctacgtattagtcatcgctattaccatggtcgaggtgagccc
cacgttctgcttcactctccccatctcccccccctccccacccccaattttgtatttatttattt
tttaattattttgtgcagcgatgggggcggggggggggggggcgcgcgccaggcggggcggggcg
gggcgaggggcggggcggggcgaggcggaaaggtgcggcggcagccaatcagagcggcgcgctcc
gaaagtttccttttatggcgaggcggcggcggcggcggccctataaaaagcgaagcgcgcggcgg
gcgggagtcgctgcgttgccttcgccccgtgccccgctccgcgccgcctcgcgccgcccgccccg
gctctgactgaccgcgttactcccacaggtgagcgggcgggacggcccttctcctccgggctgta
attagcgcttggtttaatgacggctcgtttcttttctgtggctgcgtgaaagccttaaagggctc
cgggagggccctttgtgcgggggggagcggctcggggggtgcgtgcgtgtgtgtgtgcgtgggga
gcgccgcgtgcggctccgcgctgcccggcggctgtgagcgctgcgggcgcggcgcggggctttgt
gcgctccgcagtgtgcgcgaggggagcgcggccgggggcggtgccccgcggtgcggggggggctg
cgaggggaacaaaggctgcgtgcggggtgtgtgcgtgggggggtgagcagggggtgtgggcgcgg
cggtcgggctgtaacccccccctgcacccccctccccgagttgctgagcacggcccggcttcggg
tgcggggctccgtacggggcgtggcgcggggctcgccgtgccgggcggggggtggcggcaggtgg
gggtgccgggcggggcggggccgcctcgggccggggagggctcgggggaggggcgcggcggcccc
cggagcgccggcggctgtcgaggcgcggcgagccgcagccattgccttttatggtaatcgtgcga
gagggcgcagggacttcctttgtcccaaatctgtgcggagccgaaatctgggaggcgccgccgca
ccccctctagcgggcgcggggcgaagcggtgcggcgccggcaggaaggaaatgggcggggagggc
cttcgtgcgtcgccgcgccgccgtccccttctccctctccagcctcggggctgtccgcgggggga
cggctgccttcgggggggacggggcagggcggggttcggcttctggcgtgtgaccggcggctcta
gagcctctgctaaccatgttcatgccttcttctttttcctacagctcctgggcaacgtgctggtt
attgtgctgtctcatcattttggcaaagaattgattaattcgagcgaacgcgtcgccaccatgaa
ctccttctccacaagcgccttcggtccagttgccttctccctgggcctgctcctggtgttgcctg
ctgccttccctgccccagtcttcacactcgaagatttcgttggggactggcgacagacagccggc
tacaacctggaccaagtccttgaacagggaggtgtgtccagtttgtttcagaatctcggggtgtc
cgtaactccgatccaaaggattgtcctgagcggtgaaaatgggctgaagatcgacatccatgtca
tcatcccgtatgaaggtctgagcggcgaccaaatgggccagatcgaaaaaatttttaaggtggtg
taccctgtggatgatcatcactttaaggtgatcctgcactatggcacactggtaatcgacggggt
tacgccgaacatgatcgactatttcggacggccgtatgaaggcatcgccgtgttcgacggcaaaa
agatcactgtaacagggaccctgtggaacggcaacaaaattatcgacgagcgcctgatcaacccc
gacggctccctgctgttccgagtaaccatcaacggagtgaccggctggcggctgtgcgaacgcat
tctggcggctagcgctactaacttcagcctgctgaagcaggctggagacgtggaggagaaccctg
gacctggaagcggagagggcagaggaagtctgctaacatgcggtgacgtcgaggagaatcctgga
cctggatccggaatggtgagcaagggcgaggagctgttcaccggggtggtgcccatcctggtcga
gctggacggcgacgtaaacggccacaagttcagcgtgtccggcgagggcgagggcgatgccacct
acggcaagctgaccctgaagttcatctgcaccaccggcaagctgcccgtgccctggcccaccctc
gtgaccaccctgacctacggcgtgcagtgcttcagccgctaccccgaccacatgaagcagcacga
cttcttcaagtccgccatgcccgaaggctacgtccaggagcgcaccatcttcttcaaggacgacg
gcaactacaagacccgcgccgaggtgaagttcgagggcgacaccctggtgaaccgcatcgagctg
aagggcatcgacttcaaggaggacggcaacatcctggggcacaagctggagtacaactacaacag
ccacaacgtctatatcatggccgacaagcagaagaacggcatcaaggtgaacttcaagatccgcc
acaacatcgaggacggcagcgtgcagctcgccgaccactaccagcagaacacccccatcggcgac
ggccccgtgctgctgcccgacaaccactacctgagcacccagtccgccctgagcaaagaccccaa
cgagaagcgcgatcacatggtcctgctggagttcgtgaccgccgccgggatcactctcggcatgg
acgagctgtacaagtaaaatcaacctctggattacaaaatttgtgaaagattgactggtattctt
aactatgttgctccttttacgctatgtggatacgctgctttaatgcctttgtatcatgctattgc
ttcccgtatggctttcattttctcctccttgtataaatcctggttgctgtctctttatgaggagt
tgtggcccgttgtcaggcaacgtggcgtggtgtgcactgtgtttgctgacgcaacccccactggt
tggggcattgccaccacctgtcagctcctttccgggactttcgctttccccctccctattgccac
ggcggaactcatcgccgcctgccttgcccgctgctggacaggggctcggctgttgggcactgaca
attccgtggtgttgtcggggaaatcatcgtcctttccttggctgctcgcctgtgttgccacctgg
attctgcgcgggacgtccttctgctacgtcccttcggccctcaatccagcggaccttccttcccg
cggcctgctgccggctctgcggcctcttccgcgtcttcgccttcgccctcagacgagtcggatct
ccctttgggccgcctccccgcgctcgctttcttgctgtccaatttctattaaaggttcctttgtt
ccctaagtccaactactaaactgggggatattatgaagggccttgagcatctggattctgcctaa
taaaaaacatttattttcattgcaagctcgctttcttgctgtccaatttctattaaaggttcctt
tgttccctaagtccaactactaaactgggggatattatgaagggccttgagcatctggattctgc
ctaataaaaaacatttattttcattgcaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaaa
aaaaactcggaaggacatggtgtggaaagtccccaggctccccagcaggcagaagtatgcaaagc
atgcatctcaattagtcagcaaccaggtgtggaaagtccccaggctccccagcaggcagaagtat
gcaaagcatgcatctcaattagtcagcaaccaaattaaagtaacccataacttcgtatagcatac
attatacgaagttatgaagttcctattctctagaaagtataggaacttctagtcacctatttcag
catactacgcgcgtagtatgctgaaataggtttatcagcacacaatagtccattatacgcgcgta
taatggcaattgtgtgctgattgggttactttaatttggatccgtcgaccgatgcccttgagagc
cttcaacccagtcagctccttccggtgggcgcggggcatgactatcgtcgccgcacttatgactg
tcttctttatcatgcaactcgtaggacaggtgccggcagcgctcttccgcttcctcgctcactga
ctcgctgcgctcggtcgttcggctgcggcgagcggtatcagctcactcaaaggcggtaatacggt
tatccacagaatcaggggataacgcaggaaagaacatgtgagcaaaaggccagcaaaaggccagg
aaccgtaaaaaggccgcgttgctggcgtttttccataggctccgcccccctgacgagcatcacaa
aaatcgacgctcaagtcagaggtggcgaaacccgacaggactataaagataccaggcgtttcccc
ctggaagctccctcgtgcgctctcctgttccgaccctgccgcttaccggatacctgtccgccttt
ctcccttcgggaagcgtggcgctttctcatagctcacgctgtaggtatctcagttcggtgtaggt
cgttcgctccaagctgggctgtgtgcacgaaccccccgttcagcccgaccgctgcgccttatccg
gtaactatcgtcttgagtccaacccggtaagacacgacttatcgccactggcagcagccactggt
aacaggattagcagagcgaggtatgtaggcggtgctacagagttcttgaagtggtggcctaacta
cggctacactagaagaacagtatttggtatctgcgctctgctgaagccagttaccttcggaaaaa
gagttggtagctcttgatccggcaaacaaaccaccgctggtagcggtggtttttttgtttgcaag
cagcagattacgcgcagaaaaaaaggatctcaagaagatcctttgatcttttctacggggtctga
cgctcagtggaacgaaaactcacgttaagggattttggtcatgagattatcaaaaaggatcttca
cctagatccttttaaattaaaaatgaagttttaaatcaatctaaagtatatatgagtaaacttgg
tctgacagttaccaatgcttaatcagtgaggcacctatctcagcgatctgtctatttcgttcatc
catagttgcctgactccccgtcgtgtagataactacgatacgggagggcttaccatctggcccca
gtgctgcaatgataccgcgagacccacgctcaccggctccagatttatcagcaataaaccagcca
gccggaagggccgagcgcagaagtggtcctgcaactttatccgcctccatccagtctattaattg
ttgccgggaagctagagtaagtagttcgccagttaatagtttgcgcaacgttgttgccattgcta
caggcatcgtggtgtcacgctcgtcgtttggtatggcttcattcagctccggttcccaacgatca
aggcgagttacatgatcccccatgttgtgcaaaaaagcggttagctccttcggtcctccgatcgt
tgtcagaagtaagttggccgcagtgttatcactcatggttatggcagcactgcataattctctta
ctgtcatgccatccgtaagatgcttttctgtgactggtgagtactcaaccaagtcattctgagaa
tagtgtatgcggcgaccgagttgctcttgcccggcgtcaatacgggataataccgcgccacatag
cagaactttaaaagtgctcatcattggaaaacgttcttcggggcgaaaactctcaaggatcttac
cgctgttgagatccagttcgatgtaacccactcgtgcacccaactgatcttcagcatcttttact
ttcaccagcgtttctgggtgagcaaaaacaggaaggcaaaatgccgcaaaaaagggaataagggc
gacacggaaatgttgaatactcatactcttcctttttcaatattattgaagcatttatcagggtt
attgtctcatgagcggatacatatttgaatgtatttagaaaaataaacaaataggggttccgcgc
acatttccccgaaaagtgccacctgacgcgccctgtagcggcgcattaagcgcggcgggtgtggt
ggttacgcgcagcgtgaccgctacacttgccagcgccctagcgcccgctcctttcgctttcttcc
cttcctttctcgccacgttcgccggctttccccgtcaagctctaaatcgggggctccctttaggg
ttccgatttagtgctttacggcacctcgaccccaaaaaacttgattagggtgatggttcacgtag
tgggccatcgccctgatagacggtttttcgccctttgacgttggagtccacgttctttaatagtg
gactcttgttccaaactggaacaacactcaaccctatctcggtctattcttttgatttataaggg
attttgccgatttcggcctattggttaaaaaatgagctgatttaacaaaaatttaacgcgaattt
taacaaaatattaacgcttacaatttgccattcgccattcaggctgcgcaactgttgggaagggc
gatcggtgcgggcctcttcgctattacgccagcccaagctaccatgataagtaagtaatattaag
gtacgtggaggttttacttgctttaaaaaacctcccacacctccccctgaacctgaaacataaaa
tgaatgcaattgttgttgttaacttgtttattgcagcttataatggttacaaataaagcaatagc
atcacaaatttcacaaataaagcatttttttcactgcattctagttgtggtttgtccaaactcat
caatgtatcttatggtactgtaactgagctaacataa
4-2 pGL2-SS*-CAG [E1 X3 + CBA promoter + introne]-SecNLuc-
2A-eGFP-WPRE-3′UTR(108 to 120 polyA)-SS*
SEQ ID NO: 41
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacacccctgggtcgacggga
ctttccggggcggggcacgtggtgcacgggactttccgtgcacgtgcacgggactttccgggact
ttccgggactttccgtgcaccacgtggggactttccgtgcacgggactttccggggcggggcacg
tggtgcacgggactttccgtgcacgtgcacgggactttccgggactttccgggactttccgtgca
ccacgtggggactttccgtgcacgggactttccggggcggggcacgtggtgcacgggactttccg
tgcacgtgcacgggactttccgggactttccgggactttccgtgcaccacgtggggactttccgt
gcacgtcgaggtgagccccacgttctgcttcactctccccatctcccccccctccccacccccaa
ttttgtatttatttattttttaattattttgtgcagcgatgggggcggggggggggggggcgcgc
gccaggcggggcggggcggggcgaggggcggggcggggcgaggcggaaaggtgcggcggcagcca
atcagagcggcgcgctccgaaagtttccttttatggcgaggcggcggcggcggcggccctataaa
aagcgaagcgcgcggcgggcgggagtcgctgcgttgccttcgccccgtgccccgctccgcgccgc
ctcgcgccgcccgccccggctctgactgaccgcgttactcccacaggtgagcgggcgggacggcc
cttctcctccgggctgtaattagcgcttggtttaatgacggctcgtttcttttctgtggctgcgt
gaaagccttaaagggctccgggagggccctttgtgcgggggggagcggctcggggggtgcgtgcg
tgtgtgtgtgcgtggggagcgccgcgtgcggctccgcgctgcccggcggctgtgagcgctgcggg
cgcggcgcggggctttgtgcgctccgcagtgtgcgcgaggggagcgcggccgggggcggtgcccc
gcggtgcggggggggctgcgaggggaacaaaggctgcgtgcggggtgtgtgcgtgggggggtgag
cagggggtgtgggcgcggcggtcgggctgtaacccccccctgcacccccctccccgagttgctga
gcacggcccggcttcgggtgcggggctccgtacggggcgtggcgcggggctcgccgtgccgggcg
gggggtggcggcaggtgggggtgccgggcggggcggggccgcctcgggccggggagggctcgggg
gaggggcgcggcggcccccggagcgccggcggctgtcgaggcgcggcgagccgcagccattgcct
tttatggtaatcgtgcgagagggcgcagggacttcctttgtcccaaatctgtgcggagccgaaat
ctgggaggcgccgccgcaccccctctagcgggcgcggggcgaagcggtgcggcgccggcaggaag
gaaatgggcggggagggccttcgtgcgtcgccgcgccgccgtccccttctccctctccagcctcg
gggctgtccgcggggggacggctgccttcgggggggacggggcagggcggggttcggcttctggc
gtgtgaccggcggctctagagcctctgctaaccatgttcatgccttcttctttttcctacagctc
ctgggcaacgtgctggttattgtgctgtctcatcattttggcaaagaattgattaattcgagcga
acgcgtcgccaccatgaactccttctccacaagcgccttcggtccagttgccttctccctgggcc
tgctcctggtgttgcctgctgccttccctgccccagtcttcacactcgaagatttcgttggggac
tggcgacagacagccggctacaacctggaccaagtccttgaacagggaggtgtgtccagtttgtt
tcagaatctcggggtgtccgtaactccgatccaaaggattgtcctgagcggtgaaaatgggctga
agatcgacatccatgtcatcatcccgtatgaaggtctgagcggcgaccaaatgggccagatcgaa
aaaatttttaaggtggtgtaccctgtggatgatcatcactttaaggtgatcctgcactatggcac
actggtaatcgacggggttacgccgaacatgatcgactatttcggacggccgtatgaaggcatcg
ccgtgttcgacggcaaaaagatcactgtaacagggaccctgtggaacggcaacaaaattatcgac
gagcgcctgatcaaccccgacggctccctgctgttccgagtaaccatcaacggagtgaccggctg
gcggctgtgcgaacgcattctggcggctagcgctactaacttcagcctgctgaagcaggctggag
acgtggaggagaaccctggacctggaagcggagagggcagaggaagtctgctaacatgcggtgac
gtcgaggagaatcctggacctggatccggaatggtgagcaagggcgaggagctgttcaccggggt
ggtgcccatcctggtcgagctggacggcgacgtaaacggccacaagttcagcgtgtccggcgagg
gcgagggcgatgccacctacggcaagctgaccctgaagttcatctgcaccaccggcaagctgccc
gtgccctggcccaccctcgtgaccaccctgacctacggcgtgcagtgcttcagccgctaccccga
ccacatgaagcagcacgacttcttcaagtccgccatgcccgaaggctacgtccaggagcgcacca
tcttcttcaaggacgacggcaactacaagacccgcgccgaggtgaagttcgagggcgacaccctg
gtgaaccgcatcgagctgaagggcatcgacttcaaggaggacggcaacatcctggggcacaagct
ggagtacaactacaacagccacaacgtctatatcatggccgacaagcagaagaacggcatcaagg
tgaacttcaagatccgccacaacatcgaggacggcagcgtgcagctcgccgaccactaccagcag
aacacccccatcggcgacggccccgtgctgctgcccgacaaccactacctgagcacccagtccgc
cctgagcaaagaccccaacgagaagcgcgatcacatggtcctgctggagttcgtgaccgccgccg
ggatcactctcggcatggacgagctgtacaagtaaaatcaacctctggattacaaaatttgtgaa
agattgactggtattcttaactatgttgctccttttacgctatgtggatacgctgctttaatgcc
tttgtatcatgctattgcttcccgtatggctttcattttctcctccttgtataaatcctggttgc
tgtctctttatgaggagttgtggcccgttgtcaggcaacgtggcgtggtgtgcactgtgtttgct
gacgcaacccccactggttggggcattgccaccacctgtcagctcctttccgggactttcgcttt
ccccctccctattgccacggcggaactcatcgccgcctgccttgcccgctgctggacaggggctc
ggctgttgggcactgacaattccgtggtgttgtcggggaaatcatcgtcctttccttggctgctc
gcctgtgttgccacctggattctgcgcgggacgtccttctgctacgtcccttcggccctcaatcc
agcggaccttccttcccgcggcctgctgccggctctgcggcctcttccgcgtcttcgccttcgcc
ctcagacgagtcggatctccctttgggccgcctccccgcgctcgctttcttgctgtccaatttct
attaaaggttcctttgttccctaagtccaactactaaactgggggatattatgaagggccttgag
catctggattctgcctaataaaaaacatttattttcattgcaagctcgctttcttgctgtccaat
ttctattaaaggttcctttgttccctaagtccaactactaaactgggggatattatgaagggcct
tgagcatctggattctgcctaataaaaaacatttattttcattgcaaaaaaaaaaaaaaaaaaaa
aaaaaaaaaaaaaaaaaaaaaaaaaaaaaaaaaaaaaaaaaaaaaaaaaaaaaaaaaaaaaaaaa
aaaaaaaaaaaaaaaaaaaaaaactcggaaggacatggtgtggaaagtccccaggctccccagca
ggcagaagtatgcaaagcatgcatctcaattagtcagcaaccaggtgtggaaagtccccaggctc
cccagcaggcagaagtatgcaaagcatgcatctcaattagtcagcaaccaaattaaagtaaccca
taacttcgtatagcatacattatacgaagttatgaagttcctattctctagaaagtataggaact
tctagtcacctatttcagcatactacgcgcgtagtatgctgaaataggtttatcagcacacaata
gtccattatacgcgcgtataatggcaattgtgtgctgattgggttactttaatttggatccgtcg
accgatgcccttgagagccttcaacccagtcagctccttccggtgggcgcggggcatgactatcg
tcgccgcacttatgactgtcttctttatcatgcaactcgtaggacaggtgccggcagcgctcttc
cgcttcctcgctcactgactcgctgcgctcggtcgttcggctgcggcgagcggtatcagctcact
caaaggcggtaatacggttatccacagaatcaggggataacgcaggaaagaacatgtgagcaaaa
ggccagcaaaaggccaggaaccgtaaaaaggccgcgttgctggcgtttttccataggctccgccc
ccctgacgagcatcacaaaaatcgacgctcaagtcagaggtggcgaaacccgacaggactataaa
gataccaggcgtttccccctggaagctccctcgtgcgctctcctgttccgaccctgccgcttacc
ggatacctgtccgcctttctcccttcgggaagcgtggcgctttctcatagctcacgctgtaggta
tctcagttcggtgtaggtcgttcgctccaagctgggctgtgtgcacgaaccccccgttcagcccg
accgctgcgccttatccggtaactatcgtcttgagtccaacccggtaagacacgacttatcgcca
ctggcagcagccactggtaacaggattagcagagcgaggtatgtaggcggtgctacagagttctt
gaagtggtggcctaactacggctacactagaagaacagtatttggtatctgcgctctgctgaagc
cagttaccttcggaaaaagagttggtagctcttgatccggcaaacaaaccaccgctggtagcggt
ggtttttttgtttgcaagcagcagattacgcgcagaaaaaaaggatctcaagaagatcctttgat
cttttctacggggtctgacgctcagtggaacgaaaactcacgttaagggattttggtcatgagat
tatcaaaaaggatcttcacctagatccttttaaattaaaaatgaagttttaaatcaatctaaagt
atatatgagtaaacttggtctgacagttaccaatgcttaatcagtgaggcacctatctcagcgat
ctgtctatttcgttcatccatagttgcctgactccccgtcgtgtagataactacgatacgggagg
gcttaccatctggccccagtgctgcaatgataccgcgagacccacgctcaccggctccagattta
tcagcaataaaccagccagccggaagggccgagcgcagaagtggtcctgcaactttatccgcctc
catccagtctattaattgttgccgggaagctagagtaagtagttcgccagttaatagtttgcgca
acgttgttgccattgctacaggcatcgtggtgtcacgctcgtcgtttggtatggcttcattcagc
tccggttcccaacgatcaaggcgagttacatgatcccccatgttgtgcaaaaaagcggttagctc
cttcggtcctccgatcgttgtcagaagtaagttggccgcagtgttatcactcatggttatggcag
cactgcataattctcttactgtcatgccatccgtaagatgcttttctgtgactggtgagtactca
accaagtcattctgagaatagtgtatgcggcgaccgagttgctcttgcccggcgtcaatacggga
taataccgcgccacatagcagaactttaaaagtgctcatcattggaaaacgttcttcggggcgaa
aactctcaaggatcttaccgctgttgagatccagttcgatgtaacccactcgtgcacccaactga
tcttcagcatcttttactttcaccagcgtttctgggtgagcaaaaacaggaaggcaaaatgccgc
aaaaaagggaataagggcgacacggaaatgttgaatactcatactcttcctttttcaatattatt
gaagcatttatcagggttattgtctcatgagcggatacatatttgaatgtatttagaaaaataaa
caaataggggttccgcgcacatttccccgaaaagtgccacctgacgcgccctgtagcggcgcatt
aagcgcggcgggtgtggtggttacgcgcagcgtgaccgctacacttgccagcgccctagcgcccg
ctcctttcgctttcttcccttcctttctcgccacgttcgccggctttccccgtcaagctctaaat
cgggggctccctttagggttccgatttagtgctttacggcacctcgaccccaaaaaacttgatta
gggtgatggttcacgtagtgggccatcgccctgatagacggtttttcgccctttgacgttggagt
ccacgttctttaatagtggactcttgttccaaactggaacaacactcaaccctatctcggtctat
tcttttgatttataagggattttgccgatttcggcctattggttaaaaaatgagctgatttaaca
aaaatttaacgcgaattttaacaaaatattaacgcttacaatttgccattcgccattcaggctgc
gcaactgttgggaagggcgatcggtgcgggcctcttcgctattacgccagcccaagctaccatga
taagtaagtaatattaaggtacgtggaggttttacttgctttaaaaaacctcccacacctccccc
tgaacctgaaacataaaatgaatgcaattgttgttgttaacttgtttattgcagcttataatggt
tacaaataaagcaatagcatcacaaatttcacaaataaagcatttttttcactgcattctagttg
tggtttgtccaaactcatcaatgtatcttatggtactgtaactgagctaacataa
4-3 pGL2-SS*-CAG [E2(U100) + CBA promoter + introne]-
SecNLuc-2A-eGFP-WPRE-3′UTR(108 to 120 polyA)-SS*
SEQ ID NO: 42
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacacccctgggtcgactggg
actttccactagacatgacacagcaatctgatatgcttgcgtgagaagaggattcatatcctggg
actttccacagattttaccggaagttgttagatgcttgcgtgagaagatctaacatgacacagca
atccttagtgggactttccaagtatgtggggcggggagtatacatgacacagcaattgatcatta
ccggaagtttataggtgggactttccagacctatgcttgcgtgagaagaaaggtctgggactttc
cagtcgaggtgagccccacgttctgcttcactctccccatctcccccccctccccacccccaatt
ttgtatttatttattttttaattattttgtgcagcgatgggggcggggggggggggggcgcgcgc
caggcggggcggggcggggcgaggggcggggcggggcgaggcggaaaggtgcggcggcagccaat
cagagcggcgcgctccgaaagtttccttttatggcgaggcggcggcggcggcggccctataaaaa
gcgaagcgcgcggcgggcgggagtcgctgcgttgccttcgccccgtgccccgctccgcgccgcct
cgcgccgcccgccccggctctgactgaccgcgttactcccacaggtgagcgggcgggacggccct
tctcctccgggctgtaattagcgcttggtttaatgacggctcgtttcttttctgtggctgcgtga
aagccttaaagggctccgggagggccctttgtgcgggggggagcggctcggggggtgcgtgcgtg
tgtgtgtgcgtggggagcgccgcgtgcggctccgcgctgcccggcggctgtgagcgctgcgggcg
cggcgcggggctttgtgcgctccgcagtgtgcgcgaggggagcgcggccgggggcggtgccccgc
ggtgcggggggggctgcgaggggaacaaaggctgcgtgcggggtgtgtgcgtgggggggtgagca
gggggtgtgggcgcggcggtcgggctgtaacccccccctgcacccccctccccgagttgctgagc
acggcccggcttcgggtgcggggctccgtacggggcgtggcgcggggctcgccgtgccgggcggg
gggtggcggcaggtgggggtgccgggcggggcggggccgcctcgggccggggagggctcggggga
ggggcgcggcggcccccggagcgccggcggctgtcgaggcgcggcgagccgcagccattgccttt
tatggtaatcgtgcgagagggcgcagggacttcctttgtcccaaatctgtgcggagccgaaatct
gggaggcgccgccgcaccccctctagcgggcgcggggcgaagcggtgcggcgccggcaggaagga
aatgggcggggagggccttcgtgcgtcgccgcgccgccgtccccttctccctctccagcctcggg
gctgtccgcggggggacggctgccttcgggggggacggggcagggcggggttcggcttctggcgt
gtgaccggcggctctagagcctctgctaaccatgttcatgccttcttctttttcctacagctcct
gggcaacgtgctggttattgtgctgtctcatcattttggcaaagaattgattaattcgagcgaac
gcgtcgccaccatgaactccttctccacaagcgccttcggtccagttgccttctccctgggcctg
ctcctggtgttgcctgctgccttccctgccccagtcttcacactcgaagatttcgttggggactg
gcgacagacagccggctacaacctggaccaagtccttgaacagggaggtgtgtccagtttgtttc
agaatctcggggtgtccgtaactccgatccaaaggattgtcctgagcggtgaaaatgggctgaag
atcgacatccatgtcatcatcccgtatgaaggtctgagcggcgaccaaatgggccagatcgaaaa
aatttttaaggtggtgtaccctgtggatgatcatcactttaaggtgatcctgcactatggcacac
tggtaatcgacggggttacgccgaacatgatcgactatttcggacggccgtatgaaggcatcgcc
gtgttcgacggcaaaaagatcactgtaacagggaccctgtggaacggcaacaaaattatcgacga
gcgcctgatcaaccccgacggctccctgctgttccgagtaaccatcaacggagtgaccggctggc
ggctgtgcgaacgcattctggcggctagcgctactaacttcagcctgctgaagcaggctggagac
gtggaggagaaccctggacctggaagcggagagggcagaggaagtctgctaacatgcggtgacgt
cgaggagaatcctggacctggatccggaatggtgagcaagggcgaggagctgttcaccggggtgg
tgcccatcctggtcgagctggacggcgacgtaaacggccacaagttcagcgtgtccggcgagggc
gagggcgatgccacctacggcaagctgaccctgaagttcatctgcaccaccggcaagctgcccgt
gccctggcccaccctcgtgaccaccctgacctacggcgtgcagtgcttcagccgctaccccgacc
acatgaagcagcacgacttcttcaagtccgccatgcccgaaggctacgtccaggagcgcaccatc
ttcttcaaggacgacggcaactacaagacccgcgccgaggtgaagttcgagggcgacaccctggt
gaaccgcatcgagctgaagggcatcgacttcaaggaggacggcaacatcctggggcacaagctgg
agtacaactacaacagccacaacgtctatatcatggccgacaagcagaagaacggcatcaaggtg
aacttcaagatccgccacaacatcgaggacggcagcgtgcagctcgccgaccactaccagcagaa
cacccccatcggcgacggccccgtgctgctgcccgacaaccactacctgagcacccagtccgccc
tgagcaaagaccccaacgagaagcgcgatcacatggtcctgctggagttcgtgaccgccgccggg
atcactctcggcatggacgagctgtacaagtaaaatcaacctctggattacaaaatttgtgaaag
attgactggtattcttaactatgttgctccttttacgctatgtggatacgctgctttaatgcctt
tgtatcatgctattgcttcccgtatggctttcattttctcctccttgtataaatcctggttgctg
tctctttatgaggagttgtggcccgttgtcaggcaacgtggcgtggtgtgcactgtgtttgctga
cgcaacccccactggttggggcattgccaccacctgtcagctcctttccgggactttcgctttcc
ccctccctattgccacggcggaactcatcgccgcctgccttgcccgctgctggacaggggctcgg
ctgttgggcactgacaattccgtggtgttgtcggggaaatcatcgtcctttccttggctgctcgc
ctgtgttgccacctggattctgcgcgggacgtccttctgctacgtcccttcggccctcaatccag
cggaccttccttcccgcggcctgctgccggctctgcggcctcttccgcgtcttcgccttcgccct
cagacgagtcggatctccctttgggccgcctccccgcgctcgctttcttgctgtccaatttctat
taaaggttcctttgttccctaagtccaactactaaactgggggatattatgaagggccttgagca
tctggattctgcctaataaaaaacatttattttcattgcaagctcgctttcttgctgtccaattt
ctattaaaggttcctttgttccctaagtccaactactaaactgggggatattatgaagggccttg
agcatctggattctgcctaataaaaaacatttattttcattgcaaaaaaaaaaaaaaaaaaaaaa
aaaaaaaaaaaaaaaaaaaaaaaaaaaaaaaaaaaaaaaaaaaaaaaaaaaaaaaaaaaaaaaaa
aaaaaaaaaaaaaaaaaaaaactcggaaggacatggtgtggaaagtccccaggctccccagcagg
cagaagtatgcaaagcatgcatctcaattagtcagcaaccaggtgtggaaagtccccaggctccc
cagcaggcagaagtatgcaaagcatgcatctcaattagtcagcaaccaaattaaagtaacccata
acttcgtatagcatacattatacgaagttatgaagttcctattctctagaaagtataggaacttc
tagtcacctatttcagcatactacgcgcgtagtatgctgaaataggtttatcagcacacaatagt
ccattatacgcgcgtataatggcaattgtgtgctgattgggttactttaatttggatccgtcgac
cgatgcccttgagagccttcaacccagtcagctccttccggtgggcgcggggcatgactatcgtc
gccgcacttatgactgtcttctttatcatgcaactcgtaggacaggtgccggcagcgctcttccg
cttcctcgctcactgactcgctgcgctcggtcgttcggctgcggcgagcggtatcagctcactca
aaggcggtaatacggttatccacagaatcaggggataacgcaggaaagaacatgtgagcaaaagg
ccagcaaaaggccaggaaccgtaaaaaggccgcgttgctggcgtttttccataggctccgccccc
ctgacgagcatcacaaaaatcgacgctcaagtcagaggtggcgaaacccgacaggactataaaga
taccaggcgtttccccctggaagctccctcgtgcgctctcctgttccgaccctgccgcttaccgg
atacctgtccgcctttctcccttcgggaagcgtggcgctttctcatagctcacgctgtaggtatc
tcagttcggtgtaggtcgttcgctccaagctgggctgtgtgcacgaaccccccgttcagcccgac
cgctgcgccttatccggtaactatcgtcttgagtccaacccggtaagacacgacttatcgccact
ggcagcagccactggtaacaggattagcagagcgaggtatgtaggcggtgctacagagttcttga
agtggtggcctaactacggctacactagaagaacagtatttggtatctgcgctctgctgaagcca
gttaccttcggaaaaagagttggtagctcttgatccggcaaacaaaccaccgctggtagcggtgg
tttttttgtttgcaagcagcagattacgcgcagaaaaaaaggatctcaagaagatcctttgatct
tttctacggggtctgacgctcagtggaacgaaaactcacgttaagggattttggtcatgagatta
tcaaaaaggatcttcacctagatccttttaaattaaaaatgaagttttaaatcaatctaaagtat
atatgagtaaacttggtctgacagttaccaatgcttaatcagtgaggcacctatctcagcgatct
gtctatttcgttcatccatagttgcctgactccccgtcgtgtagataactacgatacgggagggc
ttaccatctggccccagtgctgcaatgataccgcgagacccacgctcaccggctccagatttatc
agcaataaaccagccagccggaagggccgagcgcagaagtggtcctgcaactttatccgcctcca
tccagtctattaattgttgccgggaagctagagtaagtagttcgccagttaatagtttgcgcaac
gttgttgccattgctacaggcatcgtggtgtcacgctcgtcgtttggtatggcttcattcagctc
cggttcccaacgatcaaggcgagttacatgatcccccatgttgtgcaaaaaagcggttagctcct
tcggtcctccgatcgttgtcagaagtaagttggccgcagtgttatcactcatggttatggcagca
ctgcataattctcttactgtcatgccatccgtaagatgcttttctgtgactggtgagtactcaac
caagtcattctgagaatagtgtatgcggcgaccgagttgctcttgcccggcgtcaatacgggata
ataccgcgccacatagcagaactttaaaagtgctcatcattggaaaacgttcttcggggcgaaaa
ctctcaaggatcttaccgctgttgagatccagttcgatgtaacccactcgtgcacccaactgatc
ttcagcatcttttactttcaccagcgtttctgggtgagcaaaaacaggaaggcaaaatgccgcaa
aaaagggaataagggcgacacggaaatgttgaatactcatactcttcctttttcaatattattga
agcatttatcagggttattgtctcatgagcggatacatatttgaatgtatttagaaaaataaaca
aataggggttccgcgcacatttccccgaaaagtgccacctgacgcgccctgtagcggcgcattaa
gcgcggcgggtgtggtggttacgcgcagcgtgaccgctacacttgccagcgccctagcgcccgct
cctttcgctttcttcccttcctttctcgccacgttcgccggctttccccgtcaagctctaaatcg
ggggctccctttagggttccgatttagtgctttacggcacctcgaccccaaaaaacttgattagg
gtgatggttcacgtagtgggccatcgccctgatagacggtttttcgccctttgacgttggagtcc
acgttctttaatagtggactcttgttccaaactggaacaacactcaaccctatctcggtctattc
ttttgatttataagggattttgccgatttcggcctattggttaaaaaatgagctgatttaacaaa
aatttaacgcgaattttaacaaaatattaacgcttacaatttgccattcgccattcaggctgcgc
aactgttgggaagggcgatcggtgcgggcctcttcgctattacgccagcccaagctaccatgata
agtaagtaatattaaggtacgtggaggttttacttgctttaaaaaacctcccacacctccccctg
aacctgaaacataaaatgaatgcaattgttgttgttaacttgtttattgcagcttataatggtta
caaataaagcaatagcatcacaaatttcacaaataaagcatttttttcactgcattctagttgtg
gtttgtccaaactcatcaatgtatcttatggtactgtaactgagctaacataa
4-4 pGL2-SS*-CAG [E1 X3 + CBA promoter + UTR1]-SecNLuc-
2A-eGFP-WPRE-3′UTR(108 to 120 polyA)-SS*
SEQ ID NO: 43
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacacccctgggtcgacggga
ctttccggggcggggcacgtggtgcacgggactttccgtgcacgtgcacgggactttccgggact
ttccgggactttccgtgcaccacgtggggactttccgtgcacgggactttccggggcggggcacg
tggtgcacgggactttccgtgcacgtgcacgggactttccgggactttccgggactttccgtgca
ccacgtggggactttccgtgcacgggactttccggggcggggcacgtggtgcacgggactttccg
tgcacgtgcacgggactttccgggactttccgggactttccgtgcaccacgtggggactttccgt
gcacgtcgaggtgagccccacgttctgcttcactctccccatctcccccccctccccacccccaa
ttttgtatttatttattttttaattattttgtgcagcgatgggggcggggggggggggggcgcgc
gccaggcggggcggggcggggcgaggggcggggcggggcgaggcggaaaggtgcggcggcagcca
atcagagcggcgcgctccgaaagtttccttttatggcgaggcggcggcggcggcggccctataaa
aagcgaagcgcgcggcgggcgctgccttctccctcctgtgagtttggtaagtcgacgggccgggc
ctgggccgggtccgggccgggtcgttggatccccactacagcccgatactcaagcttgacgaatt
cgagtatccaaggtagtggactagtgtgacgctgctgacccctttctttcccttctgcaggttgg
tgtacagtagcttccaaattgattaattcgagcgaacgcgtcgccaccatgaactccttctccac
aagcgccttcggtccagttgccttctccctgggcctgctcctggtgttgcctgctgccttccctg
ccccagtcttcacactcgaagatttcgttggggactggcgacagacagccggctacaacctggac
caagtccttgaacagggaggtgtgtccagtttgtttcagaatctcggggtgtccgtaactccgat
ccaaaggattgtcctgagcggtgaaaatgggctgaagatcgacatccatgtcatcatcccgtatg
aaggtctgagcggcgaccaaatgggccagatcgaaaaaatttttaaggtggtgtaccctgtggat
gatcatcactttaaggtgatcctgcactatggcacactggtaatcgacggggttacgccgaacat
gatcgactatttcggacggccgtatgaaggcatcgccgtgttcgacggcaaaaagatcactgtaa
cagggaccctgtggaacggcaacaaaattatcgacgagcgcctgatcaaccccgacggctccctg
ctgttccgagtaaccatcaacggagtgaccggctggcggctgtgcgaacgcattctggcggctag
cgctactaacttcagcctgctgaagcaggctggagacgtggaggagaaccctggacctggaagcg
gagagggcagaggaagtctgctaacatgcggtgacgtcgaggagaatcctggacctggatccgga
atggtgagcaagggcgaggagctgttcaccggggtggtgcccatcctggtcgagctggacggcga
cgtaaacggccacaagttcagcgtgtccggcgagggcgagggcgatgccacctacggcaagctga
ccctgaagttcatctgcaccaccggcaagctgcccgtgccctggcccaccctcgtgaccaccctg
acctacggcgtgcagtgcttcagccgctaccccgaccacatgaagcagcacgacttcttcaagtc
cgccatgcccgaaggctacgtccaggagcgcaccatcttcttcaaggacgacggcaactacaaga
cccgcgccgaggtgaagttcgagggcgacaccctggtgaaccgcatcgagctgaagggcatcgac
ttcaaggaggacggcaacatcctggggcacaagctggagtacaactacaacagccacaacgtcta
tatcatggccgacaagcagaagaacggcatcaaggtgaacttcaagatccgccacaacatcgagg
acggcagcgtgcagctcgccgaccactaccagcagaacacccccatcggcgacggccccgtgctg
ctgcccgacaaccactacctgagcacccagtccgccctgagcaaagaccccaacgagaagcgcga
tcacatggtcctgctggagttcgtgaccgccgccgggatcactctcggcatggacgagctgtaca
agtaaaatcaacctctggattacaaaatttgtgaaagattgactggtattcttaactatgttgct
ccttttacgctatgtggatacgctgctttaatgcctttgtatcatgctattgcttcccgtatggc
tttcattttctcctccttgtataaatcctggttgctgtctctttatgaggagttgtggcccgttg
tcaggcaacgtggcgtggtgtgcactgtgtttgctgacgcaacccccactggttggggcattgcc
accacctgtcagctcctttccgggactttcgctttccccctccctattgccacggcggaactcat
cgccgcctgccttgcccgctgctggacaggggctcggctgttgggcactgacaattccgtggtgt
tgtcggggaaatcatcgtcctttccttggctgctcgcctgtgttgccacctggattctgcgcggg
acgtccttctgctacgtcccttcggccctcaatccagcggaccttccttcccgcggcctgctgcc
ggctctgcggcctcttccgcgtcttcgccttcgccctcagacgagtcggatctccctttgggccg
cctccccgcgctcgctttcttgctgtccaatttctattaaaggttcctttgttccctaagtccaa
ctactaaactgggggatattatgaagggccttgagcatctggattctgcctaataaaaaacattt
attttcattgcaagctcgctttcttgctgtccaatttctattaaaggttcctttgttccctaagt
ccaactactaaactgggggatattatgaagggccttgagcatctggattctgcctaataaaaaac
atttattttcattgcaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaactcggaa
ggacatggtgtggaaagtccccaggctccccagcaggcagaagtatgcaaagcatgcatctcaat
tagtcagcaaccaggtgtggaaagtccccaggctccccagcaggcagaagtatgcaaagcatgca
tctcaattagtcagcaaccaaattaaagtaacccataacttcgtatagcatacattatacgaagt
tatgaagttcctattctctagaaagtataggaacttctagtcacctatttcagcatactacgcgc
gtagtatgctgaaataggtttatcagcacacaatagtccattatacgcgcgtataatggcaattg
tgtgctgattgggttactttaatttggatccgtcgaccgatgcccttgagagccttcaacccagt
cagctccttccggtgggcgcggggcatgactatcgtcgccgcacttatgactgtcttctttatca
tgcaactcgtaggacaggtgccggcagcgctcttccgcttcctcgctcactgactcgctgcgctc
ggtcgttcggctgcggcgagcggtatcagctcactcaaaggcggtaatacggttatccacagaat
caggggataacgcaggaaagaacatgtgagcaaaaggccagcaaaaggccaggaaccgtaaaaag
gccgcgttgctggcgtttttccataggctccgcccccctgacgagcatcacaaaaatcgacgctc
aagtcagaggtggcgaaacccgacaggactataaagataccaggcgtttccccctggaagctccc
tcgtgcgctctcctgttccgaccctgccgcttaccggatacctgtccgcctttctcccttcggga
agcgtggcgctttctcatagctcacgctgtaggtatctcagttcggtgtaggtcgttcgctccaa
gctgggctgtgtgcacgaaccccccgttcagcccgaccgctgcgccttatccggtaactatcgtc
ttgagtccaacccggtaagacacgacttatcgccactggcagcagccactggtaacaggattagc
agagcgaggtatgtaggcggtgctacagagttcttgaagtggtggcctaactacggctacactag
aagaacagtatttggtatctgcgctctgctgaagccagttaccttcggaaaaagagttggtagct
cttgatccggcaaacaaaccaccgctggtagcggtggtttttttgtttgcaagcagcagattacg
cgcagaaaaaaaggatctcaagaagatcctttgatcttttctacggggtctgacgctcagtggaa
cgaaaactcacgttaagggattttggtcatgagattatcaaaaaggatcttcacctagatccttt
taaattaaaaatgaagttttaaatcaatctaaagtatatatgagtaaacttggtctgacagttac
caatgcttaatcagtgaggcacctatctcagcgatctgtctatttcgttcatccatagttgcctg
actccccgtcgtgtagataactacgatacgggagggcttaccatctggccccagtgctgcaatga
taccgcgagacccacgctcaccggctccagatttatcagcaataaaccagccagccggaagggcc
gagcgcagaagtggtcctgcaactttatccgcctccatccagtctattaattgttgccgggaagc
tagagtaagtagttcgccagttaatagtttgcgcaacgttgttgccattgctacaggcatcgtgg
tgtcacgctcgtcgtttggtatggcttcattcagctccggttcccaacgatcaaggcgagttaca
tgatcccccatgttgtgcaaaaaagcggttagctccttcggtcctccgatcgttgtcagaagtaa
gttggccgcagtgttatcactcatggttatggcagcactgcataattctcttactgtcatgccat
ccgtaagatgcttttctgtgactggtgagtactcaaccaagtcattctgagaatagtgtatgcgg
cgaccgagttgctcttgcccggcgtcaatacgggataataccgcgccacatagcagaactttaaa
agtgctcatcattggaaaacgttcttcggggcgaaaactctcaaggatcttaccgctgttgagat
ccagttcgatgtaacccactcgtgcacccaactgatcttcagcatcttttactttcaccagcgtt
tctgggtgagcaaaaacaggaaggcaaaatgccgcaaaaaagggaataagggcgacacggaaatg
ttgaatactcatactcttcctttttcaatattattgaagcatttatcagggttattgtctcatga
gcggatacatatttgaatgtatttagaaaaataaacaaataggggttccgcgcacatttccccga
aaagtgccacctgacgcgccctgtagcggcgcattaagcgcggcgggtgtggtggttacgcgcag
cgtgaccgctacacttgccagcgccctagcgcccgctcctttcgctttcttcccttcctttctcg
ccacgttcgccggctttccccgtcaagctctaaatcgggggctccctttagggttccgatttagt
gctttacggcacctcgaccccaaaaaacttgattagggtgatggttcacgtagtgggccatcgcc
ctgatagacggtttttcgccctttgacgttggagtccacgttctttaatagtggactcttgttcc
aaactggaacaacactcaaccctatctcggtctattcttttgatttataagggattttgccgatt
tcggcctattggttaaaaaatgagctgatttaacaaaaatttaacgcgaattttaacaaaatatt
aacgcttacaatttgccattcgccattcaggctgcgcaactgttgggaagggcgatcggtgcggg
cctcttcgctattacgccagcccaagctaccatgataagtaagtaatattaaggtacgtggaggt
tttacttgctttaaaaaacctcccacacctccccctgaacctgaaacataaaatgaatgcaattg
ttgttgttaacttgtttattgcagcttataatggttacaaataaagcaatagcatcacaaatttc
acaaataaagcatttttttcactgcattctagttgtggtttgtccaaactcatcaatgtatctta
tggtactgtaactgagctaacataa
4-5-pGL2-SS*-CAG [E2 (U100) + CBA promoter + UTR1]-SecNLuc-
2A-eGFP-WPRE-3′UTR (108 to 120 polyA)-SS*
SEQ ID NO: 44
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacacccctgggtcgactggg
actttccactagacatgacacagcaatctgatatgcttgcgtgagaagaggattcatatcctggg
actttccacagattttaccggaagttgttagatgcttgcgtgagaagatctaacatgacacagca
atccttagtgggactttccaagtatgtggggcggggagtatacatgacacagcaattgatcatta
ccggaagtttataggtgggactttccagacctatgcttgcgtgagaagaaaggtctgggactttc
cagtcgaggtgagccccacgttctgcttcactctccccatctcccccccctccccacccccaatt
ttgtatttatttattttttaattattttgtgcagcgatgggggcggggggggggggggcgcgcgc
caggcggggcggggcggggcgaggggcggggcggggcgaggcggaaaggtgcggcggcagccaat
cagagcggcgcgctccgaaagtttccttttatggcgaggcggcggcggcggcggccctataaaaa
gcgaagcgcgcggcgggcgctgccttctccctcctgtgagtttggtaagtcgacgggccgggcct
gggccgggtccgggccgggtcgttggatccccactacagcccgatactcaagcttgacgaattcg
agtatccaaggtagtggactagtgtgacgctgctgacccctttctttcccttctgcaggttggtg
tacagtagcttccaaattgattaattcgagcgaacgcgtcgccaccatgaactccttctccacaa
gcgccttcggtccagttgccttctccctgggcctgctcctggtgttgcctgctgccttccctgcc
ccagtcttcacactcgaagatttcgttggggactggcgacagacagccggctacaacctggacca
agtccttgaacagggaggtgtgtccagtttgtttcagaatctcggggtgtccgtaactccgatcc
aaaggattgtcctgagcggtgaaaatgggctgaagatcgacatccatgtcatcatcccgtatgaa
ggtctgagcggcgaccaaatgggccagatcgaaaaaatttttaaggtggtgtaccctgtggatga
tcatcactttaaggtgatcctgcactatggcacactggtaatcgacggggttacgccgaacatga
tcgactatttcggacggccgtatgaaggcatcgccgtgttcgacggcaaaaagatcactgtaaca
gggaccctgtggaacggcaacaaaattatcgacgagcgcctgatcaaccccgacggctccctgct
gttccgagtaaccatcaacggagtgaccggctggcggctgtgcgaacgcattctggcggctagcg
ctactaacttcagcctgctgaagcaggctggagacgtggaggagaaccctggacctggaagcgga
gagggcagaggaagtctgctaacatgcggtgacgtcgaggagaatcctggacctggatccggaat
ggtgagcaagggcgaggagctgttcaccggggtggtgcccatcctggtcgagctggacggcgacg
taaacggccacaagttcagcgtgtccggcgagggcgagggcgatgccacctacggcaagctgacc
ctgaagttcatctgcaccaccggcaagctgcccgtgccctggcccaccctcgtgaccaccctgac
ctacggcgtgcagtgcttcagccgctaccccgaccacatgaagcagcacgacttcttcaagtccg
ccatgcccgaaggctacgtccaggagcgcaccatcttcttcaaggacgacggcaactacaagacc
cgcgccgaggtgaagttcgagggcgacaccctggtgaaccgcatcgagctgaagggcatcgactt
caaggaggacggcaacatcctggggcacaagctggagtacaactacaacagccacaacgtctata
tcatggccgacaagcagaagaacggcatcaaggtgaacttcaagatccgccacaacatcgaggac
ggcagcgtgcagctcgccgaccactaccagcagaacacccccatcggcgacggccccgtgctgct
gcccgacaaccactacctgagcacccagtccgccctgagcaaagaccccaacgagaagcgcgatc
acatggtcctgctggagttcgtgaccgccgccgggatcactctcggcatggacgagctgtacaag
taaaatcaacctctggattacaaaatttgtgaaagattgactggtattcttaactatgttgctcc
ttttacgctatgtggatacgctgctttaatgcctttgtatcatgctattgcttcccgtatggctt
tcattttctcctccttgtataaatcctggttgctgtctctttatgaggagttgtggcccgttgtc
aggcaacgtggcgtggtgtgcactgtgtttgctgacgcaacccccactggttggggcattgccac
cacctgtcagctcctttccgggactttcgctttccccctccctattgccacggcggaactcatcg
ccgcctgccttgcccgctgctggacaggggctcggctgttgggcactgacaattccgtggtgttg
tcggggaaatcatcgtcctttccttggctgctcgcctgtgttgccacctggattctgcgcgggac
gtccttctgctacgtcccttcggccctcaatccagcggaccttccttcccgcggcctgctgccgg
ctctgcggcctcttccgcgtcttcgccttcgccctcagacgagtcggatctccctttgggccgcc
tccccgcgctcgctttcttgctgtccaatttctattaaaggttcctttgttccctaagtccaact
actaaactgggggatattatgaagggccttgagcatctggattctgcctaataaaaaacatttat
tttcattgcaagctcgctttcttgctgtccaatttctattaaaggttcctttgttccctaagtcc
aactactaaactgggggatattatgaagggccttgagcatctggattctgcctaataaaaaacat
ttattttcattgcaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaactcggaagg
acatggtgtggaaagtccccaggctccccagcaggcagaagtatgcaaagcatgcatctcaatta
gtcagcaaccaggtgtggaaagtccccaggctccccagcaggcagaagtatgcaaagcatgcatc
tcaattagtcagcaaccaaattaaagtaacccataacttcgtatagcatacattatacgaagtta
tgaagttcctattctctagaaagtataggaacttctagtcacctatttcagcatactacgcgcgt
agtatgctgaaataggtttatcagcacacaatagtccattatacgcgcgtataatggcaattgtg
tgctgattgggttactttaatttggatccgtcgaccgatgcccttgagagccttcaacccagtca
gctccttccggtgggcgcggggcatgactatcgtcgccgcacttatgactgtcttctttatcatg
caactcgtaggacaggtgccggcagcgctcttccgcttcctcgctcactgactcgctgcgctcgg
tcgttcggctgcggcgagcggtatcagctcactcaaaggcggtaatacggttatccacagaatca
ggggataacgcaggaaagaacatgtgagcaaaaggccagcaaaaggccaggaaccgtaaaaaggc
cgcgttgctggcgtttttccataggctccgcccccctgacgagcatcacaaaaatcgacgctcaa
gtcagaggtggcgaaacccgacaggactataaagataccaggcgtttccccctggaagctccctc
gtgcgctctcctgttccgaccctgccgcttaccggatacctgtccgcctttctcccttcgggaag
cgtggcgctttctcatagctcacgctgtaggtatctcagttcggtgtaggtcgttcgctccaagc
tgggctgtgtgcacgaaccccccgttcagcccgaccgctgcgccttatccggtaactatcgtctt
gagtccaacccggtaagacacgacttatcgccactggcagcagccactggtaacaggattagcag
agcgaggtatgtaggcggtgctacagagttcttgaagtggtggcctaactacggctacactagaa
gaacagtatttggtatctgcgctctgctgaagccagttaccttcggaaaaagagttggtagctct
tgatccggcaaacaaaccaccgctggtagcggtggtttttttgtttgcaagcagcagattacgcg
cagaaaaaaaggatctcaagaagatcctttgatcttttctacggggtctgacgctcagtggaacg
aaaactcacgttaagggattttggtcatgagattatcaaaaaggatcttcacctagatcctttta
aattaaaaatgaagttttaaatcaatctaaagtatatatgagtaaacttggtctgacagttacca
atgcttaatcagtgaggcacctatctcagcgatctgtctatttcgttcatccatagttgcctgac
tccccgtcgtgtagataactacgatacgggagggcttaccatctggccccagtgctgcaatgata
ccgcgagacccacgctcaccggctccagatttatcagcaataaaccagccagccggaagggccga
gcgcagaagtggtcctgcaactttatccgcctccatccagtctattaattgttgccgggaagcta
gagtaagtagttcgccagttaatagtttgcgcaacgttgttgccattgctacaggcatcgtggtg
tcacgctcgtcgtttggtatggcttcattcagctccggttcccaacgatcaaggcgagttacatg
atcccccatgttgtgcaaaaaagcggttagctccttcggtcctccgatcgttgtcagaagtaagt
tggccgcagtgttatcactcatggttatggcagcactgcataattctcttactgtcatgccatcc
gtaagatgcttttctgtgactggtgagtactcaaccaagtcattctgagaatagtgtatgcggcg
accgagttgctcttgcccggcgtcaatacgggataataccgcgccacatagcagaactttaaaag
tgctcatcattggaaaacgttcttcggggcgaaaactctcaaggatcttaccgctgttgagatcc
agttcgatgtaacccactcgtgcacccaactgatcttcagcatcttttactttcaccagcgtttc
tgggtgagcaaaaacaggaaggcaaaatgccgcaaaaaagggaataagggcgacacggaaatgtt
gaatactcatactcttcctttttcaatattattgaagcatttatcagggttattgtctcatgagc
ggatacatatttgaatgtatttagaaaaataaacaaataggggttccgcgcacatttccccgaaa
agtgccacctgacgcgccctgtagcggcgcattaagcgcggcgggtgtggtggttacgcgcagcg
tgaccgctacacttgccagcgccctagcgcccgctcctttcgctttcttcccttcctttctcgcc
acgttcgccggctttccccgtcaagctctaaatcgggggctccctttagggttccgatttagtgc
tttacggcacctcgaccccaaaaaacttgattagggtgatggttcacgtagtgggccatcgccct
gatagacggtttttcgccctttgacgttggagtccacgttctttaatagtggactcttgttccaa
actggaacaacactcaaccctatctcggtctattcttttgatttataagggattttgccgatttc
ggcctattggttaaaaaatgagctgatttaacaaaaatttaacgcgaattttaacaaaatattaa
cgcttacaatttgccattcgccattcaggctgcgcaactgttgggaagggcgatcggtgcgggcc
tcttcgctattacgccagcccaagctaccatgataagtaagtaatattaaggtacgtggaggttt
tacttgctttaaaaaacctcccacacctccccctgaacctgaaacataaaatgaatgcaattgtt
gttgttaacttgtttattgcagcttataatggttacaaataaagcaatagcatcacaaatttcac
aaataaagcatttttttcactgcattctagttgtggtttgtccaaactcatcaatgtatcttatg
gtactgtaactgagctaacataa
4-6-pGL2-SS*-CMV enhancer-EF1-UTR1-SecNLuc-2A-eGFP-WPRE-
3′UTR(108 to 120 polyA)-SS*
SEQ ID NO: 45
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacacccctgggtcgacgaca
ttgattattgactagttattaatagtaatcaattacggggtcattagttcatagcccatatatgg
agttccgcgttacataacttacggtaaatggcccgcctggctgaccgcccaacgacccccgccca
ttgacgtcaataatgacgtatgttcccatagtaacgccaatagggactttccattgacgtcaatg
ggtggagtatttacggtaaactgcccacttggcagtacatcaagtgtatcatatgccaagtacgc
cccctattgacgtcaatgacggtaaatggcccgcctggcattatgcccagtacatgaccttatgg
gactttcctacttggcagtacatctacgtattagtcatcgctattaccatggggcagagcgcaca
tcgcccacagtccccgagaagttggggggaggggtcggcaattgaaccggtgcctagagaaggtg
gcgcggggtaaactgggaaagtgatgtcgtgtactggctccgcctttttcccgagggtgggggag
aaccgtatataagtgcagtagtcgccgtgaacgttctttttcgcaacgggtttgccgccagaaca
cagctgccttctccctcctgtgagtttggtaagtcgacgggccgggcctgggccgggtccgggcc
gggtcgttggatccccactacagcccgatactcaagcttgacgaattcgagtatccaaggtagtg
gactagtgtgacgctgctgacccctttctttcccttctgcaggttggtgtacagtagcttccaaa
ttgattaattcgagcgaacgcgtcgccaccatgaactccttctccacaagcgccttcggtccagt
tgccttctccctgggcctgctcctggtgttgcctgctgccttccctgccccagtcttcacactcg
aagatttcgttggggactggcgacagacagccggctacaacctggaccaagtccttgaacaggga
ggtgtgtccagtttgtttcagaatctcggggtgtccgtaactccgatccaaaggattgtcctgag
cggtgaaaatgggctgaagatcgacatccatgtcatcatcccgtatgaaggtctgagcggcgacc
aaatgggccagatcgaaaaaatttttaaggtggtgtaccctgtggatgatcatcactttaaggtg
atcctgcactatggcacactggtaatcgacggggttacgccgaacatgatcgactatttcggacg
gccgtatgaaggcatcgccgtgttcgacggcaaaaagatcactgtaacagggaccctgtggaacg
gcaacaaaattatcgacgagcgcctgatcaaccccgacggctccctgctgttccgagtaaccatc
aacggagtgaccggctggcggctgtgcgaacgcattctggcggctagcgctactaacttcagcct
gctgaagcaggctggagacgtggaggagaaccctggacctggaagcggagagggcagaggaagtc
tgctaacatgcggtgacgtcgaggagaatcctggacctggatccggaatggtgagcaagggcgag
gagctgttcaccggggtggtgcccatcctggtcgagctggacggcgacgtaaacggccacaagtt
cagcgtgtccggcgagggcgagggcgatgccacctacggcaagctgaccctgaagttcatctgca
ccaccggcaagctgcccgtgccctggcccaccctcgtgaccaccctgacctacggcgtgcagtgc
ttcagccgctaccccgaccacatgaagcagcacgacttcttcaagtccgccatgcccgaaggcta
cgtccaggagcgcaccatcttcttcaaggacgacggcaactacaagacccgcgccgaggtgaagt
tcgagggcgacaccctggtgaaccgcatcgagctgaagggcatcgacttcaaggaggacggcaac
atcctggggcacaagctggagtacaactacaacagccacaacgtctatatcatggccgacaagca
gaagaacggcatcaaggtgaacttcaagatccgccacaacatcgaggacggcagcgtgcagctcg
ccgaccactaccagcagaacacccccatcggcgacggccccgtgctgctgcccgacaaccactac
ctgagcacccagtccgccctgagcaaagaccccaacgagaagcgcgatcacatggtcctgctgga
gttcgtgaccgccgccgggatcactctcggcatggacgagctgtacaagtaaaatcaacctctgg
attacaaaatttgtgaaagattgactggtattcttaactatgttgctccttttacgctatgtgga
tacgctgctttaatgcctttgtatcatgctattgcttcccgtatggctttcattttctcctcctt
gtataaatcctggttgctgtctctttatgaggagttgtggcccgttgtcaggcaacgtggcgtgg
tgtgcactgtgtttgctgacgcaacccccactggttggggcattgccaccacctgtcagctcctt
tccgggactttcgctttccccctccctattgccacggcggaactcatcgccgcctgccttgcccg
ctgctggacaggggctcggctgttgggcactgacaattccgtggtgttgtcggggaaatcatcgt
cctttccttggctgctcgcctgtgttgccacctggattctgcgcgggacgtccttctgctacgtc
ccttcggccctcaatccagcggaccttccttcccgcggcctgctgccggctctgcggcctcttcc
gcgtcttcgccttcgccctcagacgagtcggatctccctttgggccgcctccccgcgctcgcttt
cttgctgtccaatttctattaaaggttcctttgttccctaagtccaactactaaactgggggata
ttatgaagggccttgagcatctggattctgcctaataaaaaacatttattttcattgcaagctcg
ctttcttgctgtccaatttctattaaaggttcctttgttccctaagtccaactactaaactgggg
gatattatgaagggccttgagcatctggattctgcctaataaaaaacatttattttcattgcaaa
aaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaactcggaaggacatggtgtggaaagt
ccccaggctccccagcaggcagaagtatgcaaagcatgcatctcaattagtcagcaaccaggtgt
ggaaagtccccaggctccccagcaggcagaagtatgcaaagcatgcatctcaattagtcagcaac
caaattaaagtaacccataacttcgtatagcatacattatacgaagttatgaagttcctattctc
tagaaagtataggaacttctagtcacctatttcagcatactacgcgcgtagtatgctgaaatagg
tttatcagcacacaatagtccattatacgcgcgtataatggcaattgtgtgctgattgggttact
ttaatttggatccgtcgaccgatgcccttgagagccttcaacccagtcagctccttccggtgggc
gcggggcatgactatcgtcgccgcacttatgactgtcttctttatcatgcaactcgtaggacagg
tgccggcagcgctcttccgcttcctcgctcactgactcgctgcgctcggtcgttcggctgcggcg
agcggtatcagctcactcaaaggcggtaatacggttatccacagaatcaggggataacgcaggaa
agaacatgtgagcaaaaggccagcaaaaggccaggaaccgtaaaaaggccgcgttgctggcgttt
ttccataggctccgcccccctgacgagcatcacaaaaatcgacgctcaagtcagaggtggcgaaa
cccgacaggactataaagataccaggcgtttccccctggaagctccctcgtgcgctctcctgttc
cgaccctgccgcttaccggatacctgtccgcctttctcccttcgggaagcgtggcgctttctcat
agctcacgctgtaggtatctcagttcggtgtaggtcgttcgctccaagctgggctgtgtgcacga
accccccgttcagcccgaccgctgcgccttatccggtaactatcgtcttgagtccaacccggtaa
gacacgacttatcgccactggcagcagccactggtaacaggattagcagagcgaggtatgtaggc
ggtgctacagagttcttgaagtggtggcctaactacggctacactagaagaacagtatttggtat
ctgcgctctgctgaagccagttaccttcggaaaaagagttggtagctcttgatccggcaaacaaa
ccaccgctggtagcggtggtttttttgtttgcaagcagcagattacgcgcagaaaaaaaggatct
caagaagatcctttgatcttttctacggggtctgacgctcagtggaacgaaaactcacgttaagg
gattttggtcatgagattatcaaaaaggatcttcacctagatccttttaaattaaaaatgaagtt
ttaaatcaatctaaagtatatatgagtaaacttggtctgacagttaccaatgcttaatcagtgag
gcacctatctcagcgatctgtctatttcgttcatccatagttgcctgactccccgtcgtgtagat
aactacgatacgggagggcttaccatctggccccagtgctgcaatgataccgcgagacccacgct
caccggctccagatttatcagcaataaaccagccagccggaagggccgagcgcagaagtggtcct
gcaactttatccgcctccatccagtctattaattgttgccgggaagctagagtaagtagttcgcc
agttaatagtttgcgcaacgttgttgccattgctacaggcatcgtggtgtcacgctcgtcgtttg
gtatggcttcattcagctccggttcccaacgatcaaggcgagttacatgatcccccatgttgtgc
aaaaaagcggttagctccttcggtcctccgatcgttgtcagaagtaagttggccgcagtgttatc
actcatggttatggcagcactgcataattctcttactgtcatgccatccgtaagatgcttttctg
tgactggtgagtactcaaccaagtcattctgagaatagtgtatgcggcgaccgagttgctcttgc
ccggcgtcaatacgggataataccgcgccacatagcagaactttaaaagtgctcatcattggaaa
acgttcttcggggcgaaaactctcaaggatcttaccgctgttgagatccagttcgatgtaaccca
ctcgtgcacccaactgatcttcagcatcttttactttcaccagcgtttctgggtgagcaaaaaca
ggaaggcaaaatgccgcaaaaaagggaataagggcgacacggaaatgttgaatactcatactctt
cctttttcaatattattgaagcatttatcagggttattgtctcatgagcggatacatatttgaat
gtatttagaaaaataaacaaataggggttccgcgcacatttccccgaaaagtgccacctgacgcg
ccctgtagcggcgcattaagcgcggcgggtgtggtggttacgcgcagcgtgaccgctacacttgc
cagcgccctagcgcccgctcctttcgctttcttcccttcctttctcgccacgttcgccggctttc
cccgtcaagctctaaatcgggggctccctttagggttccgatttagtgctttacggcacctcgac
cccaaaaaacttgattagggtgatggttcacgtagtgggccatcgccctgatagacggtttttcg
ccctttgacgttggagtccacgttctttaatagtggactcttgttccaaactggaacaacactca
accctatctcggtctattcttttgatttataagggattttgccgatttcggcctattggttaaaa
aatgagctgatttaacaaaaatttaacgcgaattttaacaaaatattaacgcttacaatttgcca
ttcgccattcaggctgcgcaactgttgggaagggcgatcggtgcgggcctcttcgctattacgcc
agcccaagctaccatgataagtaagtaatattaaggtacgtggaggttttacttgctttaaaaaa
cctcccacacctccccctgaacctgaaacataaaatgaatgcaattgttgttgttaacttgttta
ttgcagcttataatggttacaaataaagcaatagcatcacaaatttcacaaataaagcatttttt
tcactgcattctagttgtggtttgtccaaactcatcaatgtatcttatggtactgtaactgagct
aacataa
3 copies of Enhancer-1
SEQ ID NO: 46
gggactttccggggcggggcacgtggtgcacgggactttccgtgcacgtgcacgggactttccgg
gactttccgggactttccgtgcaccacgtggggactttccgtgcacgggactttccggggcgggg
cacgtggtgcacgggactttccgtgcacgtgcacgggactttccgggactttccgggactttccg
tgcaccacgtggggactttccgtgcacgggactttccggggcggggcacgtggtgcacgggactt
tccgtgcacgtgcacgggactttccgggactttccgggactttccgtgcaccacgtggggacttt
ccgtgcac
chimeric intron
SEQ ID NO: 47
ggagtcgctgcgttgccttcgccccgtgccccgctccgcgccgcctcgcgccgcccgccccggctct
gactgaccgcgttactcccacaggtgagcgggcgggacggcccttctcctccgggctgtaattagcg
cttggtttaatgacggctcgtttcttttctgtggctgcgtgaaagccttaaagggctccgggagggc
cctttgtgcgggggggagcggctcggggggtgcgtgcgtgtgtgtgtgcgtggggagcgccgcgtgc
ggctccgcgctgcccggcggctgtgagcgctgcgggcgcggcgcggggctttgtgcgctccgcagtg
tgcgcgaggggagcgcggccgggggcggtgccccgcggtgcggggggggctgcgaggggaacaaagg
ctgcgtgcggggtgtgtgcgtgggggggtgagcagggggtgtgggcgcggcggtcgggctgtaaccc
ccccctgcacccccctccccgagttgctgagcacggcccggcttcgggtgcggggctccgtacgggg
cgtggcgcggggctcgccgtgccgggcggggggtggcggcaggtgggggtgccgggcggggcggggc
cgcctcgggccggggagggctcgggggaggggcgcggcggcccccggagcgccggcggctgtcgagg
cgcggcgagccgcagccattgccttttatggtaatcgtgcgagagggcgcagggacttcctttgtcc
caaatctgtgcggagccgaaatctgggaggcgccgccgcaccccctctagcgggcgcggggcgaagc
ggtgcggcgccggcaggaaggaaatgggcggggagggccttcgtgcgtcgccgcgccgccgtcccct
tctccctctccagcctcggggctgtccgcggggggacggctgccttcgggggggacggggcagggcg
gggttcggcttctggcgtgtgaccggcggctctagagcctctgctaaccatgttcatgccttcttct
ttttcctacag

The disclosure is not to be limited in scope by the specific aspects described herein. Indeed, various modifications of the disclosure in addition to those described will become apparent to those skilled in the art from the foregoing description and accompanying figures. Such modifications are intended to fall within the scope of the appended claims.

Other aspects are within the following claims.

1-135. (canceled)

136. An expression vector comprising:
(a) a backbone sequence,
(b) a sequence comprising:
(i) an expression cassette comprising a nucleic acid sequence of interest,
(ii) a first target sequence for a first recombinase flanking the 5′ side of the expression cassette,
(iii) a second target sequence for the first recombinase flanking the 3′ side of the expression cassette, and
(iv) one or more additional target sequences for one or more additional recombinases integrated within the first and second target sequences in non-binding regions for the first recombinase, and
(c) one or more of:
(i) an endonuclease target sequence integrated within the first and/or second target sequences for the first recombinase in non-binding regions for the first recombinase and the one or more additional recombinases, wherein the endonuclease target sequence is between the backbone sequence and cleavage sites for the first recombinase and the one or more additional recombinases,
(ii) a synthetic enhancer comprising a nucleic acid sequence at least about 90% identical to SEQ ID NO: 12 integrated between the 3′ end of the first target sequence for the first recombinase and the 5′ end of another enhancer or a promoter in the expression cassette,
(iii) a cytomegalovirus (CMV) enhancer integrated between the 3′ end of the first target sequence for the first recombinase and the 5′ end of a promoter in the expression cassette,
(iv) a 5′ untranslated region (5′UTR) comprising an intron, wherein the 5′UTR is integrated in the expression cassette between a promoter and the nucleic acid sequence of interest,
(v) a vertebrate chromatin insulator integrated in the expression cassette between the nucleic acid of interest and a polyadenylation signal,
(vi) a woodchuck hepatitis virus post-transcriptional regulatory element (WPRE) integrated in the expression cassette between the nucleic acid of interest and a polyadenylation signal,
(vii) a scaffold/matrix attachment region (S/MAR) integrated in the expression cassette between the nucleic acid of interest and a polyadenylation signal, or
(viii) a DNA nuclear targeting sequence (DTS) integrated within the first and/or second target sequences for the first recombinase in non-binding regions for the first recombinase and the one or more additional recombinases, wherein the DTS is between the expression cassette and cleavage sites for the first recombinase and the one or more additional recombinases.

137. The expression vector of claim 136, wherein the endonuclease target sequence of (c)(i) is for:
(a) a homing endonuclease,
(b) I-AniI, I-CeuI, I-ChuI, I-CpaI, I-CpaII, I-CreI, I-DmoI, H-DreI, I-HmuI, I-HmuII, I-LlaI, I-MsoI, PI-PfuI, PI-PkoII, I-PorI, I-PpoI, PI-PspI, I-ScaI, I-SceI, PI-SceI, I-SceII, I-SecIII, I-SceIV, I-SceV, I-SceVI, I-SceVII, I-Ssp6803I, I-TevI, I-TevII, I-TevIII, PI-TliI, PI-TliII, I-Tsp061I, or I-Vdi141I,
(c) I-SceI,
(d) PI-SceI,
(e) a Cas endonuclease, or
(f) Cas9.

138. The expression vector of claim 136, wherein the synthetic enhancer of (c)(ii):
(a) comprises multiple contiguous copies of a nucleic acid sequence at least about 90% identical to SEQ ID NO:12, optionally wherein the synthetic enhancer comprises a nucleic acid sequence at least about 90% identical to SEQ ID NO:46, and/or
(b) is integrated at the 5′ end of a chicken β-actin promoter, optionally comprising a chimeric intron comprising a nucleic acid sequence at least about 90% identical to SEQ ID NO:47 integrated at the 3′ end of the chicken β-actin promoter and 5′ to the nucleic acid sequence of interest.

139. The expression vector of claim 136, wherein the CMV enhancer of (c)(iii) is integrated at the 3′ end of a synthetic enhancer comprising a nucleic acid sequence at least about 90% identical to SEQ ID NO:12 or SEQ ID NO:46, and/or wherein a CMV promoter is integrated at the 3′ end of the CMV enhancer and 5′ to the nucleic acid sequence of interest.

140. The expression vector of claim 136, comprising a nucleic acid sequence at least about 90% identical to SEQ ID NO:35, SEQ ID NO:36, SEQ ID NO:37, SEQ ID NO:38, or SEQ ID NO:39 integrated between the first target sequence for the first recombinase and the nucleic acid sequence of interest.

141. The expression vector of claim 136, wherein:
(a) (i) the intron of (c)(iv) comprises a nucleic acid sequence at least about 90% identical to SEQ ID NO:1, and/or a non-coding sequence integrated within the intron, optionally wherein a non-coding sequence is integrated between two of the nucleotides in the intron corresponding to any two nucleotides from positions 25 to 55 of SEQ ID NO:1, optionally wherein the non-coding sequence is an S/MAR, optionally wherein the S/MAR is MAR-5, or
(ii) the 5′UTR of (c)(iv) comprises a nucleic acid sequence at least about 90% identical SEQ ID NO:3 or SEQ ID NO:5,
(b) the promoter of (c)(iv) is a chicken β-actin promoter or a CMV promoter, and/or
(c) the promoter of (c)(iv) is integrated at the 3′ end of a CMV enhancer, optionally wherein the CMV enhancer is integrated at the 3′ end of a synthetic enhancer comprising a nucleic acid sequence at least about 90% identical to SEQ ID NO: 12 or SEQ ID NO:46.

142. The expression vector of claim 136, wherein:
(a) a polyadenylation signal is integrated at the 3′ end of the nucleic acid sequence of interest and comprises a nucleic acid sequence at least about 90% identical to SEQ ID NO:13, SEQ ID NO: 14, or SEQ ID NO:15,
(b) the vertebrate chromatin insulator of (c)(v) is 5′-HS4 chicken-β-globin insulator (cHS4),
(c) the S/MAR of (c)(vii) is MAR-5,
(d) the polyadenylation signal of (c)(v), (c)(vi), and/or (c)(vii) comprises a nucleic acid sequence at least about 90% identical to SEQ ID NO:13, SEQ ID NO:14, or SEQ ID NO:15, or
(e) the DTS of (c)(viii) is a SV40 enhancer sequence or is cell-specific.

143. The expression vector of claim 136, wherein:
(a) the first and second target sequences and the one or more additional target sequences are selected from the group consisting of the PY54 pal site, the N15 telRL site, the loxP site, φK02 telRL site, the FRT site, the phiC31 attP site, and the λ attP site, optionally wherein the expression vector comprises each of the target sequences, further optionally wherein the expression vector comprises the pal site and the telRL, loxP, and FRT recombinase target binding sequences integrated within the pal site, or
(b) the first and second target sequences for the first recombinase each comprise the nucleic acid sequence of SEQ ID NO:33.

144. A vector production system comprising recombinant cells encoding a recombinase under the control of an inducible promoter, wherein the recombinant cells comprise the expression vector of claim 136, and wherein the recombinase targets the first and second target sequences for the first recombinase or one of the one or more additional target sequences for the one or more additional recombinases in the expression vector, optionally wherein the recombinant cells further encode an endonuclease under the control of an inducible promoter, wherein the endonuclease targets an endonuclease target sequence in an expression vector comprising the endonuclease target sequence.

145. A method of producing a bacterial sequence-free vector comprising incubating the vector production system of claim 144 under suitable conditions for expression of the recombinase, optionally further comprising harvesting the bacterial sequence-free vector.

146. A bacterial sequence-free vector produced by the method of claim 145.

147. A bacterial sequence-free vector comprising:
(a) an expression cassette comprising a nucleic acid sequence of interest, and
(b) one or more of:
(i) a synthetic enhancer comprising a nucleic acid sequence at least about 90% identical to SEQ ID NO: 12 located 5′ to another enhancer or a promoter in the expression cassette,
(ii) a CMV enhancer located 5′ to a promoter in the expression cassette,
(iii) a 5′UTR comprising an intron, wherein the 5′UTR is integrated in the expression cassette between a promoter and the nucleic acid sequence of interest,
(iv) a vertebrate chromatin insulator integrated in the expression cassette between the nucleic acid of interest and a polyadenylation signal,
(v) a WPRE integrated in the expression cassette between the nucleic acid of interest and a polyadenylation signal,
(vi) a S/MAR integrated in the expression cassette between the nucleic acid of interest and a polyadenylation signal, or
(vii) a DTS located 5′ to the expression cassette.

148. The bacterial sequence-free vector of claim 147, wherein the synthetic enhancer of (c)(i):
(a) comprises multiple contiguous copies of a nucleic acid sequence at least about 90% identical to SEQ ID NO:12, optionally wherein the synthetic enhancer comprises a nucleic acid sequence at least about 90% identical to SEQ ID NO:46, and/or
(b) is integrated at the 5′ end of a chicken β-actin promoter, optionally comprising a chimeric intron comprising a nucleic acid sequence at least about 90% identical to SEQ ID NO:47 integrated at the 3′ end of the chicken β-actin promoter and 5′ to the nucleic acid sequence of interest.

149. The bacterial sequence-free vector of claim 147, wherein the CMV enhancer of (c)(ii) is integrated at the 3′ end of a synthetic enhancer comprising a nucleic acid sequence at least about 90% identical to SEQ ID NO: 12 or SEQ ID NO:46, and/or wherein a CMV promoter is integrated at the 3′ end of the CMV enhancer and 5′ to the nucleic acid sequence of interest.

150. The bacterial sequence-free vector of claim 147, comprising a nucleic acid sequence at least about 90% identical to SEQ ID NO:35, SEQ ID NO:36, SEQ ID NO:37, SEQ ID NO:38, or SEQ ID NO:39 located 5′ to the nucleic acid sequence of interest.

151. The bacterial sequence-free vector of claim 147, wherein:
(a) (i) the intron of (c)(iii) comprises a nucleic acid sequence at least about 90% identical to SEQ ID NO:1, and/or a non-coding sequence integrated within the intron, optionally wherein a non-coding sequence is integrated between two of the nucleotides in the intron corresponding to any two nucleotides from nucleotide positions 25 and 55 of SEQ ID NO:1, optionally wherein the non-coding sequence is an S/MAR, optionally wherein the S/MAR is MAR-5, or
(ii) the 5′UTR of (c)(iii) comprises a nucleic acid sequence at least about 90% identical SEQ ID NO:3 or SEQ ID NO:5,
(b) the promoter of (c)(iii) is a chicken β-actin promoter or a CMV promoter, and/or
(c) the promoter of (c)(iii) is integrated at the 3′ end of a CMV enhancer, optionally wherein the CMV enhancer is integrated at the 3′ end of a synthetic enhancer comprising a nucleic acid sequence at least about 90% identical to SEQ ID NO: 12 or SEQ ID NO:46.

152. The bacterial sequence-free vector of claim 147, wherein:
(a) a polyadenylation signal is integrated at the 3′ end of the nucleic acid sequence of interest and comprises a nucleic acid sequence at least about 90% identical to SEQ ID NO:13, SEQ ID NO:14, or SEQ ID NO:15,
(b) the vertebrate chromatin insulator of (c)(iv) is cHS4,
(c) the S/MAR of (c)(vi) is MAR-5,
(d) the polyadenylation signal of (c)(iv), (c)(v), or (c)(vi) comprises a nucleic acid sequence at least about 90% identical to SEQ ID NO:13, SEQ ID NO:14, or SEQ ID NO:15, or
(e) the DTS is a SV40 enhancer sequence or is cell-specific.

153. The bacterial sequence-free vector of claim 147, which is a circular covalently closed vector or a linear covalently closed vector.

154. A recombinant cell comprising the expression vector claim 136.

155. A recombinant cell comprising the bacterial sequence-free vector of claim 147.

156. A composition comprising the expression vector of claim 136.

157. A composition comprising the bacterial sequence-free vector of claim 147.

158. A method of treating a disease or disorder in a subject in need thereof, comprising administering to the subject the expression vector of claim 136.

159. A method of treating a disease or disorder in a subject in need thereof, comprising administering to the subject the bacterial sequence free vector of claim 147.

160. A method of treating a disease or disorder in a subject in need thereof, comprising administering to the subject the composition of claim 156.

161. A method of treating a disease or disorder in a subject in need thereof, comprising administering to the subject the composition of claim 157.

162. A polynucleotide comprising a nucleic acid sequence at least about 90% identical to any one of SEQ ID NOs: 1, 2, 3, 5, 12-18, 35-39, and 46.

163. An expression vector comprising the polynucleotide of claim 162.

164. An expression vector comprising: a polynucleotide comprising a nucleic acid sequence at least about 90% identical to any one of SEQ ID NOs: 2, 3, and 5, and a polynucleotide comprising a nucleic acid sequence at least about 90% identical to any one of SEQ ID NOs: 13-18.

165. A method of gene editing comprising inserting a nucleic acid sequence of interest from the expression vector of claim 136 into a target site for gene editing.

166. A method of gene editing comprising inserting a nucleic acid sequence of interest from the bacterial sequence-free vector of claim 147 into a target site for gene editing.

167. A method of gene editing comprising inserting a nucleic acid sequence of interest from the composition of claim 156 into a target site for gene editing

168. A method of gene editing comprising inserting a nucleic acid sequence of interest from the composition of claim 157 into a target site for gene editing.
